(19) United States
(12) Patent Application Publication
(10) Pub. No.: US 2005/0187266 A1
(43) Pub. Date: Aug. 25, 2005

(54) Alpha substituted carboxylic acids

(75) Inventor: Wei Guo Su, San Diego, CA (US)

Correspondence Address: Pfizer Inc., 10777 Science Center Drive, San Diego, CA, 92121, US

(73) Assignee: Pfizer Inc

(21) Appl. No.: 11/073,274

(22) Filed: Mar. 4, 2005

Related U.S. Application Data
(63) Continuation-in-part of application No. 10/825,923, filed on Apr. 15, 2004.
(60) Provisional application No. 60/463,213, filed on Apr. 15, 2003.

(51) Int. Cl. A61K 31/4245; A61K 31/421; A61K 31/4192

(52) U.S. Cl. 514/359; 514/364; 514/374; 548/143; 548/235; 548/255

(57) ABSTRACT

Alpha substituted carboxylic acids of formula (I): 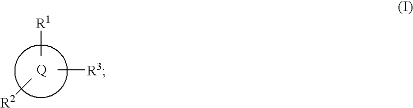
wherein R¹ and R² are as defined in the specification and R³ is 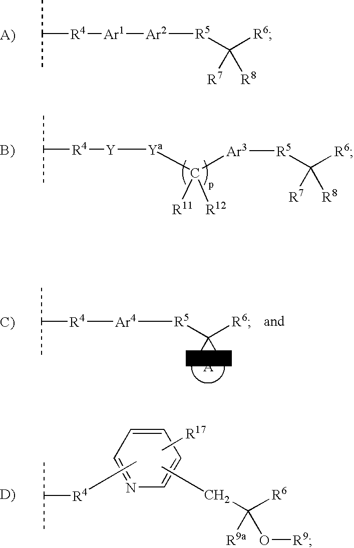
wherein Y, Ar¹, Ar², Ar³, R⁴, R⁵, R⁶, R⁷, R⁸, R⁹, R^{9a}, R¹⁰, R¹¹, R¹², R¹⁷, ring A, and p are as defined in the specification; pharmaceutical compositions containing effective amounts of said compounds or their salts are useful for treating PPAR, specifically PPAR α/γ related disorders, such as diabetes, dyslipidemia, obesity and inflammatory disorders.

This application is a continuation in part of application Ser. No. 10/825,923 filed on Apr. 15, 2004, which claims priority from a Provisional application Ser. No. 60/463,213 filed on Apr. 15, 2003, now expired.

### BACKGROUND OF THE INVENTION

This invention relates to alpha substituted carboxylic acids that modulate the activities of peroxisome proliferator-activated receptor (PPAR), preferably two or more of PPAR-α, PPAR-δ, or PPAR-γ, enabling them to be useful in modulation of blood glucose and the increase of insulin sensitivity in mammals. This invention also relates to treatment of PPAR related disorders, such as diabetes, dyslipidemia, obesity and inflammatory disorders.

Peroxisome proliferators are a structurally diverse group of compounds which, when administered to rodents, elicit dramatic increases in the size and number of hepatic and renal peroxisomes, as well as concomitant increases in the capacity of peroxisomes to metabolize fatty acids via increased expression of the enzymes required for the β-oxidation cycle. Chemicals included in this group are the fibrate class of hypolipidermic drugs, herbicides, and phthalate plasticizers (Reddy and Lalwani, Crit. Rev. Toxicol., 12:1-58 (1983)). Peroxisome proliferation can also be elicited by dietary or physiological factors such as a high-fat diet and cold acclimatization.

Insight into the mechanism whereby peroxisome proliferators exert their pleiotropic effects was provided by the identification of a member of the nuclear hormone receptor superfamily activated by these chemicals (Isseman and Green, Nature, 347-645-650 (1990)). This receptor, termed PPAR-α, was subsequently shown to be activated by a variety of medium and long-chain fatty acids and to stimulate expression of the genes encoding rat acyl-CoA oxidase and hydratase-dehydrogenase (enzymes required for peroxisomal β-oxidation), as well as rabbit cytochrome P450 4A6, a fatty acid Ω-hydroxylase.

PPAR-α activates transcription by binding to DNA sequence elements, termed peroxisome proliferator response elements (PPRE), as a heterodimer with the retinoid X receptor. The retinoid X receptor is activated by 9-cis retinoic acid (see Kliewer, et al., Nature, 358:771-774 (1992), Gearing, et al., Proc. Natl. Acad. Sci. USA, 90:1440-1444 (1993), Keller, et al., Proc. Natl. Acad. Sci. USA, 90:2160-2164 (1993), Heyman, et al., Cell, 68:397-406 (1992), and Levin, et al., Nature, 355:359-361 (1992)). Since the PPAR-α-RXR complex can be activated by peroxisome proliferators and/or 9-cis retinoic acid, the retinoid and fatty acid signaling pathways are seen to converge in modulating lipid metabolism.

Since the discovery of PPAR-α, additional isoforms of PPAR have been identified, e.g., PPAR-δ, or PPAR-γ, which are spatially differentially expressed. Each PPAR receptor shows a different pattern of tissue expression, and differences in activation by structurally diverse compounds. PPAR-γ, for instance, is expressed most abundantly in adipose tissue and at lower levels in skeletal muscle, heart, liver, intestine, kidney, vascular endothelial and smooth muscle cells as well as macrophages. Two isoforms of PPAR-γ exist, identified as γ₁ and γ₂, respectively. PPAR-γ mediates adipocyte signalling, lipid storage, and fat metabolism. Evidence gathered to date support the conclusion that PPAR-γ is the primary, and perhaps the only, molecular target mediating the insulin sensitizing action of one class of antidiabetic agents, the thiazolidine 2,4 diones.

In a monotherapeutic or combination therapy context, new and established oral antidiabetic agents are still considered to have non-uniform and even limited effectiveness. The effectiveness of oral antidiabetic therapies may be limited, in part, because of poor or limited glycemic control, or poor patient compliance due to unacceptable side effects. These side effects include edema, weight gain, or even more serious complications. For instance, hypoglycemia is observed in some patients taking sulfonylureas. Metformin, a substituted biguanide, can cause diarrhea and gastrointestinal discomfort. Finally, edema, weight gain, and in some cases, hepatoxicity, have been linked to the administration of some thiazolidine 2,4 dione antidiabetic agents. Combination therapy using two or more of the above agents is common, but generally only leads to incremental improvements in glycemic control.

As a result, there is a need for antidiabetic agents that display combined PPAR-α and PPAR-γ activation which should lead to the discovery of efficacious glucose and triglyceride lowering drugs that have great potential in the treatment of type 2 diabetes and the metabolic syndrome (i.e., impaired glucose tolerance, insulin resistance, hypertriglyceridemia and/or obesity).

### SUMMARY OF THE INVENTION

The present invention provides novel compounds of Formula (I):

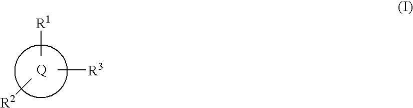

or a pharmaceutically acceptable salt or solvate thereof, wherein:

Ring Q is (C₆-C₁₀)aryl or (4-10)-membered heterocyclyl;

R¹ is H, halo, (C₁-C₈)alkyl, (C₁-C₈)alkoxy, CN, CF₃, —O—CF₃, —O—SO₂—(C₁-C₈)alkyl, —O—SO₂—(CR¹¹R¹²)ₜ(C₆-C₁₀)aryl, —(CR¹¹R¹²)ₜ(C₃-C₁₀)cycloalkyl-(CR¹¹R¹²)ₜ, —(CR¹¹R¹²)ₜ(C₃-C₁₀)cycloalkyl-(CR¹¹R¹²)ₜ—O—, —(CR¹¹R¹²)ₜ(C₆-C₁₀)aryl-(CR¹¹R¹²)ₜ, —(CR¹¹R¹²)ₜ(C₆-C₁₀)aryl-(CR¹¹R¹²)ₜ—O—, —(CR¹¹R¹²)ₜ—(4-10)-membered heterocyclyl-(CR¹¹R¹²)ₜ, or —(CR¹R¹²)ₜ— (4-10)-membered heterocyclyl-(CR¹¹R¹²)ₜ—O—; wherein the ring carbon atoms of R¹ are optionally substituted by 1 to 3 R¹³ groups; and the ring nitrogen atoms of R¹ are optionally substituted by 1 to 3 (C₁-C₈)alkyl;

R² is H, (C₁-C₈)alkyl, —(CR¹¹R¹²)ₜ—(C₃-C₁₀)cycloalkyl, —(CR¹¹R¹²)ₜ—(C₆-C₁₀)aryl, or —(CR¹R¹²)₁(4-10)-membered heterocyclyl; and wherein the carbon atoms of R² are optionally substituted by 1 to 3 R¹³ groups; and the ring nitrogen atoms of R² are optionally substituted by 1 to 3 (C₁-C₈)alkyl;

R³ is selected from the group consisting of:

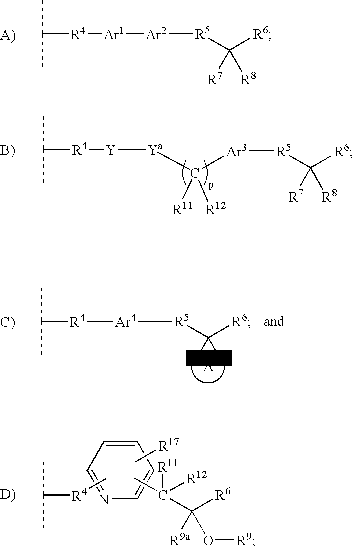

Y is —(C═O)— or —SO₂—;

Y″ is NR¹⁰ or —O—;

p is 0, 1, or 2;

each q, r, and t are independently 0, 1, 2, 3, 4, or 5;

each n is independently 0, 1, 2, 3, or 4;

each k is independently 1, 2, or 3;

each m and s are independently 0, 1, 2, or 3;

each j is 0, 1, or 2;

Each R⁴ is —(CR¹¹R¹²)ₙ—, —(CR¹¹R¹²)ₙ—S—(CR¹¹R¹²)ₙ—, —(CR¹¹R¹²)ₙ—NR¹⁰—, —(CR¹¹R¹²ₙ—NR¹⁰—(CR¹¹R¹²)ₙ—O—, —(CR¹¹R¹²)ₙ—O—(CR¹¹R¹²)ₖ—NR¹⁰—, —(CR¹¹R¹²)ₙ—O—(CR¹¹R¹²)ₙ—, —(CR¹¹R¹²)ₙ—O—(CR¹¹R¹²)ₖ—O—(CR¹¹R¹²)ₙ—, —(CR¹¹R¹²)ₙ—CR¹¹═CR¹²—(CR¹¹R¹²)ₙ—, or —CH═CH—(CR¹¹R¹²)—O—(CH₂)ₙ—;

Each R⁵ is a bond or —(CR¹¹R¹²)ₘ-Z-(CR¹¹R¹²)ₛ; wherein Z is —CR¹¹R¹²—, —O—, —NR^{10a}, or —S(O)ⱼ—;

Each R⁶ is —(C═O)—OH, —(C═O)-OM⁺, —(C═O)—(C₁-C₈)alkyl, —(C═O)—O—(C₁-C₈)alkyl, —(C═O)—NR¹⁰R¹¹, —(C═O)—NR¹⁰—SO₂—R¹¹, —SO₂—NH—R¹⁰, —NH—SO₂—R¹⁰, —(C═O)—NH—C≡N, or R⁶ has a formula:

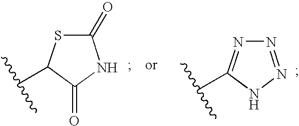

M⁺ is an alkali metal cation or an alkaline earth metal cation;

Each R⁷ and R⁸ is independently H, (C₁-C₈)alkyl, (C₁-C₈)alkoxy, —(CR¹¹R¹²)ₜ(C₃-C₁₀)cycloalkyl, —(CR¹¹R¹²)ₜ(C₆-C₁₀)aryl, —(CR¹¹R¹²)ₜ(C₆-C₁₀)aryl-O—, —(CR¹¹R¹²)ₜ(4-10)-membered heterocyclyl or —(CR¹¹R¹²)ₜ(4-10)-membered heterocyclyl-O—;

Or R⁷ and R⁸ may optionally be taken together with the carbon to which they are attached to form a (C₃-C₁₀)cycloalkyl or a (3-10)-membered heterocyclyl;

Each of Ar¹, Ar², Ar³, and Ar⁴ represents (C₆-C₁₀)aryl or (5-10)-membered heterocyclyl wherein the ring carbon atoms of each of Ar¹, Ar, Ar³, and Ar⁴ are optionally substituted by 1 to 3 R¹³ groups;

Ring A represents a 3, 4, 5, 6 or 7-membered ring optionally containing 1 to 4 heteroatoms which may be the same or different and which are selected from —N(R^{10a})—, O, and S(O)ⱼ, wherein j is 0, 1, or 2, with the proviso that the ring does not contain two adjacent O or S(O)ⱼ atoms, and wherein the carbon atoms of the ring A moiety are optionally substituted by 1 to 3 R¹³ groups;

R⁹ is (C₁-C₈)alkyl, —(CR¹¹R¹²)ₜ(C₆-C₁₀)aryl or —(CR¹¹R¹²)ₜ(4-10)-membered heterocyclyl, wherein t is independently 0, 1, 2, 3, 4, or 5, wherein said R⁹ groups are substituted with 1 to 3 groups independently selected from (CR¹¹R¹²)_{q}NR¹⁰R¹¹, —(CR¹¹R¹²)_{q}NR¹⁰(C₁-C₆)alkanoyl —(CR¹¹R¹²)_{q}O(CR¹¹R¹²)ᵣR¹⁰, and —(CR¹¹R¹²)_{q}R¹⁰; and wherein the heterocyclyl, aryl and alkyl moieties of the foregoing groups are optionally substituted with 1 to 3 R¹³ groups;

R^{9a} and R¹⁰ are independently H or (C₁-C₈)alkyl;

R¹¹ and R¹² are independently H, (C₁-C₈)alkyl, hydroxy, or (C₁-C₆)alkoxy;

R^{10a} is selected from H, (C₁-C₈)alkyl, —(C═O)—R⁴, —SO₂NR¹⁵R¹⁶, or —S(O)ⱼ(C₁-C₆)alkyl;

Each R¹³ and R^{13a} are independently selected from the group consisting of halo, cyano, nitro, trifluoromethoxy, trifluoromethyl, azido, hydroxy, (C₁-C₆)alkoxy, (C₁-C₁₀)alkyl, (C₂-C₆)alkenyl, (C₂-C₆)alkynyl, —O—(CR¹¹R¹²)ₖ—O—(CR¹¹R¹²)ₙ—, —(C═O)—R¹⁴, —(C═O)—O—R¹⁵, —O—(C═O)—R¹⁵, —NR¹⁵(C═O)—R¹⁶, —NR¹⁵(C═O)—O—R¹⁶, —(C═O)—NR¹⁵R¹⁶, —NR¹⁵R¹⁶, —NR¹⁵OR¹⁶, —SO₂NR¹⁵R¹⁶, —S(O)ⱼ(C₁-C₆)alkyl, —O—SO₂—R¹⁴, —NR¹⁵—SO₂—R¹⁶, R¹⁵—(CR¹¹CR¹²)ₜ(C₆-C₁₀aryl), —(CR¹¹R¹²)ₜ(4-10)-membered heterocyclyl, —(CR¹¹R¹²)_{q}(C═O)(CR¹¹R¹²)ₜ(C₆-C₁₀)aryl, —(CR¹¹R¹²)_{q}(C═O)(CR¹¹R¹²)ₜ(4-10)-membered heterocyclyl, —(CR¹¹R¹²)ₜO(CR¹¹R¹²)_{q}(C₆-C₁₀)aryl, —(CR¹¹R¹²)ₜO(CR¹¹R¹²)_{q}(⁴-10)-membered heterocyclyl, —(CR¹¹R¹²)_{q}SO₂(CR¹¹R¹²)ₜ(C₆-C₁₀)aryl, and —(CR¹¹R¹²)_{q}SO₂(CR¹¹R¹²)ₜ(4-10)-membered heterocyclyl; 1 or 2 ring carbon atoms of the heterocyclic moieties of the foregoing R¹³ and R^{13a} groups are optionally substituted with an oxo (═O) moiety, and the alkyl, alkenyl, alkynyl, aryl and heterocyclic moieties of the foregoing R¹³ and R^{13a} groups are optionally substituted with 1 to 3 substituents independently selected from halo, cyano, nitro, trifluoromethyl, trifluoromethoxy, azido, —OR¹⁵, —(C═O)—R¹⁵, —(C═O)—O—R¹⁵—O—(C═O)—R¹⁵, —NR¹⁵(C═O)—R¹⁶, —(C═O)—NR¹⁵R¹⁶, —NR¹⁵R¹⁶, —NR¹⁵OR¹⁶, (C₁-C₆)alkyl, (C₂-C₆)alkenyl, (C₂-C₆)alkynyl, —(CR¹¹R¹²)ₜ(C₆-C₁₀)aryl, and —(CR¹¹R¹²)ₜ(4-10)-membered heterocyclyl;

each R¹⁴, R¹⁵, and R¹⁶ is independently selected from H, (C₁-C₆)alkyl, —(CR¹¹R¹²)ₜ(C₆-C₁₀)aryl, and —(CR¹¹R¹²)ₜ(4-10)-membered heterocyclyl; 1 or 2 ring carbon atoms of the heterocyclic group are optionally substituted with an oxo (═O) moiety, and the alkyl, aryl and heterocyclic moieties of the foregoing R¹⁴, R¹⁵ and R¹⁶ groups are optionally substituted with 1 to 3 substituents independently selected from halo, cyano, nitro, —NR¹¹R¹², trifluoromethyl, trifluoromethoxy, (C₁-C₆)alkyl, (C₂-C₆)alkenyl, (C₂-C₆)alkynyl, hydroxy, and (C₁-C₆) alkoxy;

R¹⁷ is H, (C₁-C₈)alkyl, —O—(C₁-C₈)alkyl, halo, CN, OH, CF₃, or —O—CF₃;

and wherein any of the above-mentioned substituents comprising a CH₃ (methyl), CH₂ (methylene), or CH (methine) group which is not attached to a halo, SO or SO₂ group or to a N, O or S atom optionally bears on said group a substituent selected from hydroxy, halo, (C₁-C₄)alkyl, (C₁-C₄)alkoxy, —NH₂, —NH(C₁-C₈)alkyl, and —N((C₁-C₈)alkyl)₂.

In one embodiment, the invention relates to compounds of the Formula I wherein R³ is

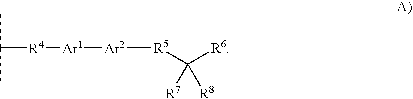

In another embodiment, the invention relates to compounds of the Formula I wherein R³ is

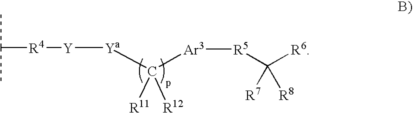

Within this embodiment, preferred —R⁴—Y—Y″— is —(CR¹¹R¹²)ₙ—O—(CR¹¹R¹²)ₙ—(C═O)—NR¹⁰— or —(CR¹¹R¹²) —NR¹⁰—(C═O)—O—.

In another embodiment, the invention relates to compounds of the Formula I wherein R³ is

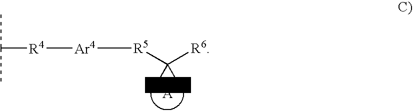

In another embodiment, the invention relates to compounds of the Formula I wherein R³ is

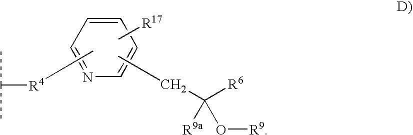

In another embodiment, the invention relates to compounds of the Formula I wherein ring Q is selected from the group consisting of

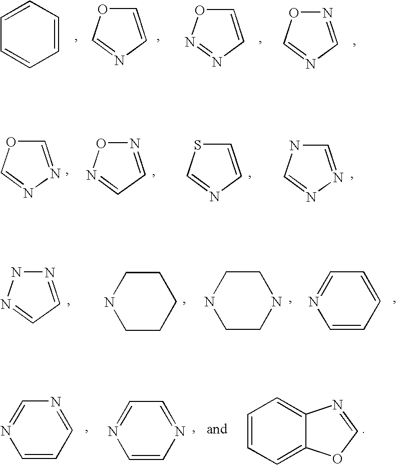

In another embodiment, the invention relates to compounds of the Formula I wherein R¹ is H, halo, (C₁-C₈)alkyl, (C₁-C₈)alkoxy, CF₃, —O—CF₃, —O—SO₂—(C₁-C₈)alkyl, —O—SO₂—(CR¹¹R¹²)ₜ(C₆-C₁₀)aryl, or —(CR¹¹R¹²)ₜ(C₆-C₁₀)aryl-O—, wherein the ring carbon atoms of R¹ are optionally substituted by 1 to 3 R¹³ groups.

In another embodiment, the invention relates to compounds of the Formula I wherein R² is H, phenyl,

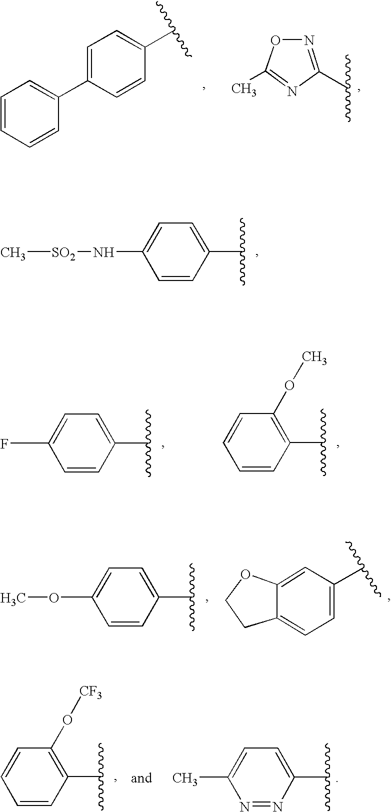

In another embodiment, the invention relates to compounds of the Formula I wherein said 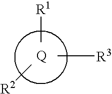
is selected from the group consisting of:

In another embodiment, the invention relates to compounds of the Formula I wherein said 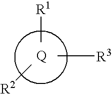
is selected from the group consisting of:

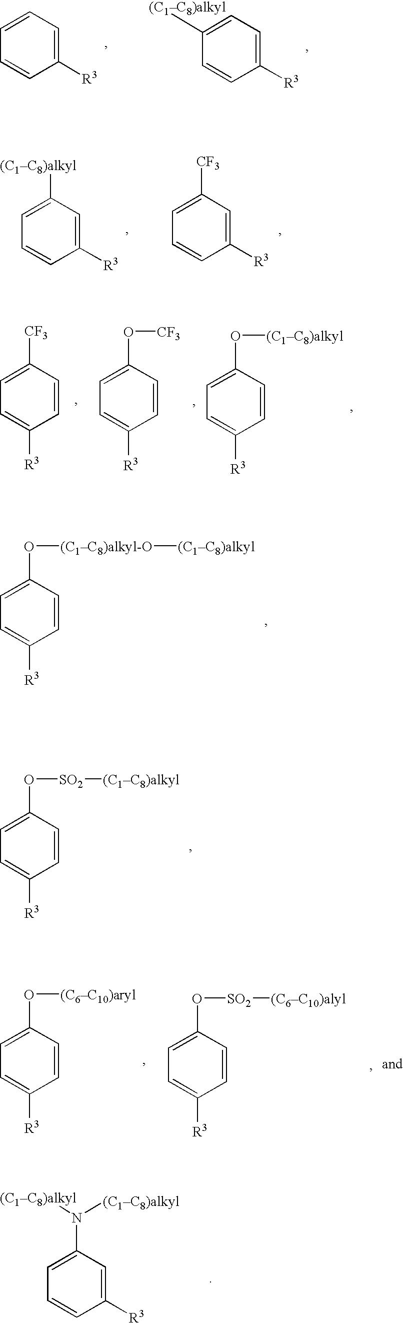

In another embodiment, the invention relates to compounds of the Formula I wherein said 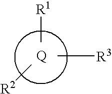
is selected from the group consisting of:

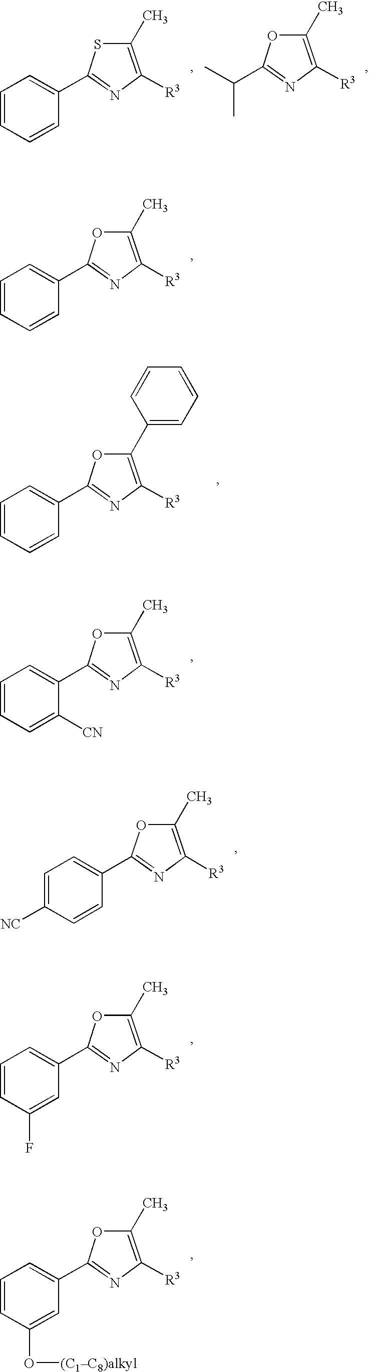

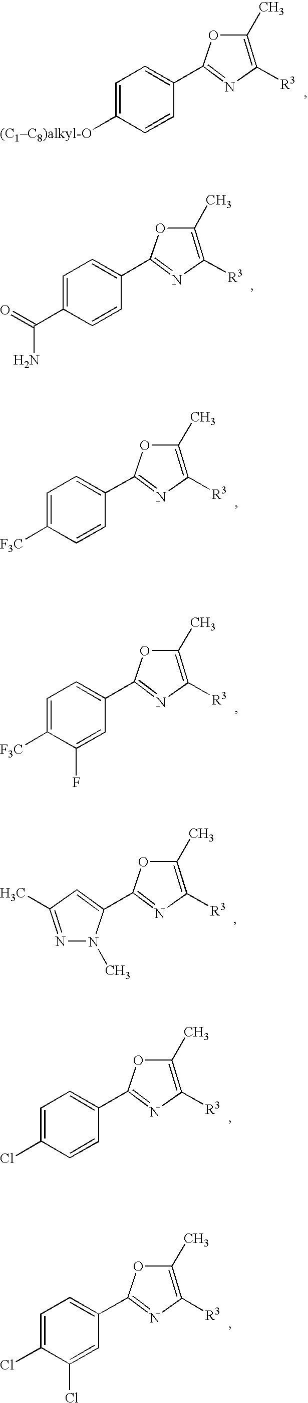

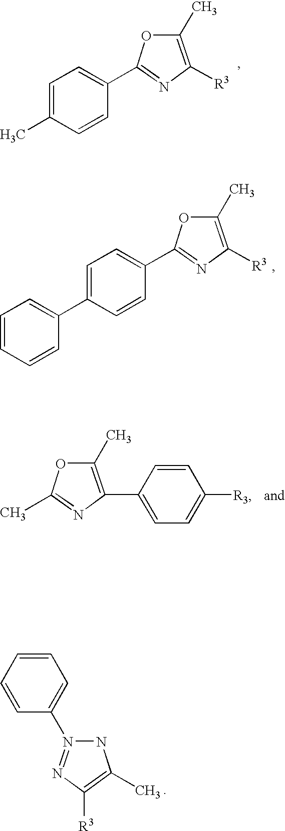

In another embodiment, the invention relates to compounds of the Formula I wherein said 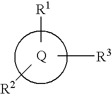
is selected from the group consisting of:

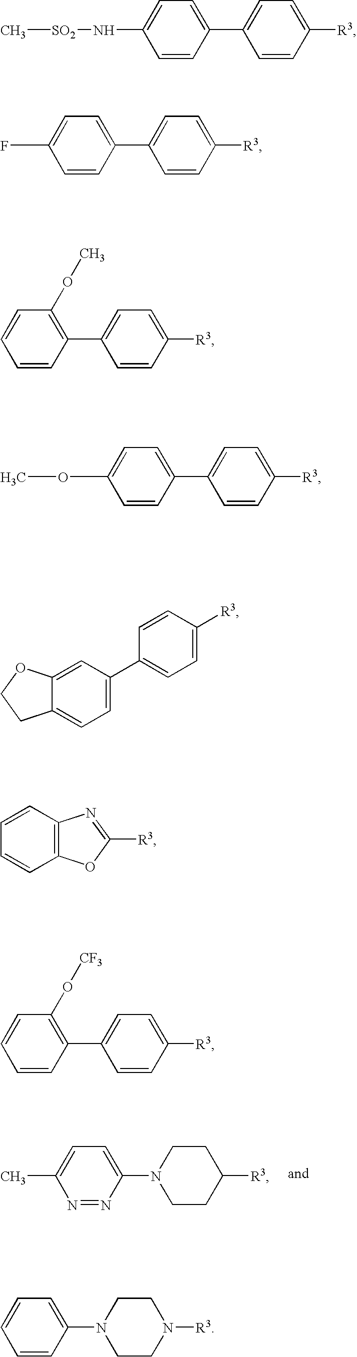

In another embodiment, the invention relates to compounds of the Formula I wherein R⁴ is —CH₂—O—, —CH₂—O—CH₂—, —CH₂—CH₂—O—, —CH═CH—CH₂—O—, or —CH₂—CH₂—CH₂—O—.

In another embodiment, the invention relates to compounds of the Formula I wherein R⁴ is —(CH₂)ₙ—; wherein n is independently 0, 1, 2, or 3.

In another embodiment, the invention relates to compounds of the Formula I wherein R⁵ is a bond or —(CR¹¹R¹²)ₘ-Z-(CR¹¹R¹²)ₛ; wherein Z is —O—, —NR^{10a}—, or —S(O)ⱼ—wherein each m and s are independently 0, 1, 2, or 3; and wherein j is 0, 1, or 2.

In another embodiment, the invention relates to compounds of the Formula I wherein R⁵ is a bond, —O—, —CH₂—, —C(CH₃)H—, —C(OH)H—, or —C(O—(C₁-C₈)alkyl)H—.

In another embodiment, the invention relates to compounds of the Formula I wherein R⁶ is —(C═O)—OH.

In another embodiment, the invention relates to compounds of the Formula I wherein R⁶ is —(C═O)-OM⁺, wherein M⁺ is selected from the group consisting of Ca⁺⁺, Li⁺, Na⁺ and K⁺.

In another embodiment, the invention relates to compounds of the Formula I wherein each R⁷ and R⁶ is independently H, (C₁-C₈)alkyl, or (C₁-C₈)alkoxy.

In another embodiment, the invention relates to compounds of the Formula I wherein each R⁷ and R⁸ are taken together with the carbon to which they are attached to form a (3-7)-membered heterocyclyl.

In another embodiment, the invention relates to compounds having a formula:

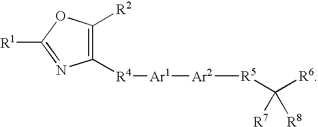

Within this embodiment, the invention relates to compounds wherein said —Ar¹—Ar²— is selected from the group consisting of:

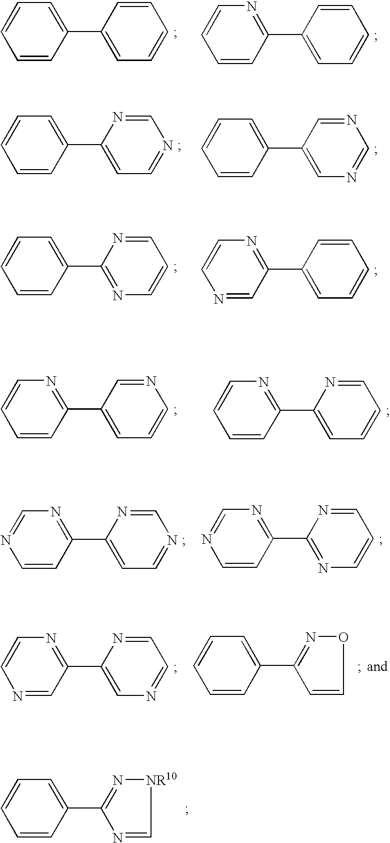

wherein the ring carbon atoms of each of Ar¹ and Ar² are optionally substituted by 1 to 3 R¹³ groups selected from the group consisting of halo, (C₁-C₈)alkyl, and (C₁-C₈)alkoxy.

Preferably, said —Ar¹—Ar²— is selected from the group consisting of:

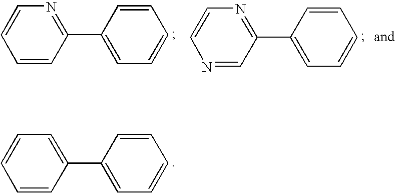

Within this embodiment, specific compounds of the present invention are selected from the group consisting of
2-Methyl-2-({3′-[2-(5-methyl-2-phenyl-1,3-oxazol-4-yl)ethoxy]-1,1′-biphenyl-3-yl}oxy)propanoic acid;
2-Methyl-2-[(3′-{[4-(trifluoromethyl)benzyl]oxy}-1,1′-biphenyl-3-yl)oxy]propanoic acid;
2-Methyl-2-[(3′-{2-[1-(6-methylpyridazin-3-yl)piperidin-4-yl]ethoxy}-1,1′-biphenyl-3-yl)oxy]propanoic acid;
1-({3′-[2-(5-Methyl-2-phenyl-1,3-oxazol-4-yl)ethoxy]-1,1′-biphenyl-3-yl}oxy)cyclobutanecarboxylic acid;
2-({3′-[2-(5-Methyl-2-phenyl-1,3-oxazol-4-yl)ethoxy]-1,1′-biphenyl-3-yl}oxy)butanoic acid;
2-(3-{6-[2-(5-Methyl-2-phenyl-1,3-oxazol-4-yl)ethoxy]pyridin-2-yl}phenoxy)butanoic acid;
1-(3-{6-[2-(5-Methyl-2-phenyl-1,3-oxazol-4-yl)ethoxy]pyridin-2-yl}phenoxy)cyclobutanecarboxylic acid;
2-Methyl-2-(3-{6-[2-(5-methyl-2-phenyl-1,3-oxazol-4-yl)ethoxy]pyridin-2-yl}phenoxy)propanoic acid;
2-Methyl-2-(3-{6-[2-(5-methyl-2-phenyl-1,3-oxazol-4-yl)ethoxy]pyrazin-2-yl}phenoxy) propanoic acid; and

and pharmaceutically acceptable salts thereof.

Within this embodiment, a specific compound of the present invention is 1-({3′-[2-(5-Methyl-2-phenyl-1,3-oxazol-4-yl)ethoxy]-1,1′-biphenyl-3-yl}oxy)cyclobutanecarboxylic acid or the pharmaceutically acceptable salts thereof.

Within this embodiment, a specific compound of the present invention is 2-({3′-[2-(5-Methyl-2-phenyl-1,3-oxazol-4-yl)ethoxy]-1,1′-biphenyl-3-yl}oxy)butanoic acid or the pharmaceutically acceptable salts thereof.

Within this embodiment, a specific compound of the present invention is 2-(3-{6-[2-(5-Methyl-2-phenyl-1,3-oxazol-4-yl)ethoxy]pyridin-2-yl}phenoxy)butanoic acid or the pharmaceutically acceptable salts thereof.

Within this embodiment, a specific compound of the present invention is 1-(3-{6-[2-(5-Methyl-2-phenyl-1,3-oxazol-4-yl)ethoxy]pyridin-2-yl}phenoxy)cyclobutanecarboxylic acid or the pharmaceutically acceptable salts thereof.

Within this embodiment, a specific compound of the present invention is 1-[(3′-{[2-(3-fluorophenyl)-5-methyl-1,3-oxazol-4-yl]methoxy}biphenyl-3-yl)oxy]cyclobutanecarboxylic acid or the pharmaceutically acceptable salts thereof.

Within this embodiment, a specific compound of the present invention is 1-({3′-[3-(5-methyl-2-phenyl-1,3-oxazol-4-yl)propoxy]biphenyl-3-yl}oxy)cyclobutanecarboxylic acid or the pharmaceutically acceptable salts thereof.

Within this embodiment, a specific compound of the present invention is 1-[(3′-{[5-(4-methoxyphenyl)-1,2,4-oxadiazol-3-yl]methoxy}biphenyl-3-yl)oxy]cyclobutanecarboxylic acid or the pharmaceutically acceptable salts thereof.

Within this embodiment, a specific compound of the present invention is 2-[(3′-{2-[2-(3-Fluorophenyl)-5-methyl-1,3-oxazol-4-yl]ethoxy}biphenyl-3-yl)oxy]-2-methylpropanoic acid or the pharmaceutically acceptable salts thereof.

Within this embodiment, a specific compound of the present invention is 2-methyl-2-({3′-[(5-methyl-2-phenyl-1,3-oxazol-4-yl)methoxy]biphenyl-3-yl}oxy)propanoic acid or the pharmaceutically acceptable salts thereof.

Within this embodiment, a specific compound of the present invention is 2-ethoxy-3-{3′-[2-(5-methyl-2-phenyl-1,3-oxazol-4-yl)ethoxy]biphenyl-3-yl}propanoic acid or the pharmaceutically acceptable salts thereof.

In another embodiment, the invention relates to compounds having a formula:

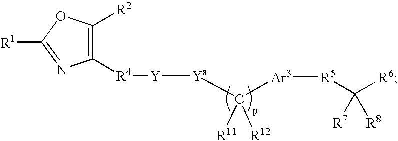

wherein Y is —(C═O)— or —SO₂—, Y″ is NR¹⁰, and p is 1.

Preferably, each of R¹¹ and R¹² are independently H.

Preferably, Ar³ is phenyl.

Within this embodiment, specific compounds of the present invention are selected from the group consisting of
1-(3-{[({2-[3-(Trifluoromethyl)phenyl]ethoxy}carbonyl)amino]methyl}phenoxy) cyclobutanecarboxylic acid;
2-(3-{[({2-[3-(Trifluoromethyl)phenyl]ethoxy}carbonyl)amino]methyl}phenoxy)butanoic acid;
2-Methyl-2-(3-{[({2-[3-(trifluoromethyl)phenyl]ethoxy}carbonyl)amino]methyl}phenoxy) propanoic acid;
2-Methyl-2-{3-[({[2-(5-methyl-2-phenyl-1,3-oxazol-4-yl)ethoxy]carbonyl}amino)methyl]phenoxy}propanoic acid;
2-Methyl-2-(3-{[({[4-(5-methyl-1,2,4-oxadiazol-3-yl)benzyl]oxy}carbonyl)amino]methyl}phenoxy)propanoic acid;
2-{3-[({[2-(5-Methyl-2-phenyl-1,3-oxazol-4-yl)ethoxy]carbonyl}amino)methyl]phenoxy}butanoic acid;
1-{3-[({[2-(5-Methyl-2-phenyl-1,3-oxazol-4-yl)ethoxy]carbonyl}amino)methyl]phenoxy}cyclobutanecarboxylic acid;
1-{3-[({[3-(5-Methyl-2-phenyl-1,3-oxazol-4-l)propoxy]carbonyl}amino)methyl]phenoxy}cyclobutanecarboxylic acid;
2-{3-[({[3-(5-Methyl-2-phenyl-1,3-oxazol-4-yl)propoxy]carbonyl}amino)methyl]phenoxy}butanoic acid;
2-Methyl-2-{3-[({[3-(5-methyl-2-phenyl-1,3-oxazol-4-yl)propoxy]carbonyl}amino)methyl]phenoxy}propanoic acid;

and pharmaceutically acceptable salts thereof.

Within this embodiment, a specific compound of the present invention is 2-Methyl-2-{3-[({[2-(5-methyl-2-phenyl-1,3-oxazol-4-yl)ethoxy]carbonyl}amino)methyl]phenoxy}propanoic acid or the pharmaceutically acceptable salts thereof.

Within this embodiment, a specific compound of the present invention is 2-methyl-2-{3-[({[(5-methyl-2-phenyl-1,3-oxazol-4-yl)methoxy]carbonyl}amino)methyl]phenoxy}propanoic acid or the pharmaceutically acceptable salts thereof.

Within this embodiment, a specific compound of the present invention is 2-methyl-2-{4-[({[3-(5-methyl-2-phenyl-1,3-oxazol-4-yl)propoxylcarbonyl}amino)methyl]phenoxy}propanoic acid or the pharmaceutically acceptable salts thereof.

Within this embodiment, a specific compound of the present invention is 2-{3-fluoro-4-[({[2-(5-methyl-2-phenyl-1,3-oxazol-4-yl)ethoxy]carbonyl}amino)methyl]phenoxy}-2-methylpropanoic acid or the pharmaceutically acceptable salts thereof.

Within this embodiment, a specific compound of the present invention is 2-{3-[({f2-(5-Methyl-2-phenyl-1,3-oxazol-4-yl)ethoxy]carbonyl}amino)methyl]phenoxy}butanoic acid or the pharmaceutically acceptable salts thereof.

Within this embodiment, a specific compound of the present invention is 2-{3-[({[(5-methyl-2-phenyl-1,3-oxazol-4-yl)methoxy]carbonyl}amino)methyl]phenoxy}butanoic acid or the pharmaceutically acceptable salts thereof.

Within this embodiment, a specific compound of the present invention is 1-{3-[({[2-(5-Methyl-2-phenyl-1,3-oxazol-4-yl)ethoxy]carbonyl}amino)methyl]phenoxy}cyclobutanecarboxylic acid or the pharmaceutically acceptable salts thereof.

Within this embodiment, a specific compound of the present invention is 2-methyl-2-(3-{[({[2-(5-methyl-2-phenyl-1,3-oxazol-4-yl)ethyl]amino}carbonyl)oxy]methyl}phenoxy) propanoic acid or the pharmaceutically acceptable salts thereof.

Within this embodiment, a specific compound of the present invention is 2-ethoxy-3-{3-[({[3-(5-methyl-2-phenyl-1,3-oxazol-4-yl)propoxy]carbonyl}amino)methyl]phenyl}propanoic acid or the pharmaceutically acceptable salts thereof.

Within this embodiment, a specific compound of the present invention is 2-ethoxy-3-{3-[({[2-(5-methyl-2-phenyl-1,3-oxazol-4-yl)ethoxy]carbonyl}amino)methyl]phenyl}propanoic acid or the pharmaceutically acceptable salts thereof.

In another embodiment, the invention relates to compounds having a formula:

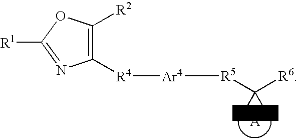

In another embodiment, ring A is selected from the group consisting of cyclopropyl, cyclobutyl, cyclopentyl and cyclohexyl.

In another embodiment, ring A is selected from the group consisting of

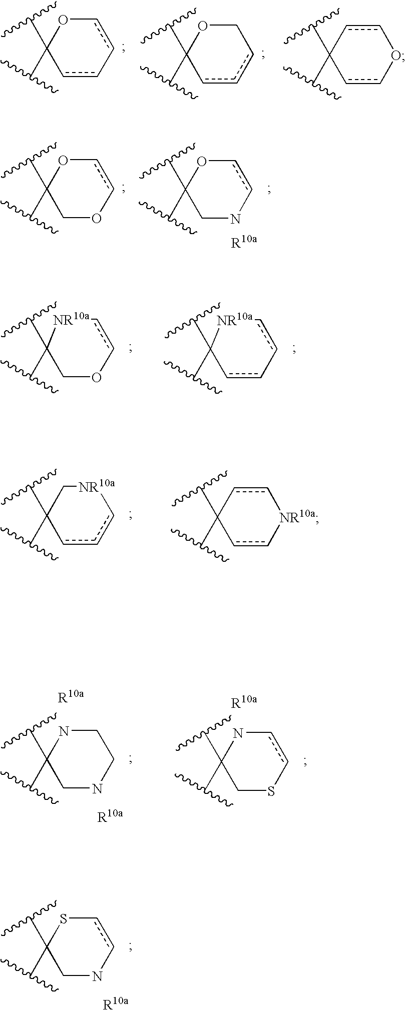

wherein --- is an optional double bond.

In another embodiment, ring A is selected from the group consisting of

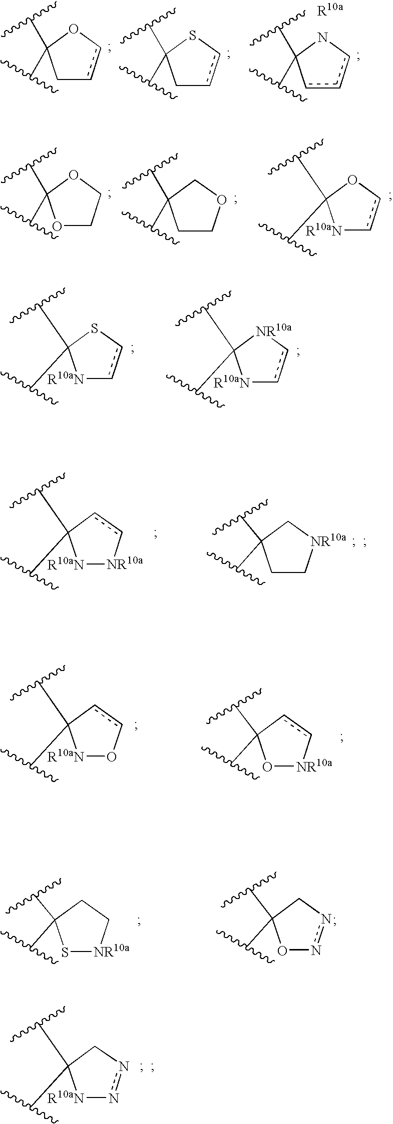

wherein --- is an optional double bond.

In another embodiment, ring A is selected from the group consisting of

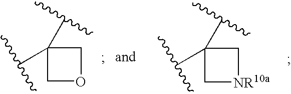

wherein --- is an optional double bond.

Within this embodiment, Ar⁴ is phenyl, naphthyl, pyridinyl, pyrimidinyl, or pyrazinyl. Within this embodiment, specific compounds of the present invention are selected from the group consisting of
1-{4-[2-(5-methyl-2-phenyl-1,3-oxazol-4-yl)ethoxy]benzyl}cyclohexanecarboxylic acid;
1-{4-[2-(5-methyl-2-phenyl-1,3-oxazol-4-yl)ethoxy]benzyl}cyclopentanecarboxylic acid;
1-{4-[3-(5-methyl-2-phenyl-1,3-oxazol-4-yl)propoxy]benzyl}cyclopentanecarboxylic acid;
4-{4-[(5-methyl-2-phenyl-1,3-oxazol-4-yl)methoxy]benzyl}tetrahydro-2H-pyran-4-carboxylic acid;
4-{4-[2-(5-methyl-2-phenyl-1,3-oxazol-4-yl)ethoxy]benzyl}tetrahydro-2H-pyran-4-carboxylic acid;
1-{4-[2-(4′-methoxy-1,1′-biphenyl-4-yl)ethoxy]benzyl}cyclobutanecarboxylic acid;
1-{4-[2-(4′-fluoro-1,1′-biphenyl-4-yl)ethoxy]benzy}cyclobutanecarboxylic acid;
1-{4-[2-(2′-methoxy-1,1′-biphenyl-4-yl)ethoxy]benzyl}cyclobutanecarboxylic acid;
1-(4-{2-[3′-(trifluoromethoxy)-1,1′-biphenyl-4-yl]ethoxy}benzyl)cyclobutanecarboxylic acid;
1-(4-{2-[4-(6-methoxypyridin-3-yl)phenyl]ethoxy}benzyl)cyclobutanecarboxylic acid;
1-(4-{2-[4′-(methylsulfonyl)-1,1′-biphenyl-4-yl]ethoxy}benzyl)cyclobutanecarboxylic acid;
1-(4-{2-[4-(2,3-dihydro-1-benzofuran-6-yl)phenyl]ethoxy}benzyl)cyclobutanecarboxylic acid;
1-[4-(2-{4′-[(methylsulfonyl)amino]-1,1′-biphenyl-4-yl}ethoxy)benzyl]cyclobutane carboxylic acid;
1-{4-[3-(5-methyl-2-phenyl-1,3-oxazol-4-yl)propoxy]benzyl}cyclobutanecarboxylic acid;
1-{4-[(5-methyl-2-phenyl-1,3-oxazol-4-yl)methoxy]benzyl}cyclobutanecarboxylic acid;
1-{3-[2-(5-methyl-2-phenyl-1,3-oxazol-4-yl)ethoxy]benzyl}cyclobutanecarboxylic acid;
1-{4-[2-(5-methyl-2-phenyl-1,3-oxazol-4-yl)ethoxy]benzyl}cyclobutanecarboxylic acid;
1-{4-[(2,5-diphenyl-1,3-oxazol-4-yl)methoxy]benzyl}cyclobutanecarboxylic acid;
1-{4-[3-(2,5-diphenyl-1,3-oxazol-4-yl)propoxy]benzyl}cyclobutanecarboxylic acid;
1-{4-[(2,5-diphenyl-1,3-oxazol-4-yl)methoxy]phenoxy}cyclobutanecarboxylic acid;
1-{4-[3-(2,5-diphenyl-1,3-oxazol-4-yl)propoxy]phenoxy}cyclobutanecarboxylic acid;
1-(4-{2-[2-(1,1′-biphenyl-4-yl)-5-methyl-1,3-oxazol-4-yl]ethoxy}phenoxy)cyclobutane carboxylic acid;
1-{4-[2-(5-methyl-2-phenyl-1,3-oxazol-4-yl)ethoxy]phenoxy}cyclobutanecarboxylic acid;
1-{4-[3-(5-methyl-2-phenyl-1,3-oxazol-4-yl)propoxy]phenoxy}cyclobutanecarboxylic acid;
1-{4-[(5-methyl-2-phenyl-1,3-oxazol-4-yl)methoxy]phenoxy}cyclobutanecarboxylic acid;
1-({6-[2-(5-methyl-2-phenyl-1,3-oxazol-4-yl)ethoxy]pyridin-3-yl}methyl)cyclobutane carboxylic acid;
1-(hydroxy{6-[2-(5-methyl-2-phenyl-1,3-oxazol-4-yl)ethoxy]pyridin-3-yl}methyl) cyclobutanecarboxylic acid;
1-({6-[2-(5-methyl-2-phenyl-1,3-oxazol-4-yl)ethoxy]pyridin-3-yl}methyl) cyclopentanecarboxylic acid;
1-({6-[2-(5-methyl-2-phenyl-1,3-oxazol-4-yl)ethoxy]pyridin-3-yl}methyl) cyclohexanecarboxylic acid;
2-({6-[2-(5-methyl-2-phenyl-1,3-oxazol-4-yl)ethoxy]pyridin-3-yl}methyl) tetrahydrofuran-2-carboxylic acid;
2-({5-[2-(5-methyl-2-phenyl-1,3-oxazol-4-yl)ethoxy]pyridin-2-yl}methyl) tetrahydrofuran-2-carboxylic acid;

and the pharmaceutically acceptable salts thereof.

Within this embodiment, a specific compound of the present invention is 1-{4-[3-(5-methyl-2-phenyl-1,3-oxazol-4-yl)propoxy]benzyl}cyclobutanecarboxylic acid or the pharmaceutically acceptable salts thereof.

Within this embodiment, a specific compound of the present invention is 1-{4-[2-(5-methyl-2-phenyl-1,3-oxazol-4-yl)ethoxy]benzyl}cyclobutanecarboxylic acid or the pharmaceutically acceptable salts thereof.

Within this embodiment, a specific compound of the present invention is 2-({6-[2-(5-methyl-2-phenyl-1,3-oxazol-4-yl)ethoxy]pyridin-3-yl}methyl)tetrahydrofuran-2-carboxylic acid or the pharmaceutically acceptable salts thereof.

Within this embodiment, a specific compound of the present invention is 2-({5-[2-(5-methyl-2-phenyl-1,3-oxazol-4-yl)ethoxy]pyridin-2-yl}methyl)tetrahydrofuran-2-carboxylic acid or the pharmaceutically acceptable salts thereof.

Within this embodiment, a specific compound of the present invention is 2-({6-[2-(5-methyl-2-phenyl-1,3-oxazol-4-yl)ethoxy]pyridin-3-yl}methyl)tetrahydro-2Hpyran-2-carboxylic acid or the pharmaceutically acceptable salts thereof.

Within this embodiment, a specific compound of the present invention is 2-[(6-{2-[2-(3-chlorophenyl)-5-methyl-1,3-oxazol-4-yl]ethoxy}pyridin-3-yl)methyl]tetrahydrofuran-2-carboxylic acid or the pharmaceutically acceptable salts thereof.

Within this embodiment, a specific compound of the present invention is 2-[(6-{2-[2-(3-methoxyphenyl)-5-methyl-1,3-oxazol-4-yl]ethoxy}pyridin-3-yl)methyl]tetrahydrofuran-2-carboxylic acid or the pharmaceutically acceptable salts thereof.

Within this embodiment, a specific compound of the present invention is 2-{5-[2-(5-Methyl-2-phenyl-oxazol-4-yl)-ethoxy]-pyrazin-2-ylmethyl}-tetrahydro-furan-2-carboxylic acid or the pharmaceutically acceptable salts thereof.

Within this embodiment, a specific compound of the present invention is -{4-[2-(5-methyl-2-phenyl-1,3-oxazol-4-yl)ethoxy]benzyl}tetrahydrofuran-2-carboxylic acid or the pharmaceutically acceptable salts thereof.

Within this embodiment, a specific compound of the present invention is 2-{6-[2-(5-Methyl-2-phenyl-oxazol-4-yl)-ethoxy]-naphthalen-2-ylmethyl}-tetrahydro-furan-2-carboxylic acid or the pharmaceutically acceptable salts thereof.

In another embodiment, the invention relates to compounds having a formula:

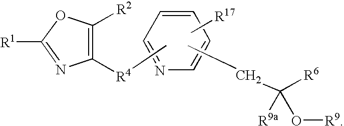

Within this embodiment, preferably the invention relates to compounds having a formula:

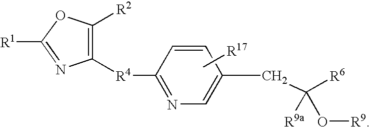

Within this embodiment, preferably the invention relates to compounds having a formula:

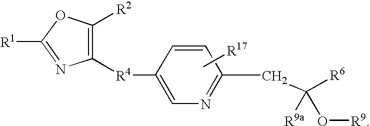

Within this embodiment, preferably R⁹ is methyl, ethyl, or benzyl. Preferably R¹⁷ is H.

Within this embodiment, a specific compound of the present invention is 2-ethoxy-3-{6-[2-(5-methyl-2-phenyl-1,3-oxazol-4-yl)ethoxy]pyridin-3-yl}propanoic acid or the pharmaceutically acceptable salts thereof.

Within this embodiment, a specific compound of the present invention is 2-methoxy-3-(6-{2-[5-methyl-2-(3-methylphenyl)-1,3-oxazol-4-yl]ethoxy}pyridin-3-yl)propanoic acid or the pharmaceutically acceptable salts thereof.

Within this embodiment, a specific compound of the present invention is 2-methoxy-3-{6-[2-(4-phenoxyphenyl)ethoxy]pyridin-3-yl}propanoic acid or the pharmaceutically acceptable salts thereof.

Within this embodiment, a specific compound of the present invention is 2-ethoxy-3-[6-(2-{4-[(phenylsulfonyl)oxy]phenyl}ethoxy)pyridin-3-yl]propanoic acid or the pharmaceutically acceptable salts thereof.

Within this embodiment, a specific compound of the present invention is 2-Ethoxy-3-{5-[2-(5-methyl-2-phenyl-oxazol-4-yl)-ethoxy]-pyridin-2-yl}-propionic acid or the pharmaceutically acceptable salts thereof.

Within this embodiment, a specific compound of the present invention is 2-Methoxy-2-methyl-3-{6-[3-(5-methyl-2-phenyl-oxazol-4-yl)-propoxy]-pyridin-3-yl}-propionic acid or the pharmaceutically acceptable salts thereof.

Within this embodiment, a specific compound of the present invention is 2-Methoxy-2-methyl-3-{5-[2-(5-methyl-2-phenyl-oxazol-4-yl)-ethoxy]-pyridin-2-yl}-propionic acid or the pharmaceutically acceptable salts thereof.

Within this embodiment, a specific compound of the present invention is 3-(6-{2-[2-(4-Chloro-phenyl)-5-methyl-oxazol-4-yl]-ethoxy}-pyridin-3-yl)-2-methoxy-2-methyl-propionic acid or the pharmaceutically acceptable salts thereof.

Within this embodiment, a specific compound of the present invention is 2-Methoxy-2-methyl-3-{6-[2-(5-methyl-2-phenyl oxazol-4-yl)-ethoxy]-pyridin-3-yl}-propionic acid or the pharmaceutically acceptable salts thereof.

Within this embodiment, a specific compound of the present invention is 2-Methoxy-3-(6-{2-[2-(3-methoxy-phenyl)-5-methyl-oxazol-4-yl]-ethoxy}-pyridin-3-yl)-2-methyl-propionic acid or the pharmaceutically acceptable salts thereof.

The present invention also provides a method of treating non-insulin dependent diabetes mellitus in a mammal comprising administering to the mammal in need thereof a therapeutically effective amount of a compound of Formula (I). In one embodiment, said mammal has an impaired glucose tolerance.

The present invention also provides a method of treating polycystic ovarian syndrome in a mammal comprising administering to the mammal in need thereof a therapeutically effective amount of a compound of Formula (I).

The present invention also provides a method of treating obesity in a mammal comprising administering to the mammal in need thereof a therapeutically effective amount of a compound of Formula (I).

The present invention also provides a method of reducing body weight in an obese mammal comprising administering to the mammal in need thereof a therapeutically effective amount of a compound of Formula (I).

The present invention also provides a method of treating hyperglycemia in a mammal comprising administering to the mammal in need thereof a therapeutically effective amount of a compound of Formula (I).

The present invention also provides a method of treating hyperlipidemia in a mammal comprising administering to the mammal in need thereof a therapeutically effective amount of a compound of Formula (I).

The present invention also provides a method of treating hypercholesteremia in a mammal comprising administering to the mammal in need thereof a therapeutically effective amount of a compound of Formula (I).

The present invention also provides a method of treating atherosclerosis in a mammal comprising administering to the mammal in need thereof a therapeutically effective amount of a compound of Formula (I).

The present invention also provides a method of treating hypertriglyceridemia in a mammal comprising administering to the mammal in need thereof a therapeutically effective amount of a compound of Formula (I).

The present invention also provides a method of treating hyperinsulinemia in a mammal comprising administering to the mammal in need thereof a therapeutically effective amount of a compound of Formula (I).

The present invention also provides a method of treating a patient suffering from abnormal insulin and/or evidence of glucose disorders associated with circulating glucocorticoids, growth hormone, catecholamines, glucagon, or parathyroid hormone, comprising administering to said patient a therapeutically effective amount of a compound of Formula (I).

The present invention also provides a method of treating insulin resistance syndrome in humans comprising administering to a patient in need of treatment a therapeutically effective amount of a compound of Formula (I).

The present invention also provides a method of treating PPAR-related disorders in humans comprising administering to a patient in need of treatment a therapeutically effective amount of a compound of Formula (I).

The present invention also provides a method of modulating PPAR activity in a mammal, comprising administering to a mammal a therapeutically effective amount of a compound of Formula (I).

The present invention also provides a method of lowering blood glucose in a mammal, comprising administering to a mammal an amount of a compound of Formula (I) effective to lower blood glucose levels.

The present invention also provides a method of modulating fat cell differentiation in a mammal, comprising administering to a mammal a therapeutically effective amount of a compound of Formula (I).

The present invention also provides a method of modulating processes mediated by PPAR in a mammal, comprising administering to a mammal a therapeutically effective amount of a compound of Formula (I).

The present invention also provides a method of increasing insulin sensitivity in mammals, comprising administering to a mammal a therapeutically effective amount of a compound of Formula (I).

The present invention also provides a method of treating metabolic syndromes selected from the group consisting of galactosemia, maple syrup urine disease, phenylketonuria, hypersarcosinemia, thymine uraciluria, sulfinuria, isovaleric acidemia, saccharopinuria, 4-hydroxybutyric aciduria, glucose-6-phosphate dehydrogenase deficiency, and pyruvate dehydrogenase deficiency, comprising administering to a mammal a therapeutically effective amount of a compound of Formula (I).

The present invention also provides a composition comprising at least one compound of Formula (i) and a pharmaceutically acceptable carrier thereof. Exemplary pharmaceutically acceptable carriers include carriers suitable for oral, intravenous, subcutaneous, intramuscular, intracutaneous, and the like administration. Administration in the form of creams, lotions, tablets, dispersible powders, granules, syrups, elixirs, sterile aqueous or non-aqueous solutions, suspensions or emulsions, and the like, is contemplated.

The PPAR agonists of the present invention may be administered in combination with other agents such as α-glucosidase inhibitors, aldose reductase inhibitors, biguanide preparations, statin base compounds, squalene synthesis inhibitors, fibrate base compounds, LDL catabolism promoters and angiotensin-converting enzyme inhibitors.

In the above description, an α-glucosidase inhibitor is a medicament having action in inhibiting a digestive enzyme such as amylase, maltase, α-dextrinase or sucrase, thereby retarding the digestion of starch or sucrose. Examples of α-glucosidase inhibitors include acarbose, N-(1,3-dihydroxy-2-propyl)variolamine (common name: voglibose) and miglitol.

In the above description, an aldose reductase inhibitor is a medicament which inhibits a rate-limiting enzyme of the first step of the polyol pathway, thereby inhibiting diabetic complications. Examples include tolrestat, epalrestat, 2,7-difluoro-spiro(9H-fluoren-9,4′-imidazolidine)-2′,5′-dione (common name: imirestat), 3-[(4-bromo-2-fluorophenyl)methyl]-7-chloro-3,4-dihydro-2,4-dioxo-1 (2H)-quinozolineacetic acid (common name: zenarestat), 6-fluoro-2,3-dihydro-2,5′-dioxo-spiro[4H-1-benzopyran-4,4′-imidazolidine]-2-carboxamide (SNK-860), zopolrestat, sorbinil and 1-[(3-bromo-2-benzofuranyl)sulfonyl]-2,4-imidazolidinedione (M-16209).

In the above description, a biguanide preparation is a medicament having effects in anaerobic glycolysis promotion, insulin action reinforcement at the periphery, intestinal glucose absorption inhibition, hepatic gluconeogenesis inhibition and fatty-acid oxidation inhibition and examples include phenformin, metformin and buformin.

In the above description, a statin base compound is a medicament which inhibits hydroxymethylglutaryl CoA (HMG-CoA) reductase, thereby lowering the blood cholesterol level and examples include pravastatin and the sodium salt thereof, simvastatin, lovastatin, atorvastatin and fluvastatin.

In the above description, a squalene synthesis inhibitor is a medicament for inhibiting squalene synthesis, thereby lowering the blood cholesterol level and examples include monopotassium (S)-α-[bis(2,2-dimethyl-1-oxopropoxy)methoxy]phosphinyl-3-phenoxybenzene-butanesulfonate (BMS-1 88494).

In the above description, a fibrate base compound is a medicament for inhibiting synthesis and secretion of triglycerides in the liver and activating lipoprotein lipase, thereby lowering the triglyceride level in the blood. Examples include bezafibrate, beclobrate, binifibrate, ciprofibrate, clinofibrate, clofibrate, clofibric acid, ethofibrate, fenofibrate, gemfibrozil, nicofibrate, pirifibrate, ronifibrate, simfibrate and theofibrate.

In the above description, a LDL catabolism promoter is a medicament for increasing LDL (low-density lipoprotein) receptors, thereby lowering the blood cholesterol level and examples include compounds described in Japanese Patent Application Kokai Hei 7-316144 or salts thereof, more specifically, N-[2-[4-bis(4-fluorophenyl)methyl-1-piperazinyl]ethyl]-7,7-diphenyl-2,4,6-heptatrienoic amide.

The above-described statin base compounds, squalene synthesis inhibitors, fibrate base compounds and LDL catabolism promoters can be replaced with another chemical effective for lowering the blood cholesterol or triglyceride level. Examples of such a medicament include nicotinic acid derivative preparations such as nicomol and niceritrol; antioxidants such as probucol; and ion exchange resin preparations such as cholestyramine.

In the above description, an angiotensin-converting enzyme inhibitor is a medicament for inhibiting angiotensin-converting enzyme, thereby lowering the blood pressure and at the same time, partially lowering the blood sugar level of a patient suffering from diabetes. Examples include captopril, enalapril, alacepril, delapril, ramipril, lisinopril, imidapril, benazepril, ceronaprill cilazapril, enalaprilat, fosinopril, moveltipril, perindopril, quinapril, spirapril, temocapril and trandolapril.

For the preparation of oral liquids, suitable carriers include emulsions, solutions, suspensions, syrups, and the like, optionally containing additives such as wetting agents, emulsifying and suspending agents, sweetening, flavoring and perfuming agents, and the like.

For the preparation of fluids for parenteral administration, suitable carriers include sterile aqueous or non-aqueous solutions, suspensions, or emulsions. Examples of non-aqueous solvents or vehicles are propylene glycol, polyethylene glycol, vegetable oils, such as olive oil and corn oil, gelatin, and injectable organic esters such as ethyl oleate. Such dosage forms may also contain adjuvants such as preserving, wetting, emulsifying, and dispersing agents. They may be sterilized, for example, by filtration through a bacteria-retaining filter, by incorporating sterilizing agents into the compositions, by irradiating the compositions, or by heating the compositions. They can also be manufactured in the form of sterile water, or some other sterile injectable medium immediately before use.

### Definitions

For purposes of the present invention, as described and claimed herein, the following terms are defined as follows:

The term “halo”, as used herein, unless otherwise indicated, means fluoro, chloro, bromo or iodo. Preferred halo groups are fluoro, chloro and bromo.

The term “alkyl”, as used herein, unless otherwise indicated, includes saturated monovalent hydrocarbon radicals having straight or branched moieties.

The term “alkenyl”, as used herein, unless otherwise indicated, includes alkyl moieties having at least one carbon-carbon double bond wherein alkyl is as defined above and including E and Z isomers of said alkenyl moiety.

The term “alkynyl”, as used herein, unless otherwise indicated, includes alkyl moieties having at least one carbon-carbon triple bond wherein alkyl is as defined above.

The term “alkoxy”, as used herein, unless otherwise indicated, includes O-alkyl groups wherein alkyl is as defined above.

The term “Me” means methyl, “Et” means ethyl, and “Ac” means acetyl.

The term “cycloalkyl”, as used herein, unless otherwise indicated refers to a non-aromatic, saturated or partially saturated, monocyclic or fused, spiro or unfused bicyclic or tricyclic hydrocarbon referred to herein containing a total of from 3 to 10 carbon atoms, preferably 5-8 ring carbon atoms. Exemplary cycloalkyls include monocyclic rings having from 3-7, preferably 3-6, carbon atoms, such as cyclopropyl, cyclobutyl, cyclopentyl, cyclohexyl, cycloheptyl and the like. Illustrative examples of cycloalkyl are derived from, but not limited to, the following:

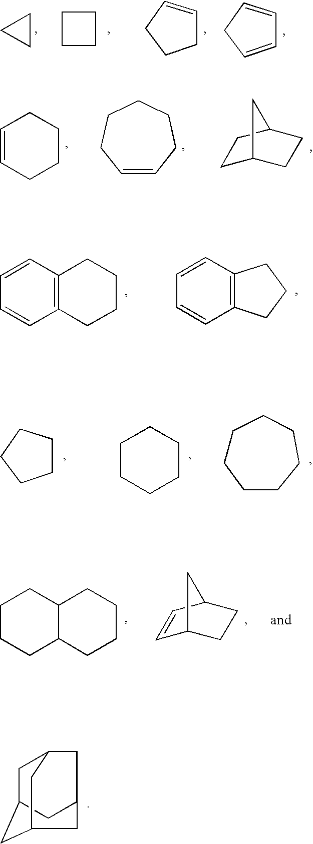

The term “aryl”, as used herein, unless otherwise indicated, includes an organic radical derived from an aromatic hydrocarbon by removal of one hydrogen, such as phenyl or naphthyl.

The term “4-10 membered heterocyclic”, as used herein, unless otherwise indicated, includes aromatic and non-aromatic heterocyclic groups containing one to four heteroatoms each selected from O, S and N, wherein each heterocyclic group has from 4-10 atoms in its ring system, and with the proviso that the ring of said group does not contain two adjacent O or S atoms. Non-aromatic heterocyclic groups include groups having only 4 atoms in their ring system, but aromatic heterocyclic groups must have at least 5 atoms in their ring system. The heterocyclic groups include benzo-fused ring systems. An example of a 4 membered heterocyclic group is azetidinyl (derived from azetidine). An example of a 5 membered heterocyclic group is thiazolyl and an example of a 10 membered heterocyclic group is quinolinyl. Examples of non-aromatic heterocyclic groups are pyrrolidinyl, tetrahydrofuranyl, dihydrofuranyl, tetrahydrothienyl, tetrahydropyranyl, dihydropyranyl, tetrahydrothiopyranyl, piperidino, morpholino, thiomorpholino, thioxanyl, piperazinyl, azetidinyl, oxetanyl, thietanyl, homopiperidinyl, oxepanyl, thiepanyl, oxazepinyl, diazepinyl, thiazepinyl, 1,2,3,6-tetrahydropyridinyl, 2-pyrrolinyl, 3-pyrrolinyl, indolinyl, 2H-pyranyl, 4H-pyranyl, dioxanyl, 1,3-dioxolanyl, pyrazolinyl, dithianyl, dithiolanyl, dihydropyranyl, dihydrothienyl, dihydrofuranyl, pyrazolidinyl, imidazolinyl, imidazolidinyl, 3-azabicyclo[3.1.0]hexanyl, 3-azabicyclo[4.1.0]heptanyl, 3H-indolyl and quinolizinyl. Examples of aromatic heterocyclic groups are pyridinyl, imidazolyl, pyrimidinyl, pyrazolyl, triazolyl, pyrazinyl, tetrazolyl, furyl, thienyl, isoxazolyl, thiazolyl, oxazolyl, isothiazolyl, pyrrolyl, quinolinyl, isoquinolinyl, indolyl, benzimidazolyl, benzofuranyl, cinnolinyl, indazolyl, indolizinyl, phthalazinyl, pyridazinyl, triazinyl, isoindolyl, pteridinyl, purinyl, oxadiazolyl, thiadiazolyl, furazanyl, benzofurazanyl, benzothiophenyl, benzothiazolyl, benzoxazolyl, quinazolinyl, quinoxalinyl, naphthyridinyl, and furopyridinyl. The foregoing groups, as derived from the groups listed above, may be C-attached or N-attached where such is possible. For instance, a group derived from pyrrole may be pyrrol-1-yl (N-attached) or pyrrol-3-yl (C-attached). Further, a group derived from imidazole may be imidazol-1-yl (N-attached) or imidazol-3-yl (C-attached). The 4-10 membered heterocyclic may be optionally substituted on any ring carbon, sulfur, or nitrogen atom(s) by one to two oxo, per ring. An example of a heterocyclic group wherein 2 ring carbon atoms are substituted with oxo moieties is 1,1-dioxo-thiomorpholinyl. Other Illustrative examples of 4-10 membered heterocyclic are derived from, but not limited to, the following:

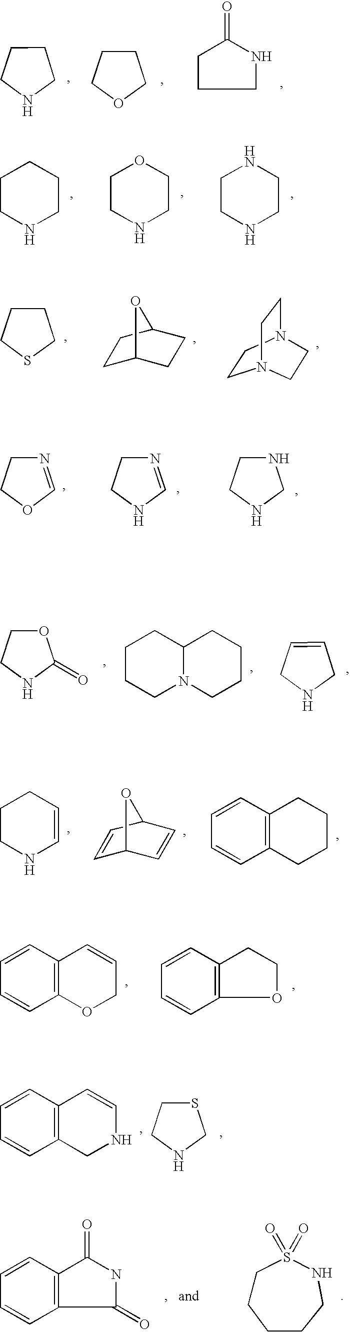

Unless otherwise indicated, the term “oxo” refers to ═O.

The term “—Ar¹—Ar²—”, as used herein, unless otherwise indicated include two rings without any limitation of the order of attachments to R⁴ and R⁵. For example, if —Ar¹—Ar²— is defined as 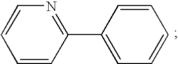
then the —Ar¹—A²— groups can be 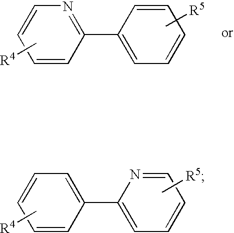
and if —Ar¹—A²— is defined as 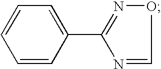
then the —Ar¹—Ar²— groups can be

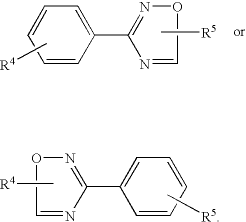

The phrase “pharmaceutically acceptable salt(s)”, as used herein, unless otherwise indicated, includes salts of acidic or basic groups which may be present in the compounds of formula (I). The compounds of formula (I) that are basic in nature are capable of forming a wide variety of salts with various inorganic and organic acids. The acids that may be used to prepare pharmaceutically acceptable acid addition salts of such basic compounds of formula (I) are those that form non-toxic acid addition salts, i.e., salts containing pharmacologically acceptable anions, such as the acetate, benzenesulfonate, benzoate, bicarbonate, bisulfate, bitartrate, borate, bromide, calcium edetate, camsylate, carbonate, chloride, clavulanate, citrate, dihydrochloride, edetate, edislyate, estolate, esylate, ethylsuccinate, fumarate, gluceptate, gluconate, glutamate, glycollylarsanilate, hexylresorcinate, hydrabamine, hydrobromide, hydrochloride, iodide, isothionate, lactate, lactobionate, laurate, malate, maleate, mandelate, mesylate, methylsulfate, mucate, napsylate, nitrate, oleate, oxalate, pamoate (embonate), palmitate, pantothenate, phospate/diphosphate, polygalacturonate, salicylate, stearate, subacetate, succinate, tannate, tartrate, teoclate, tosylate, triethiodode, and valerate salts.

In the compounds of formula (I), where terms such as (CR¹¹R¹²)_{q} or (CR¹¹R¹²)ₜ are used, R¹¹ and R¹² may vary with each iteration of q or t above 1. For instance, where q or t is 2 the terms (CR¹¹R¹²)_{q} or (CR¹¹R¹²)ₜ may equal —CH₂CH₂—, or —CH(CH₃)C(CH₂CH₃)(CH₂CH₂CH₃)—, or any number of similar moieties falling within the scope of the definitions of R¹¹ and R¹². Further, as noted above, any substituents comprising a CH₃ (methyl), CH₂ (methylene), or CH (methine) group which is not attached to a halogeno, SO or SO₂ group or to a N, O or S atom optionally bears on said group a substituent selected from hydroxy, C₁-C₄ alkoxy and amines.

The term “treating”, as used herein, unless otherwise indicated, means reversing, alleviating, inhibiting the progress of, or preventing the disorder or condition to which such term applies, or one or more symptoms of such disorder or condition. The term “treatment”, as used herein, unless otherwise indicated, refers to the act of treating as “treating” is defined immediately above.

The term “modulate” or “modulating”, as used herein, refers to the ability of a modulator for a member of the steroid/thyroid superfamily to either directly (by binding to the receptor as a ligand) or indirectly (as a precursor for a ligand or an inducer which promotes production of ligand from a precursor) induce expression of gene(s) maintained under hormone expression control, or to repress expression of gene(s) maintained under such control.

The term “obesity” or “obese”, as used herein, refers generally to individuals who are at least about 20-30% over the average weight for his/her age, sex and height. Technically, “obese” is defined, for males, as individuals whose body mass index is greater than 27.8 kg/m, and for females, as individuals whose body mass index is greater than 27.3 kg/m². Those of skill in the art readily recognize that the invention method is not limited to those who fall within the above criteria. Indeed, the method of the invention can also be advantageously practiced by individuals who fall outside of these traditional criteria, for example, by those who may be prone to obesity.

The term “Inflammatory disorders”, as used herein, refers to disorders such as rheumatoid arthritis, ankylosing spondylitis, psoriatic arthritis, psoriasis, chondrocalcinosis, gout, inflammatory bowel disease, ulcerative colitis, Crohn's disease, fibromyalgia, and cachexia.

The phrase “therapeutically effective amount”, as used herein, refers to that amount of drug or pharmaceutical agent that will elicit the biological or medical response of a tissue, system, animal, or human that is being sought by a researcher, veterinarian, medical doctor or other.

The phrase “amount . . . effective to lower blood glucose levels,” as used herein, refers to levels of compound sufficient to provide circulating concentrations high enough to accomplish the desired effect. Such a concentration typically falls in the range of about 10 nM up to 2 μM; with concentrations in the range of about 100 nM up to 500 nM being preferred. As noted previously, since the activity of different compounds which fall within the definition of Formula (I) as set forth above may vary considerably, and since individual subjects may present a wide variation in severity of symptoms, it is up to the practitioner to determine a subject's response to treatment and vary the dosages accordingly.

The phrase “insulin resistance”, as used herein, refers to the reduced sensitivity to the actions of insulin in the whole body or individual tissues, such as skeletal muscle tissue, myocardial tissue, fat tissue or liver tissue. Insulin resistance occurs in many individuals with or without diabetes mellitus.

The phrase “insulin resistance syndrome”, as used herein, refers to the cluster of manifestations that include insulin resistance, hyperinsulinemia, non insulin dependent diabetes mellitus (NIDDM), arterial hypertension, central (visceral) obesity, and dyslipidemia.

The phrase “in combination with”, as used herein, means that the alpha substituted carboxylic acids compound of Formula (I) may be administered shortly before, shortly after, concurrently, or any combination of before, after, or concurrently, with such other agents as described in the previous paragraphs. Thus, the alpha substituted carboxylic acids compound of Formula (I) and the other agents may be administered simultaneously as either as a single composition or as two separate compositions or sequentially as two separate compositions.

Certain compounds of formula (I) may have asymmetric centers and therefore exist in different enantiomeric forms. All optical isomers and stereoisomers of the compounds of formula (I), and mixtures thereof, are considered to be within the scope of the invention. With respect to the compounds of formula (I), the invention includes the use of a racemate, one or more enantiomeric forms, one or more diastereomeric forms, or mixtures thereof. The compounds of formula (I) may also exist as tautomers. This invention relates to the use of all such tautomers and mixtures thereof.

Certain functional groups contained within the compounds of the present invention can be substituted for bioisosteric groups, that is, groups which have similar spatial or electronic requirements to the parent group, but exhibit differing or improved physicochemical or other properties. Suitable examples are well known to those of skill in the art, and include, but are not limited to moieties described in Patini, et al., Chem. Rev, 1996, vol. 96, pp. 3147-3176 and references cited therein.

The subject invention also includes isotopically-labelled compounds, which are identical to those recited in Formula (I), but for the fact that one or more atoms are replaced by an atom having an atomic mass or mass number different from the atomic mass or mass number usually found in nature. Examples of isotopes that can be incorporated into compounds of the invention include isotopes of hydrogen, carbon, nitrogen, oxygen, phosphorous, fluorine and chlorine, such as ²H, 3H, ¹³C, ¹⁴C, ¹⁵N, ¹⁸O, ¹⁷O, ³¹P, ³²P, ³⁵S, ¹⁸F, and ³⁶Cl, respectively. Compounds of the present invention, prodrugs thereof, and pharmaceutically acceptable salts of said compounds or of said prodrugs which contain the aforementioned isotopes and/or other isotopes of other atoms are within the scope of this invention. Certain isotopically-labelled compounds of the present invention, for example those into which radioactive isotopes such as ³H and ¹⁴C are incorporated, are useful in drug and/or substrate tissue distribution assays. Tritiated, i.e., ³H, and carbon-14, i.e., ¹⁴C, isotopes are particularly preferred for their ease of preparation and detectability. Further, substitution with heavier isotopes such as deuterium, i.e., ²H, can afford certain therapeutic advantages resulting from greater metabolic stability, for example increased in vivo half-life or reduced dosage requirements and, hence, may be preferred in some circumstances. Isotopically labelled compounds of Formula (I) of this invention and prodrugs thereof can generally be prepared by carrying out the procedures disclosed in the Schemes and/or in the Examples and Preparations below, by substituting a readily available isotopically labelled reagent for a non-isotopically labelled reagent.

This invention also encompasses pharmaceutical compositions containing and methods of treating bacterial infections through administering prodrugs of compounds of the formula 1. Compounds of formula 1 having free amino, amido, hydroxy or carboxylic groups can be converted into prodrugs. Prodrugs include compounds wherein an amino acid residue, or a polypeptide chain of two or more (e.g., two, three or four) amino acid residues is covalently joined through an amide or ester bond to a free amino, hydroxy or carboxylic acid group of compounds of formula 1. The amino acid residues include but are not limited to the 20 naturally occurring amino acids commonly designated by three letter symbols and also includes 4-hydroxyproline, hydroxylysine, demosine, isodemosine, 3-methylhistidine, norvalin, beta-alanine, gamma-aminobutyric acid, citrulline homocysteine, homoserine, omithine and methionine sulfone. Additional types of prodrugs are also encompassed. For instance, free carboxyl groups can be derivatized as amides or alkyl esters. Free hydroxy groups may be derivatized using groups including but not limited to hemisuccinates, phosphate esters, dimethylaminoacetates, and phosphoryloxymethyloxycarbonyls, as outlined in Advanced Drug Delivery Reviews, 1996, 19, 115. Carbamate prodrugs of hydroxy and amino groups are also included, as are carbonate prodrugs, sulfonate esters and sulfate esters of hydroxy groups. Derivatization of hydroxy groups as (acyloxy)methyl and (acyloxy)ethyl ethers wherein the acyl group may be an alkyl ester, optionally substituted with groups including but not limited to ether, amine and carboxylic acid functionalities, or where the acyl group is an amino acid ester as described above, are also encompassed. Prodrugs of this type are described in J. Med. Chem., 1996, 39, 10. Free amines can also be derivatized as amides, sulfonamides or phosphonamides. All of these prodrug moieties may incorporate groups including but not limited to ether, amine and carboxylic acid functionalities.

Other aspects, advantages, and preferred features of the invention will become apparent from the detailed description below.

### DETAILED DESCRIPTION AND PREFERRED EMBODIMENTS OF THE INVENTION

The following reaction Scheme illustrates the preparation of the compounds of the present invention. Unless otherwise indicated, R—R¹⁷, Q, Y, Ar¹—Ar⁴, and Ring A, in the reaction scheme and discussion that follow are as defined above.

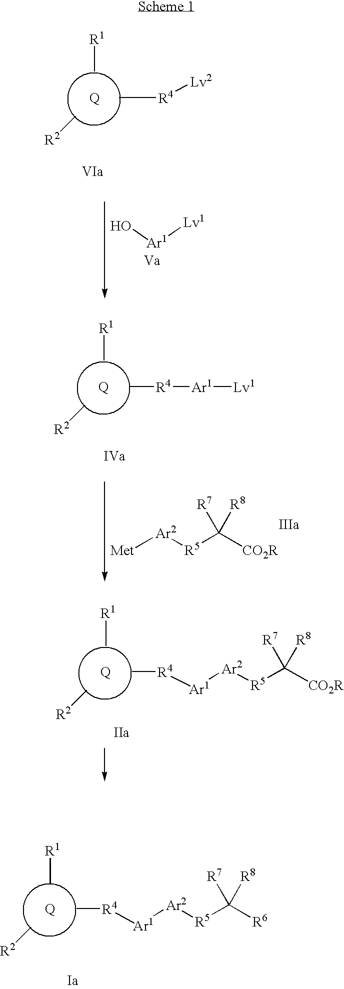

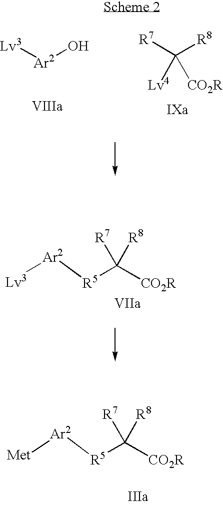

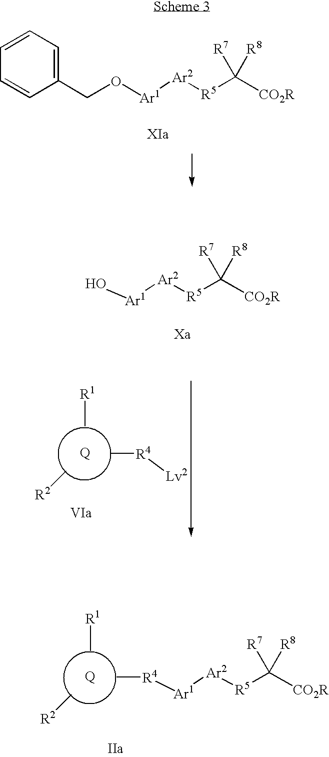

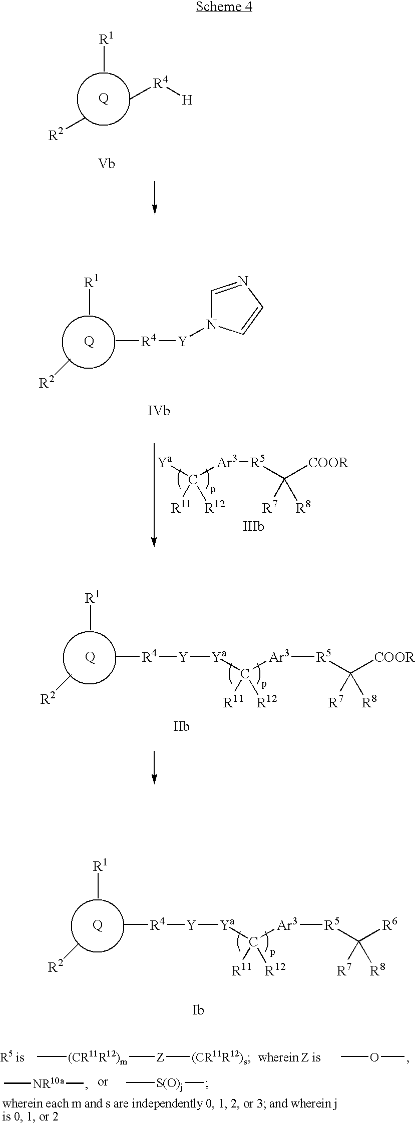

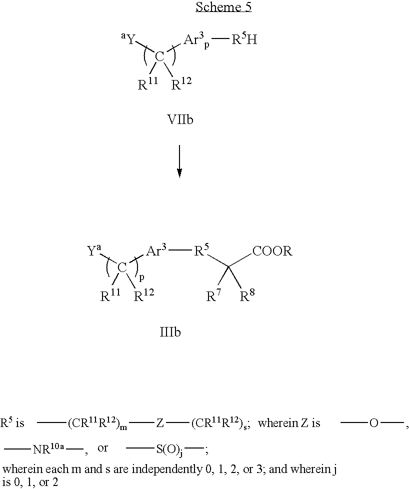

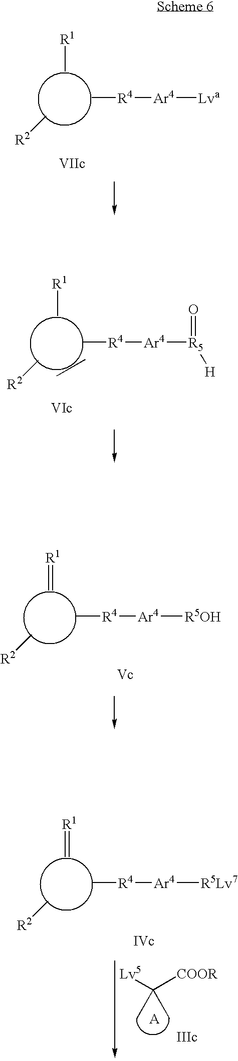

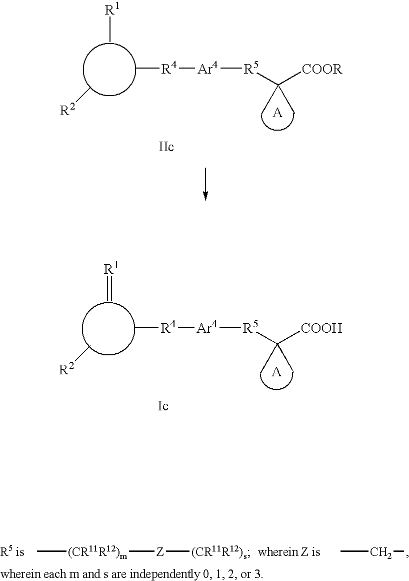

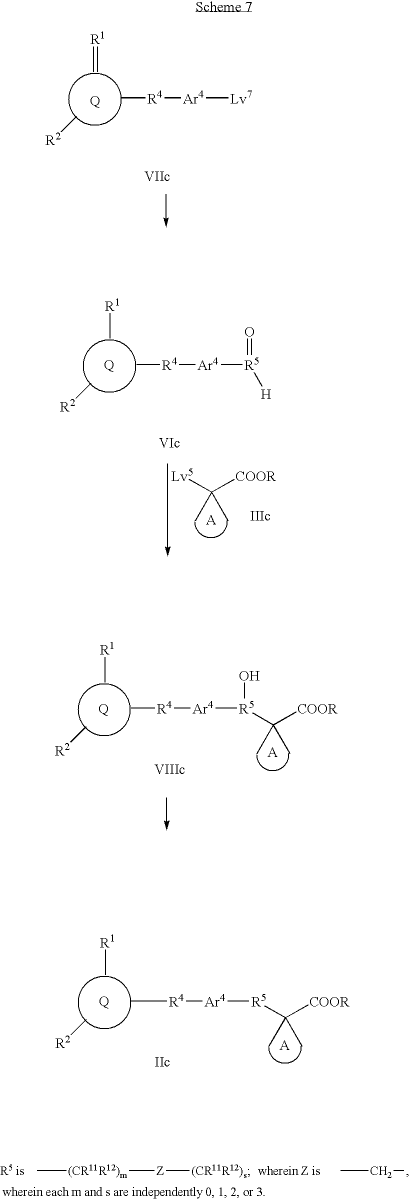

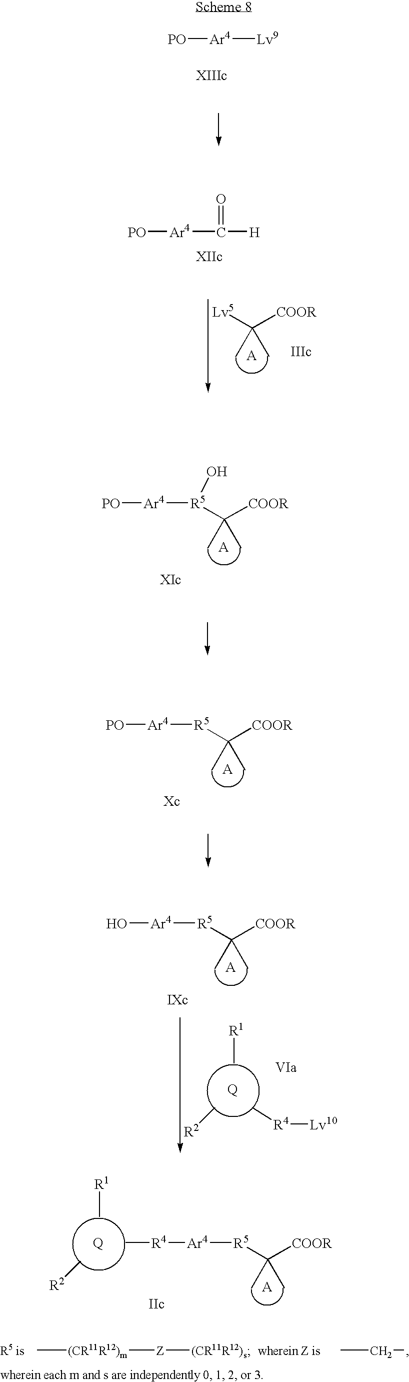

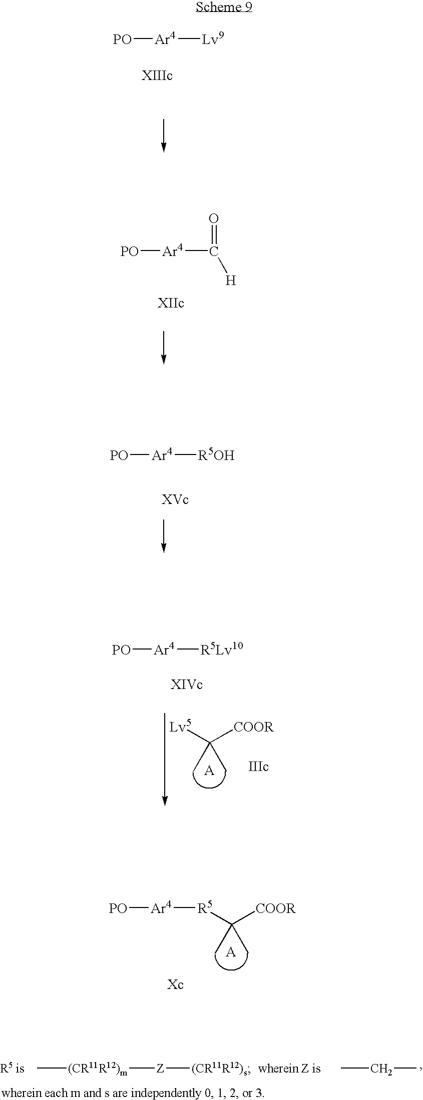

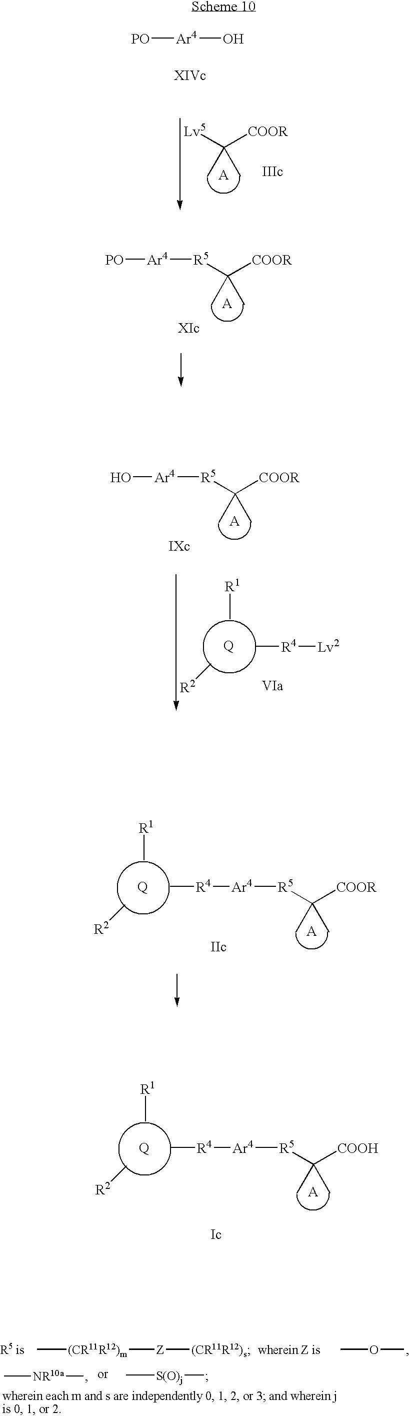

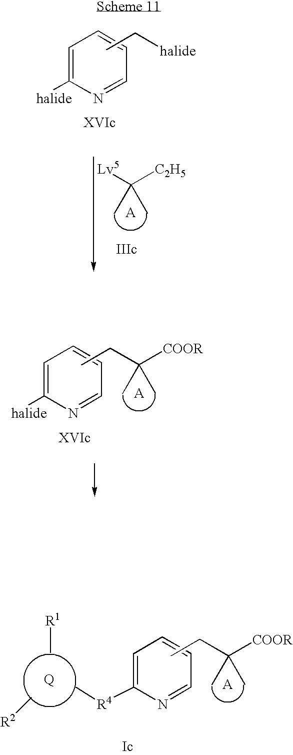

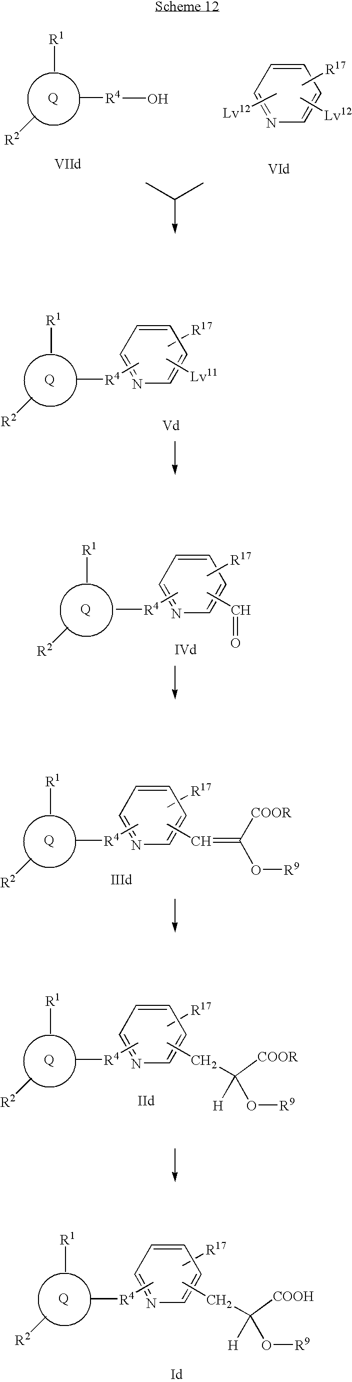

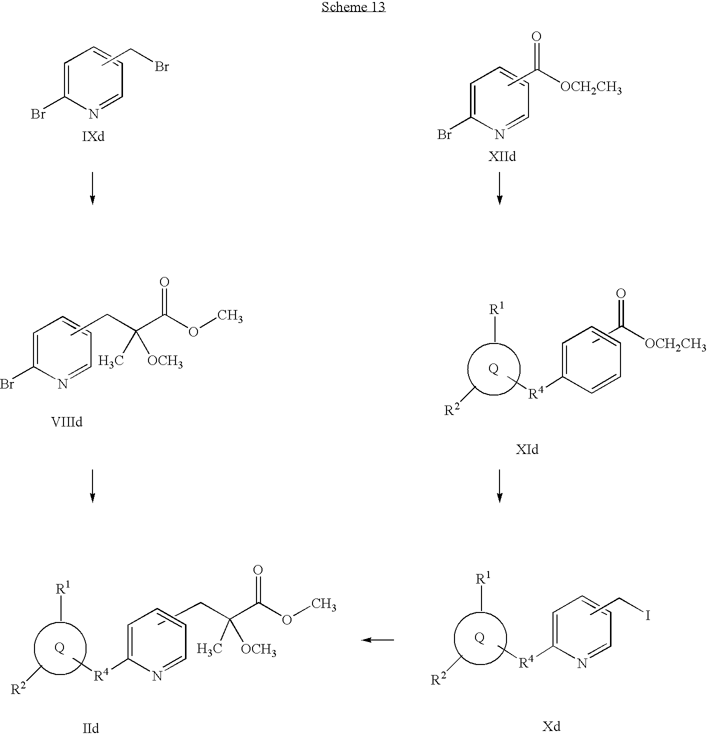

Referring to Scheme 1 above, the compound of formula Ia may be prepared by hydrolysis of compounds IIa, wherein the group CO₂R is a hydrolyzable ester group such as methyl ester (CO₂—CH₃) or ethyl ester (CO₂—CH₂CH₃), by alkali metal hydroxides (e.g. NaOH, LiOH, KOH) in a suitable solvent (e.g. aqueous THF, aqueous methanol or combinations thereof) at a temperature between 0 and 100 degrees or by heating in a microwave synthesizer. Compounds of formula IIa may be prepared by a coupling reaction of compound IVa, wherein Lv¹ is Cl, Br, I, or triflate, and an organometallic compound IIIa, wherein Met=boronic acid or ester, stannane etc, and the group CO₂R is as described above, mediated by a palladium(0) or other transition metal catalyst. Compound IVa can be obtained by alkylation of compound Va, wherein Lv¹ is as described above, with compound VIa, wherein Lv² is Cl, Br, I, or triflate.

Referring to Scheme 2 above, the compound of formula IIIa, which is used in Scheme 1, may be obtained from compound VIIa, wherein Lv³ is Cl, Br, I, or triflate, by palladium(0) mediated coupling reactions with a reagent such as pinacolatodiborane. Compound VIIa, wherein Lv³ is as described above, can be obtained by alkylation of compound VIIIa, wherein Lv³ is as described above, with compound IXa, wherein Lv⁴ is Cl, Br, I, or triflate.

Referring to Scheme 3 above, esters IIa, which is used in Scheme 1, wherein the group CO₂R is as described above, may also be prepared by alkylation of compound Xa, wherein the group CO₂R is as described above, with compound VIa, wherein Lv² is as described above in the description of Scheme 1. Compounds Xa may be obtained from compound XIa, wherein the group CO₂R is as described above, by reacting compound XIa with a deprotecting agent, such as with hydrogen gas over a metal catalyst (e.g. palladium on carbon) in a suitable solvent (e.g. THF, methanol, ethanol) at a temperature between 0 degrees Celcius and 100 degrees Celcius.

Compounds XIa are commercially available or can be made by those skilled in the art.

Referring to Scheme 4 above, compounds of formula Ib; wherein R⁵ is —(CR¹¹R¹²)ₘ-Z-(CR¹¹R¹²)ₛ; wherein Z is —O—, —NH^{1a}, or —S(O)ⱼ—; wherein each m and s are independently 0, 1, 2, or 3; and wherein j is 0, 1, or 2; may be prepared by hydrolysis of compounds IIb, wherein R⁵ is as described in the compounds of formula Ib and the group CO₂R is as described above, by an alkali metal hydroxide (e.g. Na OH, LiOH, KOH) in a suitable solvent (e.g. aqueous THF, aqueous methanol or combinations thereof) at a temperature 0 degrees Celcius and 100 degrees Celcius. Compounds of formula lib, wherein R⁵ is as described in the compounds of formula Ib, may be prepared by reaction of compounds IIIb, wherein R⁵ is as described in the compounds of formula IIb and the group CO₂R is as described above, with an activated acylating agent such as VIb in a suitable solvent (e.g. THF, acetonitrile, dioxane, toluene) at a temperature between 0 degrees Celcius and 100 degrees Celcius. Compounds IVb may be obtained from compound Vb by reacting compound Vb with compound VIb, wherein Lv⁶ is a leaving group. Suitable compound VIb includes N,N′-carbonyl diimidazole.

Compounds Vb and VIb are commercially available or can be made by those skilled in the art.

Referring to Scheme 5 above, compounds of formula IIIb, which is used in Scheme 4, wherein R⁵ is as described in the description of Scheme 4 and the group CO₂R is as described above, may be prepared by reacting compound VIIb wherein R⁵ is as described in the previous paragraph, with an appropriate electrophile of formula Lv⁶-C(R⁷R⁸)—COOR, wherein Lv⁶ is a leaving group such as halo, in the presence of a base (e.g. cesium carbonate, potassium carbonate) in a suitable solvent (e.g. THF, DMF, acetonitrile, or DMSO) at a temperature between 0 degrees Celcius and 100 degrees Celcius. Suitable electrophiles of formula Lv⁶-C(R⁷R⁸)—COOR include methyl 2-bromo-2-methyl propanoate. Compounds VIIb are commercially available or can be made by those skilled in the art.

Compounds of formula Ib, IIb, IIIb, and VIIb; wherein R¹ is —(CR¹¹R¹²)ₘ-Z-(CR¹¹R¹²)ₛ; wherein Z is —CH₂—, and wherein each m and s are as described above; can be prepared by methods known to those skilled in the art.

Referring to Scheme 6 above, compounds of formula Ic, wherein R⁵ is —(CR¹¹R¹²)ₘ-Z-(CR¹¹R¹²)ₛ; wherein Z is —CH₂—, may be prepared by hydrolysis of compounds IIc, wherein the group CO₂R is as described above, by alkali metal hydroxides (e.g. NaOH, LiOH, KOH) in a suitable solvent (e.g. aqueous THF, aqueous methanol or combinations thereof) at a temperature between 0 degrees Celcius and 100 degrees Celcius or by heating in a microwave synthesizer. Compounds IIc may be prepared by alkylation of compound IIIc, wherein Lv⁵ is a leaving group, with compound IVc (when Lv⁷ is iodide, bromide, chloride or other leaving group). Various methods can be used to effect this reaction, such as deprotonation of compound IIIc (Lv⁵=H) with a base e.g. sodium bis(trimethylsilyl)amide. Compounds IVc may be prepared from compounds Vc by reaction with a halogenation agent or halogenation system e.g. oxalyl chloride and dimethyl formamide or from another halide (e.g. reaction of compound IVc, Lv⁷=Cl with sodium iodide). Compounds Vc may be prepared from compounds VIc by reacting compounds VIIIc with a reducing agent, such as sodium borohydride. Compounds VIc may be obtained from compound VIIc (Lv⁸=Br or other halogen) by metal-halogen exchange (e.g. with butyllithium) followed by reaction with dimethyl formamide.

Alternatively, referring to Scheme 7 above, compounds of formula IIc, wherein R⁵ is —(CR¹¹R¹²)ₘ-Z-(CR¹¹R¹²)ₛ; wherein Z is —CH₂—, may be prepared by the reductive deoxygenation of compound VIIIc using a silane and an acid source, typically triethylsilane and trifluoroacetic acid. Compounds VIIIc may be obtained by addition of compound IIIc to compound VIc. Various methods can be used to effect this reaction, such as deprotonation of IIIc (Lv⁵=H) with a base e.g. lithium diisopropylamide, or Reformatsky type activation of IIIc (Lv⁵=Br) with a metal or metal salt (e.g. chromium(II) chloride). Compounds VIc may be obtained from compound VIIc (Lv⁸=Br or other halo) by metal-halogen exchange (e.g. with butyllithium) followed by reaction with dimethyl formamide.

Alternatively, referring to Scheme 8 above, compound IIc, wherein R⁵ is —(CR¹¹R¹²)ₘ-Z-(CR¹¹R¹²)ₛ; wherein Z is —CH₂—, may be obtained by reaction of compounds IXc with suitable coupling partners of formula VIa. These reactions may be effected using electrophiles VIa (e.g. Lv²=halides, sulphonate esters) in the presence of a base (e.g. cesium carbonate) or with alcohols (Lv²=OH) under Mitsunobu-type conditions (e.g. triphenyl phosphine and diethylazodicarboxylate). Compounds IXc can be prepared by deprotection of protected compounds Xc. Suitable protecting groups can include allyl, benzyl etc. Deprotection of Xc (P=allyl) can be achieved by exposure to a soluble transition metal (e.g. tetrakis(triphenylphosphine)palladium(0)) in the presence of a base e.g. morpholine.

Intermediates Xc-XIIIc may be prepared by the methods described in Scheme 6.

Alternatively, referring to Scheme 9 above, compound Xc can be prepared by reacting compound XIVc (e.g. Lv¹⁰=halides, sulphonate esters) with compound IIIc, wherein Lv⁵ is as described above. Compound XIVc can be prepared by reacting compound XVc with (C═O)Cl₂ in a polar aprotic solvents such as dimethylformamide. Compound XVc can be prepared by reacting compound XIIc with a reducing agent, such as sodium borohydride. Compounds XIIIc may be prepared by the method described in Scheme 6.

Alternatively, referring to Scheme 10 above, the compound of formula Ic, wherein R⁵ is —(CR¹¹R¹²)ₘ-Z-(CR¹¹R¹²)ₛ; wherein Z is —O—, —NR^{10a}—, or —S(O)ⱼ—; wherein each m and s are independently 0, 1, 2, or 3; and wherein j is 0, 1, or 2; may be prepared by hydrolysis of compounds IIc by alkali metal hydroxides (e.g. NaOH, LiOH, KOH) in a suitable solvent (e.g. aqueous THF, aqueous methanol or combinations thereof) at a temperature between 0 degrees Celcius and 100 degrees Celcius or by heating in a microwave synthesizer. Compounds of formula IIc may be obtained by reaction of compounds IXc with suitable coupling partners. These reactions may be effected using electrophiles (e.g. VIa; Lv²=halides, sulphonate esters) in the presence of a base (e.g. cesium carbonate, potassium carbonate or potassium t-butoxide) or with alcohols (Via; Lv²=OH) under Mitsunobu-type conditions (e.g. triphenyl phosphine and diethylazodicarboxylate). Compounds IXc may be prepared by deprotection of compounds XIc wherein P is a protecting group. Suitable P protecting groups can include allyl, benzyl etc. Deprotection of XIc (P=allyl) can be achieved by exposure to a soluble transition metal (e.g. tetrakis(triphenylphosphine)palladium(0)) in the presence of a base e.g. morpholine or by reduction (XIc; P=benzyl) with hydrogen gas over a metal catalyst (e.g. palladium on carbon) in a suitable solvent (e.g. THF, methanol, ethanol) at a temperature between 0 degrees Celcius and 100 degrees Celcius. Compounds XIc can be obtained by alkylation of compounds XIVc with compound IIIc (Lv⁵=Cl, Br, I, triflate, as described above).

Referring to Scheme 11 above, in certain cases alkylation of an enolate anion of compound IIc with a benzylhalide having a formula XVIc affords compounds XVc. Compounds XVc can be converted into compounds Ic by e.g. palladium mediated coupling reaction in a solvent known by those skilled in the art (e.g., March, Advanced organic Chemistry, Fourth Edition).

Referring to Scheme 12 above, compounds of formula Id may be prepared by hydrolysis of compounds IId by alkali metal hydroxides (e.g. NaOH, LiOH, KOH) in a suitable solvent (e.g. aqueous THF, aqueous methanol or combinations thereof) at a temperature between 0 degrees Celcius and 100 degrees Celcius. Compounds of formula IId may be prepared by reaction of compounds IIId with an appropriate hydrogenation agent such as hydrogen gas over a metal catalyst (e.g., palladium on carbon) in a suitable solvent (e.g. THF, methanol, ethanol) at a temperature between 0 degrees Celcius and 100 degrees Celcius. Compounds of formula IId may be prepared by reaction of compounds IVd with an appropriate triphenyl phosphine reagent having a formula: (C₆H₅)₃P⁺—CH(OR⁹)(COOR) Cl⁻ in a Wittig reaction. Suitable triphenyl phosphine reagents include 1,2-diethoxy-2-oxoethyl)(triphenyl) phosphonium chloride. Compounds of formula IVd may be prepared by reaction of compounds Vd as described in Scheme 9. Compounds of formula Vd may be prepared by reaction of compounds VId and VIId as described in Scheme 9.

Alternatively, compounds of formula IId may be prepared by the methods of Scheme 13. Referring to Scheme 13, alkylation of enolate anion of methyl 2-methoxy propanoate with a benzyl halide IXd affords compounds VIIId. Compounds VIIId can be elaborated to compounds IId by e.g. palladium mediated coupling reaction. Compounds IId may also be prepared from compounds Xd. Compounds Xd can be prepared from compounds XIId by a sequence of reactions such as (i) palladium mediated coupling reaction to form compounds XId, and (ii) reduction of the ester to alcohol, and (iii) halide formation to form compounds IId.

Any of the above compounds of formula I and any of the compounds in the schemes 1-13 above can be converted into another analogous compound by standard chemical manipulations. These chemical manipulations are known to those skilled in the art and include a) removal of a protecting group by methods outlined in T. W. Greene and P. G. M. Wuts, “Protective Groups in Organic Synthesis”, Second Edition, John Wiley and Sons, New York, 1991; b) displacement of a leaving group (halide, mesylate, tosylate, etc) with a primary or secondary amine, thiol or alcohol to form a secondary or tertiary amine, thioether or ether, respectively; c) treatment of phenyl (or substituted phenyl) carbamates with primary of secondary amines to form the corresponding ureas as in Thavonekham, B et. al. Synthesis (1997), 10, p1189; d) reduction of propargyl or homopropargyl alcohols or N-BOC protected primary amines to the corresponding E-allylic or E-homoallylic derivatives by treatment with sodium bis(2-methoxyethoxy)aluminum hydride (Red-Al) as in Denmark, S. E.; Jones, T. K. J. Org. Chem. (1982) 47, 4595-4597 or van Benthem, R. A. T. M.; Michels, J. J.; Speckamp, W. N. Synlett (1994), 368-370; e) reduction of alkynes to the corresponding Z-alkene derivatives by treatment hydrogen gas and a Pd catalyst as in Tomassy, B. et. al. Synth. Commun. (1998), 28, p1201 f) treatment of primary and secondary amines with an isocyanate, acid chloride (or other activated carboxylic acid derivative), alkyl/aryl chloroformate or sulfonyl chloride to provide the corresponding urea, amide, carbamate or sulfonamide; g) reductive amination of a primary or secondary amine using R¹CH(O); and h) treatment of alcohols with an isocyanate, acid chloride (or other activated carboxylic acid derivative), alkyl/aryl chloroformate or sulfonyl chloride to provide the corresponding carbamate, ester, carbonate or sulfonic acid ester.

The compounds of the present invention may have asymmetric carbon atoms. Diasteromeric mixtures can be separated into their individual diastereomers on the basis of their physical chemical differences by methods known to those skilled in the art, for example, by chromatography or fractional crystallization. Enantiomers can be separated by converting the enantiomeric mixtures into a diastereomric mixture by reaction with an appropriate optically active compound (e.g., alcohol), separating the diastereomers and converting (e.g., hydrolyzing) the individual diastereomers to the corresponding pure enantiomers; or by chromatographic separation using chiral stationary or mobile phase. All such isomers, including diastereomeric mixtures and pure enantiomers are considered as part of the invention.

The compounds of formulas (I) that are basic in nature are capable of forming a wide variety of different salts with various inorganic and organic acids. Although such salts must be pharmaceutically acceptable for administration to animals, it is often desirable in practice to initially isolate the compound of formula (I) from the reaction mixture as a pharmaceutically unacceptable salt and then simply convert the latter back to the free base compound by treatment with an alkaline reagent and subsequently convert the latter free base to a pharmaceutically acceptable acid addition salt. The acid addition salts of the base compounds of this invention are readily prepared by treating the base compound with a substantially equivalent amount of the chosen mineral or organic acid in an aqueous solvent medium or in a suitable organic solvent, such as methanol or ethanol. Upon careful evaporation of the solvent, the desired solid salt is readily obtained. The desired acid salt can also be precipitated from a solution of the free base in an organic solvent by adding to the solution an appropriate mineral or organic acid.

Those compounds of formula (I) that are acidic in nature are capable of forming base salts with various pharmacologically acceptable cations. Examples of such salts include the alkali metal or alkaline-earth metal salts and particularly, the sodium and potassium salts. These salts are all prepared by conventional techniques. The chemical bases which are used as reagents to prepare the pharmaceutically acceptable base salts of this invention are those which form non-toxic base salts with the acidic compounds of formula (I). Such non-toxic base salts include those derived from such pharmacologically acceptable cations as sodium, potassium calcium and magnesium, etc. These salts can easily be prepared by treating the corresponding acidic compounds with an aqueous solution containing the desired pharmacologically acceptable cations, and then evaporating the resulting solution to dryness, preferably under reduced pressure. Alternatively, they may also be prepared by mixing lower alkanolic solutions of the acidic compounds and the desired alkali metal alkoxide together, and then evaporating the resulting solution to dryness in the same manner as before. In either case, stoichiometric quantifies of reagents are preferably employed in order to ensure completeness of reaction and maximum yields of the desired final product.

The compounds of the present invention are modulators of PPAR, preferably PPARγ and α. The compounds of the present invention can modulate processes mediated by PPAR-γ, which refers to biological, physiological, endocrinological, and other bodily processes which are mediated by receptor or receptor combinations which are responsive to the PPAR agonists described herein (e.g., diabetes, hyperlipidemia, obesity, impaired glucose tolerance, hypertension, fatty liver, diabetic complications (e.g. retinopathy, nephropathy, neurosis, cataracts and coronary artery diseases and the like), arteriosclerosis, pregnancy diabetes, polycystic ovary syndrome, cardiovascular diseases (e.g. ischemic heart disease and the like), cell injury (e.g. brain injury induced by strokes and the like) induced by atherosclerosis or ischemic heart disease, gout, inflammatory diseases (e.g. arthrosteitis, pain, pyrexia, rheumatoid arthritis, inflammatory enteritis, acne, sunburn, psoriasis, eczema, allergosis, asthma, GI ulcer, cachexia, autoimmune diseases, pancreatitis and the like), cancer, osteoporosis and cataracts. Modulation of such processes can be accomplished in vitro or in vivo. In vivo modulation can be carried out in a wide range of subjects, such as, for example, humans, rodents, sheep, pigs, cows, and the like.

The compounds of the present invention may also be useful in the treatment of other metabolic syndromes associated with impaired glucose utilization and insulin resistance include major late-stage complications of NIDDM, such as diabetic angiopathy, atherosclerosis, diabetic nephropathy, diabetic neuropathy, and diabetic ocular complications such as retinopathy, cataract formation and glaucoma, and many other conditions linked to NIDDM, including dyslipidemia glucocorticoid induced insulin resistance, dyslipidemia, polycysitic ovarian syndrome, obesity, hyperglycemia, hyperlipidemia, hypercholesteremia, hypertriglyceridemia, hyperinsulinemia, and hypertension. Brief definitions of these conditions are available in any medical dictionary, for instance, Stedman's Medical Dictionarv (Xth Ed.).

The in vitro activity of the compounds of formula (I) may be determined by the following procedure.

### Scintillatian Proximity Assay (SPA) Assays

In the SPA assay, 3H labeled darglitazone (for PPAR-γ) or GW2331 (for PPAR-α) is bound to the PPAR protein captured on SPA polylysine beads and generates radioactive count signal that can be detected by TopCounts (Packard). The PPAR-bound 3H labeled ligand can be displaced by an unlabeled compound. The Ki of the compound can be then determined by the extent of displacement at various compound concentrations.

Reagents:

SPA polylysine beads, which can be purchased from Amersham Bioscience.

3H labeled Darglitazone for PPAR-γ.

3H labeled GW2331 for PPAR-α.

PPAR proteins.

Buffer—PBS, 10% glycerol, 14 mM beta-mercaptoethanol.

The compounds of the present invention that were tested all have Kis in at least one of the above SPA assays of between 0.3 nM to 30 μM. Certain preferred groups of compounds possess differential selectivity toward the various PPARs. One group of preferred compounds possesses selective activity towards PPAR-α over PPAR-γ. Another preferred group of compounds possesses selective activity towards towards PPAR-γ over PPAR-α. Another preferred group of compounds possesses selective activity towards both PPAR-α and PPAR-γ over PPAR-δ. Another preferred group of compounds possesses selective activity towards PPAR-δ over both PPAR-α and PPAR-γ.

The alpha substituted carboxylic acids compounds of Formula (I) may be provided in suitable topical, oral and parenteral pharmaceutical formulations for use in the treatment of PPAR mediated diseases. The compounds of the present invention may be administered orally as tablets or capsules, as oily or aqueous suspensions, lozenges, troches, powders, granules, emulsions, syrups or elixars. The compositions for oral use may include one or more agents for flavoring, sweetening, coloring and preserving in order to produce pharmaceutically elegant and palatable preparations. Tablets may contain pharmaceutically acceptable excipients as an aid in the manufacture of such tablets. As is conventional in the art these tablets may be coated with a pharmaceutically acceptable enteric coating, such as glyceryl monostearate or glyceryl distearate, to delay disintegration and absorption in the gastrointestinal tract to provide a sustained action over a longer period.

Formulations for oral use may be in the form of hard gelatin capsules wherein the active ingredient is mixed with an inert solid diluent, for example, calcium carbonate, calcium phosphate or kaolin. They may also be in the form of soft gelatin capsules wherein the active ingredient is mixed with water or an oil medium, such as peanut oil, liquid paraffin or olive oil.

Aqueous suspensions normally contain active ingredients in admixture with excipients suitable for the manufacture of an aqueous suspension. Such excipients may be a suspending agent, such as sodium carboxymethyl cellulose, methyl cellulose, hydroxypropylmethyl cellulose, sodium alginate, polyvinylpyrrolidone, gum tragacanth and gum acacia; a dispersing or wetting agent that may be a naturally occuring phosphatide such as lecithin, a condensation product of ethylene oxide and a long chain fatty acid, for example polyoxyethylene stearate, a condensation product of ethylene oxide and a long chain aliphatic alcohol such as heptadecaethylenoxycetanol, a condensation product of ethylene oxide and a partial ester derived from a fatty acid and hexitol such as polyoxyethylene sorbitol monooleate or a fatty acid hexitol anhydrides such as polyoxyethylene sorbitan monooleate.

The pharmaceutical compositions may be in the form of a sterile injectable aqueous or oleagenous suspension. This suspension may be formulated according to know methods using those suitable dispersing or wetting agents and suspending agents that have been mentioned above. The sterile injectable preparation may also be formulated as a suspension in a non toxic perenterally-acceptable diluent or solvent, for example as a solution in 1,3-butanediol. Among the accetable vehicles and solvents that may be employed are water, Ringers solution and isotonic sodium chloride solution. For this purpose any bland fixed oil may be employed including synthetic mono- or diglycerides. In addition fatty acids such as oleic acid find use in the preparation of injectables.

The alpha substituted carboxylic acids compounds of Formula (I) may also be administered in the form of suppositories for rectal administration of the drug. These compositions can be prepared by mixing the drug with a suitable non-irritating excipient that is solid at about room temperature but liquid at rectal temperature and will therefore melt in the rectum to release the drug. Such materials include cocoa butter and other glycerides.

For topical use preparations, for example, creams, ointments, jellies solutions, or suspensions, containing the compounds of the present invention are employed.

The alpha substituted carboxylic acids compounds of Formula (I) may also be administered in the form of liposome delivery systems such as small unilamellar vesicles, large unilamellar vesicles and multimellar vesicles. Liposomes can be formed from a variety of phospholipides, such as cholesterol, stearylamine or phosphatidylcholines.

Dosage levels of the compounds of the present invention are of the order of about 0.5 mg/kg body weight to about 100 mg/kg body weight. A preferred dosage rate is between about 30 mg/kg body weight to about 100 mg/kg body weight. It will be understood, however, that the specific dose level for any particular patient will depend upon a number of factors including the activity of the particular compound being administered, the age, body weight, general health, sex, diet, time of administration, route of administration, rate of excretion, drug combination and the severity of the particular disease undergoing therapy. To enhance the therapeutic activity of the present compounds they may be administered concomitantly with other orally active antidiabetic compounds such as the sulfonylureas, for example, tolbutamide and the like.

Methods of preparing various pharmaceutical compositions with a specific amount of active compound are known, or will be apparent, to those skilled in this art. For examples, see Remington's Pharmaceutical Sciences, Mack Publishing Company, Easter, Pa., 15th Edition (1975).

The examples and preparations provided below further illustrate and exemplify the compounds of the present invention and methods of preparing such compounds. It is to be understood that the scope of the present invention is not limited in any way by the scope of the following examples and preparations. In the following examples molecules with a single chiral center, unless otherwise noted, exist as a racemic mixture. Those molecules with two or more chiral centers, unless otherwise noted, exist as a racemic mixture of diastereomers. Single enantiomers/diastereomers may be obtained by methods known to those skilled in the art.

Where HPLC chromatography is referred to in the preparations and examples below, the general conditions used, unless otherwise indicated, are as follows. The column used is a ZORBAX™ RXC18 column (manufactured by Hewlett Packard) of 150 mm distance and 4.6 mm interior diameter. The samples are run on a Hewlett Packard-1100 systemA gradient solvent method is used running 100 percent ammonium acetate/acetic acid buffer (0.2 M) to 100 percent acetonitrile over 10 minutes. The system then proceeds on a wash cycle with 100 percent acetonitrile for 1.5 minutes and then 100 percent buffer solution for 3 minutes. The flow rate over this period is a constant 3 ml/minute.

In the following examples and preparations, “Et” means ethyl, “AC” means acetyl, “Me” means methyl, “ETOAC” or “ETOAc” means ethyl acetate, “THF” means tetrahydrofuran, and “Bu” means butyl.

Chiral Supercritical Fluid Chromatography (SFC) Conditions.

Single enantiomers of certain racemic compounds were obtained by SFC using a chiralpak AD-H column at 140 bar and 2.5 mL/min, chiralpak AS-H column at 140 bar and 2.5 mL/min, chiralpak OJ-H column at 140 bar and 2.5 mL/min.

Throughout the following sections, compounds of the formula below: 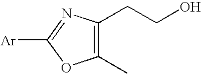
were prepared by procedures analogous to those described in Heterocycles, 2001, 55(4), 689-703.

### EXAMPLE A-1

### 2-Methyl-2-({3′-[2-(5-methyl-2-phenyl-1,3-oxazol-4-yl)ethoxy]-1,1′-biphenyl-3-yl}oxy)propanoic acid

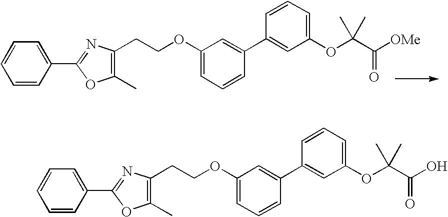

To a solution of methyl 2-methyl-2-({3′-[2-(5-methyl-2-phenyl-1,3-oxazol-4-yl)ethoxy]-1,1′-biphenyl-3-yl}oxy)propanoate (0.89 g, 1.76 mmol) in methanol (20 mL) was added water (2.6 mL) and potassium carbonate (0.73 g, 2.0 equiv). The mixture was then heated at reflux for 5 hours and allowed to cool to ambient temperature. The solution was poured into water, acidified to pH 2 with 1N hydrochloric acid and extracted with ethyl acetate (3×30 mL). The combined organics were washed with saturated aqueous sodium chloride, dried (anhydrous sodium sulfate), filtered and concentrated to dryness to give the title compound as a white crystalline solid (0.6 g, 70%). Elemental Analysis: Calcd for C₂₈H₂₇NO₅ C, 73.51; H, 5.95; N, 3.06. Found: C, 73.26; H, 6.08; N, 3.06. LRMS: 458 (M+H)⁺. ¹H NMR (CDCl₃, 400 MHz): δ 7.97 (2H, dd, J=3.0, 6.6 Hz), 7.43 (2H, d, J=2.8 Hz), 7.41 (1H, s), 7.31 (2H, t, J=8.0 Hz), 7.23 (2H, d, J=8.6 Hz), 7.17 (1H, d, J=7.6 Hz), 7.12 (1H, bs), 6.93 (1H, dd, J=1.4, 8.2 Hz), 6.87 (1H, dd, J=2.0, 8.1 Hz), 4.29 (2H, t, J=7.7 Hz), 3.07 (2H, t, J=7.7 Hz), 2.40 (3H, s), 1.63 (6H, s).

### EXAMPLE A-2

### 2-Methyl-2-[(3′-{[4-(trifluoromethyl)benzyl]oxy}-1,1′-biphenyl-3-yl)oxy]propanoic acid

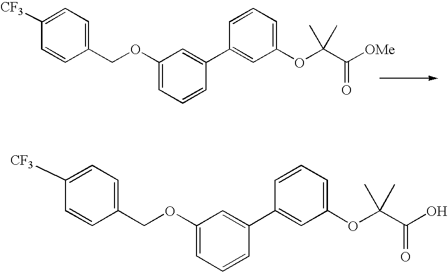

Following the procedure described in Example A-1, starting from methyl 2-methyl-2-[(3′-{[4-(trifluoromethyl)benzyl]oxy}-1,1′-biphenyl-3-yl)oxy]propanoate, the title compound was produced. LRMS: 431 (M+H)⁺.

### EXAMPLE A-3

### 2-Methyl-2-[(3′-{2-[1-(6-methylpyridazin-3-yl)piperidin-4-yl]ethoxy}-1,1′-biphenyl-3-yl)oxy]propanoic acid

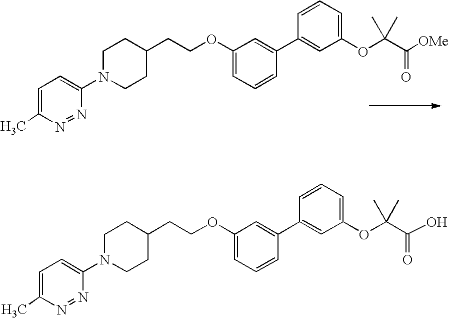

Following the procedure described in Example A-1, starting from methyl 2-methyl-2-[(3′-{2-[1-(6-methylpyridazin-3-yl)piperidin-4-yl]ethoxy}-1,1′-biphenyl-3-yl)oxy]propanoate, the title compound was produced as a pale yellow crystalline solid. LRMS: 477 (M+H)⁺. ¹H NMR (CDCl₃, 400 MHz): δ 7.27 (2H, q, J=8.1 Hz), 7.20-7.18 (2H, m), 7.12 (1H, bd, J=7.8 Hz), 7.08-7.06 (2H, m), 6.94-6.93 (1H, m), 6.91-6.90 (1H, m), 6.84 (1H, dd, J=2.0, 7.8 Hz), 4.25 (2H, bd, J=13.1 Hz), 4.04 (2H, t, J=6.1 Hz), 2.88 (2H, t, J=13.4 Hz), 2.48 (3H, s), 1.80-1.70 (5H, m), 1.65 (6H, s), 1.33-1.27 (2H, m).

### EXAMPLE A-4

### 1-({3′-[2-(5-Methyl-2-phenyl-1,3-oxazol-4-yl)ethoxy]-1,1′-biphenyl-3-yl}oxy)cyclobutanecarboxylic acid

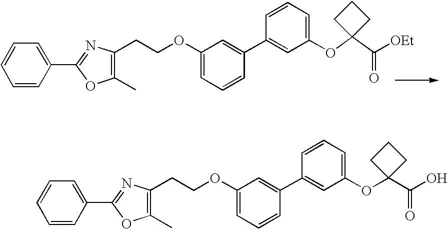

To a solution of ethyl 1-({3′-[2-(5-methyl-2-phenyl-1,3-oxazol-4-yl)ethoxy]-1,1′-biphenyl-3-yl}oxy)cyclobutanecarboxylate (0.138 g, 0.278 mmol) in tetrahydrofuran (3 mL) and methanol (1 mL) was added 2M aqueous lithium hydroxide (0.28 mL). The resulting mixture was stirred at ambient temperature for 16 hours. Water (5 mL) and diethyl ether (10 mL) were added and the resulting solution stirred for 10 minutes. The ethereal layer was removed and the aqueous layer acidified to pH 2 with 1N hydrochloric acid at 0° C. and stirred for 20 minutes. The white precipitate was collected by filtration and washed with ice-cold water. After drying at 40° C. under high vacuum the title compound was afforded as a white crystalline solid (0.091 g, 70%). LRMS: 471 (M+H)⁺. ¹H NMR (CDCl₃, 400 MHz): δ 8.03-8.00 (2H, m), 7.43 (3H, t, J=3.3 Hz), 7.30 (2H, t, J=7.8 Hz), 7.16 (2H, d, J=6.8 Hz), 7.09 (1H, t, J=2.3 Hz), 6.91-6.85 (3H, m), 4.27 (2H, t, J=7.8 Hz), 3.06 (3H, t, J=8.1 Hz), 2.83-2.76 (2H, m), 2.53-2.46 (2H, m), 2.40 (3H, s), 2.06-1.97 (2H, m).

### Large Scale Preparation of 1-({3′-[2-(5-Methyl-2-phenyl-1,3-oxazol-4-yl)ethoxy]-1,1′-biphenyl-3-yl}oxy)cyclobutanecarboxylic acid

Ethyl 1-({3′-[2-(5-methyl-2-phenyl-1,3-oxazol-4-yl)ethoxy]biphenyl-3-yl}oxy) cyclobutane carboxylate (see preparation a-4) (crude, 94 g, 148 mmol) was dissolved in THF/MeOH (600/150 mL) and followed by the addition of 2.0 M LiOH aqueous solution (190 mL). The resulting reaction mixture was heated at 55° C. for 1.5 hours and concentrated to remove organic solvent. The basic aqueous solution was diluted with water (100 mL) and extracted with EtOAc (400 mL) in order to remove non-acidic impurities. However, the lithium salt product also went into the EtOAc layer, which made the purification by extraction impossible. The EtOAc extract was concentrated and the oil residue was treated with 1 M HCl aqueous solution (200 mL). The resulting mixture was extracted with MTBE (2×300 mL). The organic extract was washed with brine, dried over sodium sulfate, and concentrated to give the crude free acid product as brown oil. Purification by silica gel column using 10-60% EtOAc in hexane gave 45.38 g (65%) of light yellow oil. 40 g of the oil was crystallized from MTBE (4 volume) to give 30.87 g of white crystalline solid: ¹H NMR (400 MHz, CDCl₃) δ 1.91 (m, 2 H), 2.30 (s, 3 H), 2.38 (m, 2 H), 2.69 (m, 2 H), 2.92 (t, 2 H), 4.10 (t, 2 H), 6.73 (m, 2 H), 6.82 (s, 1 H), 6.98 (s, 1 H), 7.02 (d, 2 H), 7.18 (m, 2 H), 7.30 (m, 3 H), 7.90 (d, 2 H). LRMS (m/z): 470.1 (M+H)⁺; Melting Point: 127° C.

### EXAMPLES A-5 TO A-28

Examples A-5 to A-28 were prepared using procedures analogous to those described for Example A-4.

| | | | MS (m/z) | |
|---|---|---|---|---|
| Ex. # | Structure | ¹H NMR | (LR or HR) | Analysis |
| A-5 | 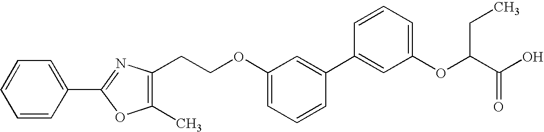 | (CDCl₃, 400 MHz) 7.95(2H, dd, J=2.9, 6.7Hz), 7.38-7.35(3H, m), 7.29-7.22(2H, m), 7.13-7.10(3H, m), 7.07(1H, t, J=2.3Hz), 6.91-6.88(1H, m), 6.81(1H, dd, J=1.8, 8.3Hz), 4.57(1H, t, J=6.2Hz), 4.23(2H, t, J=7.7Hz), 3.05-2.92(2H, m), 2.34(3H, s), 2.03-1.96(2H, m), 1.06(3H, t, J=7.5Hz). | LR 458(M + | |
| #H)⁺ | Calcd for C₂₈H₂₇NO₅.0.41H₂O C 72.34, H 6.03, N 3.01. Found: C 72.33, H 6.01, N 2.95. | | | |
| A-6 | 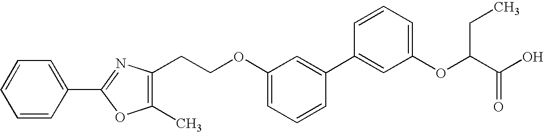 | (DMSO-d₆, 400 MHz)7.91(2H, dd, J=1.8, 7.6Hz), 7.73(1H, t, J=7.8Hz), 7.52-7.44(6H, m), 7.25(1H, t, J=8.0Hz), 6.83(1H, dd, J=2.0, 8.1Hz), 6.72(1H, d, J=8.1Hz), 4.61(2H, t, J=6.7Hz), 4.11(1H, t, J=6.3Hz), 2.98(2H, t, J=6.7Hz), 2.32(3H, s), 1.84-1.69(2H, m), 0.95(3H, t, J=7.3Hz). | LR 459 (M + | |
| #H)⁺ | Calcd for C₂₇H₂₅LiN₂O₅.2.32H₂O C 64.82, H 6.17, N 5.60. Found: C 64.83, H 5.89, N 5.52. | | | |
| A-7 | 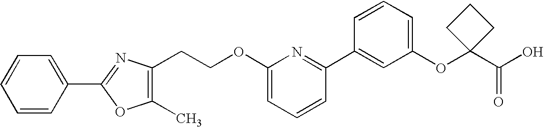 | (CDCl₃, 400 MHz) 8.02-8.00(2H, m), 7.65-7.64(1H, m), 7.61(1H, m), 7.45-7.42(3H, m), 7.40-7.30(3H, m), 7.05(1H, ddd, J=1.0, 2.5, 7.8Hz), 6.65(1H, d, J=8.1Hz), 4.74-4.69(2H, m), 3.10-3.07(2H, m), 2.83-2.76(2H, m), 2.47-2.38(2H, m), 2.40(3H, s), 2.09-1.95(2H, m). | LR 471 (M + H)⁺ | Calcd |
| #for C₂₈H₂₆N₂O₅.0.09LiCl C 70.89, H 5.52, N 5.90. Found: C 70.94, H 5.65, N 5.93. | | | | |
| A-8 | 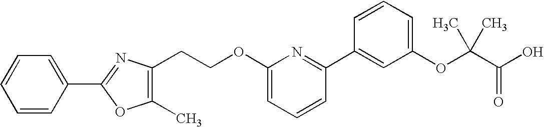 | (CDCl₃, 400 MHz) 8.11(1H, dd, J=1.5, 2.5Hz), 8.00-7.97(2H, m), 7.62(1H, t, J=7.6Hz), 7.46-7.41(4H, m), 7.35(1H, d, J=7.3Hz), 7.31(1H, t, J=7.8Hz), 7.00(1H, ddd, J=1.0, 2.8, 8.1Hz), 6.65(1H, d, J=8.3Hz), 4.74-4.69(2H, m), 3.16-3.12(2H, m), 2.41(3H, s), 1.63(6H, s). | LR 459 (M + H)⁺ | |
| A-9 | 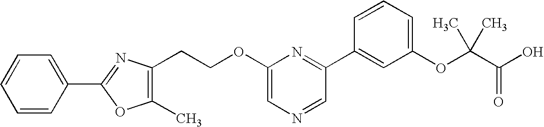 | (CDCl₃, 400 MHz) 8.63(1H, bs), 8.13(1H, bs), 8.06(1H, dd, J=1.3, 2.5Hz), 7.99-7.96(2H, m), 7.52(1H, d, J=7.8Hz), 7.46-7.42(3H, m), 7.35(1H, t, J=7.8Hz), 7.04(1H, dd, J=2.3, 8.1Hz), 4.72-4.68(2H, m), 3.16-3.12(2H, m), 2.43(3H, s), 1.63(6H, s). | LR 460 (M + H)⁺ | Calcd |
| #for C₂₆H₂₅N₃O₅.0.47H₂O C 66.73, H 5.59, N 8.98. Found: C 66.69, H 5.48, N 8.96. | | | | |
| A-10 | 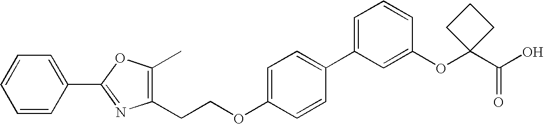 | ¹H NMR(400 MHz, CDCl₃) 1.91-2.02(m, 2H), 2.24-2.31(m, 3H), 2.38-2.49(m, 2H), 2.75(td, 2H), 2.86(t, 2H), 4.04(t, 2H), 6.58(dd, 1H), 6.75-6.86(m, 3H), 7.04(d, 1H), 7.16-7.22(m, 2H), 7.30-7.38(m, 3H), 7.90(dd, 2H). | LRMS (m/z): 470 (M + H)⁺. | |
| A-11 | 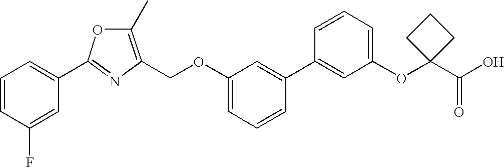 | ¹H NMR(400 MHz, CDCl₃): 1.83-2.05(m, 2H), 2.51(s, 3H), 2.50-2.58(m, 2H), 2.75-2.83(m, 2H), 5.02(s, 2H), 6.52(m, 1H), 6.96-7.56(m, 9H), 1.73-7.86(m, 2H). | | |
| A-12 | 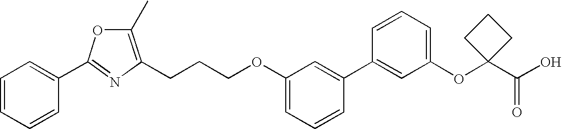 | ¹H NMR(400 MHz, CDCl₃): 1.97-2.07(m, 2H), 2.13-2.23(m, 2H), 2.29(s, 3H), 2.45-2.54(m, 2H), 2.70-2.81(m, 4H), 4.21(q, 2H), 6.63-6.65(m, 1H), 7.07-7.18(m, 2H), 7.24-7.34(m, 4H)7.37-7.45(m, 4H) 7.98(dd, 2H). | LRMS (m/z): 484.5 (M + H)⁺. | |
| A-13 | 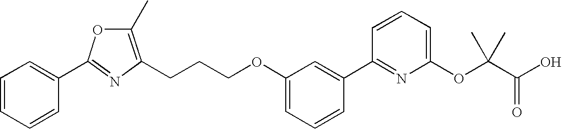 | ¹H NMR(400 MHz, CDCl₃): 1.61(s, 6H), 2.35(s, 3H), 2.78-2.89(m, 4H), 4.23-4.31(m, 2H), 6.77(d, 2H), 7.15(d, 4H), 7.36-7.45(m, 4H), 7.97(dd, 2H). | LRMS (m/z): 473.5 (M + H)⁺ | |
| A-14 | 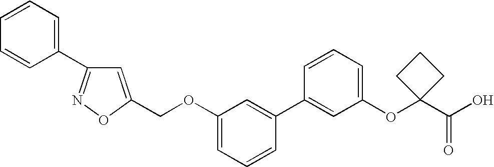 | ¹H NMR(400 MHz, CDCl₃): 1.99-2.11(m, 2H), 2.51(dq, 2H), 2.77-2.85(m, 2H), 5.25(s, 2H), 6.65-6.72(m, 2H), 6.94-6.99(m, 2H), 7.15-7.22(m, 2H), 7.35(t, 2H), 7.42-7.51(m, 2H), 7.80(dd, 4H) | LRMS (m/z): 443.0 (M + H)⁺. | |
| A-15 | 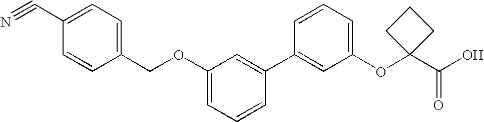 | ¹H NMR(400 MHz, CDCl₃): 1.98-2.08(m, 2H), 2.45-2.55(m, 2H), 2.74-2.83(m, 2H), 5.18(s, 2H), 6.91-6.98(m, 1H)7.13-7.21(m, 2H)7.26-7.37(m, 5H), 7.56-7.63(m, 2H), 7.69(d, 2H). | LRMS (m/z): 423 (M + H)⁺. | |
| A-16 | 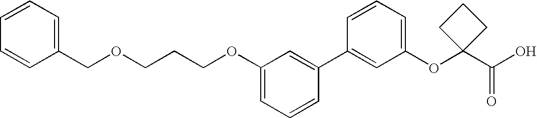 | ¹H NMR(400 MHz, CDCl₃): 1.96-2.07(m, 2H), 2.48-2.51(m, 2H), 2.73-2.82(m, 2H), 4.31-4.40(m, 4H), 6.63(dd, 1H), 6.92-7.00(m, 5H), 7.13-7.21(m, 3H), 7.25-7.36(m, 4H) | LRMS (m/z): 423 (M + H)⁺. | |
| A-17 | 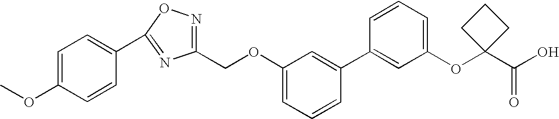 | ¹H NMR(400 MHz, CDCl₃): 1.96-2.07(m, 2H), 2.44-2.55(m, 2H), 2.74-2.82(m, 2H), 3.89(s, 1H), 5.25-5.31(s, 2H), 6.63-6.65(m, 1H), 7.00-7.10(m, 4H), 7.17(t, 2H), 7.23-7.31(m, 4H), 8.12(d, 2H). | LRMS (m/z): 473.5 (M + H)⁺. | |
| A-18 | 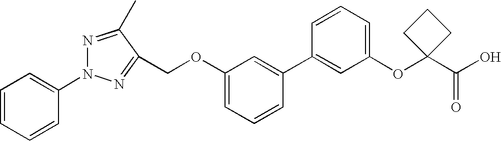 | ¹H NMR(400 MHz, CDCl₃): 2.02-2.14(m, 2H)2.44-2.54(m, 5H)2.75-2.86(m, 2H)5.26(s, 2H)6.95-7.05(m, 1H)7.16-7.23(m, 2H)7.25-7.36(m, 6H)7.46(t, 2H)8.02(d, 2H). | LRMS (m/z): 456.5 (M + H)⁺. | |
| A-19 | 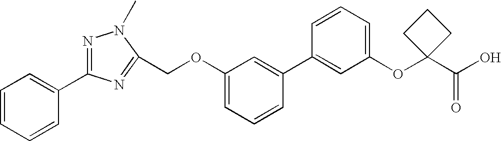 | ¹H NMR(400 MHz, CDCl₃): 1.96-2.07(m, 2H), 2.44-2.54(m, 5H), 2.74-2.82(m, 2H), 5.25(s, 2H), 6.96-7.04(m, 1H), 7.16(d, 2H), 7.22-7.28(m, 2H), 7.32(td, 4H), 7.42-7.49(m, 2H), 7.99-8.05(m, 2H) | LRMS (m/z): 456.1 (M + H)⁺. | |
| A-20 | 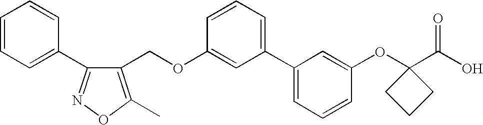 | ¹H NMR(400 MHz, CDCl₃): 1.97-2.07(m, 2H), 2.52(s, 3H), 2.64-2.65(m, 2H), 2.74-2.82(m, 2H), 4.89(s, 2H), 6.68-6.70(m, 1H), 6.83-6.85(m, 2H), 7.24-7.30(m, 3H)7.41-7.47(m, 3H), 7.58-7.62(m, 2H), 7.66-7.68(m, 2H). | LRMS (m/z): 456.2 (M + H)⁺ | |
| A-21 | 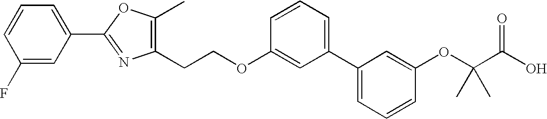 | ¹H NMR(400 MHz, CDCl₃) 1.56-1.65(m, 6H), 2.43(s, 3H), 3.09(t, 2H), 4.33(t, 2H), 6.86-6.96(m, 2H), 7.11-7.19(m, 2H), 7.20-7.26(m, 4H), 7.32(t, 2H), 7.41(td, 1H), 7.70(ddd, 1H)7.81(d, 1H). | LRMS (m/z): 476 (M + H)⁺. | |
| A-22 | 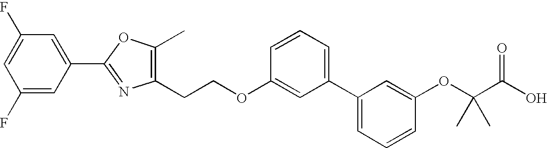 | ¹H NMR(400 MHz, CDCl₃) 1.64(s, 6H), 2.41(s, 3H), 3.06(t, 2H), 4.31(t, 2H), 6.88(dd, 3H), 7.04-7.13(m, 1H), 7.14-7.23(m, 2H), 7.23-7.34(m, 3H), 7.52(dd, 2H). | LRMS (m/z): 494 (M + H)⁺. | |
| A-23 | 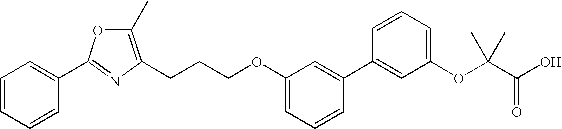 | ¹H NMR(400 MHz, CDCl₃): 1.71(s, 6H), 2.10-2.22(m, 2H), 2.35(s, 3H), 2.67-2.77(m, 2H), 3.99-4.09(m, 2H), 6.67-6.69(m, 2H), 7.04-7.13(m, 2H)7.20-7.32(m, 4H)7.37-7.44(m, 2H)7.92-8.02(m, 3H). | LRMS (m/z): 472.5 (M + H)⁺ | |
| A-24 | 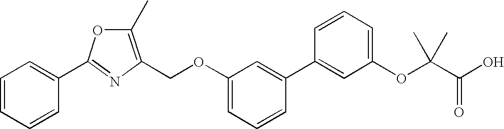 | ¹H NMR(400 MHz, CDCl₃): 1.76(s, 6H), 2.49(s, 3H), 5.16(s, 2H), 7.07-7.19(m, 2H), 7.23(t, 1H), 7.32(td, 4H), 7.36-7.47(m, 4H), 7.98(dd, 2H) | LRMS (m/z): 444.5 (M + H)⁺ | |
| A-25 | 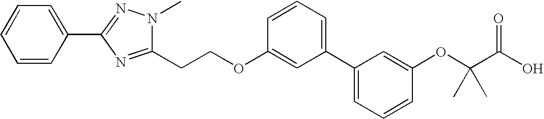 | ¹H NMR(400 MHz, CDCl₃) 1.58(s, 6H), 3.35(t, 2H), 3.99(s, 3H), 4.40(t, 2H), 6.80(d, 1H), 6.81(dd, 1H), 7.00(d, 1H), 7.11-7.20(m, 3H), 7.25(m, 2H), 7.33-7.38(m, 3H), 7.92-8.00(m, 2H). | LRMS (m/z): 458 (M + H)⁺. | |
| A-26 | 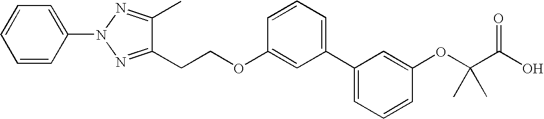 | ¹H NMR(400 MHz, CDCl₃) 1.56(s, 6H), 2.31(s, 3H), 3.11(t, 2H), 4.25(t, 2H), 6.81(m, 2H), 6.96(s, 1H), 7.06(m, 2H), 7.20(m, 4H), 7.30(t, 2H), 7.82(d, 2H). | LRMS (m/z): 458 (M + H)⁺. | |
| A-27 | 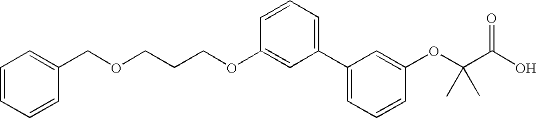 | ¹H NMR(400 MHz, CDCl₃) 1.62(s, 6H), 2.07-2.15(m, 2H), 3.60-3.71(m, 2H), 4.13(q, 2H), 4.53(s, 2H), 6.83-6.93(m, 2H), 7.01-7.09(m, 1H), 7.12(d, 1H), 7.14-7.20(m, 1H), 7.30(ddd, 8H). | LRMS (m/z): 421 (M + H)⁺. | |
| A-28 | 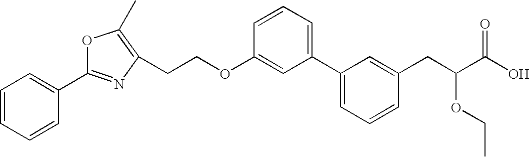 | ¹H NMR(400 MHz, CDCl₃): 1.18(t, 3H), 2.43(s, 3H), 2.96-3.08(m, 4H), 3.32-3.35(m, 2H), 3.62-3.65(m, 1H), 4.24-4.33(m, 2H), 7.07-7.19(m, 2H), 7.23(t, 1H), 7.32(td, 4H), 7.36-7.47(m, 4H), 7.98(dd, 2H). | LRMS (m/z): 472.5 (M + H)⁺. | |

### Preparations of Starting Materials for Examples A-1 to A-28 (Preparations a-1 to a-11)

### Preparation a-1

### Methyl 2-(3-iodophenoxy)-2-methylpropanoate

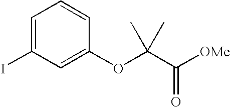

To a solution of 3-iodophenol (1.08 g, 4.9 mmol) in N,N-dimethylformamide (10 mL) was added methyl 2-bromo-2-methyl-propionate (0.76 mL, 1.2 equiv) and cesium carbonate (3.45 g, 2 equiv). The resulting mixture was heated at 90° C. for 24 hours and then allowed to cool to ambient temperature. Water was introduced and the mixture extracted with diethyl ether (3×20 mL). The combined organics were washed with water and saturated aqueous sodium chloride, dried (anhydrous sodium sulfate), filtered and concentrated. The residue was purified by silica gel chromatography using 0-30% ethyl acetate in hexanes to provide the title compound (0.83 g, 53%). LRMS: 321 (M+H)⁺. ¹H NMR (CDCl₃, 400 MHz): δ 7.22 (1H, dt, J=1.3, 7.8 Hz), 7.12 (1H, dd, J=1.6, 2.4 Hz), 6.84 (1H, t, J=8.1 Hz), 6.66 (1H, ddd, J=0.8, 2.5, 8.3 Hz), 3.66 (3H, s), 1.47 (6H, s).

### Preparation a-2

### Methyl 2-(3-iodophenoxy)butanoate

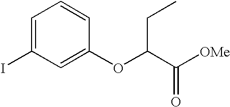

Following the procedure described in Preparation a-1, using ethyl 2-bromopropionate in place of methyl 2-bromo-2-methyl-propionate at ambient temperature, the title compound was obtained in 93% yield. LRMS: 321 (M+H)⁺. ¹H NMR (CDCl₃, 400 MHz): δ 7.30 (1H, ddd, J=1.0, 1.5, 7.8 Hz), 7.24 (1H, dd, J=1.6, 2.4 Hz), 6.97 (1H, dd, J=7.8, 8.3 Hz), 6.82 (1H, ddd, J=1.0, 2.5, 8.6 Hz), 4.53 (1H, dd, J=5.8, 6.6 Hz), 3.75 (3H, s), 2.00-1.93 (2H, m), 1.05 (3H, t, J=7.5 Hz).

### Preparation a-3

### Ethyl1-(3-bromophenoxy)cyclobutanecarboxylate

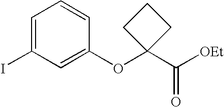

Following the procedure described in Preparation a-1, using 3-bromophenol and ethyl 1-bromocyclobutanecarboxylate as starting materials and heating in a solution of acetonitrile, the title compound was obtained in 56% yield. LRMS: 300 (M+H)⁺.

### Preparation a-4

### Ethyl 1-({3′-[2-(5-methyl-2-phenyl-1,3-oxazol-4-yl)ethoxy]biphenyl-3-yl}oxy)cyclo-butanecarboxylate

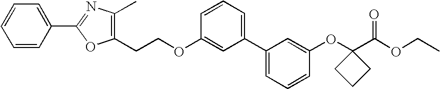

A mixture of ethyl 1-[(3′-hydroxybiphenyl-3-yl)oxy]cyclobutanecarboxylate (46.24 g, 148 mmol), 2-(5-methyl-2-phenyl-1,3-oxazol-4-yl)ethyl-4-methylbenzene-sulfonate (57.30 g, 160 mmol), and Cs₂CO₃ (62.94 g, 193 mol) in anhydrous acetonitrile (500 mL) was heated at 50° C. under nitrogen. After 8 hours, more 2-(5-methyl-2-phenyl-1,3-oxazol-4-yl)ethyl-4-methylbenzene-sulfonate (21.16 g, 59.2 mmol) was added and the heating temperature was raised to 60° C. After 17 hours, another portion of 2-(5-methyl-2-phenyl-1,3-oxazol-4-yl)ethyl-4-methylbenzene-sulfonate (5.28 g, 14.8 mmol) was added and heating was continued for 5 more hours. The solvent was removed and the residue was partitioned between brine (500 mL) and EtOAc (600 mL). The separated organic layer was washed with aqueous Na₂CO₃, water, and brine. The organic layer was dried over sodium sulfate and concentrated to give the crude product as dark brown oil (94 g, >100%), which was carried to the next step without purification. ¹H NMR (400 MHz, CDCl₃) δ 1.10 (t, 3 H), 1.91 (m, 2 H), 2.31 (s, 3 H), 2.38 (m, 2 H), 2.69 (m, 2 H), 2.92 (t, 2 H), 4.10 (q, 2 H), 4.19 (t, 2 H), 6.51 (d, 1 H), 6.79 (d, 1 H), 6.85 (s,1 H), 7.00 (s, 1 H), 7.05 (m, 2 H), 7.20 (m, 2 H), 7.31 (M, 3 H), 7.79 (m, 2 H). LRMS (m/z): 498.1 (M+H)⁺.

### Preparation a-4-1

### Ethyl 1-[(3′-hydroxybiphenyl-3-yl)oxy]cyclobutanecarboxylate

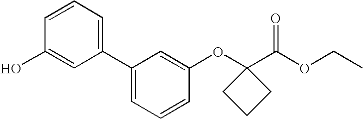

A mixture of ethyl 1-{[3′-(benzyloxy)biphenyl-3-yl]oxy}cyclobutanecarboxylate (65.43 g, 163 mmol) and 10% Pd/C (14.0 g) in EtOH/THF (600/120 mL) was reacted with hydrogen at room temperature under 50 psi on a Parr shaker for 2.5 days. The mixture was filtered through a pad of Celite and the cake was rinsed with EtOH. The filtrate was concentrated to dryness to give 46.24 g (91%) of colorless oil: ¹H NMR (400 MHz, CDCl₃) δ 1.11 (t, 3 H), 1.90 (m, 2 H), 2.41 (m, 2 H), 2.66 (m, 2 H), 4.10 (q, 2 H), 5.29 (br, 1 H), 6.59 (d, 1 H), 6.72 (d, 1 H), 6.88 (s,1 H), 6.92 (s, 1 H), 7.02 (d, 1 H), 7.08 (d, 1 H), 7.15 (m, 2 H). LRMS (m/z): 313.0 (M+H)⁺.

### Preparation a-4-2

### Ethyl 1-{[3′-(benzyloxy)biphenyl-3-yl]oxy}cyclobutanecarboxylate

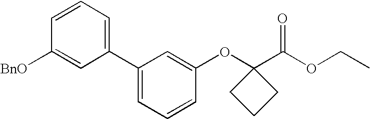

A mixture of ethyl 1-(3-iodophenoxy)cyclobutanecarboxylate (55.64 g, 161 mmol), 3-benzyloxyphenylboronic acid (40.39 g, 177 mmol), Pd(PPh₃)₄ (9.95 g, 8.61 mmol), and Na₂CO₃ (36.62 g, 345 mmol) in a solvent system of toluene/water/ethanol (240/120/60 mL) was purged with nitrogen for 10 minutes and heated at reflux under nitrogen for 17 hours. Organic solvent was removed and the residue was partitioned between brine (400 mL) and EtOAc (600 mL). The separated organic layer was washed with dilute NaOH and brine, dried over sodium sulfate, and concentrated to give 88.27 g of brown oil. Purification by silica gel column using EtOAc/hexane (1/9) afforded 44.53 g (69%) of colorless oil: ¹H NMR (400 MHz, CDCl_{3) δ} 1.10 (t, 3 H), 1.81 (m, 2 H), 2.26 (m, 2 H), 2.56 (m, 2 H), 4.02 (q, 2 H), 4.92 (s, 2 H), 6.40 (d, 1 H), 6.80 (m, 2 H), 6.96 (m, 3 H), 7.10 (m, 1 H), 7.13 (m, 2 H), 7.20 (t, 2 H), 7.28 (d, 2 H). LRMS (m/z): 403.1 (M+H)⁺.

### Preparation a-4-3

### Ethyl 1-(3-iodophenoxy)cyclobutanecarboxylate

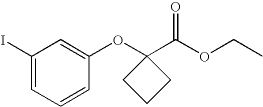

A mixture of 3-iodophenol (42.5 9, 193 mol), ethyl 1-bromocyclobutane-carboxylate (60.0 g, 290 mol), and Cs₂CO₃ (157.0 g, 482 mol) in anhydrous acetonitrile (300 mL) was heated at 55° C. under nitrogen. After 8 hours, more Cs₂CO₃ (31.4 g, 96.4 mmol) was added and the heating temperature was raised to 65° C. After 3 days, there was still phenol SM. Two more portions of Cs₂CO₃ (31.4 g, 96.4 mmol each) were added with 8 hour time interval. After heating for a total of 5 days, there was about 10% 3-iodophenol starting material left. The reaction mixture was concentrated and the residue was partitioned between water (600 mL) and EtOAc (800 mL). The separated organic layer was washed with 5 M NaOH until there was no phenol in the organic layer. The Organic layer was washed with water, dried over sodium sulfate, and concentrated to give 55.64 g (83%) of brown oil that was carried to the next step without further purification. ¹H NMR (400 MHz, CDCl_{3) δ} 1.10 (t, 3 H), 1.85 (m, 2 H), 2.26 (m, 2 H), 2.56 (m, 2 H), 4.02 (q, 2 H), 6.48 (d, 1 H), 6.80 (t, 1 H), 6.92 (s, 1 H), 7.12 (d, 1 H). LRMS (m/z): 347.0 (M+H)⁺.

### Preparation a-5

### 2-Bromo-6-[2-(5-methyl-2-phenyl-1,3-oxazol-4-yl)ethoxy]pyridine

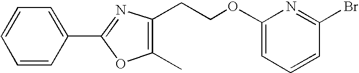

To a solution of 2-(5-methyl-2-phenyl-1,3-oxazol-4-yl)-ethanol (1.04 g, 5.1 mmol) and 2,6-dibromopyridine (1.21 g, 5.1 mmol) in anhydrous dioxane (20 mL) at 0° C. was added sodium hydride (60% in oil, 0.368 g 3 equiv). The resulting mixture was stirred at ambient temperature for 16 hours. The mixture was poured into ice-cold water and extracted with ethyl acetate (3×50 mL). The combined organics were washed with saturated aqueous sodium bicarbonate and saturated aqueous sodium chloride, dried (anhydrous sodium sulfate), filtered and concentrated. The residue was purified by silica gel chromatography using 0-50% ethyl acetate in hexanes to afford the title compound as a white crystalline solid (1.19 g, 65%). LRMS: 359 (M+H)⁺. ¹H NMR (CDCl₃, 400 MHz): 7.97 (2H, dd, J=1.8, 7.8 Hz), 7.44-7.35 (4H, m), 7.03 (1H, d, J=7.3 Hz), 6.65 (1H, d, J=8.1 Hz), 4.55 (2H, t, J=6.8 Hz), 2.97 (2H, t, J=6.8 Hz), 2.34 (3H, s).

### Preparation a-6

### 2-Chloro-6-[2-(5-methyl-2-phenyl-1,3-oxazol-4-yl)ethoxy]pyrazine

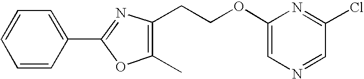

Following the procedure described in Preparation a-5, starting from 2-(5-methyl-2-phenyl-1,3-oxazol-4-yl)-ethanol and 2,6 dichloropyrazine, the title compound was obtained in 64% yield. LRMS: 316 (M+H)⁺. ¹H NMR (CDCl₃, 400 MHz): δ 8.11 (2H, d, J=10.4 Hz), 7.97 (2H, dd, J=1.9, 7.7 Hz), 7.44-7.39 (3H, m), 4.60 (2H, t, J=6.7 Hz), 2.99 (2H, t, J=6.7 Hz), 2.35 (3H, s).

### Preparation a-7

### Methyl 2-[3-(4,4,5,5-tetramethyl-1,3,2-dioxaborolan-2-yl)phenoxy]butanoate

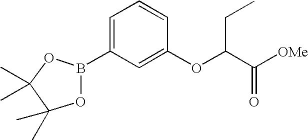

To a solution of methyl 2-(3-iodophenoxy)-2-methylpropanoate (Preparation a-2) (1.49 g, 4.65 mmol) in dimethylsulfoxide (40 mL) was added potassium acetate (1.37 g, 3 equiv), bis(pinacolato)diboron (1.3 g, 1.1 equiv) and a solution of [1,1′-bis(diphenylphosphino)-ferrocene]dichloropalladium (II) complex (0.152 g, 0.04 equiv) in dichloromethane. The resulting mixture was heated at 80° C. for 16 hours and allowed to cool to ambient temperature. Water was introduced and the mixture extracted with diethyl ether (3×30 mL). The combined organics were washed with 5% aqueous sodium bicarbonate (2×50 mL) and saturated aqueous sodium chloride, dried (anhydrous sodium sulfate), filtered and concentrated. The residue was purified by silica gel chromatography using 0-25% ethyl acetate in hexanes to provide the title compound (0.92 g, 62%). LRMS: 321 (M+H)⁺. ¹H NMR (CDCl₃, 400 MHz): δ 7.40 (1H, d, J=7.3 Hz), 7.32 (1H, d, J=2.8 Hz), 7.28 (1H, d, J=8.1 Hz), 6.97 (1H, ddd, J=1.0, 2.8, 8.1 Hz), 4.64 (1H, t, J=6.3 Hz), 3.73 (3H, s), 2.01-1.94 (2H, m), 1.32 (12H, s), 1.06 (3H, t, J=7.5 Hz).

### Preparation a-8

### Methyl 2-methyl-2-[3-(4,4,5,5-tetramethyl-1,3,2-dioxaborolan-2-yl)phenoxy]propanoate

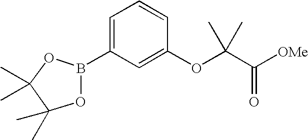

Following the procedure described in Preparation a-7, using methyl 2-(3-iodophenoxy)-2-methylpropanoate (Preparation 1) as starting material, the title compound was produced in 75% yield. LRMS: 321 (M+H)⁺. ¹H NMR (CDCl₃, 400 MHz): δ 7.42 (1H, d, J=7.1 Hz), 7.29 (1H, d, J=2.8 Hz), 7.22 (1H, t, J=7.8 Hz), 6.90 (1H, ddd, J=0.8, 2.8, 8.1 Hz), 3.75 (3H, s), 1.56 (6H, s), 1.30 (12H, s).

### Preparation a-9

### Ethyl 1-[3-(4,4,5,5-tetramethyl-1,3,2-dioxaborolan-2-yl)phenoxy]cyclobutanecarboxylate

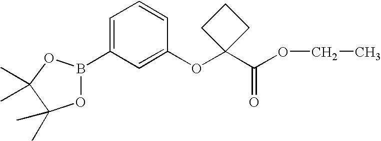

Following the procedure described in Preparation a-7, using ethyl 1-(3-bromophenoxy)cyclobutanecarboxylate as starting material, the title compound was produced in 80% yield. LRMS: 347 (M+H)⁺.

### Preparation a-10

### Methyl 2-{[3′-(benzyloxy)-1,1′-biphenyl-3-yl]oxy}-2-methylpropanoate

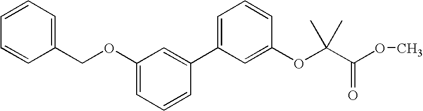

To a solution of methyl 2-(3-iodophenoxy)-2-methylpropanoate (Preparation a-1) (1.14 g, 3.56 mmol) in benzene (20 mL) was added 3-benzyloxyphenylboronic acid (0.89 g, 1.1 equiv), 2M aqueous sodium carbonate (3.56 mL) and tetrakis(triphenylphosphine) palladium (0) (0.2 g, 0.05 equiv). The resulting mixture heated at reflux for 2 hours and allowed to cool to ambient temperature. Water was added and the mixture extracted with diethyl ether (3×20 mL). The combined organics were washed with 5% aqueous sodium bicarbonate and saturated aqueous sodium chloride, dried (anhydrous sodium sulfate) and concentrated. The residue was purified by silica gel chromatography using 0-15% ethyl acetate in hexanes to give the title compound as a colorless oil (1.08 g, 81%). LRMS: 377 (M+H)⁺. ¹H NMR (CDCl₃, 400 MHz): δ 7.47-7.45 (2H, m), 7.39 (2H, t, J=7.3 Hz), 7.33 (2H, t, J=7.8 Hz), 7.28 (1H, t, J=8.0 Hz), 7.21 (1H, ddd, J=1.0, 1.5, 8.1 Hz), 7.17 (1H, dd, J=1.8, 2.3 Hz), 7.14 (1H, dm, J=7.6 Hz), 7.08 (1H, dd, J=1.8, 2.3 Hz), 6.96 (1H, ddd, J=0.8, 2.5, 8.3 Hz), 6.79 (1H, ddd, J=1.0, 2.5, 8.1 Hz), 5.11 (2H, s), 3.78 (3H, s), 1.62 (6H, s).

### Preparation a-11

### Methyl 2-methyl-2-({3′-[2-(5-methyl-2-phenyl-2H-1,2,3-triazol-4-yl)ethoxy]biphenyl-3-yl}oxy)propanoate

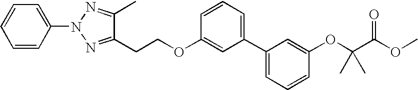

2-(5-Methyl-2-phenyl-2H-1,2,3-triazol-4-yl)ethanol (51 mg, 0.25 mmol), methyl 2-[(3′-hydroxybiphenyl-3-yl)oxy]-2-methylpropanoate (86 mg, 0.30 mmol), and Ph₃P (98 mg, 0.375 mmol) were dissolved in anhydrous THF (1 mL) and followed by the dropwise addition of diethyl azodicarboxylate (65 mg, 0.375 mmol) in anhydrous THF (1 mL) at room temperature via a syringe. The resulting reaction solution was stirred at room temperature for 18 hours and concentrated. Purification by silica gel column with 20-40% EtOAc in hexane afforded 69 mg (59%) of light yellow oil. ¹H NMR (400 MHz, CDCl_{3) δ} 1.55 (s, 6 H), 2.31 (s, 3 H), 3.10 (t, 2 H), 3.68 (s, 3 H), 4.25 (t, 2 H), 6.70 (m, 1 H), 6.82 (m, 1 H), 7.00 (s, 1 H), 7.05 (d, 1 H), 7.20 (m, 4 H), 7.32 (t, 2 H), 7.90 (d, 2 H). LRMS (m/z): 472 (M+H)⁺.

### EXAMPLE B-1

### 1-(3-{[({2-[3-(Trifluoromethyl)phenyl]ethoxy}carbonyl)amino]methyl}phenoxy)cyclobutane carboxylic acid

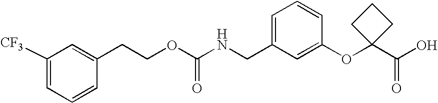

To a solution of ethyl 1-(3-{[({2-[3-(trifluoromethyl)phenyl]ethoxy}carbonyl)amino]methyl}phenoxy)cyclobutanecarboxylate (0.150 g, 0.32 mmol) in tetrahydrofuran (3 mL) and methanol (0.6 mL) at 0° C. was added 2M aqueous lithium hydroxide (0.32 mL, 2 equiv). The resulting mixture was stirred at ambient temperature for 24 hours. Water (10 mL) was added and the mixture was extracted with diethyl ether (1×15 mL, discarded). The aqueous phase was adjusted to pH 2 with 1N hydrochloric acid and extracted with ethyl acetate (3×20 mL). The combined organics were washed with saturated aqueous sodium chloride, dried (anhydrous sodium sulfate), filtered and concentrated to dryness to produce the title compound (85%). LRMS: 438 (M+H)⁺. ¹H NMR (CDCl₃, 400 MHz): δ 7.62 (1H, d, J=7.8 Hz), 7.46 (1H, t, J=7.3 Hz), 7.36 (1H, d, J=7.3 Hz), 7.31 (1H, dd, J=6.1, 7.6 Hz), 7.18 (1H, t, J=8.0 Hz), 6.84 (1H, d, J=7.6 Hz), 6.65 (1H, s), 6.56 (1H, d, J=7.8 Hz), 4.32-4.27 (4H, m), 3.11 (2H, t, J=6.8 Hz), 2.79-2.72 (2H, m), 2.49-2.41 (2H, m), 2.09-1.93 (2H, m).

### EXAMPLES B-2 TO B-29

Examples B-2 to B-4, B-6 and B-29 were prepared by procedures analogous to that used for Example B-1.

| Ex- | | | MS (m/z) |
|---|---|---|---|
| ample # | Structure | ¹H NMR | (LR or HR) |
| B-2 | 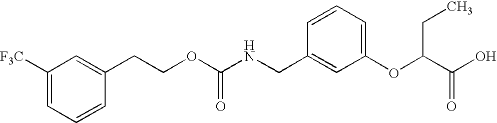 | (CDCl₃, 400 MHz) 7.62(1H, d, J=7.8Hz), 7.46(1H, t, J=7.3Hz), 7.36(1H, d, J=7.6Hz), 7.31(1H, t, J=7.6Hz), 7.22(1H, t, J=7.8Hz), 6.88(1H, d, J=7.3Hz), 6.83(1H, s), 6.78(1H, dd, J=1.9, 8.2Hz), 4.60(1H, t, J=5.8Hz), 4.36-4.29(4H, m), 3.11(2H, t, J=6.8Hz), 2.03-1.97(2H, m), | |
| #1.08(3H, t, J=7.5Hz). | LR 426(M + H)⁺ | | |
| B-3 | 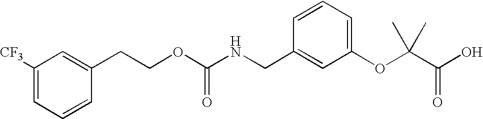 | (CDCl₃, 400 MHz) 7.62(1H, d, J=7.8Hz), 7.46(1H, t, J=7.5Hz), 7.36(1H, d, J=7.6Hz), 7.31(1H, t, J=7.6Hz), 7.21(1H, t, J=7.6Hz), 6.94(1H, d, J=7.3Hz), 6.86(1H, s), 6.81(1H, d, J=7.8Hz), 4.36-4.29(4H, m), 3.11(2H, t, J=7.0Hz), 1.58(6H, s). | LR 426(M + H)⁺ |
| B-4 | 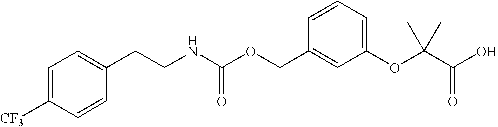 | ¹H NMR(400 MHz, CDCl₃): 1.69(s, 6H), 4.34(d, 2H), 5.05-5.17(m, 1H), 5.23(s, 2H), 6.71(dd, 1H), 6.78(s, 1H), 6.90(d, 1H), 7.18-7.23(m, 1H), 7.26-7.33(m, 1H), 7.69(d, 1H), 7.89(d, 1H), 8.73(s, 1H). | LRMS(m/z): 426.4 (M + H)⁺. |

### EXAMPLE B-5

### 2-Methyl-2-{3-[({[2-(5-methyl-2-phenyl-1,3-oxazol-4-yl)ethoxy]carbonyl}amino)methyl]phenoxy}proranoic acid

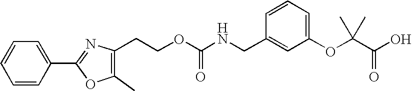

To a solution of methyl 2-methyl-2-{3-[({[2-(5-methyl-2-phenyl-1,3-oxazol-4-yl)ethoxy]carbonyl}amino)methyl]phenoxy}propanoate (0.45 g, 0.99 mmol) in tetrahydrofuran (4.0 mL) and methanol (1.0 mL) was added 2.0 M aqueous LiOH (1.6 mL). The resulting mixture was stirred 8.0 hours in ambient temperature. Concentration and purification by silica gel chromatography using 0-20% methanol in dichloromethane gave the title compound (0.20 g, 0.47 mmol, 47%). Recrystallization from methyl tert-butyl ether and hexane provided crystalline acid. LRMS: 439 (M+H)⁺. ¹H NMR (CDCl₃, 400 MHz): δ 7.96 (2H, dd, J=1.9, 7.7 Hz), 7.44-7.38 (3H, m), 7.16 (1H, t, J=8.0 Hz), 6.89 (1H, d, J=7.3 Hz), 6.77 (1H, s), 6.67 (1H, dd, J=2.3, 8.3 Hz), 4.36 (2H, t, J=6.7 Hz), 4.30 (2H, d, J=5.8 Hz), 2.84 (2H, t, J=6.7 Hz), 2.32 (3H, s), 1.56 (6H, s). M.P: 84° C.

| | | | |
|---|---|---|---|
| B-6 | 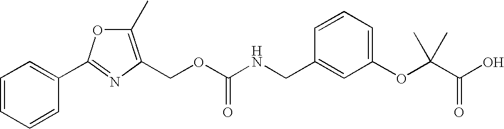 | ¹H NMR(400 MHz, CDCl₃): 1.58(d, 6H), 2.46(s, 3H), 4.32(d, 2H), 5.06(s, 2H), 6.70-6.72(m, 1H), 6.89-6.91(m, 1H), 7.23-7.28(m, 2H), 7.39-7.48(m, 2H), 7.96-8.05(m, 2H). | LRMS(m/z): 423.5(M +H)⁺ |
| B-7 | 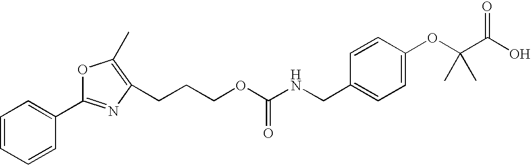 | ¹H NMR(400 MHz, CDCl₃): 1.63(m, 6H), 1.95-2.01(m, 2H), 2.34(s, 3H), 2.56-2.58(m, 2H), 4.11-4.13(m, 2H), 4.32-4.35(d, 2H), 4.89-4.92(b, 1H), 6.82-6.83(m, 2H), 7.21-7.27(m, 3H)7.36-7.44(m, 2H), 7.93-7.96(m, 2H). | LRMS(m/z): 453.5(M +H)⁺. |
| B-8 | 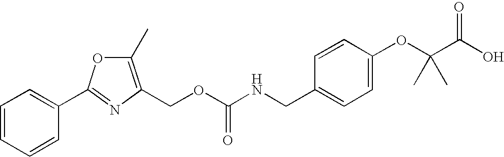 | ¹H NMR(400 MHz, CDCl₃): 1.59(s, 6H), 2.47(s, 3H), 4.24-4.32(m, 2H), 4.99-5.09(m, 3H), 6.73-6.80(m, 2H), 7.15(d, 2H), 7.21-7.27(m, 1H), 7.37-7.46(m, 2H), 7.96-8.04(m, 2H). | LRMS(m/z): 425.5(M +H)⁺ |
| B-9 | 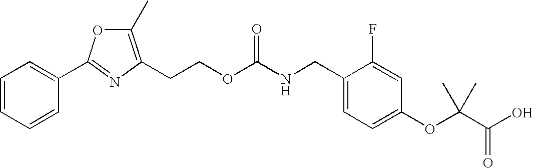 | ¹H NMR(400 MHz, CDCl₃): 1.64(m, 6H), 2.35(s, 3H), 2.82(t, 2H), 4.28-4.38(m, 4H), 6.55(d, 2H), 7.17-7.28(m, 2H), 7.36-7.45(m, 2H), 7.92-8.00(m, 2H) | LRMS(m/z): 457.5(M +H)⁺. |
| B-10 | 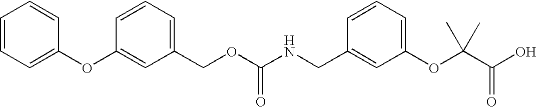 | ¹H NMR(400 MHz, CDCl₃): 1.67(s, 6H), 4.33(d, 2H), 5.03-5.14(m, 3H), 6.69(dd, 1H), 6.78(s, 1H), 6.92(dd, 2H), 7.01(d, 3H), 7.06-7.14(m, 2H), 7.18(t, 1H), 7.25-7.36(m, 3H). | LRMS(m/z): 435.5(M +H)⁺. |
| B-11 | 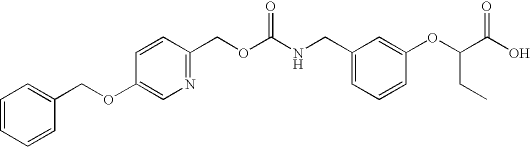 | ¹H NMR(400 MHz, CDCl₃): 1.05-1.16(m, 3H), 2.02-2.04(m, 2H), 4.11-4.13(m, 1H), 4.48-4.58(m, 2H), 4.95-5.96(m, 1H), 5.11(s, 2H), 5.21(s, 2H), 6.75(d, 1H), 6.83-6.93(m, 2H), 7.10-7.21(m, 1H), 7.30-7.41(m, 6H), 8.44(s, 2H). | LRMS(m/z): 451 |
| #(M +H)⁺ | | | |
| B-12 | 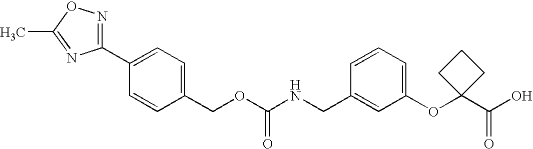 | (CDCl₃, 400 MHz) 8.06-8.00(1H, m), 8.01(1H, d, J=7.6Hz), 7.47-7.42(1H, m), 7.43(1H, d, J=7.8Hz), 7.19(1H, t, J=7.8Hz), 6.87(1H, d, J=7.1Hz), 6.68(1H, s), 6.57(1H, dd, J=2.3, 8.1Hz), 5.17(2H, s), 4.32(2H, d, J=6.1Hz), 2.80-2.75(2H, m), 2.65(3H, s), 2.49-2.41(2H, m), | |
| #2.03-1.97(2H, m). | LR 426 (M +H)⁺ | | |
| B-13 | 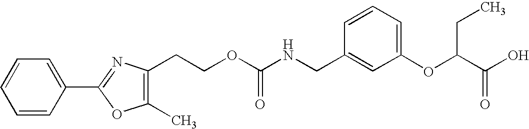 | ¹H NMR(400 MHz, CDCl₃) 0.85-0.95(m, 3H), 1.82(s, 2H), 2.13(s, 3H), 2.62-2.73(m, 2H), 3.85(d, 1H), 3.91(s, 1H), 4.30-4.41(m, 2H), 4.99(s, 1H), 6.58(d, 1H), 6.66(s, 2H), 6.94-7.06(m, 2H), 7.22(s, 3H), 7.70-7.81(m, 2H). | LRMS(m/z): 439 (M +H)⁺. |
| B-14 | 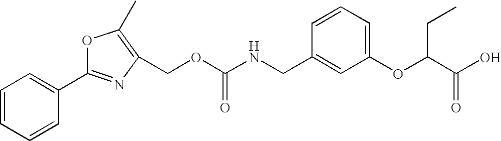 | ¹H NMR(400 MHz, CDCl₃): 1.93-2.01(m, 2H), 2.46(s, 3H), 4.34(d, 2H), 4.56-4.57(m, 1H), 5.06(s, 3H), 6.74-6.76(m, 1H), 6.82-6.84(m, 1H), 6.89-6.91(m, 1H), 7.19-7.28(m, 2H), 7.41-7.46(m, 2H), 7.98-8.04(m, 2H) | LRMS(m/z): 425.5 (M +H)⁺ |
| B-15 | 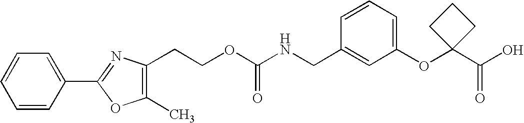 | | LR 451 (M +H)⁺ |
| B-16 | 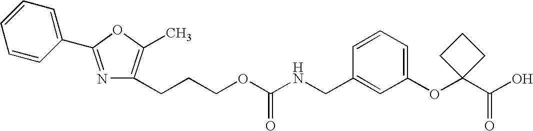 | (CDCl₃, 400 MHz) 7.96-7.94(2H, m), 7.42-7.40(3H, m), 7.19(1H, t, J=7.7Hz), 6.79(2H, d, J=7.6Hz), 6.60(1H, s), 4.32(1H, d, J=6.1Hz), 4.20(2H, s), 2.81-2.75(2H, m), 2.62-2.59(1H, m), 2.49-2.41(3H, m), 2.32-2.28(2H, m), 2.08(3H, s), 2.00-1.92(3H, m). | LR 465 (M +H)⁺ |
| B-17 | 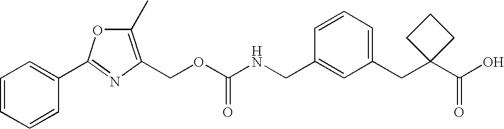 | ¹H NMR(400 MHz, CDCl₃): 1.94-2.05(m, 2H), 2.38-2.48(m, 5H), 2.68-2.76(m, 2H), 4.31(d, 2H), 5.06(s, 3H), 6.52-6.53(m, 1H), 6.65-6.67(m, 1H), 6.83-6.85(m, 1H), 7.16-7.17(m, 1H), 7.41-7.46(m, 3H), 7.99-8.03(m, 2H). | LRMS (m/z): 411.4 (M +H)⁺ |
| B-18 | 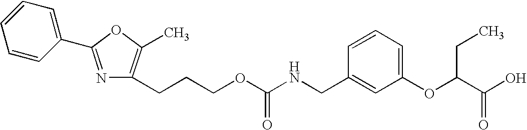 | | LR 453 (M +H)⁺ |
| B-19 | 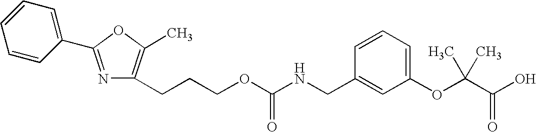 | | LR 453 (M +H)⁺ |
| B-20 | 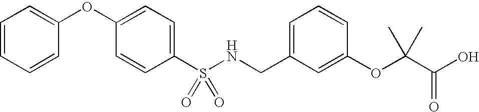 | ¹H NMR(400 MHz, CDCl₃) 1.51(s, 6H), 4.02(d, 2H), 5.12(s, 1H), 6.72-6.80(m, 2H), 6.89-6.96(m, 2H), 6.96-7.02(m, 2H), 7.09(t, 1H), 7.29(s, 2H), 7.31-7.39(m, 2H), 7.64-7.73(m, 2H). | LRMS(m/z): 442 (M +H)⁺. |
| B-21 | 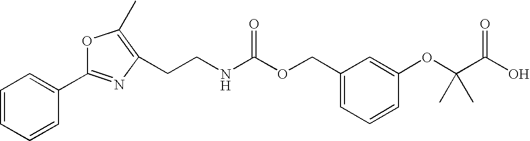 | ¹H NMR(400 MHz, CDCl₃): 1.62(s, 6H), 2.30(s, 3H), 2.69-2.71(m, 2H), 3.49-3.58(m, 2H), 5.05(s, 2H), 5.36-5.38(m, 1H), 6.73-6.75(m, 1H), 6.97-6.99(m, 1H), 7.19-7.28(m, 3H), 7.38-7.48(m, 1H), 7.96-7.98(m, 2H) | LRMS(m/z): 439.5 (M +H)⁺. |
| B-22 | 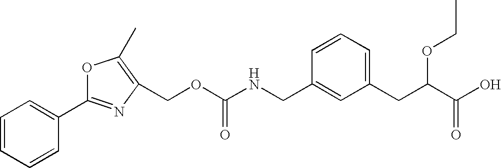 | ¹H NMR(400 MHz, CDCl₃): 1.19-1.26(m, 3H), 2.46(s, 3H), 2.93-3.02(m, 2H), 3.26-3.29(m, 2H), 3.99-4.01(m, 1H), 4.36(d, 2H), 5.06(s, 3H), 7.15(m, 3H), 7.40-7.47(m, 3H), 7.98-8.04(m, 2H). | LRMS(m/z): 439.5 (M +H)⁺. |
| B-23 | 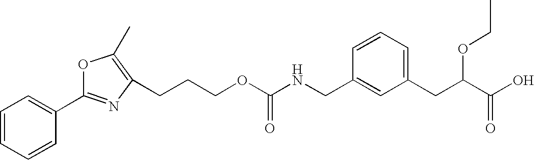 | ¹H NMR(400 MHz, CDCl₃): 1.21-1.27(m, 3H), 1.97-2.06(m, 2H), 2.34(s, 3H), 2.57(t, 2H), 2.95-3.03(m, 2H), 3.34-3.59(m, 2H), 4.11-4.20(m, 4H), 4.35(d, 2H), 4.96-4.98(m, 1H), 7.11-7.18(m, 3H), 7.21-7.27(m, 3H), 7.37-7.45(m, 2H), 7.93-7.99(m, 2H). | LRMS(m/z): 467.5 (M +H)⁺ |
| B-24 | 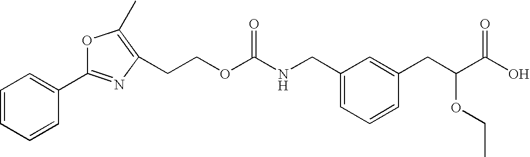 | ¹H NMR(400 MHz, CDCl₃): 1.26-1.28(m, 3H), 2.35(s, 3H), 2.86-2.88(m, 2H), 2.95-3.06(m, 1H), 3.47-3.52(m, 2H), 4.18-4.20(m, 2H), 4.29-4.40(m, 4H), 4.96-4.99(m, 1H), 7.10-7.18(m, 2H), 7.19-7.28(m, 3H), 7.35-7.46(m, 2H), 7.92-7.99(m, 2H). | LRMS(m/z): 453.5 (M +H)⁺. |
| B-25 | 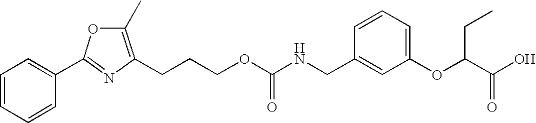 | ¹H NMR(400 MHz, CDCl₃): 1.07(t, 3H), 1.94-2.05(m, 3H), 2.17(s, 3H), 2.57(t, 2H), 4.15(t, 1H), 4.17(d, 2H), 4.33(d, 2H), 4.58(m, 1H), 6.83(m, 1H), 6.89(m, 1H), 6.95(m, 1H), 7.21-7.27(m, 3H), 7.37-7.44(m, 2H), 7.94-7.99(m, 1H). | LRMS(m/z): 453.5 (M +H)⁺ |
| B-26 | 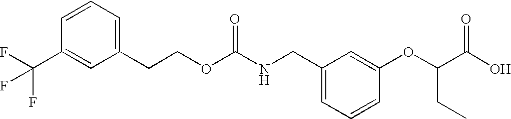 | ¹H NMR(400 MHz, CDCl₃): 1.65(s, 6H), 2.88(d, 2H), 3.46(s, 2H), 5.03(d, 3H), 6.73(s, 1H), 6.83(d, 1H), 6.89-7.00(m, 1H), 7.19-7.31(m, 3H), 7.55(t, 2H) | LRMS(m/z): 426.4 (M +H)⁺. |
| B-27 | 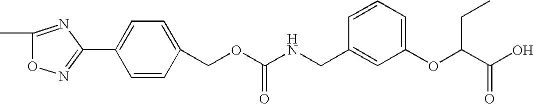 | CDCl₃): 1.07(t, 3H), 1.98(dq, 2H), 2H), 4.57-4.59(m, 1H), 5.02-5.03(m, 8(m, 1H), 6.89(m, 1H), 6.95(m, 1H), 7.48(d, 2H), 8.06(d, 2H). | LRMS(m/z): 426.5 (M +H)⁺. |
| B-28 | 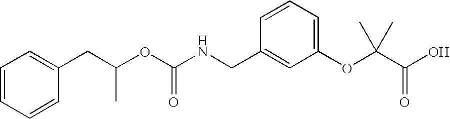 | ¹H NMR(400 MHz, CDCl₃): 1.21(m, 3H), 1.57(s, 6H), 2.63-2.84(m, 2H), 4.22(d, 2H), 4.83-4.85(b, 1H), 5.23-5.25(m, 1H), 6.84-6.87(m, 3H), 7.13(dt, 2H)7.18 7.23(m, 4H). | LRMS(m/z): 372.4 (M +H)⁺. |
| B-29 | 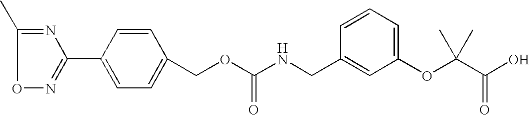 | ¹H NMR(400 MHz, CDCl₃): 1.78(s, 6H), 2.66(s, 3H), 4.34(d, 2H), 4.85-4.87(b, 1H), 5.05-5.06(s, 2H), 6.53-6.54(m, 1H), 6.67-6.69(m, 1H), 6.83-6.85(m, 1H), 7.13(dt, 1H) 7.18-7.23(m, 2H), 7.95-7.97(m, 2H). | LRMS(m/z): 426.5 (M +H)⁺. |

### Preparations of Starting Materials for Exampies B-1 to B-29 (Preparations b-1 to b-20)

### Preparation b-1

### Methyl 2-(3-cyanophenoxy)-2-methylpropanoate

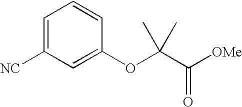

To a solution of 3-cyanophenol (5 mmol) in acetonitrile (20 mL) or any polar, aprotic solvent such as dimethyl sulfoxide, N,N-dimethylformamide, etc) was added methyl 2-bromo-2-methyl-propanoate (1.2 equiv) and cesium carbonate (2 equiv). The resulting was mixture heated at 60° C. for 6 hours and then cooled to ambient temperature. Water (20 mL) was introduced and the mixture extracted with ethyl acetate (3×20 mL). The combined organics were washed with saturated aqueous sodium bicarbonate and saturated aqueous sodium chloride, dried (anhydrous sodium sulfate), filtered, and evaporated to dryness to provide the title compound in 75% yield. LRMS: 220 (M+H)⁺. ¹H NMR (CDCl₃, 400 MHz): δ 7.30 (1H, t, J=8.0 Hz), 7.23 (1H, dt, J=1.3, 7.6 Hz), 7.05 (1H, dd, J=1.3, 2.3 Hz), 7.01 (1H, ddd, J=2.3, 2.8, 8.3 Hz), 3.73 (3H, s), 1.57 (6H, s).

### Preparations b-2 to b-3

Preparations b-2 to b-3 were prepared using procedures analogous to those described for preparation b-1

| | | | MS (m/z) |
|---|---|---|---|
| Preparation # | Structure | ¹H NMR | (LR or HR) |
| 2 | 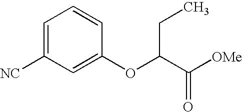 | (CDCl₃, 400 MHz) 7.33(1H, dd, J=7.6, 8.8Hz), 7.22(1H, dt, J=1.3, 7.6Hz), 7.09-7.06(2H, m), 4.56(1H, dd, J=5.6, 6.6Hz), 3.73(3H, s), 2.01-1.93(2H, m), 1.03(3H, t, J=7.5Hz). | LR 220 (M + H)⁺ |
| 3 | 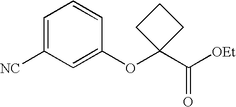 | (CDCl₃, 400 MHz) 7.31(1H, dd, J=7.6, 9.1Hz), 7.22(1H, dt, J=1.0, 7.8Hz), 6.91-6.88(2H, m), 4.20(2H, q, J=7.2Hz), 2.78-2.71(2H, m), 2.48-2.40(2H, m), 2.08-1.96(2H, m), 1.18(3H, t, J=7.2Hz). | LR 246 (M+ H)⁺ |

### Preparation b-4

### Methyl 2-[3-(aminomethyl)phenoxy]-2-methyloropanoate

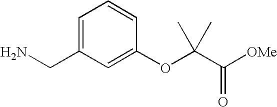

To a solution of methyl 2-(3-cyanophenoxy)-2-methylpropanoate (Preparation b-1) (4 mmol) in methanol (20 mL) was added 10% palladium on carbon (20% by weight). The resulting mixture was stirred under an atmosphere of hydrogen for 24 hours and filtered through Celite. The filtrate was concentrated and the residue taken up in ethyl acetate and washed with 1N hydrochloric acid (2×20 mL). The combined aqueous washes were adjusted to pH >10 with 4N aqueous sodium hydroxide and extracted with dichloromethane (3×20 mL). The combined organic extracts were washed with saturated aqueous sodium chloride, dried (potassium carbonate), filtered and concentrated to dryness to provide the title compound in 65% yield.

LRMS: 224 (M+H)⁺.

### Preparation b-5

### Methyl 2-methyl-2-{3-[({[2-(5-methyl-2-phenyl-1,3-oxazol-4-yl)ethoxy]carbonyl}amino)methyl]phenoxy}propanoate

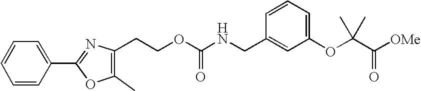

To a solution of 2-(5-methyl-2-phenyl-1,3-oxazol-4-yl)ethyl 1H-imidazole-1-carboxylate (0.48 g, 1.6 mmol) in tetrahydrofuran (3 mL) was added methyl 2-[3-(aminomethyl)phenoxy]-2-methylpropanoate (0.39 g, 1.1 equiv). The resulting mixture was heated at reflux for 16 hours and then cooled to ambient temperature. Concentration and purification by silica gel chromatography using 0-50% ethyl acetate in hexanes gave the title compound (0.39 g, 65%). LRMS: 453 (M+H)⁺. ¹H NMR (CDCl₃, 400 MHz): δ 7.96 (2H, dd, J=1.9, 7.7 Hz), 7.44-7.38 (3H, m), 7.16 (1H, t, J=8.0 Hz), 6.89 (1H, d, J=7.3 Hz), 6.77 (1H, s), 6.67 (1H, dd, J=2.3, 8.3 Hz), 4.36 (2H, t, J=6.7 Hz), 4.30 (2H, d, J=5.8 Hz), 3.75 (3H, s), 2.83 (2H, t, J=6.7 Hz), 2.32 (3H, s), 1.57 (6H, s).

### Preparation b-5-1

### 2-(5-Methyl-2-phenyl-1,3-oxazol-4-yl)ethyl 1H-imidazole-1-carboxylate

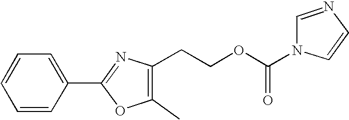

To a solution of 2-(5-methyl-2-phenyl-1,3-oxazol-4-yl)-ethanol (1.015 g, 5 mmol) in toluene (25 mL) was added potassium carbonate (1.38 g, 2 equiv) and N,N′-carbonyldiimidazole (0.97 g, 1.2 equiv). The resulting mixture was stirred at ambient temperature for 16 hours before water (20 mL) was introduced. Extraction with ethyl acetate and washing the combined organic extracts with saturated aqueous sodium chloride, drying (anhydrous sodium sulfate), filtration, and concentration to dryness afforded the title compound (100%). LRMS: 298 (M+H)⁺. ¹H NMR (CDCl₃, 400 MHz): δ 8.10 (1H, s), 7.97-7.93 (2H, m), 7.44-7.39 (5H, m), 4.68 (2H, t, J=6.7 Hz), 2.98 (2H, t, J=6.7 Hz), 2.34 (3H, s).

### Preparation b-5-2

### Methyl 2-[3-(aminomethyl)phenoxy]-2-methylpropanoate

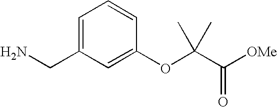

To a solution of methyl 2-(3-cyanophenoxy)-2-methylpropanoate (Preparation 1) (4 mmol) in methanol (20 mL) was added 10% palladium on carbon (10% by weight). The resulting mixture was stirred under an atmosphere of hydrogen for 16 hours and filtered through Celite. The filtrate was concentrated and the residue taken up in ethyl acetate and washed with 1N hydrochloric acid (2×20 mL). The combined aqueous washes were adjusted to pH >10 with 4N aqueous sodium hydroxide and extracted with dichloromethane (3×20 mL). The combined organic extracts were washed with saturated aqueous sodium chloride, dried (potassium carbonate), filtered and concentrated to dryness to provide the title compound in 95% yield. LRMS: 224 (M+H)⁺. ¹H NMR (CDCl₃, 400 MHz): δ 7.22 (1H, t, J=7.8 Hz), 6.91 (1H, d, J=7.6 Hz), 6.86 (1H, s), 6.72 (1H, dd, J=2.5, 8.1 Hz), 3.82 (2H, s), 3.74 (3H, s), 1.56 (6H, s).

### Preparation b-5-3

### Methyl 2-(3-cyanophenoxy)-2-methylpropanoate

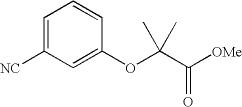

To a solution of 3-cyanophenol (5.0 mmol) in acetonitrile (20 mL) was added methyl 2-bromo-2-methyl-propanoate (1.2 equiv) and cesium carbonate (2.0 equiv). The resulting mixture was heated at 60° C. for 6 hours and then cooled to ambient temperature. Water (20 mL) was introduced and the mixture extracted with ethyl acetate (3×20 mL). The combined organics were washed with saturated aqueous sodium bicarbonate and saturated aqueous sodium chloride, dried (anhydrous sodium sulfate), filtered, and evaporated and then separated by flash column (10% acetate/hexane) to provide the title compound in 87% yield. LRMS: 220 (M+H)⁺. ¹H NMR (CDCl₃, 400 MHz): δ 7.30 (1H, t, J=8.0 Hz), 7.23 (1H, dt, J=1.3, 7.6 Hz), 7.05 (1H, dd, J=1.3, 2.3 Hz), 7.01 (1H, ddd, J=2.3, 2.8, 8.3 Hz), 3.73 (3H, s), 1.57 (6H, s).

### Preparations b-6

Preparation b-6 was prepared using procedures analogous to those described for preparation b-5

| | | | MS (m/z) |
|---|---|---|---|
| Prep # | Structure | ¹H NMR | (LR or HR) |
| b-6 | 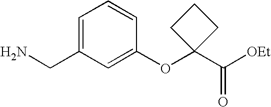 | ¹H NMR(CDCl₃, 400 MHz): δ8.10(1H, s), 7.97-7.93(2H, m), 7.44-7.39(5H, m), 4.68(2H, t, J=6.7Hz), 2.98(2H, t, J=6.7Hz), 2.34(3H, s). | LR 250(M + H)⁺ |

### Preparation b-7

### 2-(5-Methyl-2-phenyl-1,3-oxazol-4-yl)ethyl 1H-imidazole-1-carboxylate

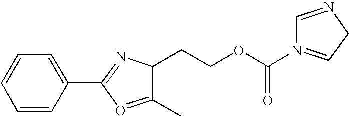

To a solution of 2-(5-methyl-2-phenyl-1,3-oxazol-4-yl)-ethanol (1.015 g, 5 mmol) in toluene (25 mL) was added potassium carbonate (1.38 g, 2 equiv) and N,N′-carbonyidiimidazole (0.97 g, 1.2 equiv). The resulting mixture was stirred at ambient temperature for 24 hours before water (20 mL) was introduced. Extraction with ethyl acetate and washing the combined organic extracts with saturated aqueous sodium chloride, drying (anhydrous sodium sulfate), filtration, and concentration to dryness afforded the title compound (100%). LRMS: 298

### Preparations b-8 to b-10

Preparations b-8 to b-10 were prepared using procedures analogous to those described for Preparation b-7

| | | | MS (m/z) |
|---|---|---|---|
| Prep # | Structure | ¹ H NMR | (LR or HR) |
| b-8 | 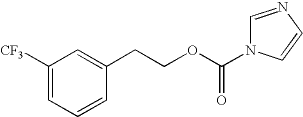 | (CDCl₃, 400 MHz) 8.10(1H, s), 7.68(1H, d, J=7.6Hz), 7.52(1H, t, J=7.6Hz), 7.40(1H, s), 7.38(1H, s), 7.24(1H, t, J=7.3Hz), 7.17-7.13(1H, m), 4.63(2H, t, J=6.8Hz), 3.29(2H, t, J=6.8Hz). | LR 285 (M + H)⁺ |
| b-9 | 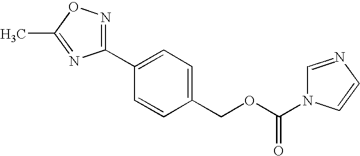 | (CDCl₃, 400 MHz) 8.49(1H, bs), 8.13(2H, d, J=8.3Hz), 7.57(1H, s), 7.54(2H, s), 7.26-7.24(1H, m), 5.52(2H, s), 2.66(3H, s). | LR 285 (M + H)⁺ |
| b-10 | 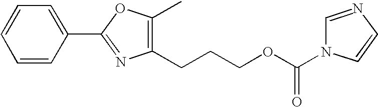 | | LR 312 (M + H)⁺ |

### Preparation b-11

### Methyl 2-methyl-2-{3-[({[2-(5-methyl-2-phenyl-1,3-oxazol-4-yl)ethoxy]carbonyl}amino)methyl]phenoxy}propanoate

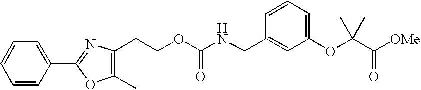

To a solution of 2-(5-methyl-2-phenyl-1,3-oxazol-4-yl)ethyl 1H-imidazole-1-carboxylate (Preparatio 7) (0.48 g, 1.6 mmol) in tetrahydrofuran (3 mL) was added methyl 2-[3-(aminomethyl)phenoxy]-2-methylpropanoate (Preparation b-4) (0.39 g, 1.1 equiv). The resulting mixture was heated at reflux for 16 hours and then cooled to ambient temperature. Concentration and purification by silica gel chromatography using 0-50% ethyl acetate in hexanes gave the title compound (0.39 g, 53%). LRMS: 453 (M+H)⁺. ¹H NMR (CDCl₃, 400 MHz): δ 7.96 (2H, dd, J=1.9, 7.7 Hz), 7.44-7.38 (3H, m), 7.16 (1H, t, J=8.0 Hz), 6.89 (1H, d, J=7.3 Hz), 6.77 (1H, s), 6.67 (1H, dd, J=2.3, 8.3 Hz), 4.36 (2H, t, J=6.7 Hz), 4.30 (2H, d, J=5.8 Hz), 3.75 (3H, s), 2.83 (2H, t, J=6.7 Hz), 2.32 (3H, s), 1.57 (6H, s).

### Preparations b-12 to b-20

Preparations b-12 to b-20 were prepared using Procedures analogous to those used for Preparation b-11

| Prep- | | | MS (m/z) |
|---|---|---|---|
| ara- | | | (LR or |
| tion # | Structure | ¹H NMR | HR) |
| b-12 | 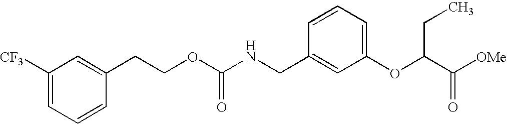 | | LR 440 (M + H)⁺ |
| b-13 | 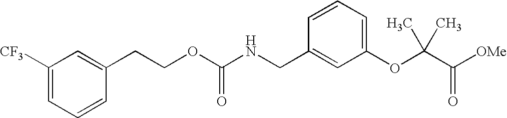 | (CDCl₃, 400 MHz) 7.63(1H, d, J=7.8Hz), 7.47(1H, t, J=7.5Hz), 7.38(1H, d, J=6.3Hz), 7.34-7.28(1H, m), 7.18(1H, dd, J=7.6, 8.1Hz), 6.89(1H, d, J=7.3Hz), 6.77(1H, s), 6.69(1H, dd, J=2.3, 8.3Hz), 4.33-4.30(4H, m), 3.75(3H, s), 3.13(2H, t, J=7.0Hz), 1.58(6H, s). | LR 440 (M + H)⁺ |
| b-14 | 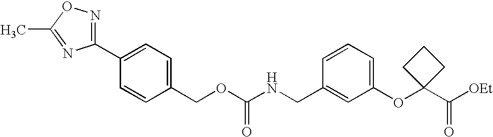 | (CDCl₃, 400 MHz) 7.99(2H, d, J=8.1Hz), 7.41(2H, m), 7.11(1H, t, J=8.1Hz), 6.79(1H, d, J=7.6Hz), 6.60(1H, s), 6.46(1H, dd, J=2.3, 8.1Hz), 5.12(2H, s), 4.27(2H, d, J=6.1Hz), 4.12(2H, q, J=7.1Hz), 2.71-2.60(2H, m), 2.59(3H, s), 2.42-2.32(2H, m), 1.98-1.88(2H, m), 1.09(3H, t, | |
| #J=7.1Hz). | LR 440 (M + H)⁺ | | |
| b-15 | 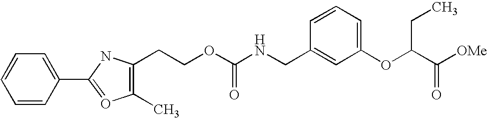 | | LR 453 (M + H)⁺ |
| b-16 | 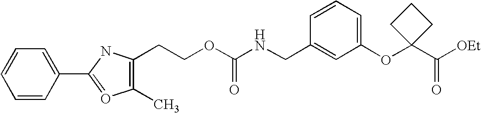 | (CDCl₃, 400 MHz) 7.96(2H, dd, J=1.9, 7.7Hz), 7.44-7.36(3H, m), 7.18-7.13(1H, m), 6.85(1H, d, J=7.6Hz), 6.66(1H, s), 6.50(1H, dt, J=1.3, 8.1Hz), 4.35(2H, t, J=7.1Hz), 4.29(2H, d, J=6.1Hz), 4.11(2H, q, J=7.1Hz), 2.83(2H, t, J=6.8Hz), 2.77-2.69(2H, m), 2.48-2.38(2H, m), 2.32(3H, | |
| #s), 2.01-1.94(2H, m), 1.16(3H, t, J=7.2Hz). | LR 479 (M + H)⁺ | | |
| b-17 | 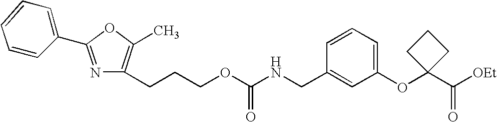 | (CDCl₃, 400 MHz) 7.96(2H, dd, J=1.8, 7.8Hz), 7.43-7.38(3H, m), 7.17(1H, t, J=7.8Hz), 6.85(1H, d, J=7.6Hz), 6.66(1H, 6.50(1H, dd, J=2.3, 8.1Hz), 4.30(2H, d, J=5.8Hz), 4.19(2H, q, J=7.3Hz), 4.18-4.08(2H, t, J=7.5Hz), 2.48-2.39(2H, m), 2.30(3H, s), 2.02-1.94(4H, m), 1.16(3H, | |
| #t, J=7.1Hz). | LR 493 (M + H)⁺ | | |
| b-18 | 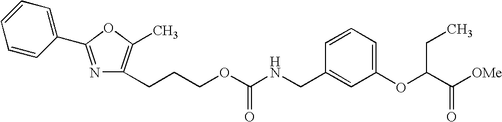 | (CDCl₃, 400 MHz) 7.96(2H, dd, J=1.8, 7.8Hz), 7.43-7.38(3H, m), 7.22(1H, t, J=8.1Hz), 6.89(1H, d, J=7.8Hz), 6.82(1H, s), 6.74(1H, dd, J=2.2, 8.2Hz), 4.56(1H, t, J=6.2Hz), 4.32(2H, d, J=5.8Hz), 4.14(2H, t, J=6.3Hz), 3.74(3H, s), 2.56(2H, t, J=7.3Hz), 2.30(3H, s), 2.03-1.93(4H, m), 1.06(3H, t, | |
| #J=7.3Hz). | LR 467 (M + H)⁺ | | |
| b-19 | 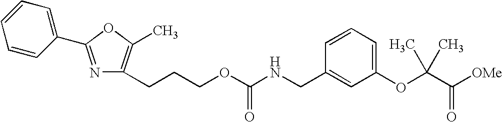 | (CDCl₃, 400 MHz) 7.96(2H, dd, J=1.8, 7.8Hz), 7.43-7.38(3H, m), 7.18(1H, t, J=8.0Hz), 6.90(1H, d, J=7.6Hz), 6.78(1H, s), 6.68(1H, dd, J=2.2, 8.0Hz), 4.31(2H, d, J=6.1Hz), 4.13(2H, t, J=6.1Hz), 3.75(3H, s), 2.56(2H, t, J=7.3Hz), 2.30(3H, s), 2.00(2H, t, J=7.1Hz), 1.58(6H, s). | LR 467 (M + |
| #H)⁺ | | | |
| b-20 | 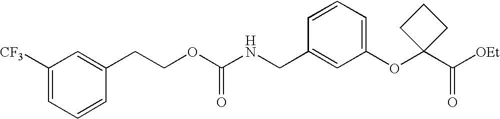 | (CDCl₃, 400 MHz) 7.63(1H, d, J=7.8Hz), 7.47(1H, t, J=7.6Hz), 7.38(1H, d, J=7.6Hz), 7.32(1H, t, J=7.7Hz), 7.16(1H, t, J=8.0Hz), 6.83(1H, d, J=7.3Hz), 6.65(1H, s), 6.51(1H, dd, J=2.3, 8.1Hz), 4.33-4.29(4H, m), 4.18(2H, q, J=7.1Hz), 3.13(2H, t, J=7.0Hz), 2.76-2.69(2H, m), 2.47-2.39(2H, m), | |
| #2.01-1.94(2H, m), 1.16(3H, t, J=7.1Hz). | LR 412 (M + H)⁺ | | |

### Preparation b-21

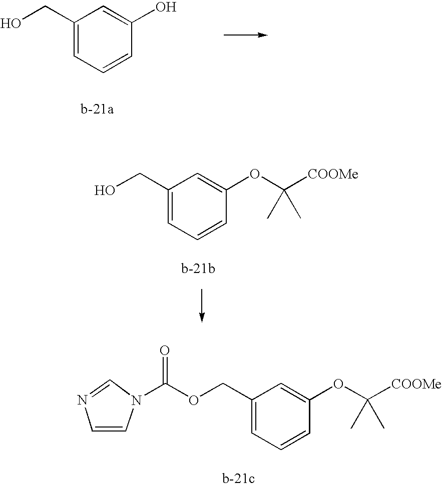 Preparation of Imidazole b-21c

To a solution of the alcohol b-21b (1 mmol) in toluene (5 ml) were added N, N″-carbonyldiimidazole (1.05 mmol) and potassium carbonate (1 mmol). The resulting solution heated at reflux for 3 hr. After cooling to room temperature, water (20 ml) was added and the mixture extracted with ethyl acetate (3×20 ml). The combined extracts were washed with brine, dried over sodium sulfate and concentrated in vacuo to provide the acyl imidazole b-21c in quatitative yield.

Preparation of Alcohol b-21b

To a solution of 3-hydroxybenzyl alcohol b-21a (1 mmol) and cesium carbonate (1 mmol) in acetonitrile (10 ml) was added methyl 2-bromo-2-methyl propionate (2 mmol). The mixture was heated under reflux for 6 hours. After cooling to room temperature, water (50 ml) was introduced and the mixture extracted with ethyl acetate (3×20 ml). Combined organics were washed with brine, dried over sodium sulfate and evaporated in vacuo. Silica gel chromatography (SGC) using 10-30% ethyl acetate-hexane gave alcohol b-21b. Yields ranged between 40-85%. 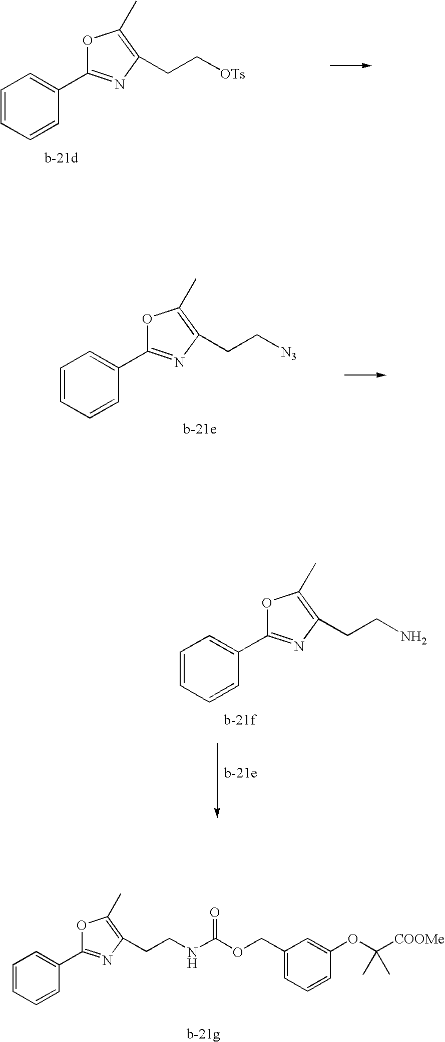
Preparation of Methyl Ester b-21a

Following the procedures described in b-11, methyl ester b-21g was prepared by reacting compound b-21f with compound b-21c in yields ranging from 60 to 90%.

Preparation of Amine b-21f

A solution of the azide b-21e (2 mmol) in ethyl acetate (20 ml) and palladium on carbon (10% by weight, 50 mg) was treated with hydrogen gas at room temperature for 4 hours. Removal of palladium by filtration through a pad of Celite and concentration produced the amine b-21f in quantitative yield.

Preparation of Azide b-21e

To a solution of the tosylate b-21d (1 mmol) in DMF (5 ml) was added sodium azide (3 mmol). The resulting mixture stirred at room temperature for 14 hours before water (50 ml) was added. Extraction with ethyl acetate (3×20 ml), washing of the combined organics with water, saturated sodium bicarbonate and brine, drying over sodium sulfate and concentration gave rise to the azide b-21e in 85% yield.

### EXAMPLE C-1

### 1-{4-[2-(5-methyl-2-phenyl-1,3-oxazol-4-yl)ethoxy]benzyl}cyclohexanecarboxylic acid

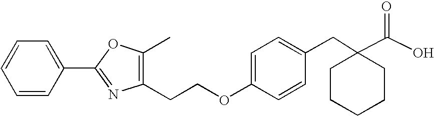

Triethylsilane (1.03 g, 8.86 mmol) was added to a solution of methyl 1-(hydroxyl{4-[2-(5-methyl-2-phenyl-1,3-oxazol-4-yl)ethoxy]phenyl}methyl)cyclohexanecarboxylate (0.797 g, 1.77 mmol) in dichloromethane (5 mL) and trifluoroacetic acid (1 mL) at room temperature. The resulting mixture was stirred for 1 hour then evaporated in vacuo and azeotroped with heptane. The residue was dissolved in tetrahydrofuran (3 mL) and water (3 mL) and lithium hydroxide monohydrate (0.223 g, 5.31 mmol) was added. The resulting mixture was stirred at room temperature for 18 hours, acidified to pH 2 with 4N hydrochloric acid and extracted with ethyl acetate. The organic phase was dried (anhydrous magnesium sulfate), filtered and evaporated to afford the title compound (0.332 g, 45%). Elemental Analysis: Calcd for C₂₆H₂₉NO₄ C, 74.44H, 6.97, N, 3.34. Found: C, 74.22, H, 6.89, N, 3.34. LRMS: 420 (M+H)⁺. ¹H NMR (DMSO-d₆, 400 MHz): δ 7.90 (2H, dd, J=1.8, 7.8 Hz), 7.51-7.46 (3H, m), 6.98 (2H, d, J=8.6 Hz), 6.80 (2H, d, J=8.6 Hz), 4.16 (2H, t, J=6.6 Hz), 2.90 (2H, t, J=6.6 Hz), 2.64 (2H, s), 2.34 (3H, s), 1.85 (2H, d, J=12.6 Hz), 1.53-1.46 (3H, m), 1.29-1.11 (5H, m).

### EXAMPLES C-2 TO C-5

Examples C-2 to C-5 were prepared by procedures analogous to those used for Example C-1 with the exception that the final hydrolysis step was carried out by dissolving the crude residue in dimethyl sulfoxide (75 mq/mL) and 6N sodium hydroxide (1 mL) and heating at 150° C. for 10 minutes in a microwave synthesizer.

| | | | MS (m/z) | |
|---|---|---|---|---|
| Ex # | Structure | ¹H NMR | (LR or HR) | Analysis |
| C-2 | 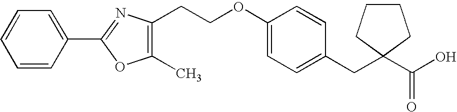 | (DMSO-d₆, 400 MHz) 7.90(2H, dd, J=1.9, 7.7Hz), 7.51-7.44(3H, m), 7.04(2H, d, J=8.6Hz), 6.80(2H, d, J=8.6Hz), 4.15(2H, t, J=6.7Hz), 2.90(2H, t, J=6.6Hz), 2.79(2H, s), 2.34(3H, s), 2.05-2.00(2H, m), 1.75-1.60(6H, m). | LR 406 (M + H)+ | Calcd |
| #for C₂₅H₂₇NO₄C 74.05, H 6.71, N 3.45. Found: C 73.83, H 6.69, N 3.36. | | | | |
| C-3 | 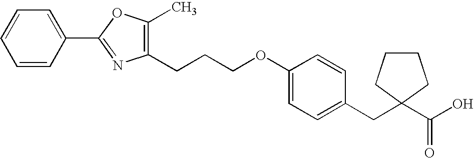 | (DMSO-d₆, 400 MHz) 7.89(2H, dd, J=1.8, 7.8Hz), 7.51-7.46(3H, m), 7.04(2H, d, J=8.6Hz), 6.81(2H, d, J=8.6Hz), 3.93(2H, t, J=6.2Hz), 2.79(2H, s), 2.60(2H, t, J=7.3Hz), 2.27(3H, s), 2.04-1.97(2H, m), 1.92-1.85(2H, m), | | |
| #1.61-1.46(6H, m). | LR 420 (M + H)⁺ | Calcd for C₂₆H₂₉NO₄C 74.44, H 6.97, N 3.34. Found: C 74.30, H 6.95, N 3.26. | | |
| C-4 | 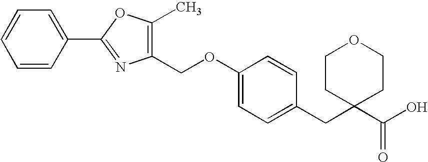 | (DMSO-d₆, 400 MHz) 7.94-7.92(2H, m), 7.54-7.48(3H, m), 7.04(2H, d, J=8.6Hz), 6.92(2H, d, J=8.6Hz), 4.95(2H, s), 3.75-3.70(2H, m), 3.28(2H, dd, J=10.0, 11.2Hz), 2.73(2H, s), 2.43(3H, s), 1.81(2H, d, J=13.1Hz), 1.49-1.42(2H, m). | LR | |
| #408 (M + H)⁺ | Calcd for C₂₄H₂₅NO₅C 70.75, H 6.18, N 3.44. Found: C 70.60, H 6.33, N 3.31. | | | |
| C-5 | 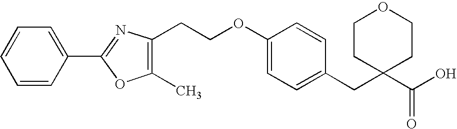 | (DMSO-d₆, 400 MHz) 7.90(2H, dd, J=1.9, 7.7Hz), 7.51-7.44(3H, m), 6.99(2H, d, J=8.6Hz), 6.81(2H, d, J=8.6Hz), 4.16(2H, t, J=6.6Hz), 3.74-3.69(2H, m), 3.27(2H, t, J=10.6Hz), 2.90(2H, t, J=6.4Hz), 2.71(2H, s), 2.34(3H, s), 1.79(2H, d, | | |
| #J=13.1Hz), 1.47-1.39(2H, m). | LR 422 (M + H)⁺ | Calcd for C₂₅H₂₇NO₅C 71.24, H 6.46, N 3.32. Found: C 71.01, H 6.47, N 3.32. | | |

### EXAMPLE C-6

### 1-{4-[2-(4′-methoxy-1,1′-biphenyl-4-yl)ethoxy]benzyl}cyclobutanecarboxylic acid

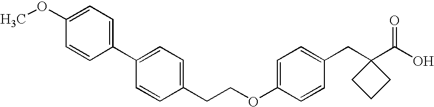

To a solution of ethyl 1-{4-[2-(4′-methoxy-1,1′-biphenyl-4-yl)ethoxy]benzyl}cyclobutanecarboxylate (Preparation 14) (0.3921 mmol, 1 equiv.) in acetonitrile (2 mL) was added 1N aqueous sodium hydroxide (7.2 mL, 8 equiv.). The resulting mixture was subjected to microwave heating (100° C.) in a Personal Chemistry Smith Synthesizer for 40 minutes. Following cooling of the reaction mixture, 1M aqueous hydrochloric acid was added until pH 1 was achieved. The mixture was extracted with ethyl acetate (3×50 mL). The combined organic extracts were then washed with saturated aqueous sodium chloride (100 mL), dried (anhydrous magnesium sulfate), filtered and concentrated in vacuo to afford the crude product. The residue was purified by trituration from diethyl ether to afford the title compound as a white crystalline solid (0.1321 g, 81%). Elemental Analysis: Calcd for C₂₇H₂₈O₄ C, 77.86, H, 6.78. Found: C, 77.65, H, 6.85. LRMS (m/z): 416 (M). ¹H NMR (Acetone-d₆, 300 MHz): δ 7.53 (2H, d, J=6.1 Hz), 7.66 (2H, d, J=5.1 Hz), 7.46 (2H, d, J=8.5 Hz), 7.13 (2H, d, J=8.7 Hz), 7.07 (2H, d, J=8.7 Hz), 6.85 (2H, d, J=8.5 Hz), 4.20 (1H, t, J=6.1 Hz), 3.86 (3H, s), 3.10 (2H, t, J=7.0 Hz), 3.00 (2H, s), 2.40-2.30 (2H, m), 2.07-1.98 (2H, m), 1.92-1.80 (2H, m).

### EXAMPLES C-7 TO C-93

Examples C-7 to C-93 were prepared by procedures analogous to those used for Example C-6 or by stirring a solution of the ester with sodium or lithium hydroxide in aqueous methanol, aqueous ethanol, aqueous tetrahydrofuran or mixtures thereof at temperatures between 20° C. and 75° C.

| | | | MS (m/z) | |
|---|---|---|---|---|
| Ex # | Structure | ¹H NMR | LR or HR) | Analysis |
| C-7 | 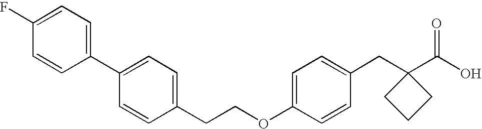 | (Acetone-d₆, 300 MHz) 7.48(2H, d, J=8.9Hz), 7.58(2H, d, J=8.7Hz), 7.41(2H, d, J=8.5Hz), 7.13(2H, t, J=8.9Hz), 7.07(2H, d, J=8.7Hz), 6.78(2H, d, J=8.7Hz), 4.14(2H, t, J=7.0Hz), 3.03(2H, t, J=7.0Hz), 2.97(2H, s), 2.40-2.30(2H, m), 2.01-1.92 (2H, m), 1.85-1.72(2H, m). | LR 404 (M)⁻ | Calcd |
| #for C₂₆H₂₅FO₃C 77.21, H 6.23. Found: C 77.13, H 6.28. | | | | |
| C-8 | 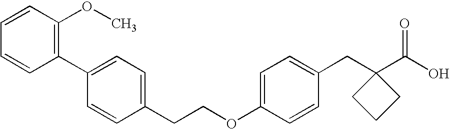 | (Acetone-d₆, 300 MHz) 7.58(2H, d, J=8.3Hz), 7.41(2H, d, J=8.3Hz), 7.34(1H, t, J=7.9Hz), 7.21-7.15(2H, m), 7.12(2H, d, J=8.7Hz), 6.90(1H, ddd, J=0.94, 2.64, 8.3Hz), 6.83(2H, d, J=8.7Hz), 4.19(2H, t, J=6.9Hz), 3.85(3H, s), 3.09(2H, t, J=6.8Hz), 3.02(2H, s), 2.39-2.30(2H, m), 2.07-1.98(2H, m), 1.89-1.81(2H, m) | LR 416 | |
| #(M)⁻ | Calcd for C₂₇H₂₈O₄C 77.86, H 6.78. Found: C 77.67, H 6.67. | | | |
| C-9 | 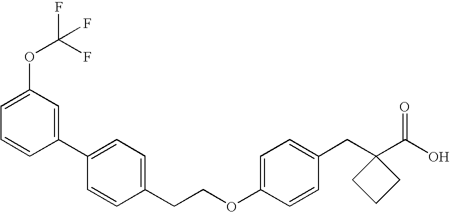 | (Acetone-d₆, 300 MHz) 7.69(1H, dt, J=0.9, 8.3Hz), 7.65(2H, d, J=8.3Hz), 7.58(2H, t, J=8.1Hz), 7.47(2H, d, J=8.3Hz), 7.31(1H, dt, J=1.1, 8.1Hz), 7.13(2H, d, J=8.7Hz), 6.83(2H, d, J=8.7Hz), 4.21(2H, t, J=6.8Hz), 3.12(2H, t, J=6.8Hz), 3.03(2H, s), 2.40-2.30(2H, m), 2.08-1.98(2H, | | |
| #m), 1.92-1.79(2H, m) | LR 470 (M)⁻ | Calcd for C₂₇H₂₅F₃O₄C 68.93, H 5.36. Found: C 69.04, H 5.47. | | |
| C-10 | 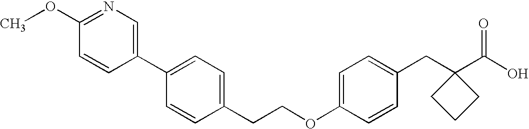 | (Acetone-d₆, 300 MHz) 8.42(1H, d, J=2.6Hz), 7.93(1H, dd, J=2.6, 8.7Hz), 7.57(2H, d, J=8.3Hz), 7.44(2H, d, J=8.3Hz), 7.13(2H, d, J=8.9Hz), 6.84(2H, d, J=8.7Hz), 6.85-6.81(1H, m), 4.20(2H, t, J=6.8Hz), 3.91(3H, s), 3.10(2H, t, J=7.0Hz), 3.03(2H, s), 2.40-2.30(2H, m), 2.07-1.99(2H, m), | | |
| #1.90-1.82(2H, m). | LR 417 (M)⁻ | Calcd for C₂₆H₂₇NO₄C 74.80, H 6.52, H 3.35. Found: C 74.67, H 6.46, N 3.31. | | |
| C-11 | 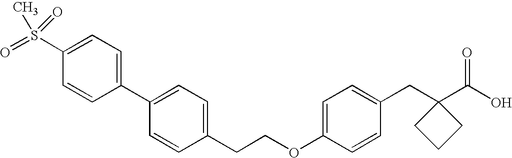 | (Acetone-d₆, 300 MHz) 8.01(2H, d, J=8.5Hz), 7.92(2H, d, J=8.5Hz), 7.70(2H, d, J=8.5Hz), 7.50(2H, d, J=8.1Hz), 7.13(2H, d, J=8.7Hz), 6.84(2H, d, J=8.7Hz), 4.22(2H, t, J=6.8Hz), 3.15(3H, s), 3.13(2H, t, J=6.8Hz), 3.03(2H, s), 2.40-2.30(2H, m), 2.07-1.98(2H, m), 1.92-1.79(2H, m). | LR 482 (M)⁻ | Calcd |
| #for C₂₇H₂₈O₅S C 69.80, H 6.07. Found: C 69.41, H 6.12. | | | | |
| C-12 | 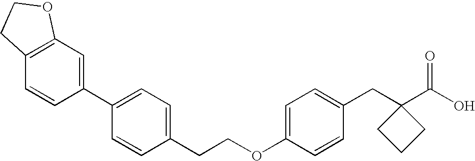 | (Acetone-d₆, 300 MHz) 7.53-7.48(1H, m), 7.52(2H, d, J=8.3Hz), 7.38-7.34(1H, m), 7.37(2H, d, J=8.3Hz), 7.13(2H, d, J=8.7Hz), 6.83(2H, d, J=8.7Hz), 6.78(1H, d, J=8.3Hz), 4.57(2H, t, J=8.7Hz), 4.18(2H, t, J=6.8Hz), 3.25(2H, t, J=8.7Hz), 3.08(2H, t, J=7.0Hz), 3.03(2H, s), 2.40-2.30(2H, m), 2.07-1.99(2H, m), | | |
| #1.92-1.79(2H, m). | LR 428 (M)⁻ | Calcd for C₂₈H₂₈O₄C 78.48, H 6.59. Found: C 78.30, H 6.62. | | |
| C-13 | 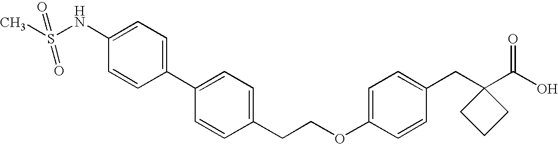 | (Acetone-d₆, 300 MHz) 7.65(2H, d, J=8.7Hz), 7.59(2H, d, J=8.1Hz), 7.42(4H, dd, J=1.5, 8.1Hz), 7.13(2H, d, J=8.5Hz), 6.84(2H, d, J=8.9Hz), 4.20(2H, t, J=6.8Hz), 3.10(2H, t, J=6.8Hz), 3.03(2H, s), 3.01(3H, s), 2.40-2.30(2H, m), 2.07-1.99(2H, m), 1.92-1.81(2H, m). | LR 479 (M)⁻ | Calcd |
| #for C₂₇H₂₉NO₅S C 67.62, H, 6.09, N 2.92. Found: C 67.36, H 6.11, N 2.85. | | | | |
| C-14 | 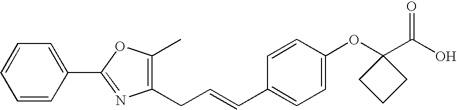 | ¹H NMR(400 MHz, CDCl₃) 2.29(s, 3H), 2.39(m, 2H), 2.75(m, 2H), 3.00(q, 2H), 3.30(d, 2H), 6.00(td, 1H), 6.25(d, 1H), 6.56(d, 2H), 7.08(d, 2H), 7.35(m, 3H), 7.90(m, 2H). | LRMS (m/z): 390 (M + H)⁺. | |
| C-15 | 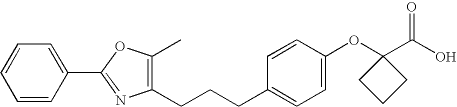 | ¹H NMR(400 MHz, CDCl₃) 1.77 1.89(m, 2H), 1.93 2.05(m, 2H), 2.27(s, 3H), 2.40 2.51(m, 6H), 2.71 2.80(m, 2H), 6.63(d, 2H), 6.97(d, 2H), 7.40(dd, 3H), 7.92(m, 2H). | LRMS (m/z): 392 (M + H)⁺. | |
| C-16 | 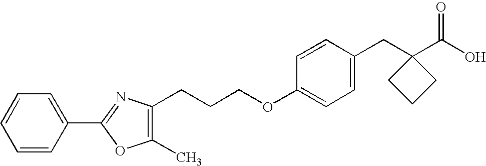 | (DMSO-d₆, 400 MHz) 12.13(1H, s), 7.86(2H, m), 7.49-7.40(3H, m), 7.01(2H, d, J=8.8Hz) 6.78(2H, t, J=8.6Hz), 3.90(2H, t, J=6.3Hz), 2.89(2H, s), 2.56(2H, t, J=7.3Hz), 2.24(3H, s), 2.24-2.15(2H, m) 2.00-1.67(6H, m). | 406.2002. | Calcd for C₂₅H₂₇NO₄C 74.05, H 6.71, N 3.45. Found: |
| #C 73.75, H 6.64, N 3.44. | | | | |
| C-17 | 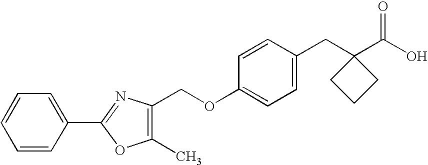 | (DMSO-d₆, 400 MHz)12.04(1H, s), 7.79(2H, m), 7.40-7.34(3H, m), 6.95(2H, d, J=8.6Hz), 6.78(2H, t, J=8.6Hz), 4.80(2H, s), 2.80(2H, s), 2.28(3H, s), 2.12-2.06(2H, m) 1.83-1.57(4H, m). | 378.1692. | Calcd for C₂₃H₂₃NO₄C 73.19, H 6.14, N 3.71. Found: C 73.15, H 6.26, N 3.75. |
| C-18 | 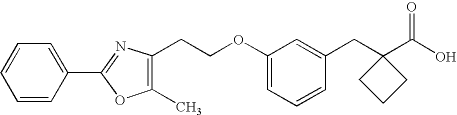 | (CDCl₃, 400 MHz) 8.00-7.97(2H, m), 7.49-7.40(3H, m), 7.17(1H, t, J=7.8Hz), 7.03(1H, s), 6.78(2H, t, J=7.1Hz), 4.24(2H, t, J=8.1Hz), 3.14(2H, s), 2.90(2H, t, J=7.8Hz), 2.51-2.40(2H, m), 2.36(3H, s), 2.07-1.83(4H, m). | 392.1859. | Calcd for C₂₄H₂₅NO₄0.2H₂O C 72.96, H |
| #6.48, N 3.55. Found: C 72.66, H 6.65, N 3.47. | | | | |
| C-19 | 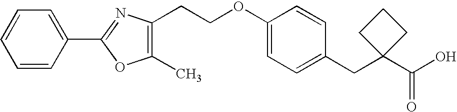 | (DMSO-d₆, 400 MHz) 7.90(2H, dd, J=1.9, 7.7Hz), 7.51-7.44(3H, m), 7.05(2H, d, J=8.6Hz), 6.81(2H, d, J=8.6Hz), 4.15(2H, t, J=6.7Hz), 6.81(2H, d, J=8.6Hz), 4.15(2H, t, J=6.8Hz), 2.34(3H, s), 2.25-2.18(2H, m), 1.95-1.88(2H, m), 1.86-1.71(2H, m). | LR 392 (M + H)⁺ | Calcd for C₂₄H₂₅NO₄C 73.64, H 6.44, N 3.58. Found: |
| #C 73.49, H 6.46, N 3.54. | | | | |
| C-20 | 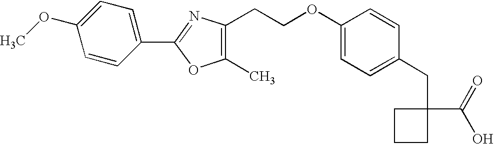 | ¹H NMR(MSO-d₆, 400 MHz): 7.83(2H, d, J=9.1Hz), 7.06-7.02(4H, m), 6.82(2H, d, J=8.6Hz), 4.17(2H, t, J=6.3Hz), 3.80(3H, s), 3.10(2H, t, J=6.3Hz), 2.92(2H, s), 2.25-2.17(2H, m), 2.08(3H, s), 1.95-1.69(4H,sd,21 m). | 422.1961 | |
| C-21 | 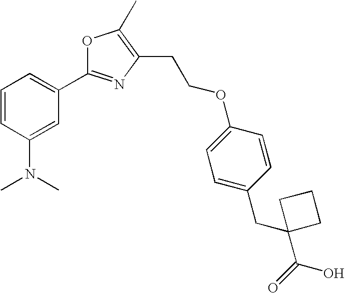 | (DMSO-d6, 400 MHz) 7.25-7.24(1H, m), 7.19-7.17(2H, m), 7.00-6.97(2H, m), 6.76-6.75(1H, m), 6.72-6.70(2H, m), 4.10(2H, t, J=6.5Hz), 2.90(6H, s), 2.85(2H, t, J=6.5Hz), 2.25(3H, s), 2.24-2.21(2H, m), 1.95-1.92(2H, m), 1.78-1.74(2H, m). | 435.2270. LR 435 (M + H)⁺ | |
| C-22 | 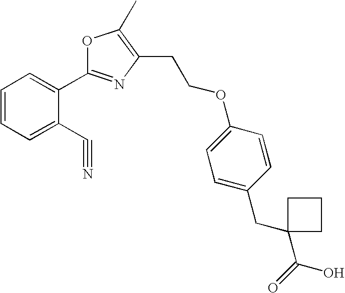 | (CDCl₃, 400 MHz) 8.13-8.11(1H, m), 7.77-7.75(1H, m), 7.66-7.62(1H, m), 7.49-7.45(1H, m), 7.09-7.07(2H, m), 6.81-6.79(2H, m), 4.23(2H, t, J=6.3Hz), 3.03(2H, s), 2.99(2H, t, J=6.3Hz), 2.46-2.38(2H, m), 2.40(3H, s), 2.09-2.02(2H, m), 1.90-1.86(2H, m). | HR 417.1813. LR 418 (M + H)⁺ | |
| C-23 | 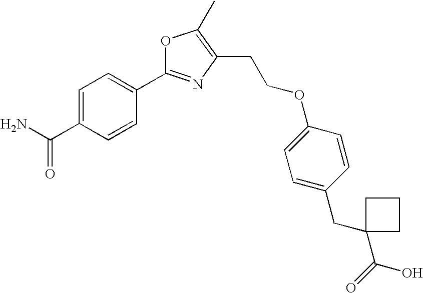 | (DMSO-d6, 400 MHz) 12.04(1H, s), 7.89-7.84(4H, m), 6.96-6.94(2H, m), 6.72-6.70(2H, m), 4.06(2H, t, J=6.5Hz), 3.06-3.05(1H, m), 2.82-2.80(4H, m), 2.26(3H, s), 2.15-2.08(2H, m), 1.83-1.79(2H, m), 1.69-1.65(2H, m). | HR 435.1922. LR 435 (M + H)⁺ | |
| C-24 | 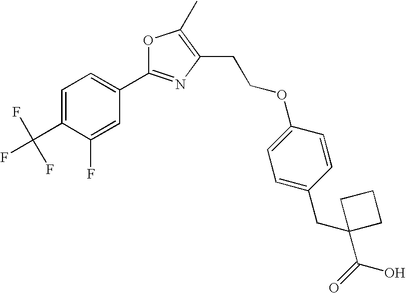 | (CDCl₃, 300 MHz) 7.84-7.76(2H, m), 7.66-7.61(1H, m), 7.09-7.06(2H, m), 6.80-6.77(2H, m), 4.19(2H, t, J=6.5Hz), 3.03(2H, s), 2.96(2H, t, J=6.4Hz), 2.47-2.40(2H, m), 2.37(3H, s), 2.10-2.01(2H, m), 1.95-1.81(2H, m). | HR 478.1624. LR 478 (M + H)⁺ | |
| C-25 | 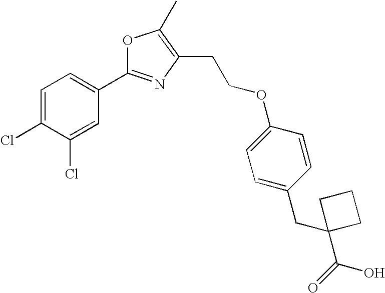 | (CDCl₃, 300 MHz) 8.06-8.05(1H, d), 7.80-7.77(1H, m), 7.50-7.47(1H, d), 7.09-7.06(2H, m), 6.80-6.77(2H, m), 4.19(2H, t, J=6.5Hz), 3.03(2H, s), 2.94(2H, t, J=6.5Hz), 2.44-2.38(2H, m), 2.35(3H, s), 2.08-2.01(2H, m), 1.92-1.84(2H, m). | HR 460.1089. LR 461 (M + H)⁺ | |
| C-26 | 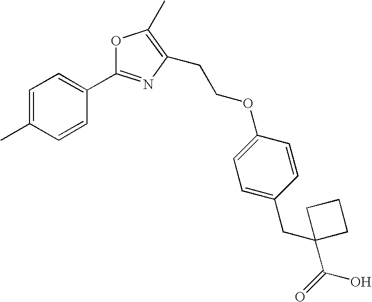 | (CDCl₃, 300 MHz) 7.87-7.85(2H, m), 7.24-7.21(2H, m), 7.09-7.07(2H, m), 6.81-6.78(2H, m), 4.20-4.17(2H, m), 3.04(2H, s), 2.98-2.94(2H, m), 2.48-2.41(2H, m), 2.38(3H, s), 2.35(3H, s), 2.10-2.02(2H, m), 1.90-1.83(2H, m). | HR 406.2014 LR 406 (M + H)⁺ | |
| C-27 | 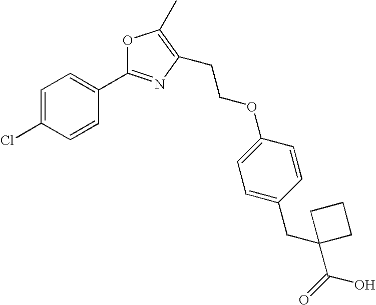 | (CDCl₃, 300 MHz) 7.91-7.88(2H, m), 7.40-7.37(2H, m), 7.09-7.06(2H, m), 6.80-6.77(2H, m), 4.18(2H, t, J=6.5Hz), 3.03(2H, s), 2.95(2H, t, J=6.5Hz), 2.44-2.41(2H, m), 2.35(3H, s), 2.08-2.01(2H, m), 1.90-1.87(2H, m). | HR 426.1471. LR 426 (M + H)⁺ | |
| C-28 | 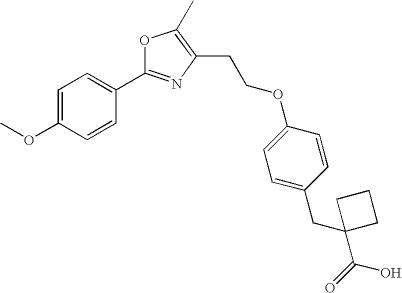 | (CDCl₃, 300 MHz) 7.92-7.89(2H, m), 7.10-7.07(2H, m), 6.95-6.92(2H, m), 6.80-6.77(2H, m), 4.17(2H, t, J=6.5Hz), 3.85(3H, s), 3.04(2H, s), 2.95(2H, t, J=6.5Hz), 2.48-2.38(2H, m), 2.34(3H, s), 2.10-2.02(2H, m), 1.94-1.83(2H, m). | HR 422.1948. LR 423 (M + H)⁺ | |
| C-29 | 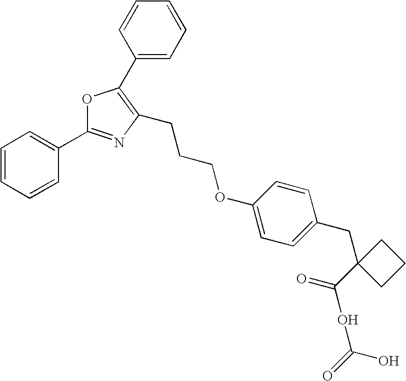 | (CDCl₃, 300 MHz) 7.57-7.55(1H, m), 7.51-7.49(1H, m), 7.35-7.30(1H, m), 7.10-7.07(2H, m), 6.97-6.93(1H, m), 6.81-6.78(2H, m), 4.19(2H, t, J=6.5Hz), 3.87(3H, s), 3.04(2H, s), 2.96(2H, t, J=6.5Hz), 2.48-2.38(2H, m), 2.36(3H, s), 2.11-2.02(2H, m), 1.94-1.85(2H, m). | HR 422.1947. LR | |
| #422 (M + H)⁺ | | | | |
| C-30 | 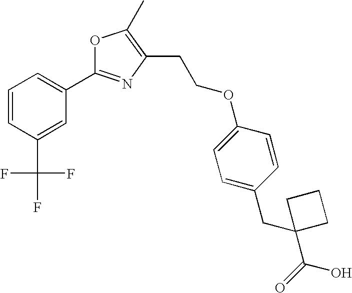 | (CDCl₃, 400 MHz) 8.23(1H, s), 8.15-8.13(1H, m), 7.64-7.63(1H, m), 7.54-7.50(1H, m), 7.07-7.05(2H, m), 6.99-6.96(1H, m), 6.77-6.75(2H, m), 6.70-6.68(1H, m), 4.19(2H, t, J=6.5Hz). | HR 460.1728. LR 460 (M + H)⁺ | |
| C-31 | 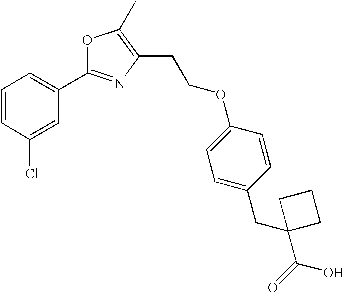 | (CDCl₃, 300 MHz) 7.96(1H, s), 7.86-7.83(1H, m), 7.36-7.33(2H, m), 7.09-7.06(2H, m), 6.79-6.76(2H, m), 4.18(2H, t, J=6.5Hz), 3.03(2H, s), 2.95(2H, t, J=6.5Hz), 2.47-2.38(2H, m), 2.35(3H, s), 2.08-2.01(2H, m), 1.91-1.84(2H, m). | HR 426.1465. LR 426 (M + H)⁺ | |
| C-32 | 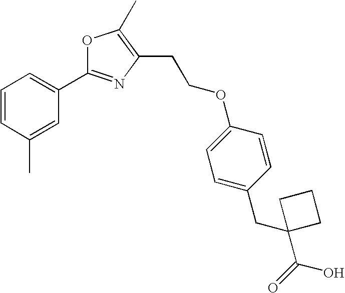 | (CDCl₃, 400 MHz) 7.81 1H, s), 7.77-7.75(1H, m), 7.33-7.29(1H, m), 7.22-7.20(1H, m), 7.09-7.07(2H, m), 6.81-6.78(2H, m), 4.19(2H, t, J=6.5Hz), 3.04(2H, s), 2.96(2H, t, J=6.5Hz), 2.44-2.42(2H, m), 2.39(3H, s), 2.35(3H, s), 2.08-2.03(2H, m), 1.89-1.87(2H, m). | HR 406.2026. LR 407 (M + H)⁺ | |
| C-33 | 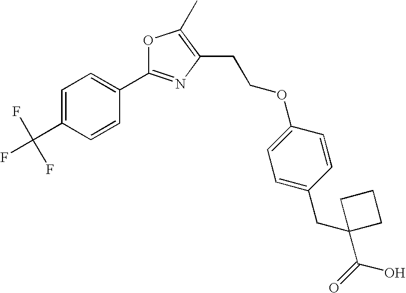 | (CDCl₃, 400 MHz) 8.08-8.06(2H, d), 7.68-7.66(2H, d), 7.09-7.07(2H, m), 6.80-6.78(2H, m), 4.91(2H, t, J=6.5Hz), 3.03(2H, s), 2.97(2H, t, J=6.5Hz), 2.46-2.38(2H, m), 2.37(3H, s), 2.09-2.02(2H, m), 1.92-1.82(2H, m). | HR 460.1723. LR 461 (M + H)⁺ | |
| C-34 | 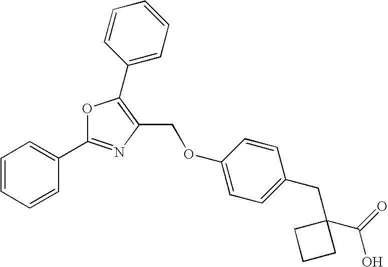 | (DMSO-d₆, 300 MHz) 12.19(1H, s), 8.12-8.11(2H, m), 7.82-7.79(2H, m), 7.59-7.44(6H, m), 7.13-6.98(4H, m), 5.17(2H, s), 2.97(2H, s), 2.30-2.21(2H, m), 2.01-1.73(4H, m). | 440.1846. | |
| C-35 | | (CDCl₃, 300 MHz) 8.09-8.07(2H, m), 7.68-7.66(2H, m), 7.46-7.26(6H, m), 7.10-6.78(4H, m), 4.01(2H, t, J=5.8Hz), 3.04-3.00(4H, m), 2.49-2.39(2H, m), 2.30-2.21(2H, m), 2.11-2.02(2H, m), 1.94-1.81(2H, m). | 468.2165. | |
| C-36 | 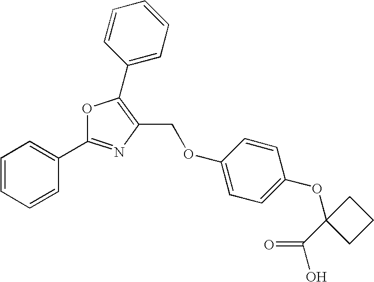 | (DMSO-d₆, 300 MHz) 12.92(1H, s), 8.12-8.09(2H, m), 7.82-7.79(2H, m), 7.60-7.44(6H, m), 7.02-6.99(2H, m), 6.64-6.60(2H, m), 5.13(2H, s), 2.68-2.59(2H, m), 2.37-2.26(2H, m), 1.95-1.84(2H, m). | 442.1639. | |
| C-37 | 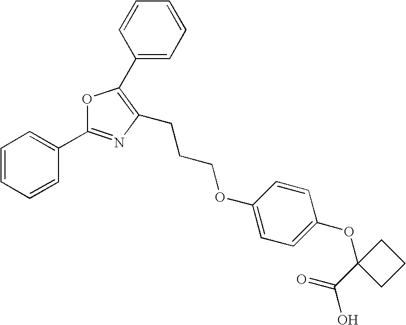 | (CDCl₃, 300 MHz) 8.10-8.06(2H, m), 7.69-7.66(2H, m), 7.46-7.32(6H, m), 6.79-6.66(4H, m), 3.91(2H, t, J=6.0Hz), 2.98(2H, t, J=7.3Hz), 2.80-2.71(2H, m), 2.50-2.40(2H, m), 2.24-2.15(2H, m), 2.06-1.93(2H, m). | 470.1952. | Calcd for C₂₉H₂₇NO₅C 74.18, H 5.80, N 2.98. Found: |
| #C 73.07, H 6.81, N 2.43. | | | | |
| C-38 | 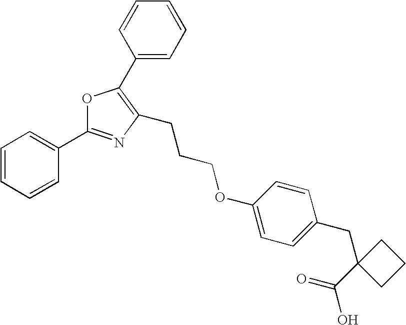 | (CDCl₃, 300 MHz) 8.09-8.07(2H, m), 7.68-7.66(2H, m), 7.46-7.26(6H, m), 7.10-6.78(4H, m), 4.01(2H, t, J=5.8Hz), 3.04-3.00(4H, m), 2.49-2.39(2H, m), 2.30-2.21(2H, m), 2.11-2.02(2H, m), 1.94-1.81(2H, m) | HR 468.2165. LR 468 (M + H)⁺ | |
| C-39 | 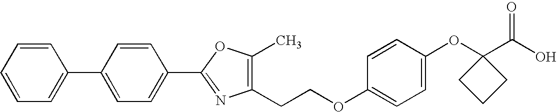 | (CDCl₃, 400 MHz) 8.03(2H, d, J=8.3Hz), 7.66(2H, d, J=8.3Hz), 7.65-7.60(2H, m), 7.49-7.32(3H, m), 6.74-6.63(4H, m), 4.03(2H, t, J=6.6Hz), 2.96(2H, t, J=6.6Hz), 2.81-2.70(2H, m), 2.49-2.35(2H, m), 2.38(3H, s), 2.10-1.90(2H, m). | 470.1948. | Calcd for C₂₉H₂₇NO₅C 74.18, H 5.80, N 2.98. Found: |
| #C 74.07, H 5.83, N 2.89. | | | | |
| C-40 | 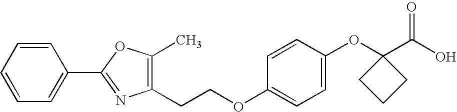 | (CDCl₃, 400 MHz) 8.00-7.93(2H, m), 7.46-7.40(3H, m), 6.73-6.62(4H, m), 4.02(2H, t, J=6.6Hz), 2.94(2H, t, J=6.6Hz), 2.80-2.69(2H, m), 2.47-2.33(2H, m), 2.36(3H, s), 2.10-1.88(2H, m). | 394.1639. | Calcd for C₂₃H₂₃NO₅C 70.21, H 5.89, N 3.56. Found: C 69.87, H 6.05, N 3.47. |
| C-41 | 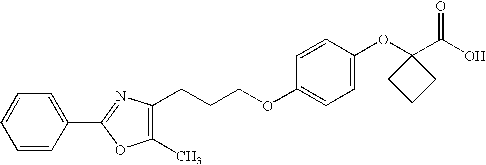 | (CDCl₃, 400 MHz) 8.01-7.94(2H, m), 7.47-7.39(3H, m), 6.82-6.65(4H, m), 3.87(2H, d, J=6.0Hz), 2.82-2.71(2H, m), 2.67-2.60(2H, m), 2.50-2.38(2H, m), 2.29(3H, s), 2.10-1.90(4H, m). | LR 408 (M + H)⁺ | Calcd for C₂₄H₂₅NO₅C 70.74, H 6.18, N 3.44. Found: C 70.52, H |
| #6.19, N 3.41. | | | | |
| C-42 | 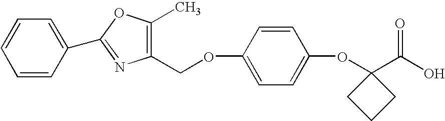 | (CDCl₃, 400 MHz) 8.04-7.97(2H, m), 7.47-7.40(3H, m), 6.86-6.63(4H, m), 4.90(2H, s), 2.78-2.70(2H, m), 2.47-2.35(2H, m), 2.41(3H, s), 2.10-1.90(2H, m). | LR 380 (M + H)⁺ | Calcd for C₂₂H₂₁NO₅C 69.64, H 5.58, N 6.69. Found: C 69.49, H 5.68, N 3.62. |
| C-43 | 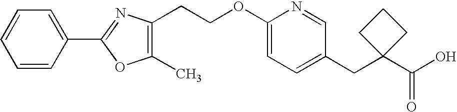 | (MeOH-d₄, 400 MHz) 7.87-7.84(3H, m), 7.46(1H, dd, J=2.3, 8.6Hz), 7.39-7.35(3H, m), 6.57(1H, d, J=8.6Hz), 4.38(2H, t, J=6.7Hz), 2.88(4H, m), 2.25(3H, s), 2.29-2.20(2H, m), 1.88-1.81(2H, m), 1.75-1.68(2H, m). | 393.1815. | |
| C-44 | 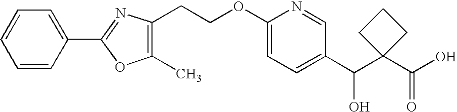 | (DMSO-d₆, 300 MHz) 8.02(1H, d, J=2.1Hz), 7.91-7.88(2H, m), 7.60(1H, dd, J=2.3, 8.5Hz), 7.52-7.47(3H, m), 6.72(1H, d, J=8.7Hz), 5.66(1H, bs), 4.73(1H, s), 4.45(2H, t, J=6.8Hz), 2.90(2H, t, J=6.7Hz), 2.40-2.16(2H, m), 2.30(3H, s), 2.14-2.02(2H, m), 1.75-1.63(1H, m), 1.56-1.42(1H, m). | LR 408 (M)⁻ | Calcd |
| #for C₂₃H₂₄N₂O₅ C 67.63, H 5.92, N 6.86. Found: C 67.51, H 6.08, N 6.75. | | | | |
| C-45 | 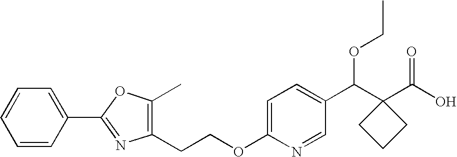 | (Methyl sulfoxide-d₆, 400 MHz): 8.02(1H, d, J=2.1Hz), 7.91-7.88(2H, m), 7.60(1H, dd, J=2.3, 8.5Hz), 7.52-7.47(3H, m), 6.72(1H, d, J=8.7Hz), 4.68(1H, s), 4.45(2H, t, J=6.8Hz), 4.01(2H, q, J=7.1Hz), 2.90(2H, t, J=6.7Hz), 2.40-2.16(2H, m), 2.30(3H, s), 2.14-2.02(2H, m), 1.75-1.63(1H, m), 1.56-1.42(1H, m), 1.29(3H, t, | | |
| #J=7.2Hz) | LR 437 (M + H)⁺ | | | |
| C-46 | 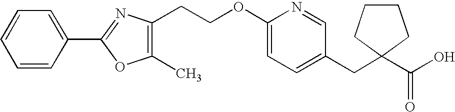 | (CDCl₃, 400 MHz) 8.00-7.93(3H, m), 7.42-7.37(4H, m), 6.63(1H, d, J=8.5Hz), 4.48(2H, t, J=6.6Hz), 2.96(2H, t, J=6.7Hz), 2.86(2H, s), 2.31(3H, s), 2.10-2.02(2H, m), 1.66-1.50(6H, m). | LR 406 (M)⁻ | |
| C-47 | 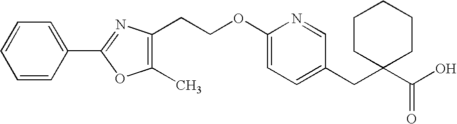 | (CDCl₃, 400 MHz) 7.97-7.93(3H, m), 7.43-7.33(4H, m), 6.63(1H, d, J=8.5Hz), 4.48(2H, t, J=6.8Hz), 2.95(2H, t, J=6.8Hz), 2.73(2H, s), 2.30(3H, s), 2.01(2H, d, J=12.6Hz), 1.59-1.50(3H, m), 1.40-1.15(5H, m). | LR 420 (M)⁻ | |
| C-48 | 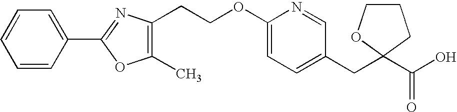 | (CDCl₃, 400 MHz) 7.99-7.94(3H, m), 7.49(1H, dd, J=2.4, 8.6Hz), 7.44-7.36(3H, m), 6.63(1H, d, J=8.5Hz), 4.50(2H, t, J=6.7Hz), 4.00-3.93(1H, m), 3.89-3.81(1H, m), 3.14(1H, d, J=14.1Hz), 2.95(2H, t, J=6.7Hz), 2.85(1H, d, J=14.1Hz), 2.31(3H, s), 1.99-1.73(4H, m). | LR 409 (M)⁺ | |
| C-49 | 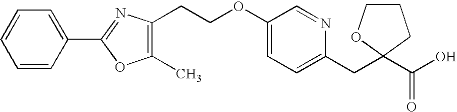 | (MeOH-d₄, 300 MHz) 8.37(1H, d, J=2.6Hz), 8.07(1H, dd, J=2.6, 8.9Hz), 7.93-7.90(2H, m), 7.79(1H, d, J=8.9Hz), 7.47-7.40(3H, m), 4.41(2H, t, J=6.2Hz), 3.92-3.86(2H, m), 3.05(2H, t, J=6.2Hz), 2.36(3H, s), 3.50(1H, d, J=14.3Hz), 3.15(1H, d, J=14.3Hz), 2.31-2.23(1H, m), 2.00-1.92(1H, m), 1.89-1.73(2H, m). | LR | |
| #408 (M)⁻ | Calcd for C₂₃H₂₅N₂O₅Cl C 62.09, H 5.66, N 6.30. Found: C 61.96, H 5.75, N 6.18. | | | |
| C-50 | 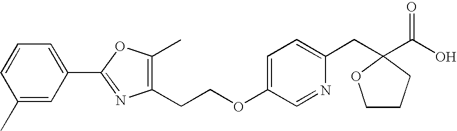 | (MeOD, 400 MHz): 8.37(1H, d, J=2.6Hz), 8.07(1H, dd, J=2.6, 8.9Hz), 7.93-7.90(2H, m), 7.79(1H, d, J=8.9Hz), 7.47-7.40(2H, m), 4.41(2H, t, J=6.2Hz), 3.92-3.86(2H, m), 3.50(1H, d, J=14.3Hz), 3.15(1H, d, J=14.3Hz), 3.05(2H, t, J=6.2Hz), 2.36(3H, s), 2.34(3H, s), 2.31-2.23(1H, m), 2.00-1.92(1H, m), 1.89-1.73(2H, | | |
| #m) | LR 423 (M + H)⁺ | | | |
| C-51 | 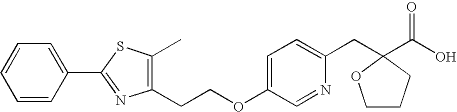 | (CDCl₃, 400 MHz): 8.03(1H, d, J=2.5Hz), 7.77-7.74(2H, m), 7.36-7.31(4H, m), 7.26(1H, d, J=8.6Hz), 4.31(2H, t, J=6.6Hz), 3.81-3.69(2H, m), 3.11(2H, t, J=6.4Hz), 3.02(1H, d, J=13.9Hz), 2.37(3H, s), 2.36-2.34(1H, m), 2.18-2.11(1H, m), 1.94-1.87(1H, m), 1.78-1.68(1H, m), | | |
| #1.60-1.53(1H, m) | LR 425 (M + H)⁺ | | | |
| C-52 | 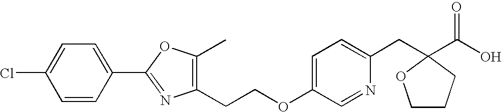 | (CDCl₃, 400 MHz): 8.04(1H, d, J=2.8Hz), 7.85-7.83(2H, m), 7.41-7.39(2H, m), 7.33(1H, dd, J=8.6, 3.0Hz), 7.27(1H, d, J=8.8Hz), 4.23(2H, t, J=6.3Hz), 3.82-3.70(2H, m), 3.17-3.15(1H, m), 3.03(1H, d, J=14.2Hz), 2.91(2H, t, J=6.3Hz), 2.29(3H, s), 2.19-2.12(1H, m), 1.96-1.88(1H, m), 1.78-1.71(1H, m), 1.61-1.54(1H, | | |
| #m) | LR 443 (M + H)⁺ | | | |
| C-53 | 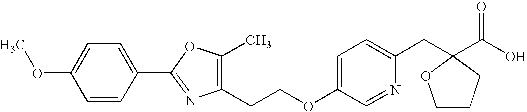 | (MeOD, 400 MHz): 8.01(1H, d, J=1.5Hz), 7.80-7.77(2H, m), 7.25-7.24(2H, m), 6.93-6.91(2H, m), 4.20(2H, t, J=6.3Hz), 3.80-3.69(2H, m), 3.75(3H, s), 3.17-3.15(1H, m), 3.01(1H, d, J=13.9Hz), 2.88(2H, t, J=6.3Hz), 2.26(3H, s), 2.17-2.11(1H, m), 1.94-1.87(1H, m), 1.75-1.66(1H, m), 1.58-1.49(1H, m) | LR 439 (M + H)⁺ | |
| C-54 | 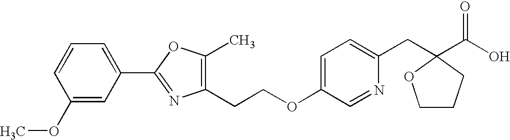 | (MeOD, 400 MHz): 8.01(1H, d, J=2.3Hz), 7.44-7.40(2H, m), 7.30-7.22(3H, m), 6.94-6.91(1H, m), 4.21(2H, t, J=6.4Hz), 3.80-3.67(2H, m), 3.75(3H, s), 3.21-3.17(1H, m), 3.01(1H, d, J=13.9Hz), 2.90(2H, t, J=6.4Hz), 2.28(3H, s), 2.17-2.11(1H, m), 1.94-1.87(1H, m), 1.77-1.67(1H, m), 1.60-1.50(1H, m) | LR 439 (M + H)⁺ | |
| C-55 | 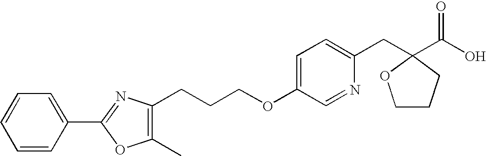 | (CDCl₃, 300 MHz): 8.43(1H, d, J=2.6Hz), 7.97-7.93(1H, m), 7.83-7.79(1H, m), 7.60-7.52(2H, m), 7.48-7.42(3H, m), 4.14(2H, t, J=6.0Hz), 4.06-3.99(2H, m), 3.62(1H, d, J=13.9Hz), 3.37(1H, d, J=13.9Hz), 2.69(2H, t, J=6.0Hz), 2.32(3H, s), 2.23-1.96(6H, m) | LR 423 (M + H)⁺ | |
| C-56 | 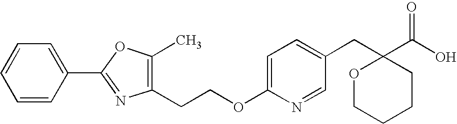 | (CDCl₃, 300 MHz): 7.93-8.03(3H, m), 7.83-7.79(1H, m), 7.37-7.54(4H, m), 6.65(1H, m), 4.50(2H, t, J=6.0Hz), 3.75(2H, m), 2.95(4H, m)2.33(3H, s)2.12(2H, m), 1.39-1.77(5H, m) | LR 423 (M + H)⁺ | |
| C-57 | 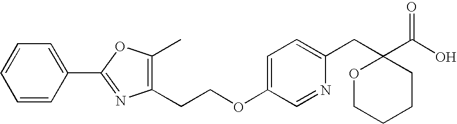 | (MeOD, 400 MHz): 7.98(1H, d, J=2.8Hz), 7.86-7.84(2H, m), 7.39-7.36(3H, m), 7.24(1H, d, J=8.8, 2.8Hz), 7.19(1H, d, J=8.6Hz), 4.21(2H, t, J=6.6Hz), 3.70-3.66(1H, m), 3.60-3.53(1H, m), 2.95(2H, s), 2.90(2H, t, J=6.4Hz), 2.28(3H, s), 2.07-2.01(1H, m), 1.61-1.58(1H, m), 1.40-1.32(4H, m) | LR 423 (M + H)⁺ | |
| C-58 | 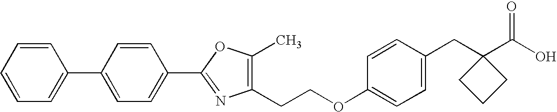 | (DMSO-d₆, 400 MHz) 8.17(2H, d, J=8.3Hz), 7.99(2H, d, J=8.3Hz), 7.92(2H, d, J=8.3Hz), 7.68(2H, t, J=8.3Hz), 7.58(1H, t, J=8.3Hz), 7.24-7.02(4H, m), 4.36(2H, t, J=6.6Hz), 3.11(2H, s), 3.11-3.07(2H, m), 2.55(3H, s), 2.44-2.37(2H, m), 2.15-1.89(4H, m). | 468.2163. | Calcd for C₃₀H₃₀NO₄.0.2H₂O C 76.47, |
| #H 6.29, N 2.97. Found: C 76.48, H 6.30, N 2.90. | | | | |
| C-59 | 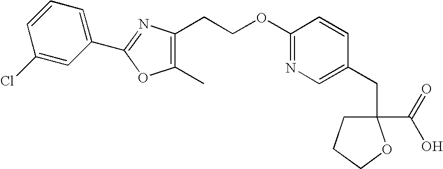 | ¹H NMR(DMSO-d₆, 400 MHz): 8.04(2H, d, J'28.3Hz), 7.95-7.93(3H, m), 7.53(1H, dd, J=8.6 and 2.3Hz), 6.69(1H, d, J=8.3Hz), 4.45(2H, t, J=6.6Hz), 3.75(2H, t, J=6.8Hz), 3.01(1H, d, J=13.9Hz), 2.93(2H, t, J=6.6Hz), 2.82(1H, d, J=14.2Hz), 2.33(3H, s), 2.13-2.07(1H, m), 1.84-1.62(3H, m | | Calcd |
| #for C₂₃H₂₄ClN₂O₅443.1368. Found: 443.1377 | | | | |
| C-60 | 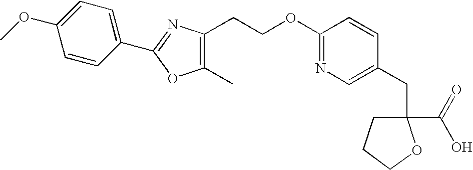 | ¹H NMR(DMSO-d₆, 400 MHz): 12.45(1H, s), 7.95(1H, d, J=2.3Hz), 7.83(2H, d, J=8.8Hz), 7.53(1H, dd, J=8.6 and 2.3Hz), 7.03(2H, d, J=8.8Hz), 6.69(1H, d, J=8.3Hz), 4.42(2H, t, J=6.8Hz), 3.80(3H, s), 3.75(2H, t, J=6.8Hz), 3.01(1H, d, J=13.9Hz), 2.87(2H, t, J=6.8Hz), 2.82(1H, d, J=14.2Hz), 2.28(3H, s), 2.14-2.07(1H, m), 1.83-1.63(3H, m) | | Calcd |
| #for C₂₄H₂₇N₂O₆439.1864. Found: 439.1874 | | | | |
| C-61 | 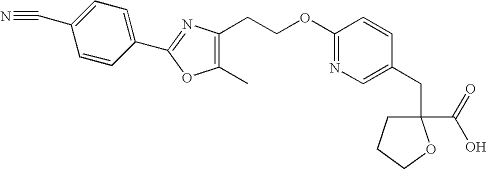 | ¹H NMR(DMSO-d₆, 400 MHz): 8.04(2H, d, J=8.3Hz), 7.95-7.93(3H, m), 7.53(1H, dd, J=8.6 and 2.3Hz), 6.69(1H, d, J=8.3Hz), 4.45(2H, t, J=6.6Hz), 3.75(2H, t, J=6.8Hz), 3.01(1H, d, J=13.9Hz), 2.93(2H, t, J=6.6Hz), 2.82(1H, d, J=14.2Hz), 2.33(3H, s), 2.13-2.07(1H, m), 1.84-1.62(3H, m) | | Calcd |
| #for C₂₄H₂₄N₃O₅434.1711. Found: 434.1705 | | | | |
| C-62 | 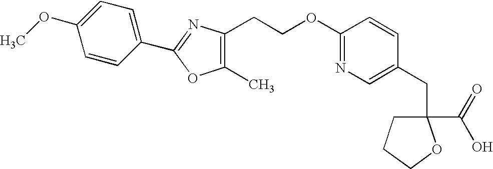 | ¹H NMR(DMSO-d₆, 400 MHz): 7.95(1H, d, J=2.0Hz), 7.53(1H, dd, J=8.3 and 2.3Hz), 7.48(1H, d, J=7.8Hz), 7.42-7.38(2H, m), 7.04(1H, dd, J=8.3 and 2.5Hz), 6.69(1H, d, J=8.3Hz), 4.43(2H, t, J=6.6Hz), 3.81(3H, s), 3.75(2H, t, J=6.8Hz), 3.01(1H, d, J=13.9Hz), 2.90(2H, t, J=6.6Hz), 2.82(1H, d, J=13.9Hz), 2.31(3H, s), | | |
| #2.13-2.07(1H, m), 1.83-1.58(3H, m) | | Calcd for C₂₄H₂₇N₂O₆439.1864. Found: 439.1874 | | |
| C-63 | 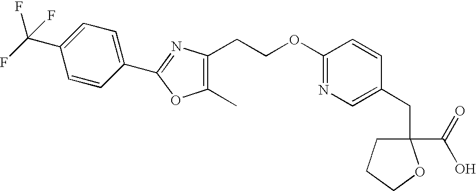 | ¹H NMR(DMSO-d₆, 400 MHz): 12.51(1H, s), 8.10(2H, d, J=8.1Hz), 7.95(1H, d, J=2.0Hz), 7.85(2H, d, J=8.3Hz), 7.53(1H, dd, J=8.3 and 2.3Hz), 6.69(1H, d, J=8.6Hz), 4.45(2H, t, J=6.8Hz), 3.75(2H, t, J=6.6Hz), 3.01(1H, d, J=13.9Hz), 2.93(2H, t, J=6.6Hz), 2.82(1H, d, J=13.9Hz), 2.34(3H, s), 2.13-2.06(1H, m), | | |
| #1.84-1.60(3H, m) | 477.1635 | | | |
| C-64 | 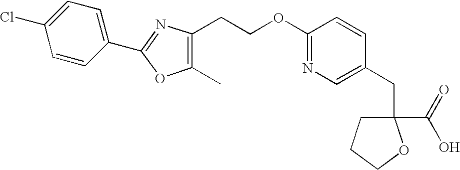 | ¹H NMR(CDCl₃, 400 MHz): 8.16(1H, s), 7.88(2H, d, J=8.6Hz), 7.81(1H, d, J=8.6Hz), 7.41(2H, d, J=8.6Hz), 6.87(1H, d, J=8.8Hz), 4.51(2H, t, J=6.1Hz), 4.03-3.90(2H, m), 3.23(1H, d, J=14.2Hz), 3.04(2H, t, J=6.1Hz), 2.86(1H, d, J=14.2Hz), 2.42-2.35(1H, m), 2.37(3H, s), 2.01-1.87(3H, m) | LR 443 (M + H)⁺ | |
| C-65 | 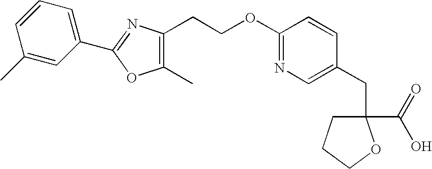 | ¹H NMR(DMSO-d₆, 400 MHz): 7.95(1H, d, J=2.0Hz), 7.73(1H, s), 7.69(1H, d, J=7.3Hz), 7.51(1H, dd, J=8.6 and 2.3Hz), 7.37(1H, t, J=7.8Hz), 7.28(1H, d, J=7.1Hz), 6.69(1H, d, 8.3Hz), 4.43(2H, t, J=6.8Hz), 3.75(2H, t, J=6.6Hz), 3.01(1H, d, J=14.2Hz), 2.89(2H, t, J=6.8Hz), 2.82(1H, d, J=14.2Hz), 2.36(3H, s), | | |
| #2.30(3H, s), 2.14-2.08(1H, m), 1.84-1.60(3H, m) | 423.1927. | Calcd for C₂₄H₂₆N₂O₅0.4H₂O C 64.23 H 6.01 N 6.19. Found: C 64.16, H 6.16 N 6.01 | | |
| C-66 | 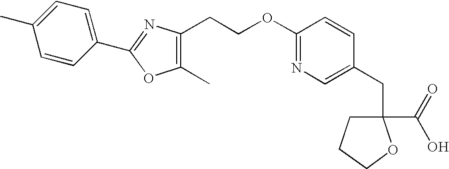 | ¹H NMR(DMSO-d₆, 400 MHz): 7.95(1H, d, J=2.3Hz), 7.79(2H, d, J=8.1Hz), 7.53(1H, dd, J=8.3 and 2.5Hz), 7.29(2H, d, J=8.1Hz), 6.68(1H, d, 8.3Hz), 4.43(2H, t, J=7.1Hz), 3.75(2H, t, J=6.8Hz), 3.01(1H, d, J=13.9Hz), 2.88(2H, t, J=6.8Hz), 2.82(1H, d, J=13.9Hz), 2.34(3H, s), 2.29(3H, s), 2.14-2.07(1H, m), | | |
| #1.84-1.60(3H, m) | 423.1929. | Calcd for C₂₄H₂₆N₂O₅0.9H₂O C 65.71 H 6.39 N 6.39. Found: C 65.61 H 6.34 N 6.20 | | |
| C-67 | 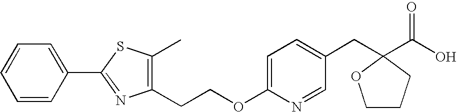 | ¹H NMR(MeOH-d₄, 400 MHz): 8.28(1H, s) 7.76(2H, dt, J=7.7, 0.2Hz)7.56(1H, d, J=9.1Hz)7.34-7.39(1H, m)7.14-7.20(2H, m)6.28(1H, d, J=9.2Hz)4.12(2H, t, J=8.0Hz)3.70-3.84(2H, m)3.14-3.20(1H, m)3.00-3.06(1H, m)2.75(2H, t, J=8.0Hz) 2.34-2.44(1H, m)2.26-2.31(3H, m) .182-2.05(3H, m) | LRMS: 426 (M + | |
| #H)⁺. | | | | |
| C-68 | 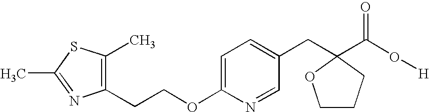 | ¹H NMR(MeOH-d₄, 400 MHz): 8.28(1H, s) 7.56(1H, d, J=9.1Hz)6.28(1H, d, J=9.2Hz) 4.12(2H, t, J=8.0Hz)3.70-3.84(2H, m)3.14-3.20(1H, m)2.99-3.06(1H, m) 2.58(2H, t, J=8.0Hz)2.48(3H, s)2.31-2.44(1H, m)2.18(3H, s)1.81-2.05(3H, m) | LR 364 (M + H)⁺ | |
| C-69 | 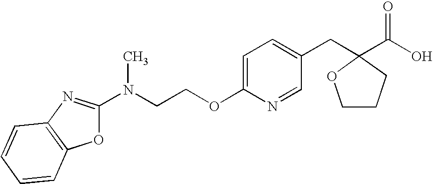 | ¹H NMR(MeOH-d₄, 400 MHz): 8.19(1H, s) 7.45(2H, dd, J=9.0, 8.4Hz)7.33(1H, d, J=7.7Hz)7.15-7.21(1H, m)6.99-7.05(1H, m)6.24(1H, d, J=9.2Hz)4.02(2H, t, J=5.2Hz)3.70-3.84(2H, m)3.42(2H, t, J=5.2Hz)3.14-3.20(1H, m)2.99-3.06(1H, m)2.93(3H, s)2.34-2.43(1H, m)1.82-2.05(3H, m) | LR 398 (M + H)⁺ | |
| C-70 | 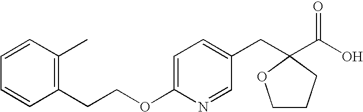 | ¹H NMR(MeOH-d₄, 400 MHz): 8.28(1H, s) 7.56(1H, d, J=9.1Hz)7.08-7.20(4H, m) 6.28(1H, d, J=9.2Hz)4.50(2H, t, J=6.9Hz) 3.70-3.84(2H, m)3.14-3.20(1H, m) 3.00-3.05(1H, m)2.90(2H, t, J=6.9Hz) 2.32-2.43(1H, m)2.28(3H, s)1.81-2.05(3H, m) | LR 342 (M + H)⁺ | |
| C-71 | 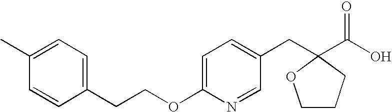 | ¹H NMR(MeOH-d₄, 400 MHz): 8.28(1H, s) 7.56(1H, d, J=9.1Hz)7.09-7.15(4H, m) 6.28(1H, d, J=9.2Hz)4.57(2H, t, J=6.5Hz) 3.70-3.84(2H, m)3.28(2H, t, J=6.5Hz) 3.14-3.20(1H, m)3.00-3.06(1H, m) 2.34-2.44(1H, m)2.27-2.31(3H, m) 1.82-2.05(3H, m) | | |
| C-72 | 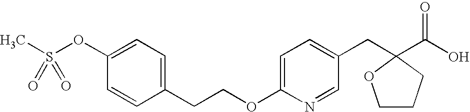 | ¹H NMR(CDCl₃, 400 MHz): 8.2-8.6(1H, br s), 8.10(1H, s), 7.58(1H, d, J=9.1Hz) 6.85-7.25(4H, m)6.70(1H, d, J=9.2Hz) 3.80-4.05(2H, m), 3.63(2H, t, J=6.5Hz) 3.35(3H, s), 2.85(2H, t, J=6.5Hz) 2.30(1H, m)1.65-2.05(3H, m) | | |
| C-73 | 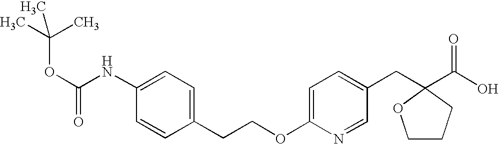 | ¹H NMR(CDCl₃, 400 MHz): 7.98(1H, s) 7.48(1H, d, J=9.1Hz)7.15-7.30(4H, m) 6.65(2H, m)4.37(2H, t, J=6.5Hz)3.80-4.03(2H, m), 2.80-3.20(4H, m), 2.35(3H, m)1.70-2.05(3H, m)1.50(9H, s) | | |
| C-74 | 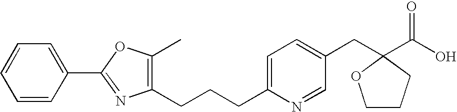 | (Acetone-d₆, 300 MHz): 8.37(1H, d, J=1.9Hz), 7.96(2H, m), 7.56(1H, dd, J=7.9, 2.3Hz), 7.45(3H, m), 7.14(1H, d, J=7.9Hz), 3.89(2H, m), 3.05(2H, m), 2.77(2H, m), 2.50(2H, t, J=7.4Hz), 2.30(3H, s) 2.16(2H, m), 1.88(4H, m) | LR 405 (M)⁻ | |
| C-75 | 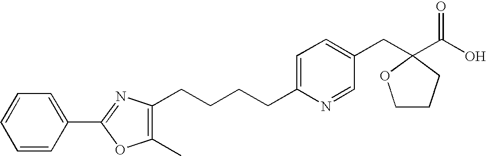 | (MeOD, 400 MHz): 8.19(1H, s), 7.82-7.80(2H, m), 7.61(1H, dd, J=7.8, 1.8Hz), 7.35-7.33(3H, m), 7.15(1H, d, J=8.1Hz), 3.83-3.73(2H, m), 3.09(1H, d, J=13.9Hz), 2.82(1H, d, J=13.9Hz), 2.69(2H, t, J=7.2Hz), 2.41(2H, t, J=6.7Hz), 2.23-2.13(1H, m), 2.20(3H, s), 1.88-1.79(1H, m), 1.78-1.53(6H, m) | LR 421 (M + | |
| #H)⁺ | | | | |
| C-76 | 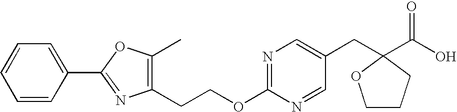 | (MeOD, 300 MHz): 8.34-8.32(2H, bm), 7.87-7.85(2H, bm), 7.8-7.36(3H, bm), 4.54(2H, bs), 3.83(2H, bs), 3.06(1H, d, J=10.7Hz), 2.93-2.89(1H, bm), 2.80(1H, d, J=10.7Hz), 2.24(3H, s), 1.92-1.77(5H, m) | LR 410 (M + H)⁺ | |
| C-77 | 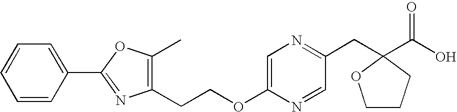 | (Acetone-d₆, 400 MHz): 8.08(1H, s), 7.95(2H, m), 7.46(3H, m), 4.57(2H, t, J=6.5Hz), 3.83(2H, m), 3.10(4H, m), 2.36(3H, s), 2.20(2H, d, J=7.0Hz), 1.75(2H, m) | LR 408 (M)⁻ | |
| C-78 | 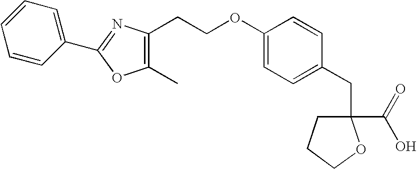 | ¹H NMR(DMSO-d₆, 400 MHz): 12.40(1H, s), 7.90(2H, dd, J=7.8 and 1.8Hz), 7.51-7.44(3H, m), 7.10(2H, d, J=8.3H), 6.81(2H, d, J=8.6), 4.16(2H, t, J=6.8Hz), 3.73(2H, t, J=6.8Hz), 2.99(1H, d, J=13.9Hz), 2.90(2H, t, J=6.6Hz), 2.81(1H, d, J=13.9Hz), 2.34(3H, s), 2.11-2.04(1H, | | |
| #m), 1.82-1.57(3H, m). | 408.1797. | Calcd for C₂₄H₂₅NO₅C 70.75 H 6.18 N 3.44. Found: C 70.53 H 6.18 N 3.31 | | |
| C-79 | 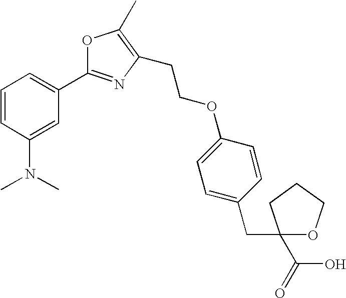 | (DMSO-d₆, 400 MHz) 7.25-7.24(1H, m), 7.19-7.17(2H, m), 7.06-7.04(2H, m), 6.76-6.75(1H, m), 6.72-6.70(2H, m), 4.11(2H, t, J=6.5Hz), 3.77-3.71(2H, m), 3.00-2.96(1H, m), 2.89(6H, s), 2.87-2.85(2H, m), 2.84-2.76(1H, m), 2.98(3H, s), 2.12-2.10(1H, m), 1.85-1.82(1H, m), | | |
| #1.71-1.56(2H, m) | HR 451.2213. LR 451 (M + H)⁺ | | | |
| C-80 | 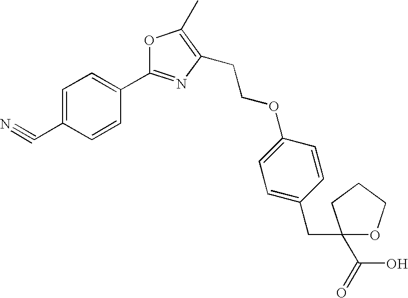 | (DMSO-d₆, 300 MHz) 12.40(1H, s), 8.14-8.11(2H, m), 8.03-8.04(2H, m), 7.19-7.16(2H, m), 6.89-6.86(2H, m), 4.24(2H, t, J=6.5Hz), 3.80(2H, t, J=6.6Hz), 3.09-3.04(1H, m), 3.00(2H, t, J=6.4Hz), 2.91-2.86(1H, m), 2.45(3H, s), 2.19-2.13(1H, m), 1.88-1.67(3H, m) | HR 433.1741. LR 433 (M + H)⁺ | |
| C-81 | 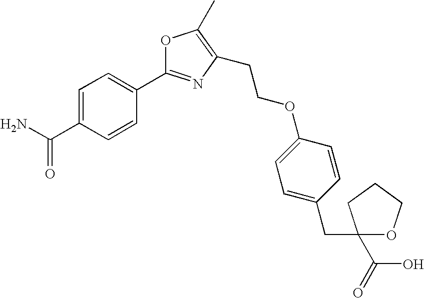 | (DMSO-d₆, 300 MHz) 8.10(1H, s), 8.03-7.96(4H, m), 7.48(1H, s), 7.15-7.12(2H, m), 6.85-6.83(2H, m), 4.20(2H, t, J=6.5Hz), 3.76(2H, t, J=6.5Hz), 3.04-3.00(1H, m), 2.95(2H, t, J=6.5Hz), 2.86-2.82(1H, m), 2.39(3H, s), 2.12(1H, m), 1.83-1.60(3H, m) | LR 451 (M + H)⁺ | |
| C-82 | 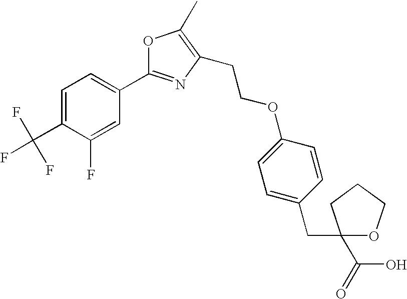 | (CDCl₃, 300 MHz) 7.85-7.77(2H, m), 7.68-7.63(1H, m), 7.15-7.12(2H, m), 6.82-6.80(2H, m), 4.22(2H, t, J=6.5Hz), 4.01-3.96(1H, m), 3.90-3.85(1H, m), 3.19-3.14(1H, m), 2.97(2H, t, J=6.5Hz), 2.90-2.85(1H, m), 2.39(3H, s), 2.36-2.32(1H, m), 2.06-1.96(1H, m), 1.87-1.77(2H, m). | HR 494.1579. LR 494 (M + | |
| #H)⁺ | | | | |
| C-83 | 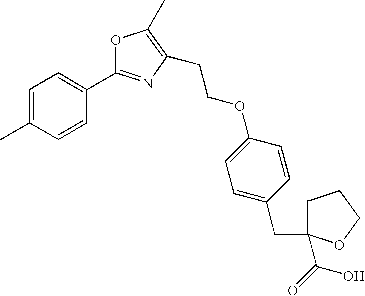 | (CDCl₃, 400 MHz) 7.87-7.84(2H, d), 7.23-7.21(2H, d), 7.14-7.12(2H, d), 6.82-6.80(2H, d), 4.19(2H, t, J=6.5Hz), 4.01-3.94(1H, m), 3.87-3.81(1H, m), 3.17-3.14(1H, m), 2.95(2H, t, J=6.5Hz), 2.90-2.87(1H, m), 2.38(3H, s), 2.35(3H, s), 2.33-2.30(1H, m), 2.03-1.95(1H, m), 1.87-1.72(2H, m). | LR 422 (M + H)⁺ | |
| C-84 | 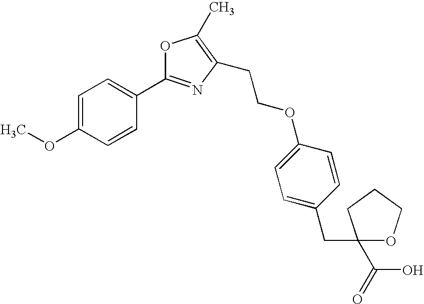 | (CDCl₃, 400 MHz) 7.92-7.90(2H, m), 7.12-7.10(2H, m), 6.94-6.92(2H, m), 6.80-6.78(2H, m), 4.18(2H, t, J=6.5Hz), 4.02-3.97(2H, m), 3.84(3H, s), 3.17-3.13(1H, m), 2.95(2H, t, J=6.5Hz), 2.90-2.86(1H, m), 2.35(3H, s), 2.03-2.00(2H, m), 1.87-1.81(2H, m). | HR 438.1913. LR 438 (M + H)⁺ | |
| C-85 | 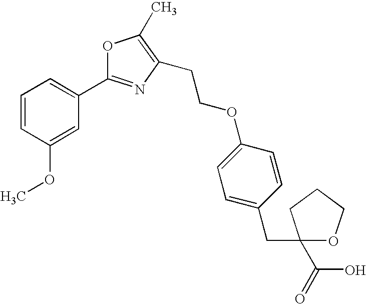 | (CDCl₃, 400 MHz) 7.57-7.55(1H, m), 7.51-7.49(1H, m), 7.35-7.30(1H, m), 7.10-7.07(2H, m), 6.97-6.93(1H, m), 6.81-6.78(2H, m), 4.19(2H, t, J=6.5Hz), 3.87(3H, s), 3.04(2H, s), 2.96(2H, t, J=6.5Hz), 2.48-2.38(2H, m)2.36(2H, s), 2.11-2.02(2H, m), 1.94-1.85(2H, m). | | |
| C-86 | 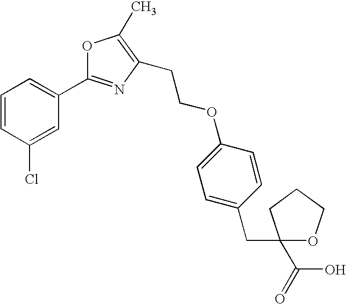 | (CDC₃, 400 MHz) 7.97(1H, s), 7.86-7.84(1H, m), 7.37-7.35(2H, m), 7.15-7.12(2H, m), 6.82-6.80(2H, m), 4.20(2H, t, J=6.5Hz), 3.99-3.95(1H, m), 3.88-3.84(1H, m), 3.18-3.14(1H, m), 2.96(2H, t, J=6.5Hz), 2.90-2.87(1H, m), 2.37(3H, s), 2.36-2.31(1H, m), 2.01-1.98(1H, m), 1.88-1.73(2H, m). | | |
| C-87 | 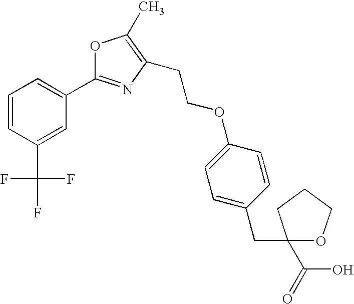 | CDCl₃, 400 MHz) 8.24(1H, s), 8.15-8.14(1H, m), 7.66-7.64(1H, m), 7.57-7.54(1H, m), 7.15-7.13(2H, m), 6.82-6.80(2H, m), 4.21(2H, t, J=6.5Hz), 3.88-3.84(2H, m), 3.18-3.15(1H, m), 2.97(2H, t, J=6.5Hz), 2.90-2.86(1H, m), 2.39(3H, s), 2.01-1.98(2H, m), 1.85-1.81(2H, m). | | |
| C-88 | 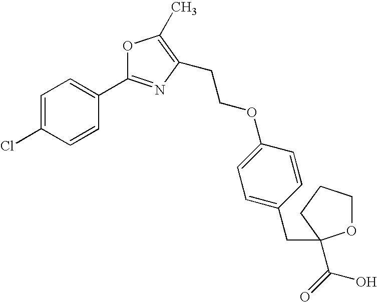 | (CDCl₃, 400 MHz) 7.91-7.89(2H, m), 7.40-7.38(2H, m), 7.14-7.13(2H, m), 6.81-6.79(2H, m), 4.19(2H, t, J=6.5Hz), 4.02-3.93(2H, m), 3.19-3.14(1H, m), 2.95(2H, t, J=6.5Hz), 2.90-2.87(1H, m), 2.36(3H, s), 2.01-1.94(2H, m), 1.87-1.81(2H, m). | | |
| C-89 | 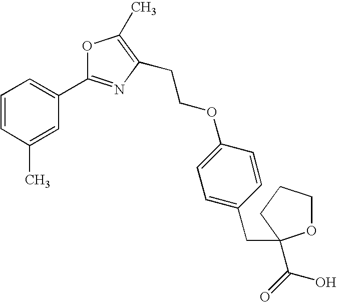 | (CDCl₃, 400 MHz) 7.81-7.75(2H, m), 7.32-7.28(1H, m), 7.22-7.201H, M0, 7.14-7.12(2H, m), 6.81-6.78(2H, m), 4.19(2H, t, J=6.5Hz), 3.98-3.81(2H, m), 3.17-3.10(1H, m), 2.96(2H, t, J=6.5Hz), 2.91-2.86(1H, m), 2.38(3H, s), 2.36(3H, s), 2.00-1.69(4H, m). | | |
| C-90 | 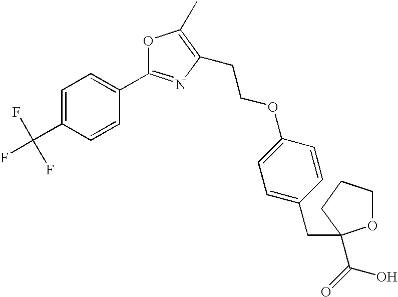 | (CDCl₃, 300 MHz) 8.09-8.06(2H, d), 7.69-7.66(2H, d), 7.15-7.12(2H, m), 6.82-6.79(2H, m), 4.21(2H, t, J=6.5Hz), 4.00-3.81(2H, m), 3.18-3.14(1H, m), 2.97(2H, t, J=6.5Hz), 2.91-2.86(1H, m), 2.39(3H, s), 2.36-2.30(1H, m), 2.04-1.72(3H, m). | HR 476.1661. LR 475 (M + H)⁺ | |
| C-91 | 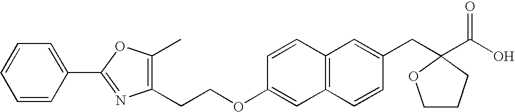 | (CDCl₃, 400 MHz): 7.99-7.97(2H, m), 7.66(1H, d, J=9.3Hz), 7.62-7.59(2H, m), 7.45-7.32(4H, m), 7.11-7.08(2H, m), 4.31(2H, t, J=6.6Hz), 3.97-3.82(2H, m), 3.34(1H, d, J=13.9Hz), 3.08-3.01(3H, m), 2.39-2.33(4H, m), 2.08-2.00(1H, m), 1.87-1.67(2H, m) | LR 458 (M + H)⁺ | |
| C-92 | 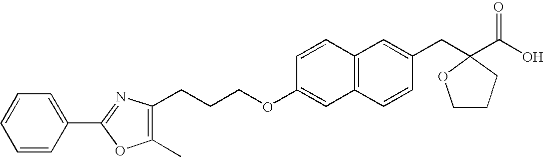 | (CDCl₃, 400 MHz): 7.98(2H, d, J=5.8Hz), 7.68(1H, d, J=9.1Hz), 7.62-7.60(2H, m), 7.42-7.33(4H, m), 7.14(1H, dd, J=2.2, 8.8Hz), 7.07(1H, s), 4.06-3.96(3H, m), 3.87(1H, q, J=7.5Hz), 3.36(1H, d, J=13.9Hz), 3.08(1H, d, J=13.9Hz), 2.73(2H, t, J=7.1Hz), 2.43-2.35(1H, m), 2.27(3H, s), 2.22-2.15(2H, m), 2.10-2.01(1H, m), | | |
| #1.88-1.72(2H, m) | LR 472 (M + H)⁺ | | | |
| C-93 | 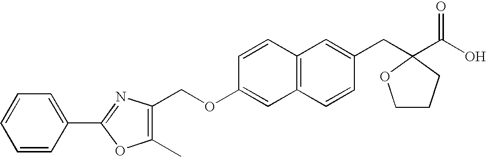 | (MeOD, 400 MHz): 7.93-7.90(2H, m), 7.63-7.56(3H, m), 7.41-7.38(3H, m), 7.31(1H, dd, J=2.5, 8.9Hz), 5.01(2H, s), 3.84-3.73(2H, m), 3.23-3.00(2H, m), 2.39(3H, s), 2.23-2.16(1H, m), 1.96-1.88(1H, m), 1.76-1.57(2H, m) | LR 444 (M + H)⁺ | |

Alternative Predarations of the enantiomers of 2-({6-[2-(5-methyl-2-phenyl-1,3-oxazol-4-yl)ethoxy]pyridin-3-yl}methyl)tetrahydrofuran-2-carboxylic acid (Examples C-48a and C-48b)

### Example C-48a

### Enantiomer 1 of 2-({6-[2-(5-methyl-2-phenyl-1,3-oxazol-4-yl)ethoxy]pyridin-3-yl}methyl)tetrahydrofuran-2-carboxylic acid

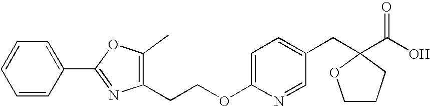

Lithium hydroxide monohydrate (993 mg, 21.1 mmol) was added to a solution of (4S)-4-benzyl-3-{[2-({6-[2-(5-methyl-2-phenyl-1,3-oxazol-4-yl)ethoxy]pyridin-3-yl}methyl)tetrahydro furan-2-yl]carbonyl}-1,3-oxazolidin-2-one (600 mg, 1.06 mmol) in a mixture of tetrahydrofuran:methanol:water (1:1:1, 12 mL). The mixture was stirred at 50° C. for 4.5 hours, then cooled to ambient temperature and stirred for 2 days. The volatile components were removed by evaporation and the residue was diluted with water (5 mL) and extracted with 1:1 hexanes:ether. The aqueous phase was acidified to pH 5 and extracted with ethyl acetate. The organic phase was washed with brine, dried over magnesium sulfate, filtered and evaporated. The residue was purified twice by flash column chromatography (95:4:1 dichloromethane:methanol:ammonium hydroxide) to yield the title compound as a colorless oil (72 mg). LRMS (m/z): 409 (M+H)⁺. ¹H NMR (CDCl₃, 300 MHz) δ 7.99-7.94 (3H, m), 7.49 (1H, dd, J=2.4, 8.6 Hz), 7.44-7.36 (3H, m), 6.63 (1H, d, J=8.5 Hz), 4.50 (2H, t, J=6.7 Hz), 4.00-3.93 (1H, m), 3.89-3.81 (1H, m), 3.14 (1H, d, J=14.1 Hz), 2.95 (2H, t, J=6.7 Hz), 2.85 (1H, d, J=14.1 Hz), 2.31 (3H, s), 1.99-1.73 (4H, m).

### EXAMPLE C-48b

### Enantiomer 2 of 2-({6-[2-(5-methyl-2-phenyl-1,3-oxazol-4-yl)ethoxy]pyridin-3-yl}methyl)tetrahydrofuran-2-carboxylic acid

Enantiomer 2 was prepared using a similar sequence of reactions to those described for enantiomer 1, except starting from (4R)-4-benzyl-1,3-oxazolidin-2-one.

### EXAMPLE C-94

### 1-{6-[2-(5-Methyl-2-phenyl-oxazol-4-yl)-ethoxy]-pyridin-3-ylmethyl}-cyclopropanecarboxylic acid

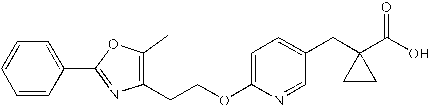

To a solution of 1-{6-[2-(5-methyl-2-phenyl-oxazol-4-yl)-ethoxy]-pyridin-3-ylmethyl}-cyclopropanecarboxylic acid tert-butyl ester (0.2017 g, 0.4642 mmol) in anisole (1.2 mL) was added trifluoroacetic acid (1.2 mL). The resulting solution was stirred at ambient temperature for 3 hours and then concentrated under reduced pressure. The crude residue was diluted with ethyl acetate (25 mL) and water (10 mL) and then basified to pH 5-6 by the addition of saturated aqueous sodium bicarbonate. The phases were separated and the aqueous layer extracted with ethyl acetate (3×25 mL). The combined organic extracts were then dried (anhydrous magnesium sulfate), filtered and concentrated in vacuo to afford the crude product. The pure acid (0.071 g, 40%) was obtained, by recrystallization from diethyl ether/hexanes, as a white solid. LRMS (m/z): 379 (M+H)⁺. ¹H NMR (MeOD, 300 MHz): δ 7.89-7.83 (3H, m), 7.53 (1H, dd, J=8.5, 1.9 Hz), 7.37-7.35 (3H, m), 6.60 (1H, d, J=8.5 Hz), 4.39 (2H, t, J=6.5 Hz), 2.87 (2H, t, J=6.4 Hz), 2.73 (2H, s), 2.23 (3H, s), 1.14-1.11 (2H, m), 0.77-0.74 (2H, m).

### EXAMPLE C-95

### 2-[2-(5-Methyl-2-phenyl-oxazol-4-yl)-ethoxy]-5-[2-(1H-tetrazol-5-yl)-tetrahydro-furan-2-ylmethy]-pyridine

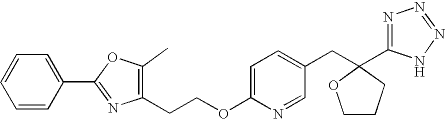

A solution of 2-{6-[2-(5-methyl-2-phenyl-oxazol-4-yl)-ethoxy]-pyridin-3-ylmethyl}-tetrahydro-furan-2-carbonitrile (0.11 g, 0.27 mmol), sodium azide (0.04 g, 0.54 mmol) and zinc bromide (0.03 g, 0.14 mmol) in water and isopropanol (1:2, 1.24 mL) was refluxed for 23 hours. After cooling to ambient temperature, the reaction was quenched with 3N hydrochloric acid (0.14 mL) and ethyl acetate (2.8 mL), and the mixture stirred until completely homogeneous. The aqueous phase was extracted with ethyl acetate (3×50 mL) and the combined organic extracts washed with water (30 mL), dried (anhydrous magnesium sulfate), filtered, and concentrated in vacuo to give the crude product. The residue was recrystallized with diethyl ether/hexanes to afford the title compound (0.052 g, 44%) as a white solid. Elemental Analysis: Calcd C₂₂H₂₃N₇_{O}₃ C, 60.96; H, 5.35; N, 22.62. Found: C, 63.50; H, 5.62; N, 18.80. ¹H NMR (CDCl₃, 300 MHz): δ 7.93 (2H, m), 7.86 (1H, s), 7.40 (3H, m), 7.11 (1H, d, J=1.7 Hz), 6.49 (1H, d, J=8.5 Hz), 4.42 (2H, t, J=6.6 Hz), 3.88 (2H, m), 3.12 (2H, m), 2.92 (2H, t, J=6.5 Hz), 2.62 (1H, m), 2.31 (3H, s), 2.23 (1H, m), 1.89 (2H, m). LRMS (m/z): 433 (M+H)⁺.

Preparations of starting materials for Examples C-1 to C-95 (Preparations c-1 to c-130)

### Predaration c-1

### 4-[2-(5-methyl-2-phenyl-1,3-oxazol-4-yl)ethoxy]benzaldehyde

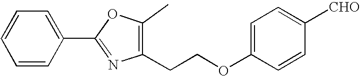

To a solution of the 4-hydroxybenzaldehyde (5.05 g, 41.4 mmol), 2-(5-methyl-2-phenyl-oxazol-4-yl)-ethan-1-ol (8.39 g, 41.4 mmol), and triphenylphosphine (10.9 g, 41.4 mmol) in anhydrous tetrahydrofuran (165 mL), under an atmosphere of nitrogen, was added diethyl azodicarboxylate (7.21 g, 41.4 mmol) dropwise. The resulting solution was stirred at ambient temperature for 8 hours, then diluted with water and extracted with ethyl acetate. The organic phase was dried (anhydrous magnesium sulfate), filtered and evaporated in vacuo. This residue was then purified by flash column chromatography (hexanes to ethyl acetate) to yield the title compound as a white crystalline solid (10.2 g, 80%). LRMS (m/z): 308 (M+H)⁺.

### Preparation c-2

### Methyl 1-(hydroxy{4-[2-(5-methyl-2-phenyl-1,3-oxazol-4-yl)ethoxy]phenyl}methyl) cyclohexane carboxylate

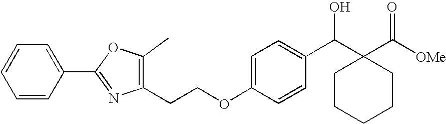

To a suspension of chromium(II) chloride (1.00 g, 8.10 mmol) and lithium iodide (0.087 g, 0.648 mmol) in tetrahydrofuran (20 mL) was added 4-[2-(5-methyl-2-phenyl-1,3-oxazol-4-yl)ethoxy]benzaldehyde (Preparation 1) (1.00 g, 3.24 mmol) and methyl 1-bromocyclohexanoate (1.07 g, 4.85 mmol). The resulting mixture was heated at 50° C. until TLC analysis indicated the reaction was complete. The mixture was cooled to ambient temperature and satuarted aqueous sodium chloride (15 mL) was added. The resulting mixture was stirred for 15 minutes, then partitioned between water and ethyl acetate. The organic phase was washed with water and dried (anhydrous magnesium sulfate), filtered and evaporated. The residue was purified by flash column chromatography (hexanes to 50% ethyl acetate/hexanes) to yield the title compound as a colorless oil (0.797 g, 55%). LRMS (m/z): 450 (M+H)⁺.

### Preparation c-3

### Ethyl 1-(hydroxy{4-[2-(5-methyl-2-phenyl-1,3-oxazol-4-yl)ethoxy]phenyl}methyl)cyclobutane carboxylate

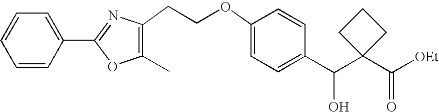

Using analogous procedures to those described for Preparation c-2, the title compound was obtained as a colorless oil. LRMS (m/z): 436 (M+H)⁺.

### Preparation c-4

### Methyl 1-[[4-(allyloxy)phenyl](hydroxy)methyl]cyclopentanecarboxylate

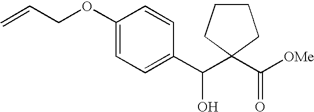

To a solution of methyl cyclopentanoate (3.84 g, 30.0 mmol), in tetrahydrofuran (30 mL) at −78° C. was added a solution of lithium diisopropylamide (15.0 mL of a 2M in tetrahydrofuran, 30.0 mmol) dropwise. The mixture was stirred for 2 hours and then 4-allyloxy benzaldehyde (2.12 g, 13.1 mmol) was added. The mixture was allowed to warm to ambient temperature and stirred for 18 hours. The mixture was diluted with water and extracted with ethyl acetate. The organic phase was washed with saturated aqueous sodium chloride and dried (anhydrous magnesium sulfate), filtered and evaporated. The residue was purified by flash column chromatography (hexanes to 50% ethyl acetate/hexanes) to yield the title compound as a colorless oil (3.67 g, 97%). LRMS (m/z): 273 (M−OH)⁺.

### Preparation c-5

### Methyl 4-[[4-(but-3-enyloxy)phenyl](hydroxy)methyl]tetrahydro-2H-pyran-4-carboxylate

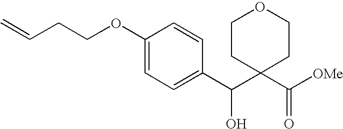

Using analogous procedures to those described for Preparation c-4, the title compound was obtained as a colorless oil. LRMS (m/z): 273 (M−OH)⁺.

### Preparation c-6

### Ethyl 1-[[4-(allyloxy)phenyl](hydroxy)methyl]cyclobutanecarboxylate

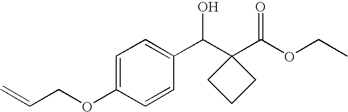

Using analogous procedures to those described for Preparation c-4, the title compound was obtained as a colorless oil. LRMS (m/z): 289 (M)⁺. ¹H NMR (CDCl₃, 300 MHz) δ 7.22 (2H, d, J=8.5 Hz), 6.85 (2H, d, J=8.7 Hz), 6.10-5.98 (1H, m), 5.39 (1H, ddd, J=1.5, 3.2, 17.3 Hz), 5.27 (1H, ddd, J=1.5, 2.8, 10.4 Hz), 4.85 (1H, d, J=6.4 Hz), 4.51 (2H, dt, J=1.5, 5.3 Hz), 4.13 (2H, dq, J=0.9, 7.2 Hz), 3.12 (1H, d, J=6.6 Hz), 2.84-2.78 (1H, m), 2.64-2.58 (1H, m), 2.35-2.29 (2H, m), 1.89-1.83 (1H, m), 1.72-1.66 (1H, m), 1.19 (3H, t, J=7.0 Hz).

### Preparation c-7

### Methyl 1-(4-hydroxybenzyl)cyclopentanecarboxylate

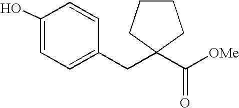

Triethylsilane (10.0 mL, 63 mmol) was added to a solution of methyl 1-[[4-(allyloxy)phenyl](hydroxy)methyl]cyclopentanecarboxylate (Preparation c-4) (3.66 g, 12.6 mmol) in dichloromethane (30 mL) and trifluoroacetic acid (30 mL) at room temperature. The resulting mixture was stirred for 1 hour then evaporated in vacuo and azeotroped with toluene. The residue was dissolved in tetrahydrofuran (32 mL) and morpholine (3.62 mL, 41.6 mmol) and tetrakis(triphenylphosphine)palladium (0) (1.46 g, 1.26 mmol) was added. The resulting mixture was stirred at room temperature for 18 hours, filtered through Celite and evaporated to dryness. The residue was dissolved in ethyl acetate and washed with 1 N hydrochloric acid then saturated sodium bicarbonate solution. The organic phase was dried (anhydrous magnesium sulfate), filtered and evaporated and the residue was purified by flash column chromatography (hexanes to ethyl acetate) to yield the title compound as a white crystalline solid (1.75 g, 59%). LRMS (m/z): 233 (M)⁻.

### Preparation c-8

### Methyl 4-(4-hydroxybenzyl)tetrahydro-2H-pyran-4-carboxylate

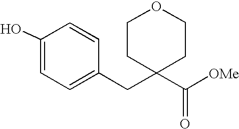

Using analogous procedures to those described for Preparation c-7, the title compound was obtained as a white crystalline solid. LRMS (m/z): 249 (M)⁻.

### Preparation c-9

### Ethyl 1-(4-hydroxybenzyl)cyclobutanecarboxylate

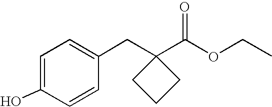

Using analogous procedures to those described for Preparation c-7, the title compound was obtained as a white crystalline solid. LRMS (m/z): 234 (M)⁻. ¹H NMR (CDCl₃, 300 MHz) δ 6.97 (2H, d, J=8.5 Hz), 6.68 (2H, d, J=8.5 Hz), 5.10 (1H, bs), 4.10 (2H, q, J=7.2 Hz), 3.00 (2H, s), 2.44-2.35 (2H, m), 2.07-1.99 (2H, m), 1.91-1.80 (2H, m), 1.20 (3H, t, J=7.2 Hz).

### Preparation c-10

### Methyl 1-{4-[2-(5-methyl-2-phenyl-1,3-oxazol-4-yl)ethoxy]benzyl cyclopentanecarboxylate

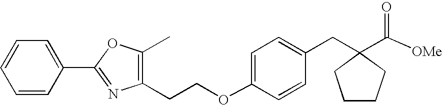

Using analogous procedures to those described for Preparation c-1-c-7, the title compound was obtained as a colorless oil. LRMS (m/z): 249 (M)⁻.

### Preparation c-11

### Methyl 1-{4-[3-(5-methyl-2-phenyl-1,3-oxazol-4-yl)propoxy]benzyl}cyclopentanecarboxylate

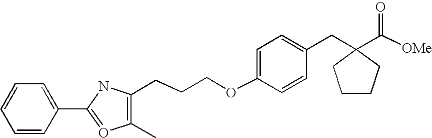

Using analogous procedures to those described for Preparation c-1-c-7, the title compound was obtained as a colorless oil. LRMS (m/z): 434 (M+H)⁺.

### Preparation c-12

### Methyl 4-{4-[2-(5-methyl-2-phenyl-1,3-oxazol-4-yl)ethoxy]benzyl}tetrahydro-2H-pyran-4-carboxylate

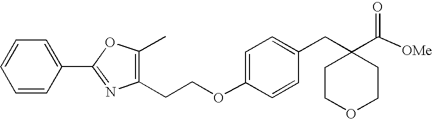

Using analogous procedures to those described for Preparation c-1-c-7, the title compound was obtained as a colorless oil. LRMS (m/z): 436 (M+H)⁺.

### Preparation c-13

### Ethyl 1-{4-[2-(4-bromophenyl)ethoxy]benzyl}cyclobutanecarboxylate

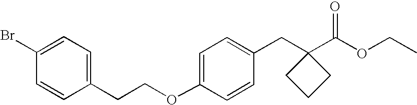

Using analogous procedures to those described for Preparation c-1-c-7, the title compound was obtained as a colorless oil. LRMS (m/z): 417 (M)⁺. ¹H NMR (CDCl₃, 300 MHz) δ 7.36 (2H, d, J=8.3 Hz), 7.08 (2H, d, J=8.3 Hz), 6.96 (2H, d, J=8.7 Hz), 6.70 (2H, d, J=8.7 Hz), 4.04 (2H, t, J=6.8 Hz), 4.03 (2H, q, J=7.2 Hz), 2.95 (2H, t, J=6.8 Hz), 2.94 (2H, s), 2.37-2.27 (2H, m), 2.00-1.91 (2H, m), 1.84-1.73 (2H, m), 1.14 (3H, t, J=7.2 Hz).

### Preparations c-14 to c-35

Preparations c-14 to c-35 were prepared using analogous procedures to those used for Preparation c-1.

| | | | MS (m/z) |
|---|---|---|---|
| Prep # | Structure | ¹H NMR | (LR or HR) |
| c-14 | 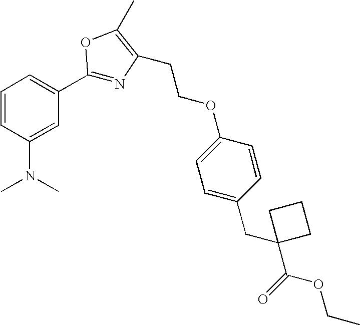 | | LR 463 (M + H)⁺ |
| c-15 | 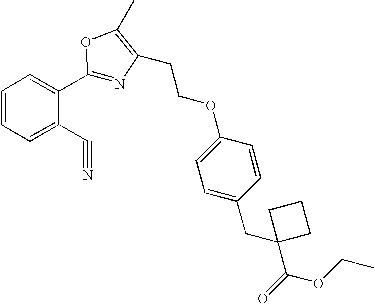 | (CDCl₃, 300 MHz) 8.14-8.11(1H, m), 7.78-7.75(1H, m), 7.67-7.62 (1H, m), 7.51-7.45(1H, m), 7.04-7.01(2H, m),6.80-6.74(2H, m ), 4.23(2H, t, J=6.5), 4.13-4.06(2H, q, J=7.1 Hz), 3.00(2H, t, J=3.3 Hz), 2.42(3H, s), 2.40-2.36(2H, m), 2.05-2.00(2H, m), 1.87-1.84 (2H, m), 1.21(3H, t, J=7.1 Hz). | LR 446 (M + H)⁺ |
| c-16 | 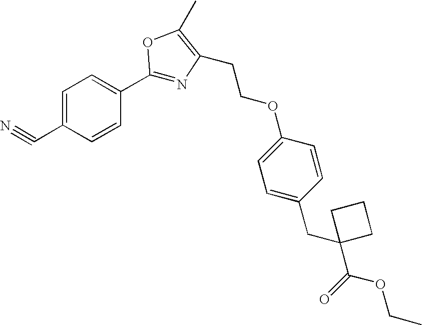 | (CDCl₃, 300 MHz), 8.07-8.04(2H, m), 7.71-7.68(2H, m), 7.03-7.01 (2H, m), 6.78-6.75(2H, m), 4.19 (2H, t, J=6.5 Hz), 4.13-4.06(3H, m), 2.99(2H, s), 2.96(2H, t, J=6.5 Hz), 2.39(3H, s), 2.38-2.34(2H, m), 2.02-1.99(2H, m), 1.87-1.82 (2H, m), 1.20(3H, t, J=6.9 Hz). | LR 445 (M + H)⁺ |
| c-17 | 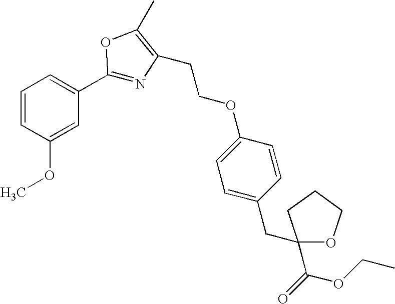 | (CDCl₃, 300 MHz), 8.08-8.05(2H, m), 7.71-7.68(2H, m), 7.16-7.13 (2H, m), 6.80-6.77(2H, m), 5.01-4.92(2H, m), 4.21(2H, t, J=6.5 Hz), 3.92-3.85(2H, m), 3.15-3.08 (1H, m), 2.97(2H, t, J=6.4 Hz), 2.95-2.88(1H, m), 2.40(3H, s), 2.28-2.21(1H, m), 2.04(3H, s), 1.91-1.86(2H, m). | LR 461 (M + H)⁺ |
| c-18 | 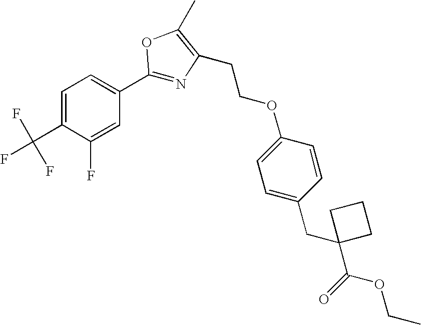 | | LR 506 (M + H)⁺ |
| c-19 | 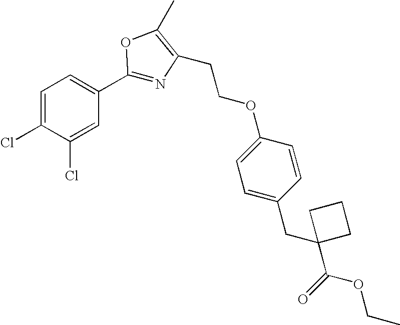 | (CDCl₃, 300 MHz), 8.05-8.04(1H, d), 7.79-7.75(1H, m), 7.47-7.44 (1H, d), 7.03-7.00(2H, m), 6.76-6.75(2H, m), 4.18(2H, t, J=6.5 Hz), 4.13-4.06(2H, q, J=7.1 Hz), 3.00(2H, s), 2.94(2h, t, J=6.5 Hz), 2.49-2.36(2H, s), 2.35(3H, s), 2.04-1.97(2H, m), 1.87-1.81 (2H, m), 1.20(3H, t, J=7.1 Hz). | LR 489 (M + H)⁺ |
| c-20 | 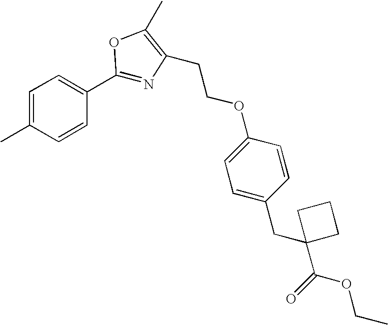 | | LR 435 (M + H)⁺ |
| c-21 | 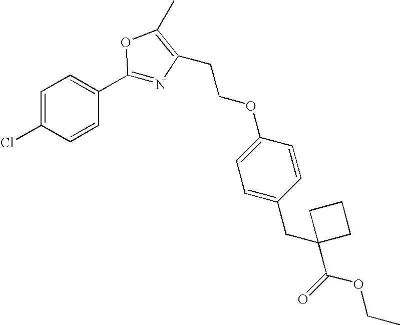 | | LR 455 (M + H)⁺ |
| c-22 | 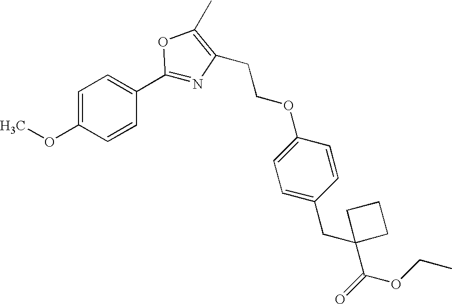 | | LR 450 (M + H)⁺ |
| c-23 | 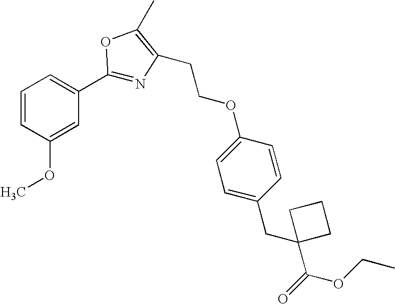 | (CDCl₃, 400 MHz), 7.57-7.56(1H, m), 7.51-7.50(1H, m), 7.34-7.30 (1H, m), 7.03-7.01(2H, m), 6.96-6.93(1H, m), 6.80-6.77(2H, m), 4.20(2H, t, J=6.6 Hz), 4.12-4.07 (2H, q, J=7.0 Hz), 3.86(3H, s), 3.00(2H, s), 2.95(2H, t, J=6.5 Hz), 2.42-2.37(2H, m), 2.35(3H, m), 2.05-1.98(2H, m), 1.88-1.81 (2H, m). | LR 450 (M + H)⁺ |
| c-24 | 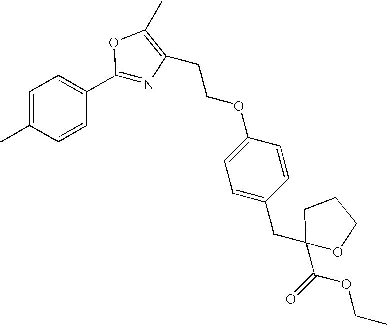 | | LR 450 (M + H)⁺ |
| c-25 | 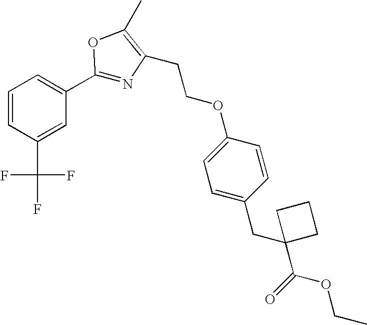 | | LR 488 (M + H)⁺ |
| c-26 | 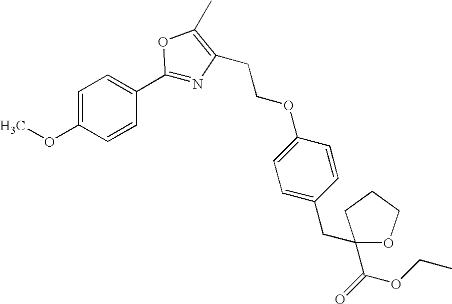 | | LR 466 (M + H)⁺ |
| c-27 | 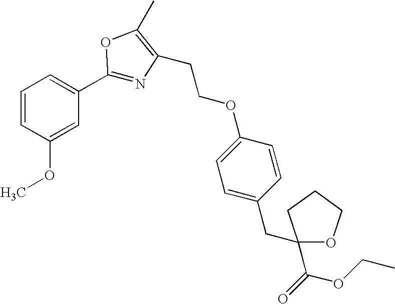 | (CDCl₃, 400 MHz), 7.57-7.56(1H, m), 7.51-7.50(1H, m), 7.34-7.30 (1H, m), 7.03-7.01(2H, m), 6.96-6.93(1H, m), 6.80-6.77(2H, m), 44.20(2H, t, J=6.5 Hz), 4.12-4.07 (2H, q, J=7.0 Hz), 3.86(3H, s), 3.00(2H, s), 2.95(2H, t, J=6.5 Hz), 2.42-2.37(2H, m), 2.35(3H, s), 2.05-1.98(2H, m), 1.86-1.81 (2H, m), 1.20(3H, t, J=7.0 Hz). | LR 450 (M + H)⁺ |
| c-28 | 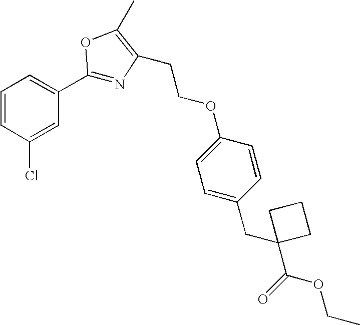 | (CDCl₃, 400 MHz), 7.97(1H, s), 7.86-7.83(1H, s), 7.35-7.33(2H, m), 7.03-7.01(2H, m), 6.79-6.77 (2H, m), 4.19(2H, t, J=6.5 Hz), 4.12-4.07(2H, q, J=7.0 Hz), 3.00 (2H, s), 2.95(2H, t, J=6.5 Hz), 2.422.38(2H, m), 2.35(3H, s), 2.05-1.99(2H, m), 1.91-1.82(2H, m), 1.20(3H, t, J=7.0 Hz) | LR 454 (M + H)⁺ |
| c-29 | 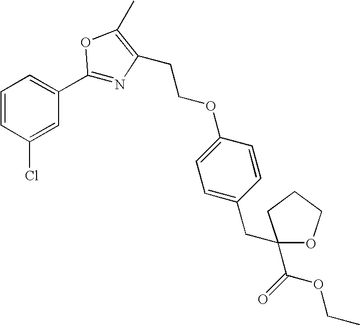 | (CDCl₃, 400 MHz), 7.97(1H, s), 7.87-7.84(1H, m), 7.36-7.35(2H, m), 7.16-7.14(2H, m), 6.81-6.78 (2H, m), 4.21(2H, t, J=6.5 Hz), 4.15-4.10(q, J=7.0 Hz), 3.91-3.87 (2H, m), 3.14-3.10(1H, m), 2.96 (2H, t, J=6.5 Hz), 2.93-2.90(1H, m), 2.37(3H, s), 2.26-2.12(2H, m), 1.89-1.76(2H, m), 1.21(3H, t, J=7.0 Hz). | LR 470 (M + H)⁺ |
| c-30 | 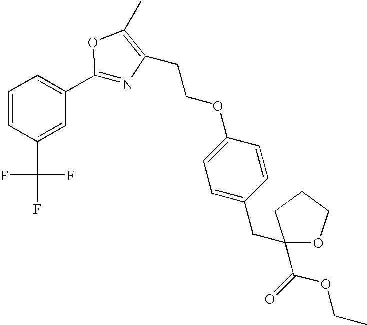 | (CDCl₃, 400 MHz), 8.24(1H, s), 8.16-8.14(1H, m), 7.66-7.64(1H, m), 7.57-7.53(1H, m), 7.16-7.14 (2H, m), 6.81-6.79(2H, m), 4.22 (2H, t, J=6.5 Hz), 4.15-4.10(2H, q, J=7.0 Hz), 3.93-3.86(2H, m), 3.14-3.11(1H, m), 2.97(2H, t, J=6.5 Hz), 2.93-2.90(1H, m), 2.39 (3H, s), 2.26-2.20(2H, m), 1.91-1.78(2H, m), 1.21(3H, t, J=7.0 Hz). | LR 504 (M + H)⁺ |
| c-31 | 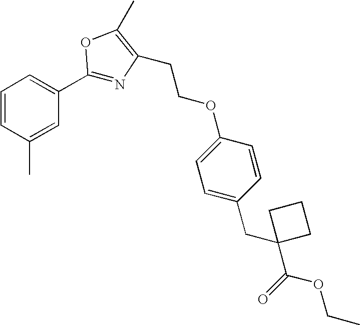 | (CDCl₃, 300 MHz), 7.81-7.75(2H, m), 7.32-7.27(1H, m), 7.21-7.19 (1H, m), 7.03-7.00(2H, m), 6.78-6.76(2H, m), 4.19(2H, t, J=6.5 Hz), 4.13-4.06(2H, q, J=7.1 Hz), 3.00(2H, s), 2.95(2H, t, J=6.5 Hz), 2.43-2.40(2H, m), 2.37(3H, s), 2.35(3H, s), 2.07-1.97(2H, m), 1.89-1.79(2H, m), 1.19(3H, t, J=7.1 Hz) | LR 434 (M + H)⁺ |
| c-32 | 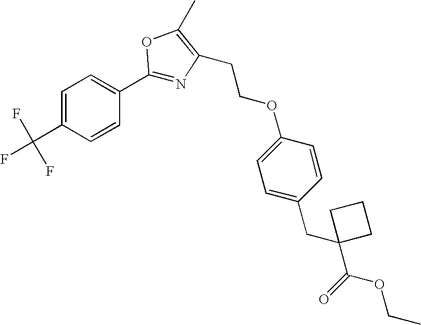 | (CDCl₃, 300 MHz), 8.09-8.06(2H, m), 7.69-7.66(2H, m), 7.04-7.01 (2H, m), 6.80-6.77(2H, m), 4.21 (2H, t, J=6.5 Hz), 4.15-4.06(2H, m), 3.00(2H, s), 2.96(2H, t, J=6.5 Hz), 2.43-2.34(5H, m), 2.01-1.97(2H, m), 1.92-1.79(2H, m), 1.20(3H, t, J=7.1 Hz) | LR 488 (M + H)⁺ |
| c-33 | 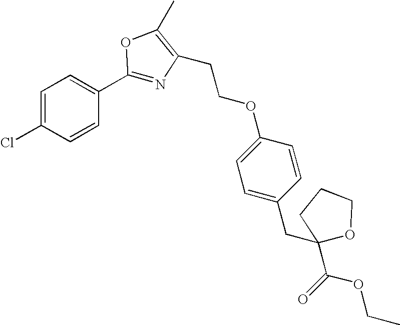 | | LR 470 ( + H)⁺ |
| c-34 | 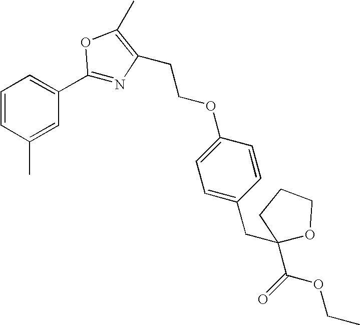 | (CDCl₃, 300 MHz), 7.81-7.75(1H, m), 7.33-7.27(2H, m), 7.22-7.19 (1H, m), 7.16-7.13(2H, m), 6.81-6.78(2H, m), 4.20(2H, t, J=6.7 Hz), 4.16-4.09(2H, q, J=7.1 Hz), 3.94-3.82(2H, m), 3.14-3.08(1H, m), 2.98-2.94(2H, t, J=6.5 Hz), 2.94-2.88(1H, m), 2.38(3H, s), 2.36(3H, s), 2.27-2.19(1H, m), 1.92-1.74(2H, m), 1.68-1.62(1H, m), 1.21(3H, t, J=7.1 Hz). | LR 450 (M + H)⁺ |
| c-35 | 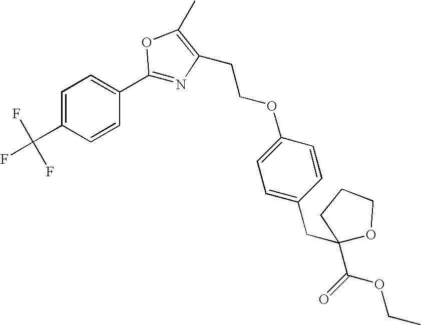 | (CDCl₃, 300 MHz), 8.09-8.07(2H, m), 7.69-7.66(2H, m), 7.16-7.14 (2H, m), 6.81-6.78(2H, m), 4.21 (2H, t, J=6.5 Hz), 4.16-4.09(2H, q, J=7.1 Hz), 3.95-3.83(2H, m), 3.15-3.10(1H, m), 2.97(2H, t, J=6.5 Hz), 2.94-2.89(1H, m), 2.39 (3H, s), 2.27-2.18(1H, m), 1.92-1.61(3H, m), 1.21(3H, t, J=7.1 Hz). | LR 504 (M + H)⁺ |

### Preparation c-36

### Ethyl 1-{4-[2-(4′-methoxy-1,1′-biphenyl-4-yl)ethoxy]benzyl}cyclobutanecarboxylate

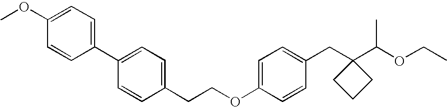

To a solution of ethyl 1-{4-[2-(4-bromophenyl)ethoxy]benzy}cyclobutanecarboxylate (Preparation c-13) (0.25 g, 0.5990 mmol), tetrakis(triphenylphosphine)palladium(0) (0.1252 g, 0.6589 mmol), benzene (1.6 mL), and 2M aqueous sodium carbonate (0.8 mL), under an atmosphere of nitrogen, was added a solution of the boronic acid (0.8640 mmol, 1.1 equiv.) in ethanol (0.4 mL). The resulting mixture was degassed and then refluxed for 16 hours followed by cooling to ambient temperature. To this was then added 30% aqueous hydrogen peroxide (0.04 mL) dropwise and the resulting solution stirred at ambient temperature for 1 hour. The solution was then extracted with ethyl acetate (3×100 mL) and the combined organic extracts washed with saturated aqueous sodium chloride (100 mL), dried (anhydrous magnesium sulfate), filtered and concentrated in vacuo to afford the crude product. The residue was purified by flash column chromatography (hexanes to 40% ethyl acetate/hexanes) to yield the pure product as a colorless oil. LRMS (m/z): 462 (M+H₂O)⁺. ¹H NMR (CDCl₃, 300 MHz) δ 7.51 (2H, d, J=5.7 Hz), 7.49 (2H, d, J=4.7 Hz), 7.32 (2H, d, J=8.1 Hz), 7.03 (2H, d, J=8.5 Hz), 6.97 (2H, d, J=8.9 Hz), 6.80 (2H, d, J=8.5 Hz), 4.15 (1H, t, J=7.2 Hz), 4.10 (2H, q, J=7.2 Hz), 3.84 (3H, s), 3.10 (2H, t, J=7.1 Hz), 3.01 (2H, s), 2.43-2.34 (2H, m), 2.07-1.98 (2H, m), 1.90-1.80 (2H, m), 1.20 (3H, t, J=7.2 Hz).

### Preparations c-37 to c-43

Preparations c-37 to c-43 were prepared using analogous procedures to those used for Preparation c-36

| Prep | | | (LR or |
|---|---|---|---|
| # | Structure | ¹H NMR | HR |
| c-37 | 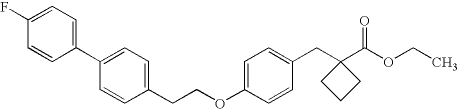 | (CDCl₃, 300 MHz), 7.46(2H, d, J=8.9 Hz), 7.41 (2H, d, J=8.3 Hz), 7.27(2H, d, J=8.1 Hz), 7.03(2H, t, J=8.9 Hz), 6.97(2H, d, J=8.5 Hz), 6.73(2H, d, J=8.7 Hz), 4.09(2H, t, J=7.0 Hz), 4.03(2H, q, J=7.0 Hz), 3.03(2H, t, J=7.0 Hz), 2.94(2H, s), 2.37-2.27(2H, m), 2.00-1.91(2H, m), 1.83-1.72(2H, m), 1.13(3H, t, J=7.1 Hz). | LR 433 (M + H)⁺ |
| c-38 | 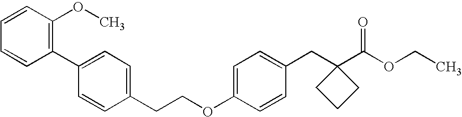 | (CDCl₃, 300 MHz), 7.48(2H, d, J=8.1 Hz), 7.34-7.29(4H, m), 7.05(2H, d, J=8.7 Hz), 6.99(2H, d, J=8.5 Hz), 6.81(2H, d, J=8.7 Hz), 4.18 (2H, t, J=7.3 Hz), 4.11(2H, q, J=7.0 Hz), 3.81 (3H, s), 3.12(2H, t, J=7.3 Hz), 3.02(2H, s), 2.44-2.35(2H, m), 2.08-1.99(2H, m), 1.91-1.80 (2H, m), 1.21(3H, t, J=7.0 Hz). | LR 467 (M + Na)⁺ |
| c-39 | 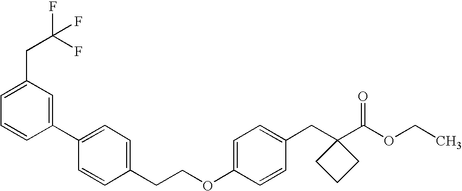 | (CDCl₃, 300 MHz), 7.53-7.49(3H, m), 7.44(2H, t, J=7.9 Hz), 7.38(2H, t, J=7.9 Hz), 7.09(1H, dm, J=8.0 Hz), 7.04(2H, d, J=8.5 Hz), 6.80 (2H, d, J=8.5 Hz), 4.17(2H, t, J=7.0 Hz), 4.10 (2H, q, J=7.0 Hz), 3.12(2H, t, J=7.0 Hz), 3.01 (2H, s), 2.44-2.35(2H, m), 2.07-1.98(2H, m), 1.90-1.83(2H, m), 1.21(3H, t, J=7.2 Hz) | LR 499 (M + H)⁺ |
| c-40 | 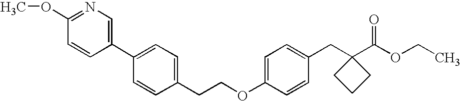 | (CDCl₃, 300 MHz), 8.36(1H, s), 7.77(1H, dd, J=2.5 and 8.7 Hz), 7.46(2H, d, J=8.1 Hz), 7.35 (2H, d, J=8.1 Hz), 7.03(2H, d, J=8.3 Hz), 6.81(1H, d, J=8.3 Hz), 6.79(2H, d, J=8.3 Hz), 4.16(2H, t, J=6.8 Hz), 4.09(2H, q, J=7.2 Hz), 3.97(3H, s), 3.10(2H, t, J=7.0 Hz), 3.00 (2H, s), 2.43-2.33(2H, m), 2.06-1.97(2H, m), 1.89-1.79(2H, m), 1.20(3H, t, J=7.1 Hz). | LR 446 (M + H)⁺ |
| c-41 | 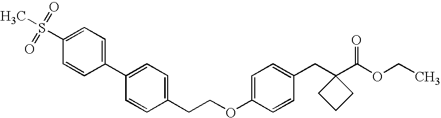 | (CDCl₃, 300 MHz), 7.99(2H, d, J=8.1 Hz), 7.75 (2H, d, J=8.1 Hz), 7.56(2H, d, J=8.1 Hz), 7.40(2H, d, J=7.9 Hz), 7.03(2H, d, J=8.5 Hz), 6.79(2H, d, J=8.5 Hz), 4.17(2H, t, J=6.8 Hz), 4.09(2H, q, J=7.2 Hz), 3.13(2H, t, J=6.9 Hz), 3.08(3H, s), 3.00(2H, s), 2.43-2.33(2H, m), 2.06-1.97(2H, m), 1.90-1.79(2H, m), 1.20 (3H, t, J=7.2 Hz). | LR 511 (M + Na)⁺ |
| c-42 | 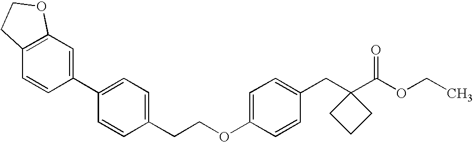 | (CDCl₃, 300 MHz), 7.48(2H, d, J=8.3 Hz), 7.41 (1H, s), 7.34-7.31(1H, m), 7.31(2H, d, J=8.3 Hz), 7.04(2H, d, J=8.7 Hz), 6.85(1H, d, J=8.5 Hz), 6.80(2H, d, J=8.l5 Hz), 4.61(2H, t, J=8.7 Hz), 4.16(2H, t, J=7.2 Hz), 4.10(2H, q, J=7.2 Hz), 3.27(2H, t, J=8.7 Hz), 3.10(2H, t, J=7.0 Hz), 3.01(2H, s), 2.44-2.35(2H, m), 2.07-1.99(2H, m), 1.91-1.83(2H, m), 1.21(3H, t, J=7.2 Hz). | LR 479 (M + Na)⁺ |
| c-43 | 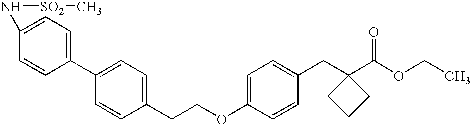 | (CDCl₃, 300 MHz), 7.57(2H, d, J=8.5 Hz), 7.50 (2H, d, J=8.5 Hz), 7.35(2H, d, J=8.5 Hz), 7.27(2H, d, J=8.5 Hz), 7.03(2H, d, J=8.7 Hz), 6.79(2H, d, J=8.7 Hz), 6.37(1H, bs), 4.16 (2H, t, J=7.0 Hz), 4.09(2H, q, J=7.0 Hz), 3.11 (2H, d, J=7.0 Hz), 3.04(3H, s), 3.00(2H, s), 2.43-2.33(2H, m), 2.06-1.97(2H, m), 1.89-1.79 (2H, m), 1.20(3H, t, J=7.2 Hz). | LR 508 (M)⁺ |

### Preparation c-44

### Methyl 4-{4-[(5-methyl-2-phenyl-1,3-oxazol-4-yl)methoxy]benzyl}tetrahydro-2H-pyran-4-carboxylate

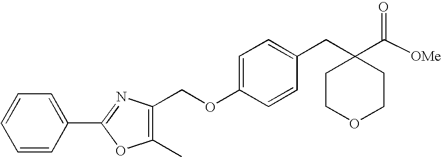

A solution of methyl 4-(4-hydroxybenzyl)tetrahydro-2H-pyran-4-carboxylate (Preparation c-8) (0.500 g, 2.0 mmol), cesium carbonate (1.96 g, 6.0 mmol) and chloride (0.458 g, 2.2 mmol) in acetonitrile was heated at 140° C. in a microwave synthesizer for 10 minutes. The mixture was cooled, filtered and the filtrate evaporated. The residue purified by flash column chromatography (hexanes to ethyl acetate) to yield the title compound as a white crystalline solid (0.827 g, 98%). LRMS (m/z): 422 (M+H)⁺.

### Preparation c-45

### 5-Bromo-2-[2-(5-methyl-2-phenyl-1,3-oxazol-4-yl)ethoxy]pyridine

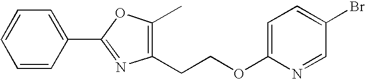

To a solution of 2,5-dibromo-pyridine (5 g, 21.1060 mmol) and 2-(5-methyl-2-phenyl-oxazol-4-yl)-ethanol (5.1472 g, 25.3271 mmol) in anhydrous tetrahydrofuran (85 mL), under an atmosphere of nitrogen, was added potassium tert-butoxide (2.8422 g, 25.3271 mmol). The resulting mixture was heated at reflux for 16 hours and then allowed to cool to ambient temperature. The mixture was evaporate to about 20 mL and partitioned between saturated aqueous ammonium chloride (50 mL) and ethyl acetate (50 mL). The layers were separated and the aqueous layer extracted with ethyl acetate (2×50 mL). The combined organic extracts were then washed with water (2×50 mL), saturated aqueous sodium chloride (50 mL), dried (anhydrous magnesium sulfate), filtered and concentrated in vacuo to afford the crude product. The residue was purified by flash column chromatography (hexanes to 20% ethyl acetate/hexanes) to yield a white crystalline solid (6.3 g, 83%). LRMS (m/z): 359 (M)⁺.

¹H NMR (CDCl₃, 400 MHz) 8.17 (1H, d, J=2.0 Hz), 7.96 (2H, dd, J=2.0, 8.1 Hz), 7.61 (1H, dd, J=2.7, 8.7 Hz), 7.43-7.38 (3H, m), 6.62 (1H, d, J=8.6 Hz), 4.52 (2H, t, J=6.8 Hz), 2.96 (2H, t, J=6.8 Hz), 2.32 (3H, s).

### Preparations c-46 to c-47

Preparations c-46 to c-47 were prepared by general procedure for Preparation c-45.

| | | | MS (m/z) |
|---|---|---|---|
| Prep # | Structure | ¹H NMR | (LR or HR) |
| c-46 | 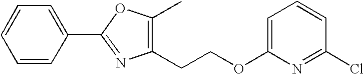 | (CDCl₃, 400 MHz): 7.97(2H, dd, J=8.0, 7.6 Hz), 7.49(1H, dd, J=8.1, 7.6 Hz), 7.43-7.38 (3H, m), 6.87(1H, d, J=7.6 Hz), 6.62(1H, d, J=8.1 Hz), 4.55(2H, t, J=7.0 Hz), 2.97-(2H, t, J=7.0 Hz), 2.34(3H, s) | LR 315 (M + H)⁺ |
| c-47 | 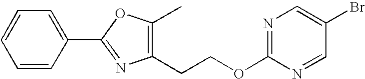 | (CDCl₃, 300 MHz): 8.51(2H, s), 7.97-7.94(2H, m), 7.45-7.38 (3H, m), 4.61(2H, t, J=6.9 Hz), 3.01(2H, t, J=6.9 Hz), 2.35(3H, s) | LR 361 (M + H)⁺ |

### Preparation c-48

### 6-[2-(5-Methyl-2-phenyl-1,3-oxazol-4-yl)ethoxy]nicotinaldehyde

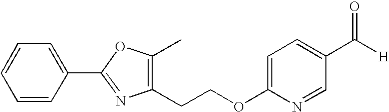

To a solution of butyllithium (27.4 mL of a 1.6M solution in hexanes, 43.8199 mmol) in anhydrous tetrahydrofuran (200 mL), under an atmosphere of nitrogen, was added a solution of 5-bromo-2-[2-(5-methyl-2-phenyl-1,3-oxazol-4-yl)ethoxy]pyridine (Preparation c-45) (14.31 g, 39.8363 mmol) in anhydrous tetrahydrofuran (170 mL) and anhydrous diethyl ether (170 mL) over a period of 45 minutes. To this solution was then added anhydrous N,N-dimethylformamide (5.7 mL) dropwise and the mixture stirred at 0° C. for 1 hour. The reaction was quenched by addition of saturated aqueous ammonium chloride (250 mL) and then ethyl acetate (250 mL). The resulting layers were separated and the aqueous layer extracted with ethyl acetate (2×250 mL). The combined organic extracts were washed with water (2×250 mL), saturated aqueous sodium chloride (250 mL), dried (anhydrous magnesium sulfate), filtered and concentrated in vacuo to afford the crude product. The residue was purified by flash column chromatography (hexanes to 50% ethyl acetate/hexanes) to yield a pale yellow crystalline solid (7.17 g, 58%). LRMS (m/z): 309 (M+H)⁺. ¹H NMR (CDCl₃, 300 MHz) δ 9.93 (1H, s), 8.61 (1H, d, J=2.3 Hz), 8.04 (1H, dd, J=2.5, 8.7 Hz), 7.98-7.95 (2H, m), 7.43-7.39 (3H, m), 6.81 (1H, d, J=8.7 Hz), 4.68 (2H, t, J=6.8 Hz), 3.00 (2H, t, J=6.8 Hz), 2.34 (3H, s).

### Preparation c-49

### Ethyl 1-(hydroxy{6-[2-(5-methyl-2-phenyl-1,3-oxazol-4-yl)ethoxy]pyridin-3-yl}methyl)cyclobutanecarboxylate

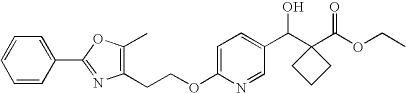

To a solution of 6-[2-(5-methyl-2-phenyl-1,3-oxazol-4-yl)ethoxy]nicotinaldehyde (Preparation c-48) (0.65 g, 2.1081 mmol), chromium (II) chloride (1 g, 8.1367 mmol), and lithium iodide (0.0784 g, 0.5859 mmol) in anhydrous tetrahdrofuran (15 mL), under an atmosphere of nitrogen, was added a solution of 1-bromo-cyclobutanecarboxylic acid ethyl ester (0.79 mL, 4.8821 mmol) in anhydrous tetrahydrofuran (5 mL) dropwise. The resulting mixture was stirred at 50° C. for 3 hours and allowed to cool to ambient temperature. The solution was then quenched by addition of water (50 mL) and the organic layer separated, which was further washed with water (2×50 mL), saturated aqueous sodium chloride (50 mL), dried (anhydrous magnesium sulfate), filtered and concentrated in vacuo to afford the crude product. The residue was purified by flash column chromatography (50% ethyl acetate/hexanes to ethyl acetate) to yield a yellow oil (0.3422 g, 37%). LRMS (m/z): 437 (M+H)⁺. ¹H NMR (CDCl₃, 400 MHz) δ 8.05 (1H, d, J=2.3 Hz), 7.98-7.95 (2H, m), 7.56 (1H, dd, J=2.5, 8.7 Hz), 7.44-7.37 (3H, m), 6.67 (1H, d, J=8.7 Hz), 4.85 (1H, d, J=6.6 Hz), 4.54 (2H, t, J=6.6 Hz), 4.18-4.07 (2H, m), 3.31 (1H, bs), 2.96 (2H, t, J=6.7 Hz), 2.46-2.30 (2H, m), 2.32 (3H, s), 2.21-2.12 (1H, m), 1.97-1.85 (1H, m), 1.79-1.65 (2H, m), 1.21 (3H, t, J=7.2 Hz).

### Preparation c-50

### 1-(Ethoxy-{6-[2-(5-methyl-2-phenyl-oxazol-4-yl)-ethoxy]-pyridin-3-yl}-methyl)-cyclobutanecarboxylic acid ethyl ester

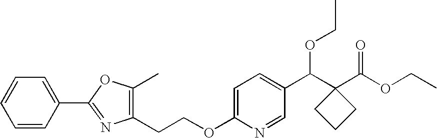

To a solution of 1-(hydroxy-{6-[2-(5-methyl-2-phenyl-oxazol-4-yl)-ethoxy]-pyridin-3-yl}-methyl)-cyclobutanecarboxylic acid ethyl ester (0.1711 g, 0.3920 mmol) in dry acetonitrile (2 mL) was added silver(I) oxide (1.8168 g, 7.8396 mmol) and iodoethane (0.64 mL, 7.8396 mmol). The resulting mixture was stirred for 5 days and concentrated under reduced pressure to afford the crude product and recovered remaining starting material. The residue was purified by flash column chromatography (hexanes to ethyl acetate) to yield the pure ester (0.0474 g, 26%) as a colorless oil. LRMS (m/z): 465 (M+H)⁺. ¹H NMR (CDCl₃, 400 MHz): δ 8.05 (1H, d, J=2.3 Hz), 7.98-7.95 (2H, m), 7.56 (1H, dd, J=2.5, 8.7 Hz), 7.44-7.37 (3H, m), 6.67 (1H, d, J=8.7 Hz), 4.65 (1H, m), 4.54 (2H, t, J=6.6 Hz), 4.18-4.07 (2H, m), 4.06 (2H, q, J=7.1 Hz), 2.96 (2H, t, J=6.7 Hz), 2.46-2.30 (2H, m), 2.32 (3H, s), 2.21-2.12 (1H, m), 1.97-1.85 (1H, m), 1.79-1.65 (2H, m), 1.42 (3H, t, J=7.1 Hz), 1.21 (3H, t, J=7.2 Hz).

### Preparation c-51

### {6-[2-(5-methyl-2-phenyl-1,3-oxazol-4-yl)ethoxy]pyridin-3-yl}methanol

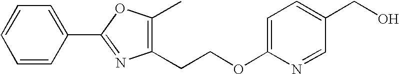

Sodium borohydride (0.480 g, 12.7 mmol) was added portionwise to a solution of 6-[2-(5-methyl-2-phenyl-1,3-oxazol-4-yl)ethoxy]nicotinaldehyde (Preparation c-48) (1.30 g, 4.22 mmol) in methanol (40 mL) at ambient temperature. The mixture was stirred for 30 minutes then evaporated. The residue was partitioned between saturated aqueous ammonium chloride and ethyl acetate. The organic phase was washed with saturated aqueous sodium chloride and dried (anhydrous magnesium sulfate), filtered and evaporated to give the title compound as a white crystalline solid (1.24 g, 100%). LRMS (m/z): 311 (M+H)⁺. ¹H NMR (CDCl₃, 300 MHz) 8.11 (1H, d, J=2.6 Hz), 8.00-7.95 (2H, m), 7.60 (1H, dd, J=2.5, 8.5 Hz), 7.45-7.38 (3H, m), 6.72 (1H, d, J=8.5 Hz), 4.61 (2H, bs), 4.56 (2H, t, J=6.8 Hz), 2.98 (2H, t, J=6.8 Hz), 2.33 (3H, s).

### Preparation c-52

### {2-[2-(5-Methyl-2-phenyl-oxazol-4-yl)-ethoxy]-pyrimidin-5-yl}-methanol

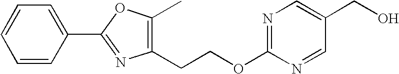

A solution of 5-bromo-2-[2-(5-methyl-2-phenyl-oxazol-4-yl)-ethoxy]-pyrimidine (1.0 g, 2.7765 mmol), tert-butyl-dimethyl-tributylstannanylmethoxy-silane (1.8 g, 4.1648 mmol), and tetrakis(triphenylphosphine)palladium(0) (0.3209 g, 0.2777 mmol) in 1,4-dioxane (2.8 mL) was heated (by microwave irradiation) at 150° C. for 2 hours. The resulting solution was allowed to cool to ambient temperature and saturated aqueous potassium fluoride (10 mL) was added followed by stirring for 30 minutes. This mixture was then extracted with ethyl acetate (3×25 mL) and the combined organic extracts dried (anhydrous magnesium sulfate), filtered and concentrated in vacuo to afford the crude product as a yellow oil. To a solution of the crude residue in dry tetrahydrofuran (24 mL) was added tetrabutylammonium fluoride (3.1 mL of a 1.0M solution in tetrahydrofuran). The resulting mixture was stirred at ambient temperature for 16 hours and concentrated under reduced pressure. The residue was purified by flash column chromatography (50% ethyl acetate/hexanes to 10% methanol/ethyl acetate) to yield the pure alcohol (0.6137 g, 71% for 2 steps) as a white solid. LRMS (m/z): 312 (M+H)⁺

### Preparation c-53

### 5-Benzyloxy-2-methyl-pyridine

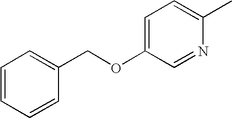

To a solution of 5-hydroxy-2-methylpyridine (20 g, 183.2677 mmol) and sodium hydroxide (8.0638 g, 201.5944 mmol) in acetone (400 mL) and water (120 mL) was added benzyl bromide (24 mL, 201.5944 mmol). The resulting mixture was refluxed for 16 hours and allowed to cool to ambient temperature. The acetone was removed in vacuo and the mixture extracted with ethyl acetate (3×150 mL). The combined organic extracts were washed with saturated aqueous sodium chloride (200 mL), dried (anhydrous magnesium sulfate), filtered and concentrated in vacuo to afford the pure product (31.35 g, 86%) as an orange oil. LRMS(m/z): 200 (M+H)⁺. ¹H NMR (CDCl₃, 300 MHz): 8.25 (1H, d, J=2.8 Hz), 7.43-7.31 (5H, m), 7.15 (1H, dd, J=8.5, 2.8Hz), 7.04 (1H, d, J=8.5 Hz), 5.06 (2H, s), 2.47 (3H, s).

### Preparation c-54

### 5-Benzyloxy-2-methyl-pyridine 1-oxide

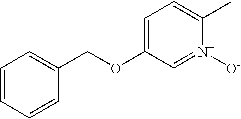

To a solution of 5-benzyloxy-2-methyl-pyridine (31.35 g, 157.34 mmol) in dry chloroform (800 mL), at ambient temperature, was added 3-chloroperoxybenzoic acid (77% max.) (38.7888 g, 173.074 mmol). The resulting mixture was stirred for 2 hours and then quenched with a solution of sodium thiosulfate (36.0805 g, 286.5 mmol) in water (500 mL) and stirred for 15 minutes. The phases were separated and the organic layer washed with water (500 mL), saturated sodium chloride (500 mL), dried (anhydrous magnesium sulfate), filtered and concentrated in vacuo to afford the crude product. The residue was recrystallized from acetone/hexanes to yield the pure product (33.1597 g, 97%) as a white solid. LRMS (m/z): 216 (M+H)⁺. ¹H NMR (CDCl₃, 300 MHz): δ 8.10-8.09 (1H, bm), 7.38-7.34 (5H, m), 7.10 (1H, d, J=8.7Hz), 6.87 (1H, dd, J=8.7, 2.3 Hz), 5.04 (2H, s), 2.43 (3H, s).

### Preparation c-55

### (5-Benzyloxy-pyridin-2-yl)-methanol

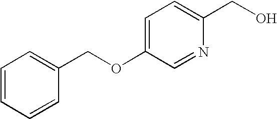

A solution of 5-benzyloxy-2-methyl-pyridine 1-oxide (0.92 g, 4.2741 mmol) in acetic anhydride (6.5 mL) was heated at 100° C. for 30 minutes. After cooling to ambient temperature, the reaction mixture was poured into ethyl acetate (50 mL), washed with saturated aqueous sodium bicarbonate (50 mL), saturated aqueous sodium chloride (50 mL), dried (anhydrous magnesium sulfate), filtered, and concentrated in vacuo to afford the crude acetate.

To the crude residue in methanol (45 mL) was added potassium carbonate (2.1784 g, 15.7719 mmol) and the solution allowed to stir at ambient temperature for 16 hours. The reaction mixture was poured into water (50 mL) and the organic removed under reduced pressure. The resulting residue was extracted with ethyl acetate (3×50 mL) and the combined organic extracts dried (anhydrous magnesium sulfate), filtered, and concentrated in vacuo to afford the crude product. The residue was purified by flash column chromatography (hexanes to 20% methanol/ethyl acetate) to yield the pure alcohol (0.5719 g, 62% for two steps) as a white solid. LRMS (m/z): 216 (M+H)⁺. ¹H NMR (CDCl₃, 300 MHz): δ 8.31 (1H, d, J=2.8 Hz), 7.44-7.31 (5H, m), 7.27 (1H, dd, J=8.7, 2.8 Hz), 7.17 (1H, d, J=8.5 Hz), 5.11 (2H, s), 4.69 (2H, s).

### Preparation c-56

### 2-Methyl-5-[2-(5-methyl-2-phenyl-oxazol-4-yl)-ethoxy]-pyridine 1-oxide

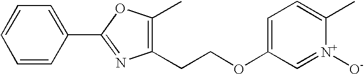

To a solution of 2-methyl-5-[2-(5-methyl-2-phenyl-oxazol-4-yl)-ethoxy]-pyridine (6.7973 g, 23.0917 mmol) in dry chloroform (140 mL), at ambient temperature, was added 3-chloroperoxybenzoic acid (77% max.) (7.7629 g, 34.6376 mmol). The resulting mixture was stirred for 2 hours and then quenched with a solution of sodium thiosulfate (4.3621 g, 34.6376 mmol) in water (25 mL) and stirred for 15 minutes. The phases were separated and the organic layer washed with water (50 mL), saturated sodium chloride (50 mL), dried (anhydrous magnesium sulfate), filtered and concentrated in vacuo to afford the crude product. This pale yellow oil (7.0689 g, 98%) was used without further purification. LRMS (m/z): 311 (M+H)⁺. ¹H NMR (CDCl₃, 300 MHz): δ 8.06 (1H, d, J=2.3 Hz), 7.96-7.93 (2H, m), 7.41-7.39 (3H, m), 7.10 (1H, d, J=8.9 Hz), 6.85 (1H, dd, J=8.8, 2.4 Hz), 4.23 (2H, t, J=6.6 Hz), 2.95 (2H, t, J=6.6 Hz), 2.43 (3H, s), 2.35 (3H, s).

### Preparation c-57

### {6-[2-(5-Methyl-2-phenyl-oxazol-4-yl)-ethoxy]-pyridin-2-yl}-methanol

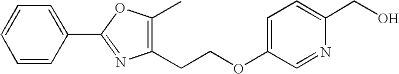

A solution of 2-methyl-5-[2-(5-methyl-2-phenyl-oxazol-4-yl)-ethoxy]-pyridine 1-oxide (3.5979 g, 11.5181 mmol) in acetic anhydride (17.5 mL) was heated at 100° C. for 30 minutes. After cooling to ambient temperature, the reaction mixture was poured into ethyl acetate (150 mL), washed with saturated aqueous sodium bicarbonate (150 mL), saturated aqueous sodium chloride (150 mL), dried (anhydrous magnesium sulfate), filtered, and concentrated in vacuo to afford the crude acetate. To the crude residue in methanol (120 mL) was added potassium carbonate (5.8705 g, 42.617 mmol) and the solution allowed to stir at ambient temperature for 16 hours. The reaction mixture was poured into water (150 mL) and the organic removed under reduced pressure. The resulting residue was extracted with ethyl acetate (3×150 mL) and the combined organic extracts dried (anhydrous magnesium sulfate), filtered, and concentrated in vacuo to afford the crude product. The residue was purified by flash column chromatography (ethyl acetate to 10% methanol/ethyl acetate) to yield the pure alcohol (1.52 g, 43% for two steps) as a pale yellow low melting solid. LRMS (m/z): 311 (M+H)⁺. ¹H NMR (CDCl₃, 300 MHz): 8.22 (1H, d, J=2.5 Hz), 7.97-7.94 (2H, m), 7.42-7.38 (3H, m), 7.20 (1H, dd, J=8.5, 2.6 Hz), 7.15 (1H, d, J=8.7 Hz), 4.67 (2H, s), 4.28 (2H, t, J=6.7 Hz), 2.98 (2H, t, J=6.7 Hz), 2.37 (3H, s).

### Preparation c-58

### 5-(chloromethyl)-2-[2-(5-methyl-2-phenyl-1,3-oxazol-4-yl)ethoxy]pyridine

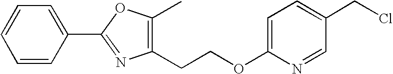

Oxalyl chloride (0.30 mL, 3.44 mmol) was added to a solution of {6-[2-(5-methyl-2-phenyl-1,3-oxazol-4-yl)ethoxy]pyridin-3-yl}methanol (Preparation c-57) (0.97 g, 3.13 mmol) in dichloromethane (30 mL) and N,N-dimethyl formamide (3 mL) at 0° C. The mixture was warmed to ambient temperature and stirred for 1 hour then evaporated. The residue was partitioned between saturated aqueous sodium bicarbonate and ethyl acetate. The organic phase was washed with saturated aqueous sodium chloride and dried (anhydrous magnesium sulfate), filtered and evaporated to give the title compound as a white crystalline solid (1.01 g, 100%). LRMS (m/z): 329 (M+H)⁺. ¹H NMR (CDCl₃, 300 MHz) δ 8.12 (1H, d, J=2.5 Hz), 7.98-7.95 (2H, m), 7.60 (1H, dd, J=2.5, 8.5 Hz), 7.45-7.37 (3H, m), 6.72 (1H, d, J=8.5 Hz), 4.57 (2H, t, J=6.8 Hz), 4.53 (2H, s), 2.97 (2H, t, J=6.8 Hz), 2.33 (3H, s).

### Preparations c-59 to c-63

Preparations c-59 to c-63 were prepared by general procedure for Preparation c-58

| | | | MS (m/z) |
|---|---|---|---|
| Prep # | Structure | ¹H NMR | (LR or HR) |
| c-59 | 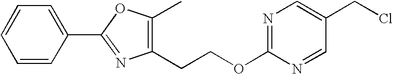 | | LR 330 (M + H)⁺ |
| c-60 | 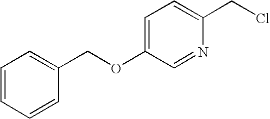 | | LR 234 (M + H)⁺ |
| c-61 | 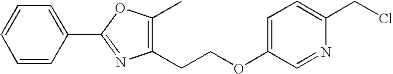 | | LR 329 (M + H)⁺ |
| c-62 | 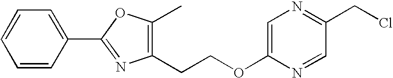 | (CDCl₃, 300 MHz): 8.16(2H, s), 7.97(2H, d, J=7.7 Hz), 7.40(3H, s), 4.61(4H, m), 2.99(2H, t, J=6.7 Hz), 2.35(3H, s) | LR 330 (M + H)⁺ |
| c-63 | 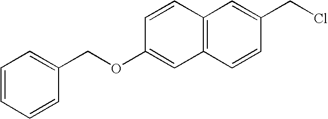 | (CDCl₃, 400 MHz): 7.75-7.72(3H, m), 7.50-7.33(6H, m), 7.24-7.22 (2H, m), 5.18(2H, s), 4.74(2H, s) | LR 282 (M + H)⁺ |

### Preparation c-64

### 5-(Iodomethyl)-2-[2-(5-methyl-2-phenyl-1,3-oxazol-4-yl)ethoxy]pyridine

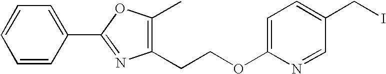

Sodium iodide (0.750 g) was added to a solution of 5-(chloromethyl)-2-[2-(5-methyl-2-phenyl-1,3-oxazol-4-yl)ethoxy]pyridine (Preparation c-58) (0.690 g, 2.10 mmol) in acetone (5 mL) and the mixture was heated at reflux for 30 minutes, cooled and evaporated. The residue was suspended in ethyl acetate and filtered through a pad of silica gel. The filtrate was evaporated to give the title compound as a yellow crystalline solid that was used directly in subsequent reactions. LRMS (m/z): 421 (M+H)⁺.

### Preparations c-65 to c-69

Preparations c-65 to c-69 were prepared by general procedure for Preparation c-64

| | | | MS (m/z) |
|---|---|---|---|
| Prep # | Structure | ¹H NMR | (LR or HR) |
| c-65 | 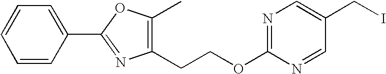 | | LR 422 (M + H)⁺ |
| c-66 | 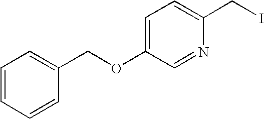 | | LR 326 (M + H)⁺ |
| c-67 | 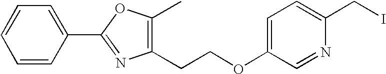 | | LR 421 (M + H)⁺ |
| c-68 | 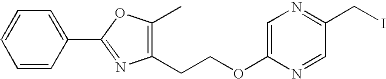 | | LR 422 (M + H)⁺ |
| c-69 | 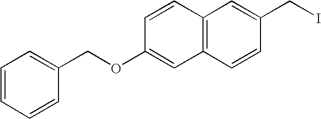 | (CDCl₃, 400 MHz): 7.76-7.66(3H, m), 7.49-7.32 (6H, m), 7.24-7.18(2H, m), 5.18(2H, s), 4.63(2H, s) | LR 375 (M + H)⁺ |

### Preparation c-70

### Ethyl 1-({6-[2-(5-methyl-2-phenyl-1,3-oxazol-4-yl)ethoxy]pyridin-3-yl}methyl)cyclobutane carboxylate

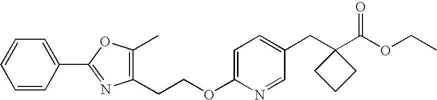

Sodium (bis)trimethylsilyl amide (3.0 mL of a 1 M solution in tetrahydrofuran, 3.0 mmol) was added dropwise to a solution of ethyl cyclobutanoate (0.41 mL, 3.0 mmol) in anhydrous tetrahydrofuran (5 mL) at −60° C. The mixture was stirred for 1 hour and then a solution of 5-(iodomethyl)-2-[2-(5-methyl-2-phenyl-1,3-oxazol-4-yl)ethoxy]pyridine (Preparation 28) (0.271 g, 0.64 mmol) in anhydrous tetrahydrofuran (4 mL) was added dropwise. The resulting mixture was stirred at −60° C. for 1 hour then quenched with saturated aqueous ammonium chloride and warmed to ambient temperature. The mixture was extracted with ethyl acetate and the organic phase dried (anhydrous magnesium sulfate), filtered and evaporated to afford a 1:1 mixture of the title compound and dimer (0.160 g) which was used directly in the subsequent step. LRMS (m/z): 421 (M+H)⁺.

### Preparation c-71

### ethyl 2-({6-[2-(5-methyl-2-phenyl-1,3-oxazol-4-yl)ethoxy]pyridin-3-yl}methyl)tetrahydrofuran-2-carboxylate

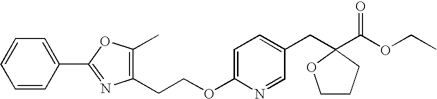

Sodium (bis)trimethylsilyl amide (3.18 mL of 1M solution in tetrahydrofuran, 3.18 mmol) was added dropwise to a solution of ethyl 2-tetrahydrofuranoate (0.458 g, 3.18 mmol) in anhydrous tetrahydrofuran (4 mL) at −50° C. The mixture was stirred for 45 minutes and then a solution of 5-(iodomethyl)-2-[2-(5-methyl-2-phenyl-1,3-oxazol-4-yl)ethoxy]pyridine (Preparation 28) (0.267 g, 0.64 mmol) in anhydrous tetrahydrofuran (2 mL) was added dropwise. The resulting mixture was stirred at −50° C. for 1.5 hours then quenched with saturated aqueous ammonium chloride and warmed to ambient temperature. The mixture was extracted with ethyl acetate and the organic phase dried (anhydrous magnesium sulfate), filtered and evaporated. The residue was purified by flash column chromatography (25% to 35% ethyl acetate/hexanes) to yield the title compound as a colorless oil (0.250 g, 90%). LRMS (m/z): 437 (M+H)⁺. ¹H NMR (CDCl₃, 300 MHz) δ 7.95 (3H, m), 7.51 (1H, dd, J=2.5, 8.5 Hz), 7.43-7.36 (3H, m), 6.61 (1H, d, J=8.5 Hz), 4.51 (2H, t, J=6.8 Hz), 4.29-4.18 (1H, m), 413 (2H, q, J=7.2 Hz), 3.95-3.82 (2H, m), 3.10 (1 H, d, J=14.1 Hz), 2.95 (2H, t, J=6.8 Hz), 2.86 (1H, d, J=14.1 Hz), 2.31 (3H, s), 2.26-2.20 (1H, m), 1.92-1.77 (2H, m), 1.70-1.61 (1H, m), 1.21 (3H, t, J=7.2 Hz).

### Preparations c-72 to c-79

Preparations c-72 to c-79 were prepared by general procedure for Preparation c-71.

| | | | MS (m/z) |
|---|---|---|---|
| | | | (LR or |
| Prep # | Structure | ¹H NMR | HR) |
| c-72 | 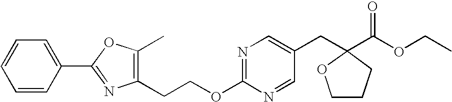 | (CDCl₃, 300 MHz): 8.38(2H, s), 7.97-7.94(2H, m), 7.44-7.38(3H, m), 4.59(2H, t, J=7.0 Hz), 4.15(2H, q, J=7.0 Hz), 3.99-3.86(2H, m), 3.13 (1H, d, J=1.43 Hz), 3.00(1H, t, J=6.9 Hz), 2.84 (1H, d, J=14.3 Hz), 2.35(3H, s), 2.26-2.20(1H, m), 1.91-1.63(4H, m), 1.23(3H, t, J=7.0 Hz) | LR 438 (M + H)⁺ |
| c-73 | 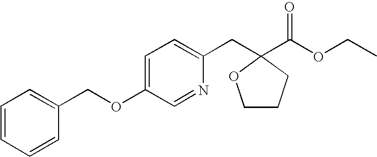 | (CDCl₃, 300 MHz): 8.27(1H, d, J=2.3 Hz), 7.42-7.32(5H, m), 7.20-7.13(2H, m), 5.06(2H, s), 4.13 (2H, q, J=13.9 Hz), 3.33(1H, d, J=13.9 Hz), 3.14(1H, d, J=13.9 Hz), 2.64-2.55(1H, m), 2.27-2.20(1H, m), 1.89-1.63(4H, m), 1.24(3H, t, J=13.9 Hz) | LR 342 (M + H)⁺ |
| c-74 | 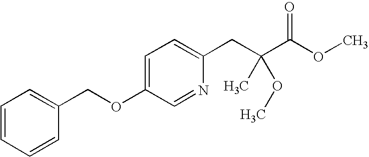 | (CDCl₃, 300 MHz): 8.27(1H, d, J=2.6 Hz), 7.42-7.29(5H, m), 7.16(1H, dd, J=8.7, 2.8 Hz), 7.14-7.08(1H, m), 5.06(2H, s), 3.71(3H, s), 3.30(3H, s), 3.16(2H, s), 1.40(3H, s) | LR 316 (M + H)⁺ |
| c-75 | 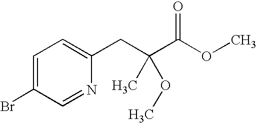 | (CDCl₃, 400 MHz): 8.14(1H, d, J=2.3 Hz), 7.41 (1H, dd, J=8.3, 2.5 Hz), 7.36(1H, dd, J=8.3, 0.8 Hz), 3.69(3H, s), 3.27(3H, s), 2.99(1H, d, J=13.9 Hz), 2.87(1H, d, J=13.9 Hz), 1.33(3H, s) | LR 289 (M + H)⁺ |
| c-76 | 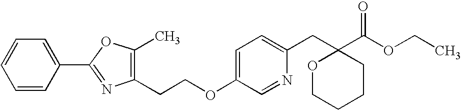 | (CDCl₃, 400 MHz): 8.17(1H, d, J=2.3 Hz), 7.96 (2H, dd, J=7.7, 1.9 Hz), 7.43-7.38(3H, m), 7.12 (1H, d, J=8.1 Hz), 7.09(1H, dd, J=8.6, 2.8 Hz), 4.24(2H, t, J=6.7 Hz), 4.15(2H, q, J=7.2 Hz), 3.88-3.84(1H, m), 3.64(1H, dt, J=11.6, 3.3 Hz), 3.07(2H, s), 2.96(2H, t, J=6.6 Hz), 2.36(3H, s), 2.22-2.18(1H, m), 1.52-1.36(5H, m), 1.20(3H, t, J=7.1 Hz) | LR 451 (M + H)⁺ |
| c-77 | 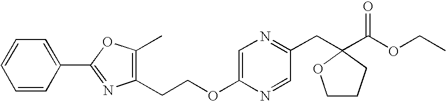 | (CDCl₃, 300 MHz): 8.09(1H, s), 7.91-8.01(3H, m), 7.34-7.45(3H, m), 4.48-4.64(2H, m), 3.84-4.24 (2H, m), 3.22(1H, d, J=15.0 Hz), 3.11(1H, d, J=15.0 Hz), 2.91-3.03(1H, m), 2.31-2.35(1H, m), 1.65-2.30(2H, m), 1.24(3H, t, J=6.6 Hz) | LR 438 (M + H)⁺ |
| c-78 | 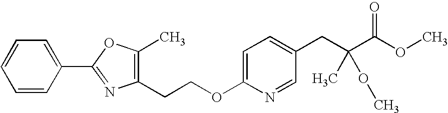 | (CDCl₃, 300 MHz): 7.96(1H, m), 7.92(2H, d, J=2.1 Hz), 7.41(4H, m), 6.63(1H, d, J=8.5 Hz), 4.52(2H, t, J=6.8 Hz), 3.70(3H, s), 3.30(3H, m), 2.93(4H, m), 2.33(3H, s), 1.33(3H, s) | LR 411 (M + H)⁺ |
| c-79 | 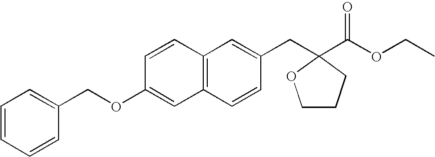 | (CDCl₃, 400 MHz): 7.69(1H, d, J=9.3 Hz), 7.64-7.62(2H, m), 7.49-7.48(2H, m), 7.42-7.32(4H, m), 7.21-7.18(2H, m), 5.17(2H, s), 4.24-4.18(2H, m), 3.96-3.86(2H, m), 3.33(1H, d, J=13.6 Hz), 3.13(1H, d, J=13.8 Hz), 2.32-2.20(1H, m), 2.03-1.89(3H, m), 1.19(3H, t, J=7.1 Hz) | LR 391 (M + H)⁺ |

### Preparation c-80

### 2-Bromo-5-(bromomethyl)pyridine

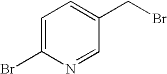

Phosphorous tribromide (100 mmol, 27.1 g, 2.0 eq.) was added carefully to 2-chloro-5-hydroxymethyl pyridine (50.0 mmol, 7.18 g, 1.0 eq.). The pyridine clumped together and the mixture was heated to 160 degrees C. Within 5 minutes of stirring at >150 degrees C. the mixture was seen to go very dark in color with gas evolution. The mixture was stirred at this same temperature for approximately 2.5 hours at which point it was cooled to room temperature. The mixture was cooled further to 0 degrees C. whereupon saturated sodium bicarbonate was added very cautiously (highly exothermic!). As foaming became less vigorous, ice was added to the mixture until foaming subsided. Solid sodium bicarbonate was then carefully added to achieve a pH of ˜8-9. The mixture was extracted with ethyl acetate and the organic layer was washed with brine and dried over anhydrous magnesium sulfate. Concentrated in vacuo to afford a dark solid. This material was dissolved in a minimal amount of DCM and purified using a Biotage Sp4 65i over a gradient of 0-100% ethyl acetate in hexanes to afford the title compound as a pale yellow solid (5.57 g, 44%). LRMS: 252 (M+H)⁺. ¹H NMR (DMSO-d₆, 400 MHz): δ 8.39 (1H, s) 7.59 (1H, d, J=8.5 Hz) 7.48 (1H, d, J=8.5 Hz) 4.46 (2H, s).

### Preparation c-81

### Ethyl 2-[(6-bromopyridin-3-yl)methyl]tetrahydrofuran-2-carboxylate

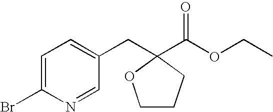

To a solution of ethyl tetrahydrofuran-2-carboxylate (52.9 mmol, 9.10 g, 1.5 eq.) cooled to −78 degrees C. in THF (90 mL) was added dropwise a solution of 2 M lithium diisopropylamide (52.9 mmol, 1.5 eq.) in a mixture of heptane/THF/ethylbenzene. The enolate was allowed to form for one hour at the same low temperature whereupon a solution of 2-bromo-5-(bromomethyl)pyridine (35.3 mmol, 8.85 g, 1.0 eq.) in THF was added dropwise. The reaction was allowed to warm slowly to room temperature overnight. The reaction was quenched with saturated ammonium chloride. The mixture was extracted with ethyl acetate and the organic extract was washed with brine. The organic layer was dried over anhydrous magnesium sulfate and concentrated in vacuo to yield a yellow oil. This crude product was purified on a Biotage Sp4 65i over a gradient of 5% to 95% ethyl acetate in hexanes to afford a golden oil (8.70 g, 78%). LRMS: 315 (M+H)⁺. ¹H NMR (DMSO-d₆, 400 MHz): δ 8.21 (1 H, s) 7.40-7.49 (2 H, m) 3.94 (2 H, q, J=7.0 Hz) 3.71-3.85 (2 H, m) 3.05-3.11 (1 H, m) 2.91-2.97 (1 H, m) 2.38-2.47 (1 H, m) 1.83-2.09 (3 H, m) 1.09 (3 H, t, J=7.0 Hz)

### Preparation c-82

### Cyclopropanecarboxylic acid tert-butyl ester

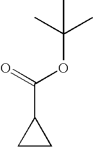

Concentrated sulfuric acid (3.45 mL, 62.7832 mmol) was added to a vigorously stirred suspension of anhydrous magnesium sulfate (30.1987 g, 251.1326 mmol) in dichloromethane (250 mL). The mixture was stirred for 15 minutes, after which cyclopropanecarboxylic acid (5 mL, 62.7832 mmol) and 2-methyl-propan-2-ol (30 mL, 313.9158 mmol) were added. The mixture was stoppered tightly and stirred at ambient temperature for 16 hours. The reaction mixture was then quenched with saturated aqueous sodium bicarbonate (450 mL) and stirred until all the magnesium sulfate had dissolved. The phases were separated and the organic phase washed with water (100 mL), saturdated aqueous sodium chloride (100 mL), dried (anhydrous magnesium sulfate), filtered and concentrated in vacuo to afford the pure ester (8.3921 g, 59.0162 mmol) as a colorless liquid. ¹H NMR (CDCl₃, 300 MHz): δ 1.45 (9H, s), 0.93-0.86 (3H, m), 0.79-0.73 (2H, m).

### Preparation c-83

### 1-{6-[2-(5-Methyl-2-phenyl-oxazol-4-yl)-ethoxy]-pyridin-3-ylmethyl}-cyclopropanecarboxylic acid tert-butyl ester

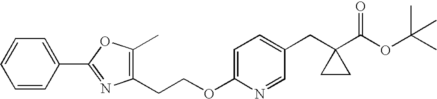

To a solution of diisopropylamine (0.14 mL, 0.9518 mmol) in dry tetrahydrofuran (2.4 mL), at 0° C. under an atmosphere of nitrogen, was added butyllithium (0.38 mL of a 2.5M solution in hexanes, 0.9518 mmol). The resulting solution was stirred for 30 minutes and then cooled to −50° C. To this was added a solution of cyclopropanecarboxylic acid tert-butyl ester (0.1269 g, 0.8924 mmol) in dry tetrahydrofuran (1 mL) and stirring was continued for 2 hours. A solution of 5-iodomethyl-2-[2-(5-methyl-2-phenyl-oxazol-4-yl)-ethoxy]-pyridine (0.25 g, 0.5949 mmol) in dry tetrahydrofuran (1 mL) was then added dropwise and the solution stirred for a further 3 hours. The reaction was quenched by the addition of saturated aqueous ammonium chloride (25 mL) and extracted with ethyl acetate (3×25 mL). The combined organic extracts were then dried (anhydrous magnesium sulfate), filtered and concentrated in vacuo to afford the crude product and remaining starting iodide. The residue was purified by flash column chromatography (hexanes to 60% ethyl acetate/hexanes) to yield the ester (0.0827 g, 32%), partially contaminated with started iodide, as a pale yellow solid. LRMS (m/z): 435 (M+H)⁺.

### Preparation c-84

### Ethyl 2-({6-[2-(5-methyl-2-phenyl-1,3-thiazol-4-yl)ethoxy]pyridin-3-yl}methyl)tetrahydrofuran-2-carboxylate

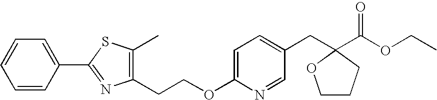

To an argon-purged solution of the bromopyridine (0.636 mmol) in toluene (12 mL) was added palladium (II) acetate (11.4 mg, 0.0508 mmol) and racemic-2-(di-t-butylphosphino)-1,1′-binaphthyl (25.4 mg, 0.0636 mmol). The activated complex was allowed to form over approximately ten minutes, at which point cesium carbonate (414 mg, 1.27 mmol) and the appropriate alcohol (0.956 mmol) were added. The mixture was heated to 115° C. and stirred at this temperature for 12-18 hours. The mixture was cooled to room temperature and filtered through a pad of silica. The filter pad was washed with 2-3 aliquots of ethyl acetate and the combined organic filtrates were combined and concentrated in vacuo. The resulting residue was either purified by flash chromatography, or used without further purification.

### Preparations c-85 to c-88

Preparations c-85 to c-88 were prepared by procedures analogous to those used for Preparation c-84

| | | | MS (m/z) |
|---|---|---|---|
| Prep # | Structure | ¹H NMR | (LR or HR) |
| c-85 | 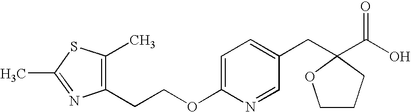 | (DMSO-d₆, 400 MHz) | LR 420 (M + H)⁺ |
| c-86 | 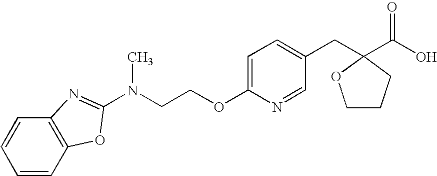 | | |
| c-87 | 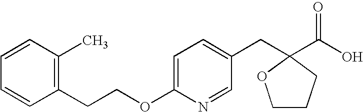 | | |
| c-88 | 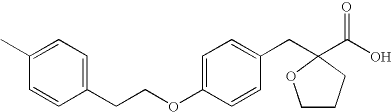 | | |

### Preparation c-89

### 5-{[tert-Butyl(diphenyl)silyl]oxy}-2-(iodomethyl)pyridine

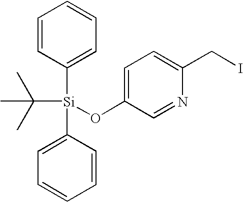

To a solution of 2-bromomethyl-5-(tert-butyl-diphenyl-silanyloxy)-pyridine (Schow, S. R.; Quinn DeJoy, S.; Wick, M. M.; Kerwar, S. S. J. Org. Chem. 1994, 59, 6850-6852) (1.2692 g, 2.9763 mmol) in acetone (15 mL) was added sodium iodide (0.8922 g, 5.9526 mmol) and the resulting heterogeneous mixture stirred for 3 hours at ambient temperature. The reaction mixture was concentrated in vacuo and the resulting residue diluted with ethyl acetate (50 mL) and washed with water (50 mL). The organic layer was further washed with saturated aqueous sodium bicarbonate (50 mL) and saturated aqueous sodium thiosulfate (50 mL). The combined aqueous layers were extracted with ethyl acetate (3×50 mL) and the combined organic extracts dried (anhydrous magnesium sulfate), filtered and concentrated in vacuo to afford the crude product. The residue was purified by flash column chromatography (hexanes to 20% ethyl acetate/hexanes) to yield a pale yellow oil (0.72 g, 51%). This compound was unstable to concentration and thus was used immediately. LRMS (m/z): 474 (M+H)⁺.

### Preparation c-90

### Ethyl tetrahydrofuran-2-carboxylate

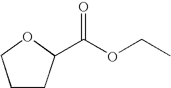

To a solution of tetrahydrofuran-2-carboxylic acid (20 g, 172.2356 mmol) in anhydrous ethanol (100 mL) was added concentrated sulfuric acid (0.46 mL). The resulting mixture was stirred at reflux for 16 hours and then allowed to cool to ambient temperature. To this was added water (100 mL) and extracted with diethyl ether (3×100 mL). The combined organic extracts were washed with saturated aqueous sodium bicarbonate (2×50 mL), saturated aqueous sodium chloride (100 mL), dried (anhydrous magnesium sulfate), filtered and concentrated in vacuo to afford the pure product as a colorless liquid (22.5964 g, 91%). LRMS (m/z): 145 (M+H)⁺. ¹H NMR (CDCl₃, 300 MHz) δ 4.38 (1H, dd, J=4.9, 8.1 Hz), 4.14 (2H, q, J=7.2 Hz), 3.99-3.92 (1H, m), 3.88-3.81 (1H, m), 2.24-2.12 (1H, m), 2.00-1.79 (3H, m), 1.22 (3H, t, J=7.2 Hz).

### Preparation c-91

### Tetrahydropyran-2-carboxylic acid ethyl ester

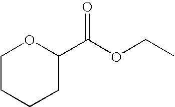

The above compound was prepared according to the procedure described in Rychnovsky, S. D.; Hata, T.; Kim, A. I.; Buckmelter, A. J. Org. Lett. 2001, 3, 807-810.

### Preparation c-92

### Ethyl 2-[(5-{[tert-butyl(diphenyl)silyl]oxy}pyridin-2-yl)methyl]tetrahydrofuran-2-carboxylate

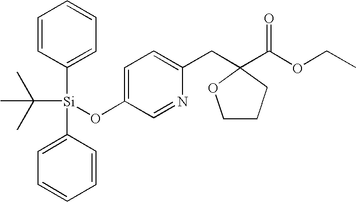

To a solution of ethyl tetrahydrofuran-2-carboxylate (Preparation 32) (1.0965 g, 7.604 mmol) in anhydrous tetrahydrofuran (7 mL) at −50° C., under an atmosphere of nitrogen, was added sodium bis(trimethylsilyl)amide (7.6 mL of a 1.0M solution in tetrahydrofuran, 7.604 mmol) dropwise. The reaction mixture was stirred for 1 hour and then a solution of 5-(tert-butyl-diphenyl-silanyloxy)-2-iodomethyl-pyridine (Preparation 31) (0.72 g, 1.5208 mmol) in anhydrous tetrahydrofuran (7 mL) was added dropwise. The resulting solution was stirred at −50° C. for 2 hours and then quenched with saturated aqueous ammonium chloride (25 mL). This was then extracted with ethyl acetate (3×25 mL), dried (anhydrous magnesium sulfate), filtered and concentrated in vacuo to afford the crude product. The residue was purified by flash column chromatography (hexanes to 40% ethyl acetate/hexanes) to yield a colorless oil (0.2438 g, 33%). LRMS (m/z): 490 (M+H)⁺. ¹H NMR (CDCl₃, 300 MHz) δ 8.07 (1H, d, J=2.5 Hz), 7.67-7.63 (4H, m), 7.41-7.31 (6H, m), 6.97 (1H, d, J=8.5 Hz), 6.86 (1H, dd, J=2.8, 8.5 Hz), 4.21 (2H, q, J=7.2 Hz), 4.09 (2H, q, J=7.2 Hz), 3.22 (1H, d, J=13.9 Hz), 3.07 (1H, d, J=13.9 Hz), 2.53-2.44 (1H, m), 2.31-2.15 (1H, m), 1.82-1.72 (1H, m), 1.60-1.46 (1H, m), 1.25 (3H, t, J=7.2 Hz), 1.09 (9H, s).

### Preparation c-93

### Ethyl 2-[(5-hydroxypyridin-2-yl)methyl]tetrahydrofuran-2-carboxylate

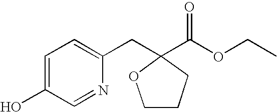

To a solution of ethyl 2-[(5-{[tert-butyl(dipheny)silyl]oxy}pyridin-2-yl)methyl]tetrahydrofuran-2-carboxylate (Preparation c-92) (0.5118 g, 1.1677 mmol) in anhydrous tetrahydrofuran (10 mL) was added tetrabutylammonium fluoride (1,3 mL of a 1.0M solution in tetrahydrofuran) dropwise. The resulting mixture was stirred at ambient temperature for 1 hour and the volatiles removed in vacuo. The residue was purified by flash column chromatography (50% ethyl acetate/hexanes to 10% methanol/ethyl acetate) to yield a colorless oil (0.2321 g, 79%).

LRMS (m/z): 252 (M+H)⁺. ¹H NMR (CDCl₃, 300 MHz) δ 8.10 (1H, d, J=2.3 Hz), 7.20 (1H, d, J=8.5 Hz), 7.14 (1H, dd, J=2.6, 8.5 Hz), 4.14 (2H, q, J=7.2 Hz), 3.88 (2H, q, J=7.8 Hz), 3.35 (1H, d, J=13.9 Hz), 3.12 (1H, d, J=13.9 Hz), 2.30-2.21 (1H, m), 2.04-1.94 (1H, m), 1.89-1.76 (1H, m), 1.75-1.63 (1H, m), 1.20 (3H, t, J=7.2 Hz).

### Preparation c-93a

### Alternative preparation of ethyl 2-[(5-hydroxypyridin-2-yl)methyl]tetrahydrofuran-2-carboxylate

To a solution of 2-(5-benzyloxy-pyridin-2-ylmethyl)-tetrahydro-furan-2-carboxylic acid ethyl ester (0.6065 g, 1.7765 mmol) in dry ethanol (10 mL) was added palladium (0.0607 g, 10 wt. % on activated carbon). The resulting solution was heated at 45° C. under an atmosphere of hydrogen for 16 hours. After cooling to ambient temperature the solution was filtered through a 3″ bed of Celite and washed with ethanol (100 mL). The filtrate was then concentrated in vacuo to afford the crude product which was used without further purification. LRMS (m/z): 252 (M+H)⁺. ¹H NMR (CDCl₃, 400 MHz): δ 8.10 (1H, d, J=2.3 Hz), 7.20 (1H, d, J=8.5 Hz), 7.14 (1H, dd, J=2.6, 8.5 Hz), 4.14 (2H, q, J=7.2 Hz), 3.88 (2H, q, J=7.8 Hz), 3.35 (1H, d, J=13.9 Hz), 3.12 (1H, d, J=13.9 Hz), 2.30-2.21 (1H, m), 2.04-1.94 (1H, m), 1.89-1.76 (1H, m), 1.75-1.63 (1H, m), 1.20 (3H, t, J=7.2 Hz).

### Preparations c-94 to c-95

Preparations c-94 to c-95 were prepared by general procedure for Preparation c-93

| Prep | | | MS (m/z) |
|---|---|---|---|
| # | Structure | ¹H NMR | (LR or HR) |
| c-94 | 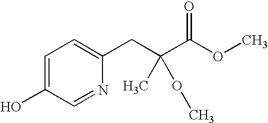 | (CDCl₃, 400 MHz): 8.24(1H, bs), 7.37-7.30(1H, bm), 7.24-7.22(1H, bm), 3.71(3H, s), 3.28(3H, s), 3.23-3.18(2H, m), 1.40(3H, s) | LR 226 (M + H)⁺ |
| c-95 | 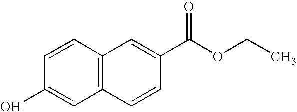 | (CDCl₃, 400 MHz): 7.64(1H, d, J=8.6 Hz), 7.61(1H, s), 7.53(1H, d, J=8.6 Hz), 7.36(1H, dd, J=1.7, 8.6 Hz), 7.05-7.01(2H, m), 5.32(1H, s), 3.99-3.88(2H, m), 3.34(1H, d, J=13.6 Hz), 3.11(1H, d, J=13.9 Hz), 2.33-2.27(1H, m), 2.00-1.93(1H, m), 1.89-1.77 (1H, m), 1.73-1.65(1H, m), 1.19(3H, t, J=7.3 Hz) | LR 301 (M + H)⁺ |

### Preparation c-96

### Ethyl 2-({5-[2-(5-methyl-2-phenyl-1,3-oxazol-4-yl)ethoxy]pyridin-2-yl}methyl)tetrahydrofuran-2-carboxylate

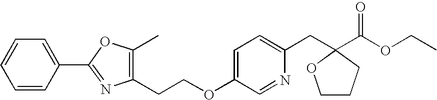

To a solution of ethyl 2-[(5-hydroxypyridin-2-yl)methyl]tetrahydrofuran-2-carboxylate (Preparation c-93) (0.2321 g, 0.9237 mmol), 2-(5-methyl-2-phenyl-oxazol-4-yl)-ethanol (0.2065 g, 1.0161 mmol), and triphenylphosphine (0.3634 g, 1.3856 mmol) in anhydrous tetrahydrofuran (10 mL), under an atmosphere of nitrogen, was added a solution of diethyl azodicarboxylate (0.22 mL, 1.3856 mmol) in anhydrous tetrahydrofuran (1 mL) dropwise. The resulting solution was stirred at ambient temperature for 16 hours and the volatiles removed in vacuo. This residue was then purified by flash column chromatography (hexanes to 50% ethyl acetate/hexanes) to yield a pale yellow oil (0.2618 g, 65%). LRMS (m/z): 437 (M+H)⁺.

¹H NMR (CDCl₃, 300 MHz) δ 8.20 (1H, d, J=2.8 Hz), 7.99 (1H, d, J=2.5 Hz), 7.96 (1H, d, J=1.7 Hz), 7.70-7.64 (1H, m), 7.49-7.39 (2H, m), 7.19 (1H, d, J=8.5 Hz), 7.11 (1H, dd, J=3.0, 8.5 Hz), 4.26 (2H, t, J=6.6 Hz), 4.17 (2H, q, J=7.2 Hz), 3.95-3.81 (2H, m), 3.33 (1H, d, J=13.8 Hz), 3.16 (1H, d, J=13.8 Hz), 2.98 (2H, t, J=6.6 Hz), 2.37 (3H, s), 2.34-2.22 (1H, m), 2.09-2.00 (1H, m), 1.87-1.76 (1H, m), 1.72-1.62 (1H, m), 1.23 (3H, t, J=7.2 Hz).

### Preparations c-97 to c-112

Preparations c-97 to c-112 were prepared by general procedure for Preparation c-96

| Prep | | | MS (m/z) |
|---|---|---|---|
| # | Structure | ¹H NMR | (LR or HR) |
| c-97 | 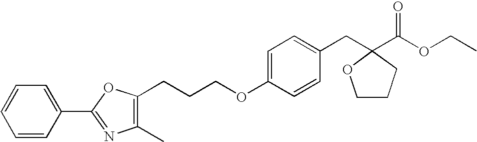 | (CDCl₃, 300 MHz): 8.19(1H, d, J=2.8 Hz), 7.97-7.93(2H, m), 7.42-7.39(3H, m), 7.18(1H, d, J=8.5 Hz), 7.10(1H, dd, J=8.5, 2.8 Hz), 4.16(2H, q, J=7.2 Hz), 3.98(2H, t, J=6.0 Hz), 3.92-3.82 (2H, m), 3.33(1H, d, J=13.8 Hz), 3.14 (1H, d, J=13.9 Hz), 2.67(2H, t, J=6.0 Hz), 2.26(3H, s), 2.19-2.10(2H, m), 1.91-1.63(4H, m), 1.22(3H, t, J=7.2 Hz) | LR 451 (M + H)⁺ |
| c-98 | 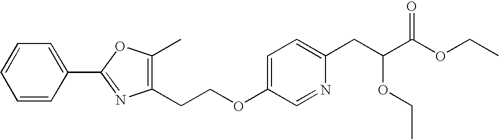 | (CDCl₃, 400 MHz): 8.20(1H, dd, J=2.0, 1.5 Hz), 7.95(2H, dd, J=7.7, 1.9 Hz), 7.43-7.36(3H, m), 7.10(2H, d, J=1.5 Hz), 4.25(2H, t, J=6.6 Hz), 4.23-4.13(3H, m), 3.63-3.55(1H, m), 3.37-3.27(1H, m), 3.15-3.02(2H, m), 2.97 (2H, t, J=6.7 Hz), 2.35(3H, s), 1.21 (3H, t, J=7.2 Hz), 1.08(3H, t, J=7.1 Hz) | LR 425 (M + H)⁺ |
| c-99 | 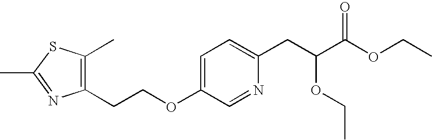 | (CDCl₃, 400 MHz): 8.19(1H, dd, J=2.0, 1.5 Hz), 7.08-7.07(2H, m), 4.26-4.23(3H, m), 4.16(2H, q, J=7.0 Hz), 3.63-3.56(1H, m), 3.36-3.28(1H, m), 3.15-3.02(4H, m), 2.58(3H, s), 2.34 (3H, s), 1.21(3H, t, J=7.1 Hz), 1.08 (3H, t, J=7.0 Hz) | LR 379 (M + H)⁺ |
| c-100 | 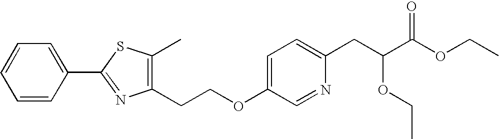 | (CDCl₃, 400 MHz): 8.22(1H, dd, J=2.4, 1.1 Hz), 7.86-7.83(2H, m), 7.42-7.36(3H, m), 7.11-7.10(2H, m), 4.35 (2H, t, J=6.8 Hz), 4.24(1H, dd, J=8.3, 5.1 Hz), 4.18(2H, q, J=7.1 Hz), 3.63-3.56(1H, m), 3.36-3.28(1H, m), 3.18(2H, t, J=6.8 Hz), 3.14(1H, dd, J=13.9, 5.1 Hz), 3.05(1H, dd, J=13.9, 8.3 Hz), 2.45(3H, s), 1.22(3H, t, J=7.1 Hz), 1.09(3H, t, J=7.1 Hz) | LR 441 (M + H)⁺ |
| c-101 | 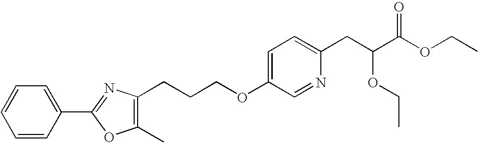 | (CDCl₃, 400 MHz): 8.22(1H, dd, J=2.0, 1.5 Hz), 7.97-7.93(2H, m), 7.43-7.37(3H, m), 7.11-7.10(2H, m), 4.25 (1H, dd, J=8.6, 5.1 Hz), 4.18(2H, q, J=7.2 Hz), 3.99(2H, t, J=6.2 Hz), 3.64-3.56(1H, m), 3.37-3.29(1H, m), 3.14 (1H, dd, J=13.9, 5.1 Hz), 3.06(1H, dd, J=13.9, 8.6 Hz), 2.68(2H, t, J=7.1 Hz), 2.26(3H, s), 2.18-2.12(2H, m), 1.22(3H, t, J=7.1 Hz), 1.09(3H, t, J=7.0 Hz) | LR 439 (M + H)⁺ |
| c-102 | 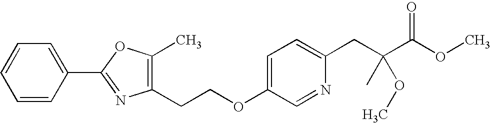 | (CDCl₃, 400 MHz): 8.18(1H, dd, J=2.3, 1.3 Hz), 7.96(2H, dd, J=7.6, 2.0 Hz), 7.54-7.51(3H, m), 7.10-7.08(2H, m), 4.25(2H, t, J=6.7 Hz), 3.29(3H, s), 3.15(2H, s), 2.96(2H, t, J=6.7 Hz), 2.36(3H, s), 2.32(3H, s), 1.38(3H, s) | LR 411 (M + H)⁺ |
| c-103 | 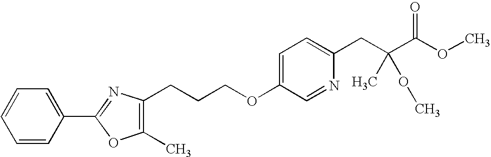 | (CDCl₃, 400 MHz): 8.20(1H, t, J=7.8 Hz), 7.97-7.93(2H, m), 7.44-7.38(3H, m), 7.09(2H, d, J=7.8 Hz), 3.99(2H, t, J=6.1 Hz), 3.72(3H, s), 3.30(3H, s), 2.68(2H, t, J=7.1 Hz), 2.62(2H, t, J=6.7 Hz), 2.27(3H, s), 2.18-2.12(2H, m), 1.40(3H, s) | LR 425 (M + H)⁺ |
| c-104 | 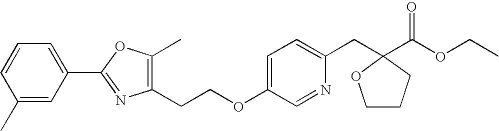 | | LR 451 (M + H)⁺ |
| c-105 | 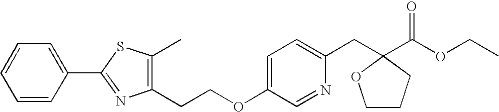 | | LR 453 (M + H)⁺ |
| c-106 | 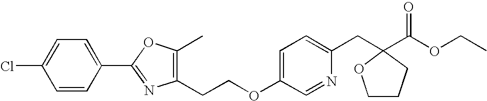 | | LR 471 (M + H)⁺ |
| c-107 | 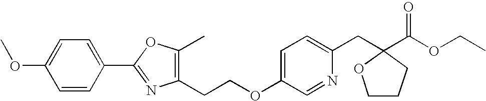 | | LR 467 (M + H)⁺ |
| c-108 | 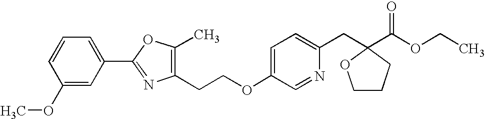 | | LR 467 (M + H)⁺ |
| c-109 | 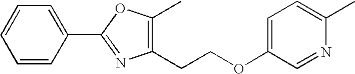 | (CDCl₃, 300 MHz): 8.18(1H, d, J=2.6 Hz), 7.97-7.94(2H, m), 7.48-7.39(3H, m), 7.10(1H, dd, J=8.7, 3.0 Hz), 7.02 (1H, d, J=8.5 Hz), 4.25(2H, t, J=6.7 Hz), 2.96(2H, t, J=6.7 Hz), 2.46(3H, s), 2.36(3H, s) | LR 295 (M + H)⁺ |
| c-110 | 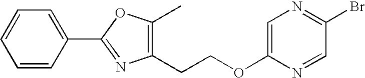 | (CDCl₃, 300 MHz): 8.16(1H, d, J=1.3 Hz), 7.96(3H, dd, J=7.4, 1.7 Hz), 7.41 (3H, dd, J=5.3, 1.9 Hz), 4.58(2H, t, J=6.7 Hz), 2.98(2H, t, J=6.8 Hz), 2.34 (3H, s) | LR 360 (M)⁻ |
| c-111 | 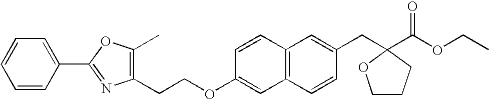 | (CDCl₃, 400 MHz): 7.99-7.97(2H, m), 7.66(1H, d, J=8.8 Hz), 7.62-7.59(2H, m), 7.45-7.35(4H, m), 7.12-7.08(2H, m), 4.35(2H, t, J=6.8 Hz), 4.16-4.09 (2H, m), 3.96-3.85(2H, m), 3.32(1H, d, J=13.6 Hz), 3.11(1H, d, J=13.9 Hz), 3.04(2H, t, J=6.8 Hz), 2.40(3H, s), 2.31-2.24(1H, m), 1.99-1.76(1H, m), 1.69-1.60(1H, m), 1.18(3H, t, J=7.1 Hz) | LR 486 (M + H)⁺ |
| c-112 | 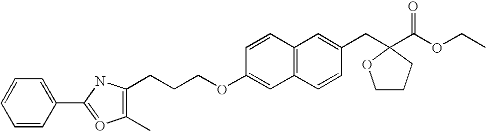 | (CDCl₃, 400 MHz): 7.99-7.97(2H, m), 7.67(1H, d, J=8.8 Hz), 7.61-7.59(2H, m), 7.45-7.35(4H, m), 7.12(1H, dd, J=2.3, 8.8 Hz), 7.08-7.07(1H, d, J=2.3 Hz), 4.16-4.06(4H, m), 3.96-3.86(2H, m), 3.33(1H, d, J=13.6 Hz), 3.11(1H,p d, J=13.6 Hz), 2.74(2H, t, J=7.1 Hz), 2.32-2.18(6H, m), 1.99-1.91(1H, m), 1.86-1.77(1H, m), 1.69-1.61(1H, m), 1.19(3H, t, J=7.1 Hz) | LR 500 (M + H)⁺ |

### Preparation c-113

### 3-(5-Methyl-2-phenyl-oxazol-4-yl)-propionaldehyde

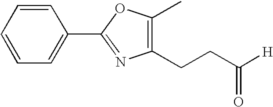

To a solution of 3-(5-methyl-2-phenyl-oxazol-4-yl)-propan-1-ol (1.0 g, 4.6026 mmol) in dichloromethane (20 mL) was added pyridinium chlorochromate (9.9213 g of ˜20 wt. % on SiO₂, 9.2051 mmol). The resulting mixture was stirred under an atmosphere of nitrogen at ambient temperature for 16 hours and the volatiles removed under reduced pressure. The residue was purified by flash column chromatography (hexanes to ethyl acetate) to yield the pure aldehyde (0.4752 g, 48%) as a colorless oil. LRMS (m/z): 216 (M+H)⁺. ¹H NMR (CDCl₃, 300 MHz): δ 9.84 (1H, s), 7.96-7.93 (2H, m), 7.42-7.37 (3H, m), 2.85 (2H, dd, J=6.0, 0.9 Hz), 2.80 (2H, d, J=6.0 Hz), 2.33 (3H, s).

### Preparation c-114

### 4-But-3-enyl-5-methyl-2-phenyl-oxazole

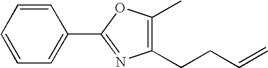

To a solution of methyl triphenylphosphonium iodide (1.7848 g, 4.4154 mmol) in dry tetrahydrofuran (95 mL), under an atmosphere of nitrogen at 0° C., was added butyllithium (1.8 mL of a 2.5M solution in hexanes, 4.4154 mmol) dropwise. The suspension dissolved and the solution turned orange. After 10 minutes a solution of 3-(5-methyl-2-phenyl-oxazol-4-yl)-propionaldehyde (0.4752 g, 2.2077 mmol) in dry tetrahydrofuran (15 mL) was added dropwise and the solution allowed to warm to ambient temperature. After 16 hours, hexanes (200 mL) was added and the precipitate filtered off. The filtrate was then extracted with water (200 mL) and the organic phase dried (anhydrous magnesium sulfate), filtered, and concentrated in vacuo to afford the crude product. The residue was purified by flash column chromatography (hexanes to ethyl acetate) to yield the pure title compound (0.291 g, 62%) as a colorless oil. LRMS (m/z): 214 (M+H)⁺. ¹H NMR (CDCl₃, 300 MHz): δ 7.99-7.96 (2H, m), 7.43-7.38 (3H, m), 5.93-5.79 (1H, m), 5.09-5.02 (1H, m), 5.00-4.95 (1H, m), 2.57 (2H, t, J=7.4 Hz), 2.42 (2H, t, J=7.4 Hz), 2.31 (3H, s).

### Preparation c-115

### 2-{6-[4-(5-Methyl-2-phenyl-oxazol-4-yl)-butyl]-pyridin-3-ylmethyl}-tetrahydro-furan-2-carboxylic acid ethyl ester

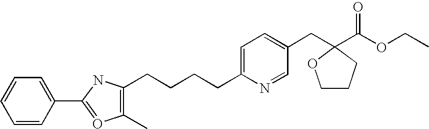

9-Borabicyclononane (5.5 mL of a 0.5M solution in tetrahydrofuran, 2.729 mmol) was added to a yellow solution of 4-but-3-enyl-5-methyl-2-phenyl-oxazole (0.291 g, 1.3645 mmol) in dry tetrahydrofuran (1.3 mL). The mixture was stirred at ambient temperature for 4 hours and then transferred to another flask containing 2-(6-bromo-pyridin-3-ylmethyl)-tetrahydro-furan-2-carboxylic acid ethyl ester (0.3297 g, 1.0496 mmol), palladium dichloride bis(diphenylphosphino)ferrocene (0.0857 g, 0.1050 mmol), cesium carbonate (0.9551 g, 2.9389 mmol), triphenylarsine (0.0322 g, 0.1050 mmol) in N,N-dimethylformamide (2.8 mL) and water (0.23 mL). The dark red mixture was stirred for 16 hours at ambient temperature under a nitrogen atmosphere. After cooling to 0° C., the reaction was quenched with 2M aqueous sodium acetate (5 mL) and 30% aqueous hydrogen peroxide (2 mL). The resulting solution was stirred for 2 hours, diluted with water (25 mL), and extracted with ethyl acetate (4×50 mL). The combined organic extracts were washed with water (25 mL), dried (anhydrous magnesium sulfate), filtered, and concentrated in vacuo to give the crude product. The residue was purified by flash column chromatography (hexanes to ethyl acetate) to afford the title compound (0.2805 g, 60%) as pale yellow oil. LRMS (m/z): 449 (M+H)⁺. ¹H NMR (CDCl₃, 300 MHz): δ 8.34 (1H, d, J=1.9 Hz), 7.95 (2H, dd, J=7.7, 1.9 Hz), 7.52 (1H, dd, J=8.0, 2.2 Hz), 7.42-7.36 (3H, m), 7.04 (1H, d, J=7.9 Hz), 4.13 (2H, q, J=7.2 Hz), 3.96-3.84 (2H, m), 3.16 (1H, d, J=13.9 Hz), 2.90 (1H, d, J=13.9 Hz), 2.77 (2H, t, J=7.3 Hz), 2.50 (2H, t, J=6.9 Hz), 2.31-2.19 (1H, m), 2.28 (3H, s), 1.92-1.64 (7H, m), 1.20 (3H, t, J=7.2 Hz).

### Preparation c-116

### 2-{6-[3-(5-methyl-2-phenyl-oxazol-4-yl)-propyl]-pyridin-3-ylmethyl}-tetrahydro-furan-2-carboxylic acid ethyl ester

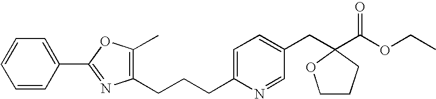

9-Borabicyclononane (4.2 mL of a 0.5M solution in tetrahydrofuran, 2.07 mmol) was added to a yellow solution of 4-allyl-5-methyl-2-phenyl-oxazole (0.21 g, 1.04 mmol) in dry tetrahydrofuran (1 mL). The mixture was stirred at ambient temperature for 4 hours and then transferred to another flask containing 2-(6-bromo-pyridin-3-ylmethyl)-tetrahydro-furan-2-carboxylic acid ethyl ester (0.25 g, 0.80 mmol), palladium dichloride bis(diphenylphosphino)ferrocene (0.07 g, 0.1 mmol), cesium carbonate (0.72 g, 2.90 mmol), triphenylarsine (0.02 g, 0.1 mmol) in N,N-dimethylformamide:water (4:1, 2.63 mL). The dark red mixture was stirred for 16 hours at ambient temperature under a nitrogen atmosphere. After cooling to 0° C., the reaction was quenched with 2M aqueous sodium acetate (4.7 mL) and 30% aqueous hydrogen peroxide (1.7 mL). The resulting solution was stirred for 2 hours, diluted with water (20 mL), and extracted with ethyl acetate (4×50 mL). The combined organic extracts were washed with water (20 mL), dried (anhydrous magnesium sulfate), filtered, and concentrated in vacuo to give the crude product. The residue was purified by flash column chromatography (40% to 90% ethyl acetate/hexanes) to afford the title compound (0.20 g, 57%) as a colorless oil. LRMS (m/z): 435 (M+H)⁺. ¹H NMR (Dimethyl sulfoxide-d₆, 400 MHz): δ 8.35 (1 H, s), 7.96 (2H, d, J=7.9 Hz), 7.55 (1H, d, J=8.3 Hz), 7.39 (3H, t, J=5.9 Hz), 7.09 (1H, d, J=8.1 Hz), 3.91 (2H, m), 3.80 (2H, t, J=10.9 Hz), 3.17 (1H, d, J=14.0 Hz), 2.91 (1H, d, J=13.94 Hz), 2.81 (2H, t), 2.53 (2H, t, J=7.3 Hz), 2.28 (3H, s), 2.09 (2H, d, J=7.5 Hz), 1.87 (4H, d, J=10.9 Hz), 1.23 (3H, m).

### Preparation c-117

### Ethyl 1-{4-[(1E)-3-(5-methyl-2-phenyl-1,3-oxazol-4-yl)prop-1-en-1-yl]phenoxy}cyclobutanecarboxylate

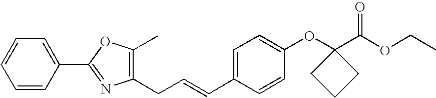

A mixture of Pd(OAc)₂ (12 mg, 0.05 mmol) and Ph₃P (26 mg, 0.05 mmol) in toluene (2 mL) was stirred under nitrogen at room temperature for 1 hour and followed by the addition of Et₃N (2 mL) and a solution of 4-allyl-5-methyl-2-phenyl-1,3-oxazole (100 mg, 0.50 mmol) and ethyl 1-(4-iodophenoxy)cyclobutanecarboxylate (173 mg, 0.50 mmol) in toluene (2 mL). The resulting reaction solution was heated at 80° C. under nitrogen for 17 hours and cooled to room temperature. After solvent removal, the residue was partitioned between EtOAc and brine. The separated organic layer was washed with brine, dried over Na₂SO₄, and concentrated to give the crude product as brown oil. Purification by silica gel column with 20% EtOAc in hexane gave 85 mg (41%) of yellow oil. ¹H NMR (400 MHz, CDCl₃) δ 1.19 (t, 3 H), 1.98 (m, 2 H), 2.38 (s, 3 H), 2.43 (m, 2 H), 2.72 (m, 2 H), 3.41 (d, 2 H), 4.18 (q, 2 H), 6.20 (td, 1 H), 6.40 (d, 1 H), 6.60 (d, 2 H), 7.25 (d, 2 H), 7.40 (d, 3 H), 8.00 (d, 2 H). LRMS (m/z): 418 (M+H)⁺.

### Preparation c-118

### Ethyl 1-{4-[3-(5-methyl-2-phenyl-1,3-oxazol-4-yl)propyl]phenoxy}cyclobutane-carboxylate

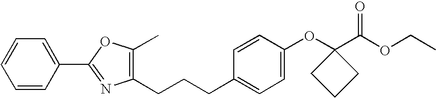

Ethyl 1-{4-[(1E)-3-(5-methyl-2-phenyl-1,3-oxazol-4-yl)prop-1-en-1-yl]phenoxy}cyclobutane carboxylate (85 mg, 0.20 mmol) was dissolved in MeOH (5 mL) and followed by the addition of 10% Pd/C (15 mg). The mixture was stirred at room temperature for 16 hours with a balloon, full of hydrogen gas, attached to the flask. The mixture was filtered through a pad of Celite and the cake was rinsed with MeOH. The filtrate was concentrated to give 85 mg (100%) of yellow oil. ¹H NMR (400 MHz, CDCl₃) δ 1.19 (t, 3 H), 1.92-2.02 (m, 4 H), 2.27 (s, 3 H), 2.39-2.51 (m, 4 H), 2.60 (t, 2 H), 2.66-2.77 (m, 2 H), 4.19 (q, 2 H), 6.60 (d, 2 H), 7.05 (d, 2 H), 7.36-7.47 (m, 3 H), 7.98 (dd, 2 H). LRMS (m/z): 420 (M+H)⁺.

### Preparation c-119

### 5-Bromo-pyrazin-2-ylamine

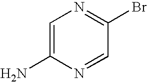

To a solution of pyrazin-2-ylamine (2.0 g, 21.03 mmol) in dry dichloromethane (120 mL) at 0° C., was added N-bromosuccinimide (3.74 g, 21.03 mmol) slowly to maintain the internal temperature below 0° C. The mixture was stirred at the same temperature for 24 hours, and then washed with saturated aqueous sodium bicarbonate (30 mL) and water (30 mL). The combined aqueous extracts were extracted with dichloromethane (3×100 mL). The combined organic extracts were. dried (anhydrous magnesium sulfate), filtered, and concentrated in vacuo to afford the crude product. The residue was purified by flash column chromatography (10% to 50% ethyl acetate/hexanes) to yield the title compound (2.57 g, 70%) as a yellow solid. LRMS (m/z): 174 (M)⁻. ¹H NMR (CDCl₃, 300 MHz): δ 8.08 (1H, d, J=1,3 Hz), 7.76 (1H, d, J=1,3 Hz).

### Preparation c-120

### 5-Bromo-pyrazin-2-ol

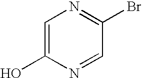

Sodium nitrite (1,35 g, 19.53 mmol) was added portionwise to concentrated sulfuric acid (9.8 mL) at 0° C. The mixture was heated at 50° C. until all of the sodium nitrite had dissolved and the mixture was again cooled to 0° C. A solution of 5-bromo-pyrazin-2-ylamine (2.57 g, 14.68 mmol) in concentrated sulfuric acid (14.7 mL) was added dropwise to the nitronium solution at 0° C. The ice bath was removed, the mixture warmed to ambient temperature and stirred for 15 minutes before heating to 45° C. for seven minutes. After cooling to ambient temperature, the mixture was poured slowly with precaution into crushed ice water (100 mL). The aqueous phase was neutralized to pH 4 with 20% aqueous sodium hydroxide then extracted with ethyl acetate (3×100 mL). The combined organic extracts were washed with water (50 mL), dried (anhydrous magnesium sulfate), filtered, and evaporated to afford the title compound (1.88 g, 73%) as a yellow solid. ¹H NMR (CDCl₃, 300 MHz): δ 8.07 (1H, s), 7.62 (1H, d, J=3.0 Hz).

### Preparation c-121

### 2-(tert-Butyl-dimethyl-silanyloxymethyl)-5-[2-(5-methyl-2-phenyl-oxazol-4-yl)-ethoxy]-pyrazine

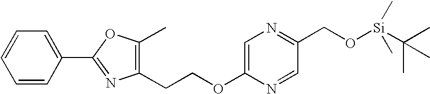

To a solution of 2-bromo-5-[2-(5-methyl-2-phenyl-oxazol-4-yl)-ethoxy]-pyrazine (0.50 g, 1.39 mmol) and tert-butyl-dimethyl-tributylstannanylmethoxy-silane (0.91 g, 2.09 mmol) in dry 1,4-dioxane (8 mL) was added tetrakistriphenylphosphine(0) palladium (0.16 g, 0.14 mmol). The mixture was degassed three times and then heated at 120° C. for 22 hours. After cooling to ambient temperature the mixture was diluted with diethyl ether (10 mL) and then quenched with saturated aqueous potassium fluoride (5 mL). The resulting mixture was stirred for 30 minutes and then extracted with ethyl acetate (3×50 mL). The organic phase was washed with water (30 mL), dried (anhydrous magnesium sulfate), filtered, and evaporated to afford the title compound without any further purification. LRMS (m/z): 426 (M+H)⁺. ¹H NMR (CDCl₃, 300 MHz): δ 8.20 (1H, s), 8.09 (1H, s), 7.97 (2H, d, J=7.4 Hz), 7.41 (3H, d, J=5.3 Hz), 4.78 (2H s), 4.58 (2H, d, J=6.6 Hz), 2.98 (2H, s), 2.34 (3H, s), 0.97 (2H, m), 0.14 (6H, m).

### Preraration c-122

### {5-[2-(5-Methyl-2-phenyl-oxazol-4-yl)-ethoxy]-pyrazin-2-yl}-methanol

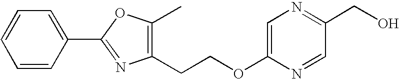

Tetrabutylammonium fluoride (2.8 mL of a 1M solution in tetrahydrofuran, 2.78 mmol) was added dropwise to a solution of 2-(tert-butyl-dimethyl-silanyloxymethyl)-5-[2-(5-methyl-2-phenyl-oxazol-4-yl)-ethoxy]-pyrazine (1.39 mmol) in dry tetrahydrofuran (20 mL). The mixture was stirred at ambient temperature for 16 hours and then quenched with water (1 mL), and acidified to pH 5 with 1M aqueous acetic acid solution. The organics were removed in vacuo and the aqueous phase extracted with dichloromethane (3×50 mL). The combined organic extracts were dried (anhydrous magnesium sulfate), filtered, and concentrated in vacuo to afford the title compound (0.0928 g, 21%). LRMS (m/z): 312 (M+H)⁺. ¹H NMR (CDCl₃, 300 MHz): δ 8.12 (2H, s), 8.05 (2H, d, J=6.0 Hz), 7.40 (3H, d, J=6.0 Hz), 4.72 (2H, s), 4.59 (2H, t, J=6.0 Hz), 2.99 (2H, t, J=6.0 Hz), 2.33 (3H, s).

### Preparation c-123

### 6-Benzyloxy-naphthalene-2-carboxylic acid benzyl ester

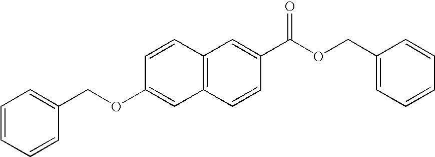

The above compound was prepared according to the procedure described in Inui, S.; Suzuki, T.; Iimura, N.; Iwane, H.; Nohira, H. Mol. Cryst. Liq. Cryst. Sci. Technol. Sect. A. 1994, 239, 1-10.

### Preparation c-124

### (6-Benzyloxy-naphthalen-2-yl)-methanol

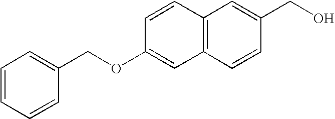

To a solution of 6-benzyloxy-naphthalene-2-carboxylic acid benzyl ester (7.09 g, 19.24 mmol) in dry tetrahydrofuran (60 mL), under an atmosphere of nitrogen at 0° C., was added diisobutylaluminum hydride (58 mL of a 1.0M solution in tetrahydrofuran, 57.73 mmol). The resulting mixture was allowed to warm to ambient temperature and stirred for 16 hours. An solution of citric acid (19 g) in water (40 mL) was added dropwise (CAUTION!: strong exotherm). The aqueous layer was then extracted with ethyl acetate (3×50 mL) and the combined organic extracts washed with saturated aqueous sodium chloride (50 mL), dried (anhydrous magnesium sulfate), and concentrated in vacuo to afford the crude product. The residue was purified by flash column chromatography (hexanes to ethyl acetate) to yield the title compound (4.37 g, 86%) as a white solid. LRMS (m/z): 287 (M+Na)⁺. ¹H NMR (CDCl₃, 400 MHz): δ 7.76-7.72 (3H, m), 7.50-7.33 (6H, m), 7.25-7.23 (2H, m), 5.18 (2H, s), 4.82 (2H, d, J=6.1 Hz).

### Preparation c-125

### 2-[6-(5-Methyl-2-phenyl-oxazol-4-ylmethoxy)-naphthalen-2-ylmethyl]-tetrahydro-furan-2-carboxylic acid ethyl ester

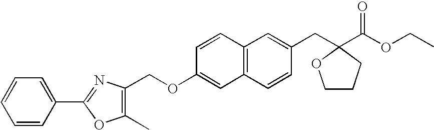

A heterogeneous mixture of 2-phenyl-4-(chloromethyl)-5-methyloxazole (0.133 g, 0.639 mmol), 2-(6-hydroxy-naphthalen-2-ylmethyl)-tetrahydro-furan-2-carboxylic acid ethyl ester (0.192 g, 0.639 mmol), and cesium carbonate (0.521 g, 1.59 mmol) in dry acetonitrile (2 mL) was heated (in a microwave) at 140° C. for 10 minutes. A second portion of 2-phenyl-4-(chloromethyl)-5-methyloxazole (0.5 eq.) was added and the mixture heated at 200° C. for a further 20 minutes. The reaction mixture was filtered through Celite and washed with acetonitrile (200 mL). The filtrate was concentrated in vacuo and the residue purified by flash column chromatography (hexanes to ethyl acetate) to yield the title compound (0.180 g, 60%) as a colorless oil.

### Preparation c-126

### (4S)-4-benzyl-3-(tetrahydrofuran-2-ylcarbonyl)-1,3-oxazolidin-2-one

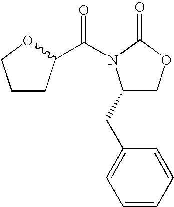

n-Butyllithium (22.6 mL of a 2.5M solution in hexanes, 56.4 mmol) was added dropwise to a solution of (4S)-4-benzyl-1,3-oxazolidin-2-one (10.0 g, 56.4 mmol) in tetrahydrofuran (200 mL) at −78° C. The mixture was stirred for 30 minutes then a solution of tetrahydrofuran-2-carbonyl chloride (9.12 g, 67.7 mmol) in tetrahydrofuran (25 mL) was added. The mixture was stirred at −78° C. for 30 minutes, warmed to 0° C. over 1 hour and quenched with saturated ammonium chloride solution. The Mixture was extracted with ethyl acetate and the organic phase was washed with brine, dried over magnesium sulfate, filtered and evaporated. The residue was purified by flash column chromatography (1:3 then 1:2 ethyl acetate:hexanes) to yield the title compound as a ca. 1:1 mixture of diastereoisomers as a colorless oil (15.0 g, 97%).

### Preparation c-127

### (4S)-4-benzyl-3-{[2-({6-[2-(5-methyl-2-phenyl-1,3-oxazol-4-yl)ethoxy]pyridin-3-yl}methyl)tetrahydrofuran-2-yl]carbonyl}-1,3-oxazolidin-2-one

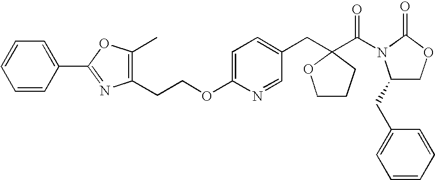

Sodium (bis)trimethylsilyl amide (3.57 mL of 1M solution in tetrahydrofuran, 3.57 mmol) was added dropwise to a solution of (4S)-4-benzyl-3-(tetrahydrofuran-2-ylcarbonyl)-1,3-oxazolidin-2-one (0.983 g, 3.57 mmol) in anhydrous tetrahydrofuran (6 mL) at −50° C. The mixture was stirred for 45 minutes and then a solution of 5-(iodomethyl)-2-[2-(5-methyl-2-phenyl-1,3-oxazol-4-yl)ethoxy]pyridine (Preparation 28) (0.500 g, 1.19 mmol) in anhydrous tetrahydrofuran (6 mL) was added dropwise. The resulting mixture was stirred at −50° C. for 1.5 hours then quenched with saturated aqueous ammonium chloride and warmed to ambient temperature. The mixture was extracted with ethyl acetate and the organic phase dried (anhydrous magnesium sulfate), filtered and evaporated. The residue was purified by flash column chromatography (1:1 ethyl acetate:hexanes) to yield the title compound as a single diastereoisomer as a colorless oil (0.617 g, 90%). LRMS (m/z): 568 (M+H)⁺. ¹H NMR (CDCl₃, 300 MHz) δ 8.01 (1H, s), 7.96 (2H, m), 7.61 (1H, dd, J=2.5, 8.5 Hz), 7.40 (3H, m), 7.28 (3H, m), 7.17 (1H, m), 6.64 (1H, d, J=8.6 Hz), 4.55 (3H, m), 4.18 (2H, m), 3.90 (1H, m), 3.79 (1H, m), 3.27 (1H, d, J=14 Hz), 3.20 (1H, m), 3.13 (1H, d, J=14 Hz), 2.96 (2H, t, J=6.8 Hz), 2.79 (1H, m), 2.32 (3H, s), 2.34 (3H, m), 2.11 (1H, m), 1.73 (1H, m), 1.54 (1H, m).

### Preparation c-128

### Tetrahydro-furan-2-carboxylic acid amide

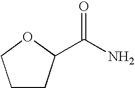

To a solution of tetrahydro-furan-2-carboxylic acid (2.42 g, 20.82 mmol) in anhydrous tetrahydrofuran (120 mL), under an atmosphere of nitrogen at 0° C., was added triethylamine (8.5 mL, 61.23 mmol) and ethyl chloroformate (2.4 mL, 25.10 mmol). White precipitation formed after the addition of ethyl chloroformate and the resulting mixture stirred for 45 minutes at 0° C. Ammonia gas was bubbled into the solution for 2 hours and the gas source removed. The reaction mixture was then allowed to warm to ambient temperature and stirred for 16 hours. The solution was adjusted to pH 1 by addition of 1 N hydrochloric acid, and then extracted with ethyl acetate (3×50 mL). The combined organic extracts were dried (anhydrous magnesium sulfate), filtered, and concentrated in vacuo to give the crude product. The residue was purified by flash column chromatography (hexanes to 10% ethyl acetate/hexanes) to afford the title compound (0.97 g, 41%) as a white solid. LRMS (m/z): 116 (M+H)⁺. ¹H NMR (CDCl₃, 300 MHz): 4.35 (1H, dd, J=8.5, 5.8 Hz), 3.92 (2H, m), 2.18 (2H, m), 1.90 (2H, m).

### Preparation c-129

### Tetrahydro-furan-2-carbonitrile

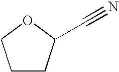

Trifluoroacetic anhydride (1.55 g, 7.38 mmol) was added slowly, with a rate of one drop every 10 seconds, to an ice-cold solution (0° C.) of tetrahydro-furan-2-carboxylic acid amide (0.77 g, 6.71 mmol) and pyridine (1.06 g, 13.42 mmol) in anhydrous 1,4-dioxane (10 mL). The addition of trifluoroacetic anhydride was monitored to keep the internal temperature below 5° C. and was completed after 20 minutes. The resulting mixture was allowed to warm to ambient temperature, and stirred for 3 hours. Chloroform (100 mL) was added to the mixture, and then extracted with water (30 mL) and saturated aqueous sodium chloride (20 mL). The organic extracts were dried (anhydrous magnesium sulfate), filtered, and concentrated in vacuo to give the crude product. The residue was purified by flash column chromatography (hexanes to 25% ethyl acetate/hexanes) to afford the title compound (0.51 g, 62%) as a colorless oil. ¹H NMR (CDCl₃, 300 MHz): 4.70 (1H, m), 3.96 (2H, m), 2.24 (2H, m), 2.08 (2H, m).

### Preparation c-130

### 2-{6-[2-(5-Methyl-2-phenyl-oxazol-4-yl)-ethoxy]-pyridin-3-ylmethyl}-tetrahydro-furan-2-carbonitrile

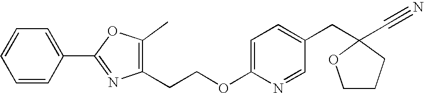

Sodium bis(trimethylsilyl)amide (1.8 mL, 1.79 mmol) was added to a solution of tetrahydro-furan-2-carbonitrile (0.17 g, 1.79 mmol) in anhydrous tetrahydrofuran (6 mL) under an atmosphere of nitrogen at −78° C. The resulting yellow solution was stirred for 50 minutes, and then a solution of 5-iodomethyl-2-[2-(5-methyl-2-phenyl-oxazol-4-yl)-ethoxy]-pyridine (0.25 g, 0.596 mmol) in anhydrous tetrahydrofuran (3 mL) was added to the enolate solution. The mixture was stirred at −78° C. for 1.5 hours, and quenched with saturated aqueous ammonium chloride (5 mL). The aqueous phase was extracted with ethyl acetate (3×50 mL), and the combined organic extracts washed with water (30 mL), dried (anhydrous magnesium sulfate), filtered, and concentrated in vacuo to give the crude product. The residue was purified by flash column chromatography (7% to 45% ethyl acetate/hexanes) to afford the title compound (0.11 g, 46%) as a white solid. LRMS (m/z): 390 (M+H)⁺. ¹H NMR (CDCl₃, 300 MHz), δ 8.03 (1H, d, J=2.5 Hz), 7.96 (2H, m), 7.56 (1H, dd, J=8.5, 2.5 Hz), 7.40 (3H, m), 6.68 (1H, d, J=8.5 Hz), 4.54 (2H, m), 3.96 (2H, m), 3.00 (4H, m), 2.33 (5H, d, J=3.2 Hz), 1.92 (2H, m).

### EXAMPLE D-1

### 2-ethoxy-3-{6-[2-(5-methyl-2-phenyl-1,3-oxazol-4-yl)ethoxy]pyridin-3-yl}propanoic acid

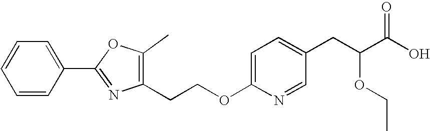

Lithium hydroxide monohydrate (180 mg, 4.31 mmol) was added to a solution of ethyl 2-ethoxy-3-{6-[2-(5-methyl-2-phenyl-1,3-oxazol-4-yl)ethoxy]pyridin-3-yl}propanoate (183 mg, 0.431 mmol) in a mixture of tetrahydrofuran:methanol:water (1:1:1, 6 mL). The mixture was stirred 18 hours then the volatile components were removed by evaporation. The aqueous phase was acidified with 3M hydrochloric acid and extracted with ethyl acetate. The organic phase was washed with brine, dried over magnesium sulfate, filtered and evaporated. The residue was purified twice by flash column chromatography (98:2 dichloromethane:methanol) to yield the title compound as a colorless glass (31 mg). LRMS (m/z): 396 (M)⁺. ¹H NMR (CDCl₃, 300 MHz) δ 7.99 (3H, m), 7.50 (1H, m), 7.40 (3H, m), 6.65 (1H, m), 4.50 (2H, t, J=7 Hz), 4.01 (1H, m), 3.64 (1H, m), 3.42 (1H, m), 2.98 (4H, m), 2.34 (3H, s), 1.16 (3H, t, J=7 Hz).

### EXAMPLE D-2 TO D-43

Examples D-2 to D-43 were prepared by procedures analogous to those used for Example D-1 by stirring a solution of the ester with sodium or lithium hydroxide in aqueous methanol, aqueous ethanol, aqueous tetrahydrofuran or mixtures thereof at temperatures between 20° C. and 75° C.

| | | | MS | |
|---|---|---|---|---|
| Ex | | | (m/z) | |
| # | Structure | ¹H NMR | LR/HR | Analysis |
| D-2 | 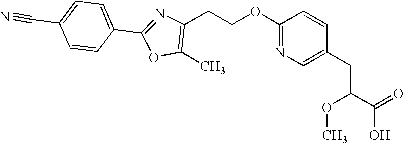 | ¹H NMR (DMSO-d₆, 400 MHz): 8.05(2H, d, J=8.6 Hz), 7.96-7.94 (3H, m), 7.55(1H, dd, J=8.3 and 2.3 Hz), 6.70(1H, d, J=8.3 Hz), 4.45(2H, t, J=6.6 Hz), 3.89(1H, dd, J=7.6 and 5.1 Hz), 3.22(3H, s), 2.95-2.87(3H, m), 2.80(1H, dd, J=14.2 and 7.8 Hz), 2.35(3H, s) | LR 408 (M + H)⁺ | |
| D-3 | 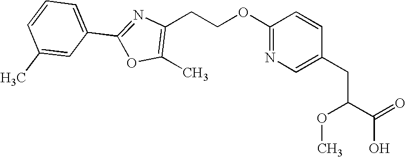 | ¹H NMR (DMSO-d₆, 400 MHz): 7.96(1H, d, J=2.3 Hz), 7.73(1H, s, 7.69(1H, d, J=7.8 Hz), 7.55 (1H, dd, J=8.6 and 2.3 Hz), 7.37 (1H, t, J=7.8 Hz), 7.28(1H, d, J=7.1 Hz), 6.70(1H, d, 8.3 Hz), 4.44 (2H, t, J=6.8 Hz), 3.89(1H, dd, J=8.1 and 4.8 Hz), 3.22(3H, s), 2.92-2.88(3H, m), 2.80(1H, d, J=14.7 and 8.12 Hz), 2.36(3H, s), 2.31(3H, s) | 397.1775 | C₂₂H₂₄N₂O₅0.5 H₂O C 65.17 H 6.22 |
| # N 6.91. Found: C 65.03 H 6.10 N 7.07 | | | | |
| D-4 | 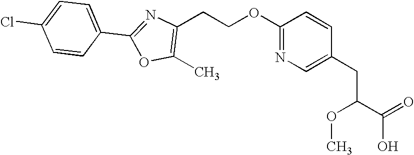 | ¹H NMR (DMSO-d₆, 400 MHz): 7.98(1H, d, J=1.8 Hz), 7.92(2H, d, J=8.6 Hz), 7.58-7.55(3H, m), 6.72(1H, d, J=8.6 Hz), 4.46(2H, t, J=6.6 Hz), 3.91(1H, dd, J=7.8 and 4.6 Hz), 3.24(3H, s), 2.94-2.89(3H, m), 2.82(1H, dd, J=14.4 and 7.6 Hz), 2.33(3H, s). | 417.1232 | |
| D-5 | 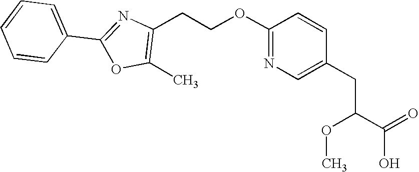 | ¹H NMR (DMSO-d₆, 400 MHz): 12.71(1H, s), 7.96(1H, d, J=2.3 Hz), 7.90(2H, dd, J=7.6 and 2.0), 7.55(1H, dd, J=8.6 and 2.3 Hz), 7.51-7.46(3H, m), 6.71(1H, d, J=8.6 Hz), 4.44(2H, t, J=6.6 Hz), 3.89(1H, dd, J=7.6 and 5.1 Hz), 3.22(3H, s), 2.92-2.88(3H, m), 2.80(1H, dd, J=14.2 and 7.6 Hz), 2.32(3H, s) | | Calcd for C₂₁H₂₂N₂O₅0.1 H₂O C 65.65 H 5.82 N 7.29. Found: C 65.45 H |
| # 5.92 N 7.26. | | | | |
| D-6 | 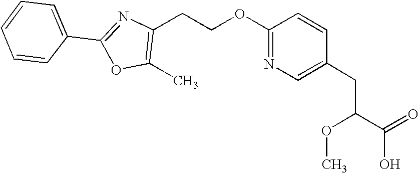 | ¹H NMR (DMSO-d₆, 400 MHz): 12.81(1H, s), 7.96(1H, d, J=2.0 Hz), 7.90(2H, dd, J=7.7 and 1.9), 7.55(1H, dd, J=8.6 and 2.3 Hz), 7.51-7.46(3H, m), 6.70(1H, d, J=8.6 Hz), 4.44(2H, t, J=6.8 Hz), 3.88(1H, dd, J=7.6 and 4.8 Hz), 3.22(3H, s), 2.92-2.87(3H, m), 2.80(1H, dd, J=14.2 and 7.8 Hz), 2.32(3H, s). | | Calcd for C₂₁H₂₂N₂O₅0.2 5H₂O C 65.19 H 5.86 N 7.24. Found: C 65.19 H |
| # 5.80 NM 7.03 | | | | |
| D-7 | 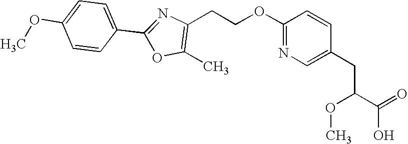 | ¹H NMR (DMSO-d₆, 400 MHz): 12.54(1H, s), 7.96(1H, d, J=2.0 Hz), 7.83(2H, d, J=9.1), 7.55 (1H, dd, J=8.6 and 2.3 Hz), 7.03 (2H, d, J=8.8 Hz), 6.70(1H, d, J=8.6 Hz), 4.43(2H, t, J=6.8 Hz), 3.90(1H, dd, J=7.8 and 4.8 Hz), 3.80(3H, s), 3.22(3H, s), 2.92-2.86(3H, m), 2.80(1H, dd, J=14.7 and 7.6 Hz), 2.29(3H, s) | Calcd for C₂₂H₂₅N₂O₆413.1707. Found:413.1717 | |
| D-8 | 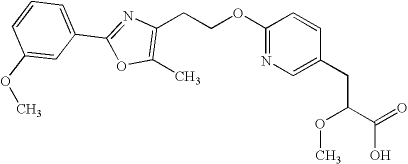 | ¹H NMR (DMSO-d₆, 400 MHz): 12.77(1H, s), 7.96(1H, d, J=2.3 Hz), 7.55(1H, dd, J=8.3 and 2.3), 7.48(1H, d, J=7.8 Hz), 7.41(1H, d, J=8.1 Hz), 7.39-7.37(1H, m), 7.04(1H, dd, J=8.1 and 2.5 Hz), 6.71(1H, d, J=8.6 Hz), 4.44(2H, t, J=6.8 Hz), 3.89(1H, dd, J=7.8 and 4.8 Hz), 3.81(3H, s), 3.22 (3H, s), 2.92-2.88(3H, m), 2.80 (1H, dd, J=14.4 and 8.0 Hz), 2.31 (3H, s). | Calcd for | |
| #C₂₂H₂₅N₂O₆413.1707. Found: 413.1715 | | | | |
| D-9 | 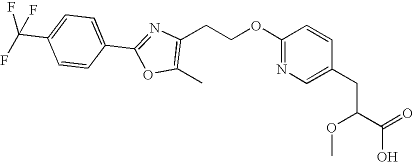 | ¹H NMR (DMSO-d₆, 400 MHz): 12.70(1H, s), 8.04(2H, d, J=8.1 Hz), 7.91(1H, d, J=2.0), 7.80 (2H, d, J=8.3 Hz), 7.49(1H, dd, J=8.3 and 2.4 Hz), 6.65(1H, d, J=8.3 Hz), 4.40(2H, t, J=6.6 Hz), 3.84(1H, dd, J=7.6 and 4.7 Hz), 3.16(3H, s), 2.90-2.82(3H, m), 2.74(1H, dd, J=14.4 and 7.8 Hz), 2.29(3H, s). | Calcd for C₂₂H₂₂F₃N₂O₅451.1476. Found: 451.1474 | |
| D-10 | 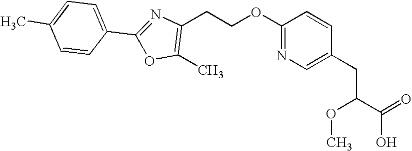 | ¹H NMR (DMSO-d₆, 400 MHz): 12.76(1H, s), 7.96(1H, d, J=2.3 Hz), 7.79(2H, d, J=8.1), 7.55 (1H, dd, J=8.6 andf 2.5 Hz), 7.29 (2H, d, J=8.1 Hz), 6.68(1H, d, J=8.3 Hz), 4.43(2H, t, J=6.8 Hz), 3.89(1H, dd, J=7.8 and 4.8 Hz), 3.22(3H, s), 2.92-2.87(3H, m), 2.80(1H, dd, J=14.2 and 7.8 Hz), 2.34(3H, s), 2.30(3H, s). | Calcd for C₂₂H₂₅N₂O₅397.1758. Found: 397.1770 | Calcd for |
| #C₂₂H₂₄N₂O₅0.3 H₂O C 65.75 H 6.17 N 6.97. Found: C 65.74 H 6.14 N 6.81 | | | | |
| D-11 | 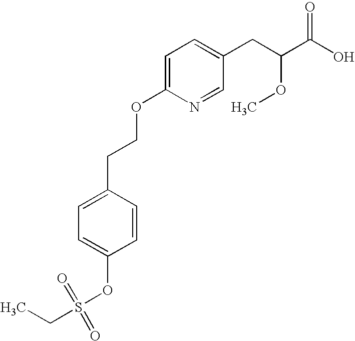 | ¹H NMR (MeOH-d₄, 400 MHz): 8.30(1H, s), 7.58(1H, d, J=9.2 Hz), 7.11(2H, d, J=8.2 Hz), 6.71(2H, d, J=8.2 Hz), 6.31(1H, d, J=9.2 Hz), 4.57(2H, t, J=6.5 Hz), 4.18(1H, dd, J=13.5, 0.2 Hz), 3.98(2H, q, J=7.7 Hz), 3.52(3H, s), 3.28(2H, t, J=6.5 Hz), 3.05-3.11(1H, m), 2.89-2.98(1H, m), 1.44(3H, t, J=7.7 Hz) | LRMS: 410 (M + H)⁺. | |
| D-12 | 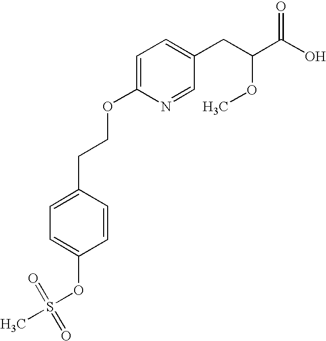 | ¹H NMR (MeOH-d₄, 400 MHz): 8.30(1H, s), 7.58(1H, d, J=9.2 Hz), 7.10(2H, d, J=8.2 Hz), 6.74(2H, d, J=8.2 Hz), 6.31(1H, d, J=9.2 Hz), 4.57(2H, t, J=6.5 Hz), 4.18 (1H, dd, J=13.5, 0.2 Hz), 3.52(3H, s), 3.34(3H, s), 3.28(2H, t, J=6.5 Hz), 3.06-3.11(1H, m), 2.89-2.98 (1H, m) | LR 396 (M + H)⁺ | |
| D-13 | 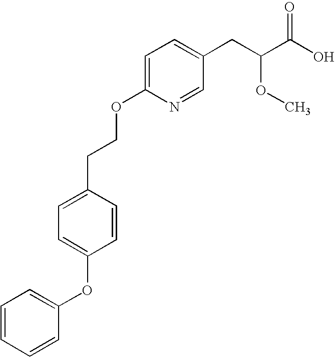 | ¹H NMR (MeOH-d₄, 400 MHz): 8.30(1H, s), 7.58(1H, d, J=9.2 Hz), 7.31-7.37(2H, m, J=8.0, 7.5 0.2, 0.2 Hz), 7.06-7.14(3H, m), 6.96-7.04(4H, m), 6.31(1H, d, J=9.2 Hz), 4.57(2H, t, J=6.5 Hz), 4.18(1H, dd, J=13.5, 0.2 Hz), 3.52 (3H, s), 3.28(2H, t, J=6.5 Hz), 3.06-3.11(1H, m), 2.89-2.97(1H, m) | LR 394 (M + H)⁺ | |
| D-14 | 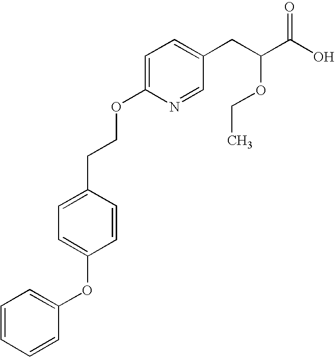 | ¹H NMR (MeOH-d₄, 400 MHz): 8.28-8.34(1H, m), 7.59(1H, m), 7.31-7.37(2H, m), 6.96-7.14(7H, m), 6.27-6.34(1H, m), 4.53-4.60(2H, m), 4.22-4.30(1H, m) 3.49-3.66(2H, m), 3.25-33.31(2H, m), 3.05-3.11(1H, m), 2.89-2.97(1H, m), 1.19(3H, t, J=7.0 Hz) | LRMS: 409 (M + H)⁺ | |
| D-15 | 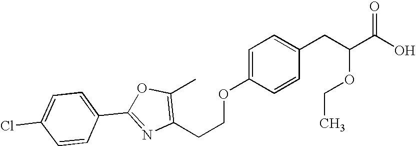 | ¹H NMR (MeOH-d₄, 400 MHz): 8.31(1H, s), 8.02(2H, d, J=8.3 Hz), 7.59(1H, d, J=9.1 Hz), 7.43(2H, d, J=8.3 Hz), 6.31(1H, d, J=9.1 Hz), 4.20-4.29(3H, m), 3.49-3.66 (2H, m), 3.05-3.11(1H, m), 2.89-2.97(1H, m), 2.70(2H, t, J=8.0 Hz), 2.19(3H, s), 1.19(3H, t, J=7.0 Hz) | LR 432 (M + H)⁺ | |
| D-16 | 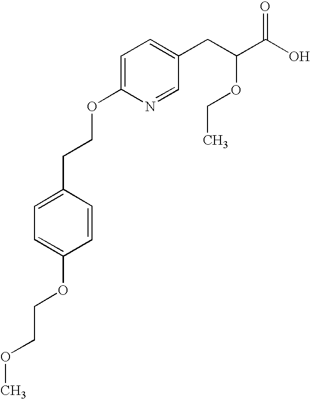 | ¹H NMR (MeOH-d₄, 400 MHz): 8.31(1H, s), 7.59(1H, d, J=9.1 Hz), 8.67(2H, d, J=8.2 Hz), 6.73(2H, d, J=8.2 Hz), 6.31(1H, d, J=9.2 Hz), 4.57(2H, t, J=6.5 Hz), 4.43(2H, s), 4.26(1H, dd, J=13.5, 0.2 Hz), 3.76(2H, s), 3.49-3.66(2H, m), 3.38(3H, s), 3.28(2H, t, J=6.5 Hz), 3.05-3.11(1H, m), 2.89-2.97 (1H, m), 1.19(3H, t, J=7.0 Hz) | LR 391 (M + H)⁺ | |
| D-17 | 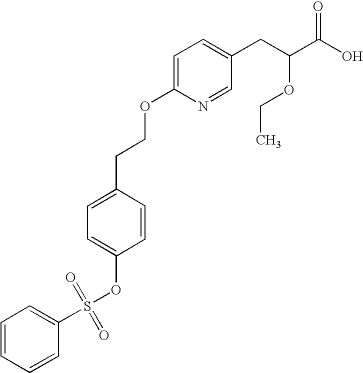 | ¹H NMR (MeOH-d₄, 400 MHz): 8.31(1H, s), 7.66-7.74(5H, m), 7.59(1H, d, J=9.1 Hz), 7.19(2H, d, J=8.2 Hz), 6.86(2H, d, J=8.3 Hz), 6.31(1H, d, J=9.2 Hz), 4.57(2H, t, J=6.5 Hz), 4.26(1H, dd, J=13.5, 0.2 Hz), 3.49-3.66(2H, m), 3.28(2H, t, J=6.5 Hz), 3.05-3.11(1H, m), 2.89-2.97(1H, m), 1.19(3H, t, J=7.0 Hz) | LR 473 (M + H)⁺ | |
| D-18 | 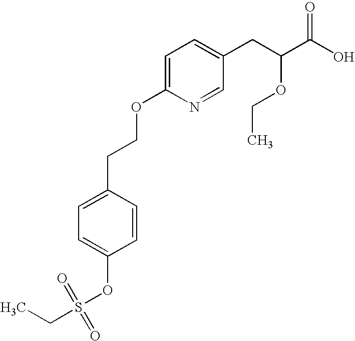 | ¹H NMR (MeOH-d₄, 400 MHz): 8.31(1H, s), 7.59(1H, d, J=9.1 Hz), 7.11(2H, d, J=8.2 Hz), 6.71(2H, d, J=8.2 Hz), 6.31(1H, d, J=9.2 Hz), 4.57(2H, t, J=6.5 Hz), 4.26(1H, dd, J=13.5, 0.2 Hz), 3.98(2H, q, J=7.7 Hz), 3.49-3.66(2H, m), 3.28 (2H, t, J=6.5 Hz), 3.05-3.11(1H, m), 2.89-2.97(1H, m), 1.44(3H, t, J=7.7 Hz), 1.19(3H, t, J=7.0 Hz) | LR 425 (M + H)⁺ | |
| D-19 | 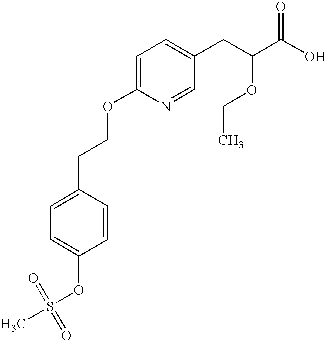 | ¹H NMR (MeOH-d₄, 400 MHz): 8.31(1H, s), 7.59(1H, d, J=9.1 Hz), 7.10(2H, d, J=8.2 Hz), 6.74(2H, d, J=8.2 Hz), 6.31(1H, d, J=9.2 Hz), 4.57(2H, t, J=6.5 Hz), 4.26(1H, dd, J=13.5, 0.2 Hz), 3.49-3.66 (2H, m), 3.34(3H, s), 3.28(2H, t, J=6.5 Hz), 3.05-3.11(1H, m), 2.89-2.97(1H, m), 1.19(3H, t, J=7.0 Hz) | LR 411 (M + H)⁺ | |
| D-20 | 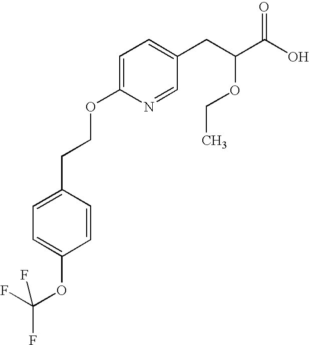 | ¹ H NMR (MeOH-d₄, 400 MHz): 8.31(1H, s), 7.59(1H, d, J=9.1 Hz), 7.34(2H, d, J=8.2 Hz), 7.24(2H, d, J=8.2 Hz), 6.31(1H, d, J=9.2 Hz), 4.57(2H, t, J=6.5 Hz), 4.26(1H, dd, J=13.5, 0.2 Hz), 3.49-3.66 (2H, m), 3.28(2H, t, J=6.5 Hz), 3.05-3.11(1H, m), 2.89-2.97(1H, m), 1.19(3H, t, J=7.0 Hz) | LR 400 (M + H)⁺ | |
| D-21 | 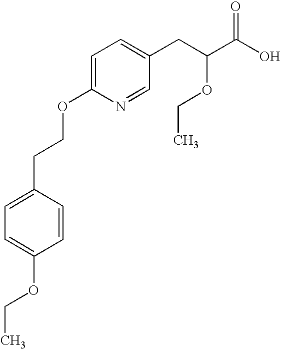 | ¹H NMR (MeOH-d₄, 400 MHz): 8.31(1H, s), 7.59(1H, d, J=9.1 Hz), 6.94(2H, d, J=8.2 Hz), 6.66(2H, d, J=8.3 Hz), 6.31(1H, d, J=9.2 Hz), 4.57(2H, t, J=6.5 Hz), 4.26(1H, dd, J=13.5, 0.2 Hz), 4.01(2H, q, J=6.9 Hz), 3.49-3.66(2H, m), 3.28 (2H, t, J=6.5 Hz), 3.05-3.11(1H, m), 2.89-2.97(1H, m), 1.40(3H, t, J=7.0 Hz), 1.19(3H, t, J=7.0 Hz) | LR 360 (M + H)⁺ | |
| D-22 | 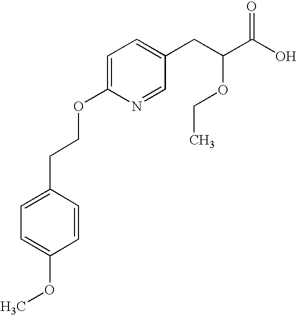 | ¹H NMR (MeOH-d₄, 400 MHz): 8.31(1H, s), 7.59(1H, d, J=9.1 Hz), 6.97(2H, d, J=8.2 Hz), 6.77(2H, d, J=8.2 Hz), 6.31(1H, d, J=9.2 Hz), 4.57(2H, t, J=6.5 Hz), 4.26(1H, dd, J=13.5, 0.2 Hz), 3.78(3H, s), 3.49-3.66(2H, m), 3.28(2H, t, J=6.5 Hz), 3.05-3.11(1H, m), 2.89-2.97(1H, m), 1.19(3H, t, J=7.0 Hz) | LR 346 (M + H)⁺ | |
| D-23 | 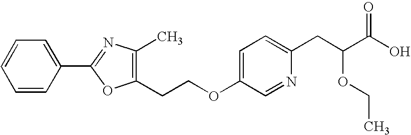 | (MeOD, 400 MHz): 8.03(1H, d, J=2.8 Hz), 7.84-7.82(2H, m), 7.35-7.34(3H, m), 7.26(1H, dd, J=8.6, 2.5 Hz), 7.16(1H, d, J=8.6 Hz), 4.20(2H, t, JU=6.4 Hz), 4.06(1H, dd, J=8.6, 4.6 Hz), 3.52-3.44(1H, m), 3.22-3.14(1H, m), 3.03(1H, dd, J=13.9, 4.6 Hz), 2.88(2H, t, J=6.3 Hz), 2.92(1H, d, J=9.1 Hz), 2.26(3H, s), 0.94(3H, t, J=7.0 Hz) | LR 397 (M + H)⁺ | |
| D-24 | 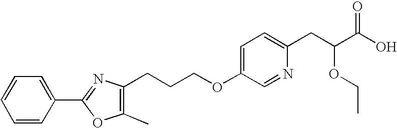 | (CDCl₃, 400 MHz): 8.25(1H, d, J=1.5 Hz), 7.95(2H, dd, J=7.6, 1.8 Hz), 7.44-7.36(3H, m), 7.28-7.22(2H, m), 4.19(1H, dd, J=7.1, 4.6 Hz), 4.02(2H, t, J=6.1 Hz), 3.80-3.72(1H, m), 3.47-3.39(1H, m), 3.34(1H, dd, J=15.2, 7.1 Hz), 3.20(1H, dd, J=15.2, 4.3 Hz), 2.67(2H, t, J=7.2 Hz), 2.27(3H, s), 2.19-2.12(2H, m), 1.17(3H, t, J=7.0 Hz) | LR 411 (M + H)⁺ | |
| D-24 | 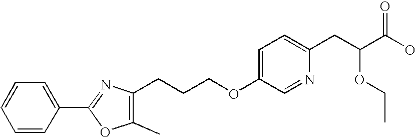 | (CDCl₃, 400 MHz): 8.25(1H, d, J=1.5 Hz), 7.95(2H, dd, J=7.6, 1.8 Hz), 7.44-7.36(3H, m), 7.28-7.22(2H, m), 4.19(1H, dd, J=7.1, 4.6 Hz), 4.02(2H, t, J=6.1 Hz), 3.80-3.72(1H, m), 3.47-3.39(1H, m), 3.34(1H, dd, J=15.2, 7.1 Hz), 3.20(1H, dd, J=15.2, 4.3 Hz), 2.67(2H, t, J=7.2 Hz), 2.27(3H, s), 2.19-2.12(2H, m), 1.17(3H, t, J=7.0 Hz) | LR 411 (M + H)⁺ | |
| D-25 | 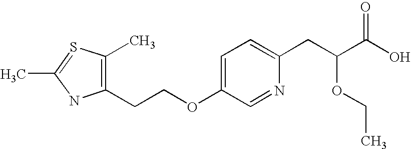 | (CDCl₃, 400 MHz): 8.16(1H, d, J=2.3 Hz), 7.24-7.19(2H, m), 4.27 (2H, t, J=6.8 Hz), 4.17(1H, dd, J=7.6, 3.8 Hz), 3.79-3.72(1H, m), 3.47-3.40(1H, m), 3.32(1H, dd, J=15.4, 7.6 Hz), 3.18(1H, dd, J=15.4, 3.5 Hz), 3.09(2H, t, J=6.7 Hz), 2.59(3H, s), 2.34(3H, s), 1.17(3H, t, J=7.0 Hz) | LR 351 (M + H)⁺ | |
| D-26 | 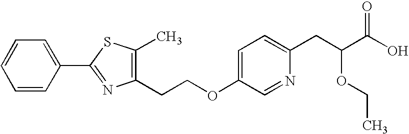 | (MeOD, 400 MHz): 8.01(1H, d, J=p 3.0 Hz), 7.74-7.71(2H, m), 7.34-7.27(3H, m), 7.24(1H, dd, J=8.6, 2.8 Hz), 7.14(1H, d, J=8.6 Hz), 4.27(2H, t, J=6.6 Hz), 4.06(1H, dd, J=8.7, 4.7 Hz), 3.51-3.44(1H, m), 3.22-3.14(1H, m), 3.08(2H, t, J=6.4 Hz), 3.02(1H, dd, J=14.2, 4.6 Hz), 2.89(1H, dd, J=13.9, 8.6 Hz), 2.34(3H, s), 0.93(3H, t, J=7.1 Hz) | LR 413 (M + H)⁺ | |
| D-27 | 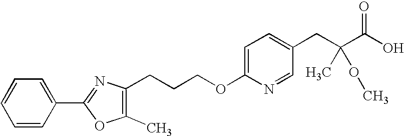 | (MeOD, 400 MHz): 7.86-7.82(3H, m), 7.47(1H, dd, J=8.6, 2.0 Hz), 7.39-7.36(3H, m), 6.62(1H, d, J=8.3 Hz), 4.14(2H, t, J=6.2 Hz), 3.22-3.20(3H, m), 2.85(2H, dd, J=22.5, 13.9 Hz), 2.60(2H, t, J=7.1 Hz), 2.19(3H, s), 2.03(2H, dd, J=12.9, 6.1 Hz), 1.26(3H, s) | LR 411 (M + H)⁺ | |
| D-28 | 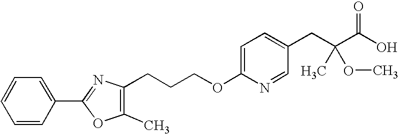 | (MeOD, 400 MHz): 8.02(1H, d, J=2.5 Hz), 7.85-7.83(2H, m), 7.39-7.33(3H, m), 7.26(1H, dd, J=8.6, 2.8 Hz), 7.20(1H, dd, J=8.6 Hz), 3.96(2H, t, J=6.1 Hz), 3.20(3H, s), 3.05(2H, dd, J=21.0, 13.9 Hz), 2.61(2H, t, J=7.2 Hz), 2.20 (3H, s), 2.07-2.01(2H, m), 1.24 (3H, s) | LR 411 (M + H)⁺ | |
| D-29 | 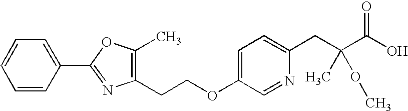 | (MeOD, 400 MHz): 8.02(1H, d, J=2.8 Hz), 7.87-7.83(2H, m), 7.39-7.36(3H, m), 7.27(1H, dd, J=8.6, 2.8 Hz), 7.19(1H, d, J=8.6 Hz), 4.22(2H, t, J=6.4 Hz), 3.19(3H, s), 3.05(2H, dd, J=21.2, 13.9 Hz), 2.91(2H, t, J=6.4 Hz), 2.29 (3H, s), 1.23(3H, s) | LR 397 (M + H)⁺ | |
| D-30 | 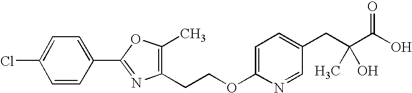 | (MeOD, 400 MHz): 7.87-7.84(3H, m), 7.48(1H, dd, J=8.6, 2.3 Hz), 7.41(2H, d, J=8.6 Hz), 6.61(1H, d, J=8.6 Hz), 4.42(2H, t, J=6.6 Hz), 3.22(3H, s), 2.91-2.88(3H, m), 2.83(1H, d, J=13.9 Hz), 2.27 (3H, s), 1.25(3H, s) | LR 431 (M + H)⁺ | |
| D-31 | 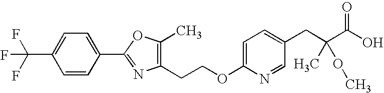 | (MeOD, 400 MHz): 8.04(2H, d, J=8.1 Hz), 7.84(1H, d, J=2.0 Hz), 7.70(2H, d, J=8.3 Hz), 7.46(1H, dd, J=8.5, 2.4 Hz), 6.60(1H, d, J=8.3 Hz), 4.43(2H, t, J=6.6 Hz), 3.22(3H, s), 2.91(2H, t, J=6.4 Hz), 2.88(1H, d, J=14.2 Hz), 2.82 (1H, d, J=14.2 Hz), 2.28(3H, s), 1.25(3H, s) | L 465 (M + H)⁺ | |
| D-32 | 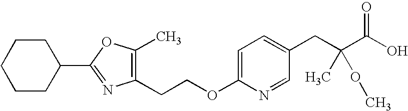 | (MeOD, 400 MHz): 7.84(1H, d, J=2.0 Hz), 7.47(1H, dd, J=8.5, 2.4 Hz), 6.59(1H, d, J=8.6 Hz), 4.32(2H, t, J=6.6 HJz), 3.23(3H, s), 2.89(1H, d, J=14.2 Hz), 2.83 (1H, d, J=13.9 Hz), 2.78(2H, t, J=6.7 Hz), 2.64(1H, tt, J=11.6, 3.5 Hz), 2.13(3H, s), 1.93-1.89 (2H, m), 1.75-1.71(2H, m), 1.65-1.62(1H, m), 1.50-1.40(2H, m), 1.37-1.27(2H, m), 1.26(3H, s), 1.23-1.19(1H, m) | LR 403 (M + H)⁺ | |
| D-33 | 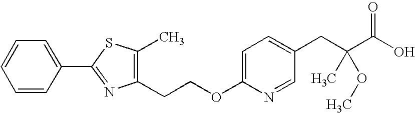 | (MeOD, 400 MHz): 7.85(1H, d, J=2.3 Hz), 7.77(2H, dd, J=7.6, 1.8 Hz), 7.47(1H, dd, J=8.6, 2.3 Hz), 7.39-7.31(3H, m), 6.61(1H, d, J=8.6 Hz), 4.49(2H, t, J=6.7 Hz), 3.23(3H, s), 3.11(2H, t, J=6.7 Hz), 2.89(1H, d, J=13.9 Hz), 2.83(1H, d, J=14.2 Hz), 2.36 (3H, s), 1.26(3H, s) | LR 413 (M + H)⁺ | |
| D-34 | 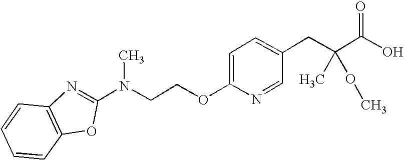 | (MeOD, 400 MHz): 7.84(1H, d, J=2.3 Hz), 7.43(1H, dd, J=8.5, 2.4 Hz), 7.17(2H, dd, J=8.1, 0.8 Hz), 7.09-7.05(1H, m), 6.94(1H, dt, J=7.8, 1.0 Hz), 6.53(1H, d, J=8.3 Hz), 4.49(2H, t, J=5.3 Hz), 3.87(2H, t, J=5.3 Hz), 3.23(3H, s), 3.18(3H, s), 2.88(1H, d, J=13.9 Hz), 2.82(1H, d, J=13.9 Hz), 1.26(3H, s) | LR 386 (M + H)⁺ | |
| D-35 | 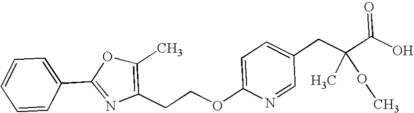 | (Dimethyl sulfoxide-d₆, 300 MHz): 7.91(3H, m), 7.49(4H, m), 7.40 (3H, m), 6.69(1H, d, J=8.5 Hz), 4.44(2H, t, J=6.7 Hz), 3.44(2H, m), 3.18(3H, s), 2.89(2H, m), 2.31 (3H, s), 1.19(3H, s) | LR 395 (M)⁺ | Calcd C₂₂H₂₄N₂O₅ C 66.65, H 6.10, N 7.07. Found: C₂₂H₂₄N₂O₅0.06H₂O C 66.16, H 6.15, N 6.96. |
| D-36 | 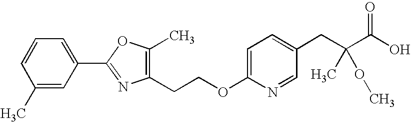 | (MeOD, 400 MHz): 7.83(1H, d, J=1.8 Hz), 7.69(1H, s), 7.65(1H, d, J=7.6 Hz), 7.46(1H, dd, J=8.6, 2.3 Hz), 7.25(1H, t, J=7.6 Hz), 7.19(1H, d, J=7.6 Hz), 6.59 (1H, d, J=8.3 Hz), 4.39(2H, t, J=6.6 Hz), 3.19(3H, s), 2.87(2H, t, J=6.4 Hz), 2.87(1H, d, J=13.9 Hz), 2.80(1H, d, J=13.9 Hz), 2.30 (3H, s), 2.24(3H, s), 1.21(3H, s) | LR 411 (M + H)⁺ | |
| D-37 | 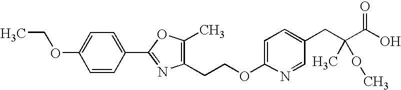 | (MeOD, 400 MHz): 7.83(1H, d, J=2.3 Hz), 7.79-7.76(2H, m), 7.46 (1H, dd, J=8.5, 2.4 Hz), 6.90(2H, d, J=8.8 Hz), 6.59(1H, d, J=8.6 Hz), 4.38(2H, t, J=6.7 Hz), 4.00 (2H, q, J=7.1 Hz), 3.21(3H, s), 2.88(1H, d, J=13.9 Hz), 2.86 (2H, t, J=6.6 Hz), 2.81(1H, d, J=13.9 Hz), 2.22(3H, s), 1.31(3H, t, J=7.0 Hz), 1.24(3H, s) | LR 441 (M + H)⁺ | |
| D-38 | 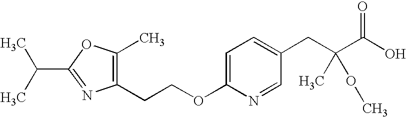 | (CDCl₃, 400 MHz): 7.93(1H, d, J=2.3 Hz), 7.45(1H, dd, J=8.5, 2.4 Hz), 6.63(1H, d, J=8.6 Hz), 4.40(2H, t, J=6.8 Hz), 3.37(3H, s), 3.05-2.96(1H, m), 2.97(1H, d, J=14.4 Hz), 2.92(1H, d, J=14.4 Hz), 2.86(2H, t, J=6.7 Hz), 2.20 (3H, s), 1.43(3H, s), 1.30(3H, s), 1.28(3H, s) | LR 363 (M + H)⁺ | |
| D-39 | 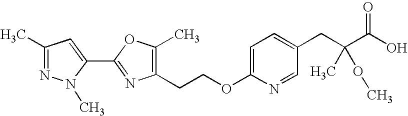 | (MeOD, 400 MHz): 7.82(1H, d, J=2.0 Hz), 7.44(1H, dd, J=8.6, 2.3 Hz), 6.57(1H, d, J=8.6 Hz), 6.45(1H, s), 4.39(2H, t, J=6.4 Hz), 4.00(3H, s), 3.20(3H, s), 2.86(2H, t, J=6.6 Hz), 2.86(1H, d, J=14.2 Hz), 2.80(1H, d, J=14.2 Hz), 2.22(3H, s), 2.14(3H, s), 1.23(3H, s) | LR 4415 (M + H)⁺ | |
| D-40 | 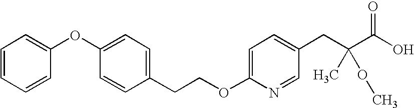 | (MeOD, 400 MHz): 7.82(1H, d, J=2.3 Hz), 7.44(1H, dd, J=8.0, 1.9 Hz), 7.34-7.29(2H, m), 7.22 (2H, d, J=8.6 Hz), 7.09-7.03(1H, m), 6.98-6.91(4H, m), 6.60(1H, d, J=8.6 Hz), 4.33(2H, t, J=7.1 Hz), 3.20(3H, s), 3.00(2H, t, J=7.0 Hz), 2.82(1H, d, J=14.2 Hz), 2.80(1H, d, J=14.2 Hz), 1.23 (3H, s) | LR 408 (M + H)⁺ | |
| D-41 | 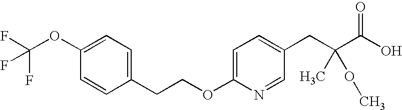 | (MeOD, 400 MHz): 7.81(1H, d, J=2.3 Hz), 7.44(1H, dd, J=8.6, 2.3 Hz), 7.27(2H, d, J=8.6 Hz), 7.07(2H, d, J=7.8 Hz), 6.57(1H, d, J=8.6 Hz), 4.33(2H, t, J=6.7 Hz), 3.20(3H, s), 2.97(2H, t, J=6.7 Hz), 2.84(1H, d, J=14.2 Hz), 2.82(1H, d, J=14.2 Hz), 1.24 (3H, s) | LR 400 (M + H)⁺ | |
| D-42 | 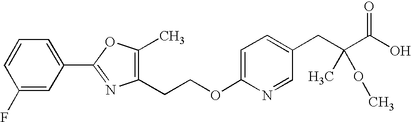 | (MeOD, 400 MHz): 7.83(1H, dd, J=1.8 Hz), 7.65(1H, d, J=7.6 Hz), 7.46(1H, dd, J=8.6, 2.3 Hz), 7.29 (1H, s), 7.25(1H, t, J=7.6 Hz), 7.19(1H, d, J=7.6 Hz), 6.59(1H, d, J=8.3 Hz), 4.39(2H, t, J=6.6 Hz), 3.19(3H, s), 2.87(2H, t, J=6.4 Hz), 2.87(1H, d, J=13.9 Hz), 2.80(1H, d, J=13.9 Hz), 2.24 (3H, s), 1.21(3H, s) | LR 415 (M + H)⁺ | |
| D-43 | 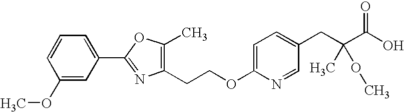 | (MeOD, 400 MHz): 7.81(1H, d, J=2.3 Hz), 7.45-7.38(2H, m), 7.32-7.24(2H, m), 6.91(1H, dd, J=8.0, 2.4 Hz), 6.58(1H, d, J=8.3 Hz), 4.38(2H, t, J=6.7 Hz), 3.74(3H, s), 3.18(3H, s), 2.86(2H, t, J=6.7 Hz), 2.85(1H, d, J=13.9 Hz), 2.79 1H, d, J=13.9 Hz), 2.22(3H, s), 1.22(3H, s) | LR 427 (M + H)⁺ | |

Preparations of starting materials for Examples D-1 to D-43 (Preparations d-1 to d-42):

### Preparation d-1

### Ethyl 2-ethoxy-3-{6-[2-(5-methyl-2-phenyl-1,3-oxazol-4-yl)ethoxy]pyridin-3-yl}acrylate

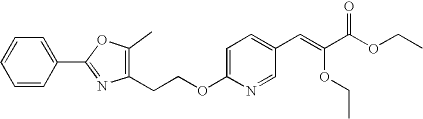

N,N,N,N-tetramethylguanidine (0.305 mL, 2.43 mmol) was added dropwise to a solution of 6-[2-(5-methyl-2-phenyl-1,3-oxazol-4-yl)ethoxy]nicotinaldehyde (250 mg, 0.811 mmol) and (1,2-diethoxy-2-oxoethyl)(triphenyl)phosphonium chloride (696 mg, 1.62 mmol) in chloroform (4 mL). The mixture was stirred for 16 hours then partitioned between saturated ammonium chloride solution and ethyl acetate. The organic phase was washed with brine, dried over magnesium sulfate, filtered and evaporated* and evaporated. The residue was purified by flash column chromatography (1:2 ethyl acetate:hexanes) to yield the title compound as a white solid (330 g, 96%). LRMS (m/z): 423 (M+H)⁺. ¹H NMR (CDCl₃, 300 MHz) δ 8.42 (1H, m), 8.10 (1H, m), 7.97 (1H, m), 7.40 (2H, m), 7.28 (3H, m), 6.90 (1H, s), 6.73 (1H, m), 4.60 (2H, t, J=7 Hz), 4.30 (2H, q, J=7 Hz), 4.01 (2H, q, J=7 Hz), 2.99 (2H, t, J=7 Hz), 2.34 (3H, s), 15 1,36 (6H, m).

### Preparation d-2

### Ethyl 2-ethoxy-3-{6-[2-(5-methyl-2-phenyl-1,3-oxazol-4-yl)ethoxy]pyridin-3-yl}propanoate

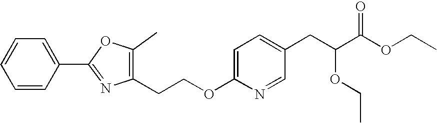

A solution of ethyl 2-ethoxy-3-{6-[2-(5-methyl-2-phenyl-1,3-oxazol-4-yl)ethoxy]pyridin-3-yl}acrylate (328 mg, 0.776 mmol) in ethanol (10 mL) was hydrogenated at 50 psi over 10% palladium on carbon (33 mg) for 3 hours. The mixture was filtered through celite and the solid was washed with ethyl acetate. The filtrate and washings were evaporated and the residue was purified by flash column chromatography (1:2 ethyl acetate:hexanes) to yield the title compound as a colorless oil (183 mg, 56%). LRMS (m/z): 425 (M+H)⁺. ¹H NMR (CDCl₃, 300 MHz) δ 7.99 (3H, m), 7.42 (4H, m), 6.65 (1H, m), 4.54 (2H, t, J=7 Hz), 4.18 (2H, q, J=7 Hz), 3.93 (1H, m), 3.63 (1H, m), 3.36 (1H, m), 2.90 (4H, m), 2.33 (3H, s), 1.25 (3H, t, J=7 Hz), 1.16 (3H, t, J=7 Hz).

### Preparation d-3

### 2-(Benzyloxy)-5-bromopyridine

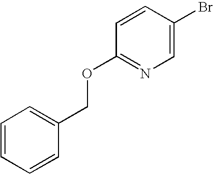

To a solution of 5-bromopyridin-2(1H)-one (100 mmol, 17.4 g, 1.0 eq.) in benzene (170 mL) was added silver (I) carbonate (67.0 mmol, 18.5 g, 0.67 eq.). The flask was wrapped with aluminum foil and then benzyl bromide (120 mmol, 20.5 g, 1.2 eq.) was added via syringe in a steady stream. The mixture was heated to 50° C. and stirred in the dark for approximately 24 hours. LC/MS of the reaction mixture indicates two peaks both with M+H=265 corresponding to the desired molecular weight. On the basis of relative polarities, the more polar peak was thought to be the N-alkylated product and consisted of approximately 20% of the total. The reaction mixture was allowed to cool to room temperature and the silver salt was removed by filtration of the mixture through a pad of celite. The filter cake was washed with benzene and the organic layer was washed twice with 2% sodium bicarbonate and twice with water. The organic layer was dried over magnesium sulfate and concentrated in vacuo. The crude residue was purified on a Biotage Sp4 65i over a gradient of 5-95% hexanes in ethyl acetate to afford the title compound as a golden oil (25.1 g, 95%). LRMS: 265 (M+H)⁺.

¹H NMR (DMSO-d₆, 400 MHz); δ 8.29 (1 H, s) 7.72 (1 H, d, J=8.5 Hz) 7.31-7.43 (5 H, m) 6.54 (1H, d, J=8.5 Hz) 5.34 (2 H, s).

### Preparation d-4

### 6-(Benzyioxy)nicotinaldehyde

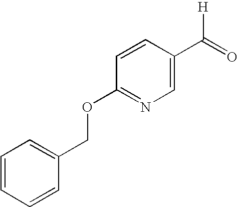

n-Butyl lithium (2.5M, 95.9 mmol, 38.4 mL, 1.05 eq.) was added dropwise via syringe to a stirred solution of 2-(benzyloxy)-5-bromopyridine (91.3 mmol, 24.1 g, 1.0 eq.) in THF (260 mL, c=0.35) cooled to −78° C. Upon completion of addition, the solution was allowed to continue stirring at the same low temperature for 1 hour. At this point, N,N-dimethylformamide (183 mmol, 13.4 g, 2.0 eq.) was added dropwise as a solution in 5 mL THF. Stirring was continued at the same low temperature for a further 30 minutes at which point the reaction was quenched by addition of 5% sodium bicarbonate. The mixture was transferred to a separatory funnel and extracted with ether (3×250 mL). The combined organic layers were washed with brine, dried over anhydrous magnesium sulfate and concentrated in vacua. The resultant yellow oil was purified on a Biotage Sp4 65i over a gradient of 0-50% hexanes in ethyl acetate to afford the title compound (14.1 g, 73%). LRMS: 214 (M+H)⁺. ¹H NMR (DMSO-d₆, 400 MHz): δ 10.02 (1 H, s) 8.86 (1 H, s) 8.03 (1 H, d, J=9.3 Hz) 7.31-7.43 (5 H, m) 6.50 (1 H, d, J=9.3 Hz) 5.33 (2 H, s).

### Preparation d-5

### Ethyl (2Z)-3-[6-(benzyloxy)pyridin-3-yl]-2-ethoxyacrylate

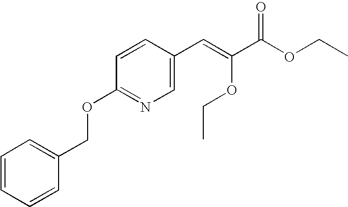

To a solution of 6-(benzyloxy)nicotinaldehyde (1.0 eq., 33.1 mmol, 7.05 g) and (1,2-diethoxy-2-oxoethyl)(triphenyl)phosphonium chloride (2.0 eq., 66.2 mmol, 28.4 g) in chloroform (165 mL, 0.2 M) was added tetramethylguanidine (3.0 eq., 99.3 mmol, 11.4 g). The flask was capped with a hollow glass stopper and stirred at room temperature overnight. TLC analysis after approximately 18 hours indicated the presence of a small amount of unreacted starting material. The reaction mixture was heated to reflux and TLC reanalyzed after 2 hours. The reaction was quenched with saturated ammonium chloride. The layers were separated and the organic layer was washed with brine, dried over magnesium sulfate and concentrated in vacua. A large amount of triphenylphosphine oxide precipitated. The residue was triturated with ether and filtered. Washed filter cake with ether and concentrated combined filtrates in vacuo to afford a pale yellow solid which was dissolved in a minimal amount of DCM and loaded onto Biotage Sp4 65i and eluted over a gradient of 10-100% hexanes to ethyl acetate. Obtained 12.3 g of a clear, colorless oil (37.6 mmol, quant.). LRMS: 328 (M+H)⁺.

¹H NMR (DMSO-d₆, 400 MHz): δ 8.33 (1 H, s) 7.92 (1 H, d, J=8.0 Hz) 7.31-7.43 (5 H, m) 6.76 (1 H, d, J=8.1 Hz) 6.60 (1 H, s) 5.37 (2 H, s) 4.23 (2 H, q, J=7.1 Hz) 3.90-3.99 (2 H, m) 1,34 (6 H, dt, J=15.8, 7.0 Hz).

### Preparation d-6

### Ethyl 2-ethoxy-3-(6-oxo-1,6-dihydropyridin-3-yl)propanoate

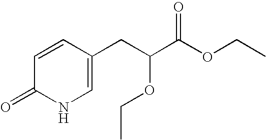

To a Parr shaker bottle containing a solution of ethyl (2Z)-3-[6-(benzyloxy)pyridin-3-yl]-2-ethoxyacrylate in ethanol was added 10% Pd on carbon (˜1.23 g). The bottle was purged with hydrogen and degassed under reduced pressure three times. The mixture was placed under 50 psi hydrogen and shaken at room temperature overnight. After ˜20 hours shaking was stopped and the bottle was degassed in vacuo. TLC analysis indicated consumption of starting material. The mixture was filtered through a pad of celite to remove palladium. The filter cake was washed with an additional portion of ethanol. This solution was concentrated in vacuo to yield the reduced and debenzylated pyridone as a golden oil. This oil was purified on a Biotage Sp4, 65i, 80 mL/min over a gradient of 0-10% MeOH in DCM to yield 2.77 g of a clear, colorless oil (11.6 mmol, 31%). LRMS: 240 (M+H)⁺. ¹H NMR (DMSO-d₆, 400 MHz): δ 7.29 (1 H, d, J=9.5 Hz) 7.19 (1 H, d, J=5.2 Hz) 6.55 (1 H, d, J=9.5 Hz) 4.20 (2 H, q, J=7.0 Hz) 4.10 (1 H, dd, J=9.6, 0.3 Hz) 3.59-3.73 (2 H, m) 2.65-2.70 (1 H, m) 2.53-2.61 (1 H, m) 1.23 (6 H, td, J=7.0, 3.6 Hz).

### Preparation d-7

### 5-Benzyloxy-pyridine-2-carbaldehyde

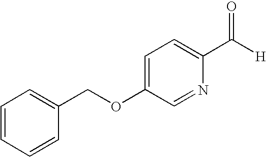

To a solution of (5-benzyloxy-pyridin-2-yl)-methanol (2.3619 g, 10.9728 mmol) in dichloromethane (120 mL) and pyridine (2.68 mL, 32.9184 mmol) was added 1,1,1-triacetoxy-1,1-dihydro-1,2-benziodoxol-3(1H)-one (6.9812 g, 16.4592 mmol). The resulting solution was stirred, under an atmosphere of nitrogen at ambient temperature, for 16 hours and then diluted with diethyl ether (100 mL) followed by partial concentration under reduced pressure. The residue was taken up in diethyl ether (150 mL), and precipitates were removed by extraction with 1:1 10% aqueous sodium thiosulfate:saturated aqueous sodium bicarbonate (2×100 mL). The organic layer was washed with water (100 mL) and saturated aqueous sodium chloride (100 mL), dried (anhydrous magnesium sulfate), filtered, and concentrated in vacuo to afford the pure title compound (1.1694 g, 50%) as a pale yellow oil. LRMS (m/z): 214 (M+H)⁺. ¹H NMR (CDCl₃, 300 MHz): δ 9.98 (1H, d, J=0.8 Hz), 8.49 (1H, d, J=2.5 Hz), 7.94 (1H, d, J=8.7 Hz), 7.44-7.40 (4H, m), 7.38-7.33 (2H, m), 5.19 (2H, s).

### Preparation d-8

### 3-(5-Benzyloxy-pyridin-2-yl)-2-ethoxy-acrylic acid ethyl ester

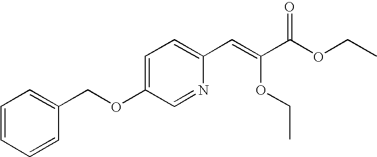

To a solution of 5-benzyloxy-pyridine-2-carbaldehyde (1.1694 g, 5.4842 mmol), (ethoxycarbonyl-methoxy-methyl)-triphenyl-phosphonium chloride (4.7043 g, 10.9684 mmol) in chloroform (30 mL), under an atmosphere of nitrogen at ambient temperature, was added tetramethylguanidine (2.1 mL, 16.4526 mmol) dropwise. The resulting solution was stirred for 16 hours and then quenched with saturated aqueous ammonium chloride (50 mL). The phases were separated and the organic phase washed with saturated aqueous sodium chloride (50 mL), dried (anhydrous magnesium sulfate), filtered and concentrated in vacuo to afford the crude product. The residue was purified by flash column chromatography (hexanes to ethyl acetate) to yield the pure title compound (1.8051 g, 100%) as a yellow oil. LRMS (m/z): 328 (M+H)⁺. ¹H NMR (CDCl₃, 300 MHz): δ 8.37 (1H, d, J=2.6 Hz), 8.18 (1H, d, J=8.9 Hz), 7.44-7.33 (5H, m), 7.25 (1H, dd, J=8.9, 3.0 Hz), 7.13 (1H, s), 5.13 (2H, s), 4.27 (2H, q, J=7.2 Hz), 4.05 (2H, q, J=7.2 Hz), 1,35 (3H, t, J=7.0 Hz), 1,34 (3H, t, J=7.0 Hz).

### Preparation d-9

### 2-Ethoxy-3-(5-hydroxy-pyridin-2-yl)-propionic acid ethyl ester

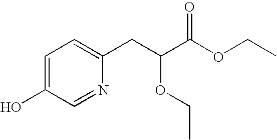

To a solution of 3-(5-benzyloxy-pyridin-2-yl)-2-ethoxy-acrylic acid ethyl ester (1.8051 g, 5.5144 mmol) in dry ethanol (40 mL) was added palladium (0.1805 g, 10 wt. % on activated carbon). The resulting solution was stirred at ambient temperature under an atmosphere of hydrogen (50 psi) for 16 hours. The resulting solution was filtered through a 3″ bed of Celite and washed with ethanol (200 mL). The filtrate was then concentrated in vacuo to afford the pure title compound (1.2231 g, 93%) as a pale yellow oil. LRMS (m/z): 240 (M+H)⁺. ¹H NMR (CDCl₃, 300 MHz): δ 8.14 (1H, s), 7.20-7.11 (2H, m), 4.19-4.10 (3H, m), 3.71 (1H, q, J=7.0 Hz), 3.63-3.53 (1H, m), 3.36-3.26 (1H, m), 3.18-3.03 (1H, m), 1.18 (3H, t, J=7.2 Hz), 1.07 (3H, t, J=7.1 Hz).

### Preparation d-10

### Ethyl 2-ethoxy-3-{6-[2-(4-phenoxyphenyl)ethoxy]pyridin-3-yl}propanoate

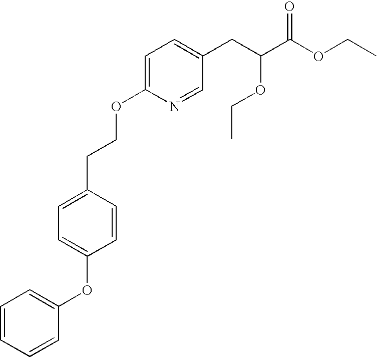

To an argon-purged solution of the appropriate bromopyridine (0.636 mmol) in toluene (12 mL) was added palladium (II) acetate (11.4 mg, 0.0508 mmol) and racemic-2-(Di-t-butylphosphino)-1,1′-binaphthyl (25.4 mg, 0.0636 mmol). The activated complex was allowed to form over approximately ten minutes, at which point cesium carbonate (414 mg, 1.27 mmol) and the appropriate alcohol (0.956 mmol) were added. The mixture was heated to 115° C. and stirred at this temperature for approximately 12-18 hours. The mixture was cooled to room temperature and filtered through a pad of silica. The filter pad was washed with 2-3 aliquots of ethyl acetate and the combined organic filtrates were combined and concentrated in vacuo.

The resulting residue was either purified by flash chromatography, or subjected to the general hydrolysis procedure.

### Preparations d-11 to d-18

Preparations d-11 to d-18 were prepared by procedures analogous to those used for Preparation d-10

| | | | MS (m/z) |
|---|---|---|---|
| Prep # | Structure | ¹H NMR | (LR or HR) |
| d-11 | 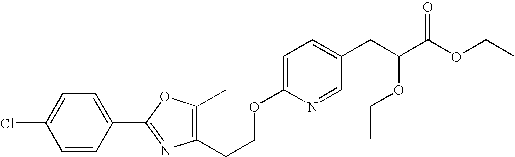 | | |
| d-12 | 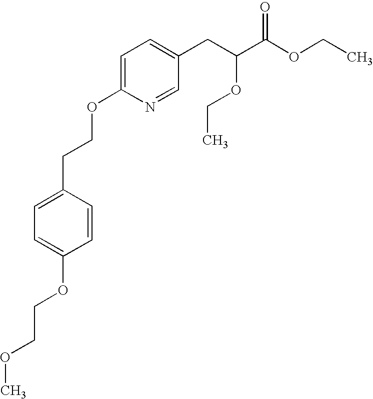 | | |
| d-13 | 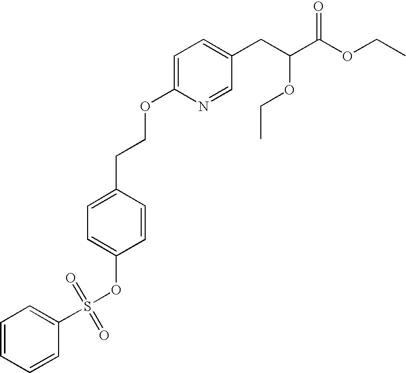 | | |
| d-14 | 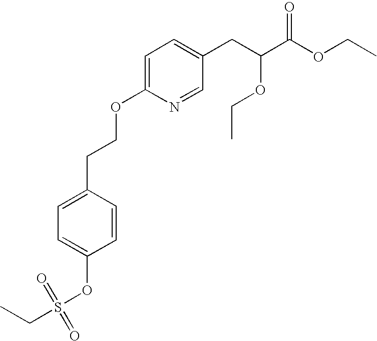 | | |
| d-15 | 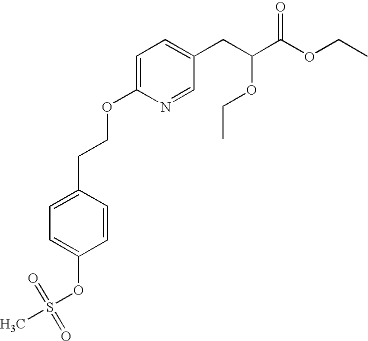 | | |
| d-16 | 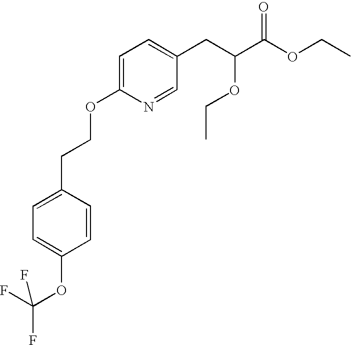 | | |
| d-17 | 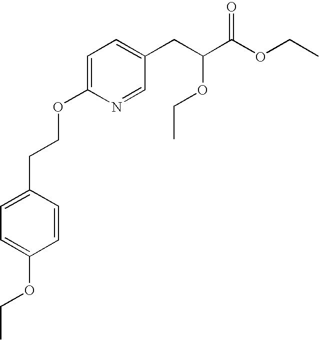 | | |
| d-18 | 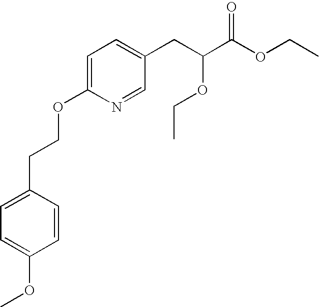 | | |

### Preparation d-19

### 2-Bromo-5-(bromomethyl)pyridine

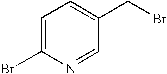

Phosphorous tribromide (100 mmol, 27.1 g, 2.0 eq.) was added carefully to 2-chloro-5-hydroxymethyl pyridine (50.0 mmol, 7.18 g, 1.0 eq.). The pyridine clumped together and the mixture was heated to 160° C. Within 5 minutes of stirring at >150° C. the mixture went very dark in color with gas evolution. The mixture was stirred at this same temperature for approximately 2.5 hours at which point it was cooled to room temperature. The mixture was cooled further to 0° C. whereupon saturated sodium bicarbonate was added very cautiously (highly exothermic!). As foaming became less vigorous, ice was added to the mixture until foaming subsided. Solid sodium bicarbonate was then carefully added to achieve a pH of ˜8-9. The mixture was extracted with ethyl acetate and the organic layer was washed with brine and dried over anhydrous magnesium sulfate. Concentrated in vacuo to afford a dark solid. This material was dissolved in a minimal amount of DCM and purified using a Biotage Sp4 65i over a gradient of 0-100% ethyl acetate in hexanes to afford the title compound as a pale yellow solid (5.57 g, 44%). LRMS: 252 (M+H)⁺. ¹H NMR (DMSO-d₆, 400 MHz): δ 8.39 (1H, s) 7.59 (1H, d, J=8.5 Hz) 7.48 (1H, d, J=8.5 Hz) 4.46 (2H, s).

### Preparation d-20

### Dimethyl [(6-bromopyridin-3-yl)methyl](methoxy)malonate

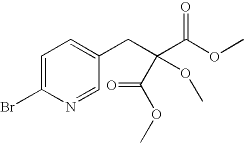

To a slurry of potassium t-butoxide (46.6 mmol, 5.22 g, 1,3 eq.) in anhydrous DMF (250 mL) cooled to 0° C. was added methoxy dimethylmalonate (46.6 mmol, 7.55 g, 1,3 eq.) via syringe in small portions. The enolate was allowed to form over approximately 30 minutes at which point 2-bromo-5-(bromomethyl)pyridine was added portionwise. The reaction mixture was allowed to warm slowly to room temperature over 3 hours. The reaction mixture was diluted with ethyl ether and transferred to a separatory funnel containing saturated ammonium chloride. The layers were shaken and separated and the organic layer was washed with water. The organic layer was then dried over anhydrous magnesium sulfate and concentrated in vacuo. The yellow oil obtained was purified on a Biotage Sp4 65i over a gradient of 0-100% ethyl acetate in hexanes to afford a colorless oil that solidified on standing (12.1 g, quant.) LRMS: 333 (M+H)⁺. ¹H NMR (DMSO-d₆, 400 MHz): δ 8.27 (1 H, s) 7.45-7.55 (2 H, m) 3.82 (6 H, s) 3.57 (3 H, s) 3.42 (2 H, s).

### Preparation d-21

### Methyl 3-(6-bromopyridin-3-yl)-2-methoxypropanoate

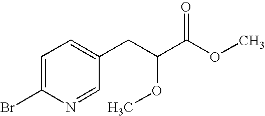

To a solution of dimethyl [(6-bromopyridin-3-yl)methyl](methoxy)malonate (3.55 mmol, 1.18 g, 1.0 eq.) in anhydrous DMF (2 mL) was added lithium bromide (3.20 mmol, 0.278 g, 0.9 eq.) followed by water (3.55 mmol, 0.064 g, 1.0 eq.). The solution was placed in a oil bath preheated to 165° C. Rapid gas evolution commenced. Bubble formation ceased within 30 minutes and LC/MS of the reaction mixture at this time indicated reaction was complete. Cooled to room temperature and diluted with water. Extracted aqueous layer with ethyl ether (4×25 mL). Combined organic layers and washed with brine. Dried organic layer over anhydrous magnesium sulfate and concentrated in vacuo to afford 536 mg of a brown oil that was a single spot by TLC. Used in next step without further purification. LRMS: 275 (M+H)⁺. ¹H NMR (DMSO-d₆, 400 MHz): δ 8.23 (1 H, s) 7.42-7.51 (2 H, m) 4.26 (1 H, d, J=8.1 Hz) 3.79 (3 H, s) 3.51 (3 H, s) 2.97-3.03 (1 H, m) 2.82-2.88 (1 H, m).

### Preparation d-22

### Ethyl 3-[6-(2-{4-[(ethylsulfonyl)oxy]pheny}ethoxy)pyridin-3-yl]-2-methoxypropanoate

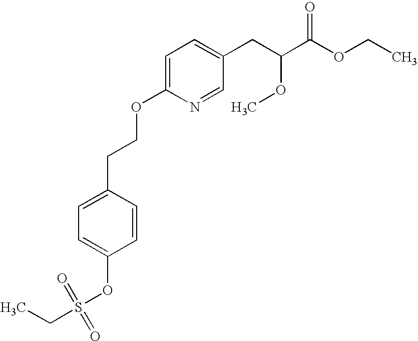

### Preparations d-23 to d-38

Preparations d-23 to d-38 were prepared by procedures analogous to those used for Preparation d-22

| | | | MS (m/z) |
|---|---|---|---|
| Prep # | Structure | ¹H NMR | (LR or HR) |
| d-23 | 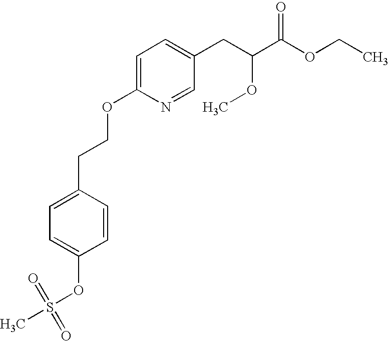 | | |
| d-24 | 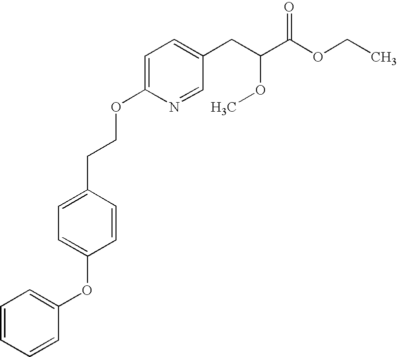 | | |
| d-25 | 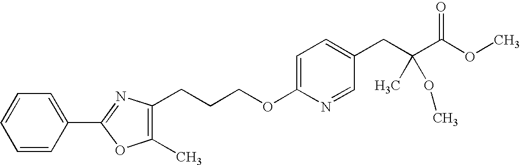 | (CDCl₃, 300 MHz): 7.90-7.99 (3H, m), 7.35-7.47 (4H, m), 6.65 (1H, d. J = 8.5 Hz), 4.27 (2H, t, J = 6.3 Hz), 4.17 (2H, t, J = 7.0 Hz), 3.29 (3H, s), 2.91 (2H, q, J = 14.1 Hz), 2.66 (2H, t, J = 7.4 Hz), 2.27 (3H, s), 2.16 (3H, s), 2.13 (2H, m), 1.25 (3H, t, J = 7.2 Hz) | LR 439 (M+H)⁺ |
| d-26 | 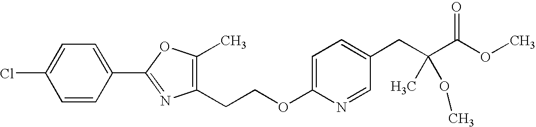 | (CDCl₃, 400 MHz): 7.91-7.88 (3H, m), 7.41 (1H, dd, J = 8.6, 2.5 Hz), 7.39-7.36 (2H, m), 6.62 (1H, d, J = 8.3 Hz), 4.51 (2H, t, J = 6.8 Hz), 3.70 (3H, s), 3.28 (3H, s), 2.97-2.93 (3H, m), 2.86 (1H, d, J = 14.2 Hz), 2.32 (3H, s), 1.32 (3H, s) | LR 445 (M+H)⁺ |
| d-27 | 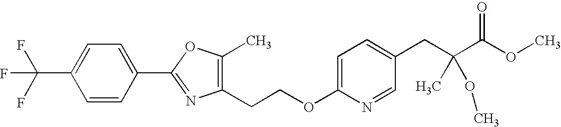 | (CDCl₃, 400 MHz): 8.07 (2H, d, J = 8.07), 7.91 (1H, d, J = 2.3 Hz), 7.66 (2H, d, J = 8.3 Hz), 7.41 (1H, dd, J = 8.5, 2.4 Hz), 6.62 (1H, d, J = 8.3 Hz), 4.52 (2H, t, J = 6.7 Hz), 3.70 (3H, s), 3.28 (3H, s), 2.97 (2H, t, J = 6.7 Hz), 2.95 (1H, d, J = 14.2 Hz), 2.86 (1H, d, J = 14.2 Hz), 2.34 (3H, s), 1.33 (3H, s) | LR 479 (M+H)⁺ |
| d-28 | 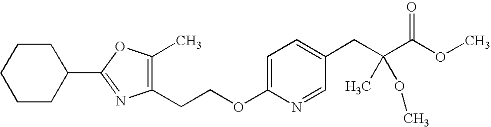 | (CDCl₃, 400 MHz): 7.90 (1H, d, J = 2.3 Hz), 7.41 (1H, dd, J =8.5, 2.4 Hz), 6.61 (1H, d, J =8.3 Hz), 4.42 (2H, t, J = 6.8 Hz), 3.71 (3H, s), 3.29 (3H, s), 2.95 (1H, d, J = 13.9 Hz), 2.86 (1H, d, J = 14.2 Hz), 2.86 (2H, t, J = 7.1 Hz), 2.67 (1H, tt, J =11.6, 3.5 Hz), 2.19 (3H, s), 2.02-1.98 (2H, m), 1.81-1.77 (2H, m), 1.69-1.65 (1H, m), 1.54-1.46 (2H, m), 1.38-1.31 | |
| #(2H, m), 1.33 (3H, s), 1.28-1.24 (1H, m) | LR 417 (M+H)⁺ | | |
| d-29 | 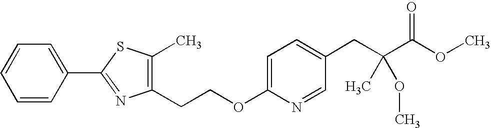 | (CDCl₃, 400 MHz): 7.92 (1H, d, J = 2.3 Hz), 7.85 (2H, dd, J =8.0, 1.6 Hz), 7.41 (1H, dd, J =8.5, 2.4 Hz), 7.39-7.35 (3H, m), 6.63 (1H, d, J = 8.6 Hz), 4.59 (2H, t, J = 7.0 Hz), 3.70 (3H, s), 3.28 (3H, s), 3.18 (2H, t, J = 7.0 Hz), 2.95 (1H, d, J = 14.2 Hz), 2.87 (1H, d, J = 14.2 Hz), 2.42 (3H, s), 1.33 (3H, s) | LR 427 (M+H)⁺ |
| d-30 | 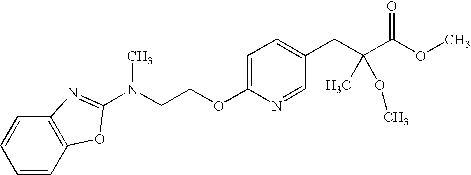 | (CDCl₃, 400 MHz): 7.90 (1H, d, J = 2.3 Hz), 7.41 (1H, dd, J =8.5, 2.4 Hz), 7.33 (1H, d, J =7.6 Hz), 7.21 (1H, d, J = 7.6 Hz), 7.13 (1H, dt, J = 7.6, 1.0 Hz), 6.97 (1H, dt, J = 7.8, 1.3 Hz), 6.60 (1H, d, J = 8.6 Hz), 4.56 (2H, t, J = 5.4 Hz), 3.92 (2H, t, J = 5.4 Hz), 3.70 (3H, s), 3.28 (6H, s), 2.95 (1H, d, J =13.9 Hz), 2.86 (1H, d, J = 14.2 Hz), 1.32 (3H, s) | |
| # LR 400 (M+H)⁺ | | | |
| d-31 | 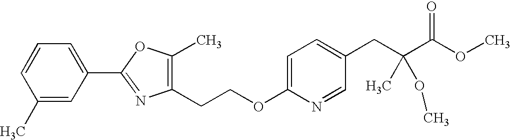 | (CDCl₃, 400 MHz): 7.91 (1H, d, J = 2.3 Hz), 7.81 (1H, s), 7.75 (1H, d, J = 7.8 Hz), 7.41 (1H, dd, J = 8.5, 2.4 Hz), 7.29 (1H, t, J = 7.7 Hz), 7.19 (1H, d, J = 7.6 Hz), 6.62 (1H, d, J = 8.3 Hz), 4.52 (2H, t, J = 6.8 Hz), 3.70 (3H, s), 3.28 (3H, s), 2.96 (2H, t, J = 6.8 Hz), 2.95 (1H, d, J =13.9 Hz), 2.86 (1H, d, J = 13.9 Hz), 2.38 (3H, s), 2.32 (3H, s), 1.33 (3H, s) | |
| # LR 425 (M+H)⁺ | | | |
| d-32 | 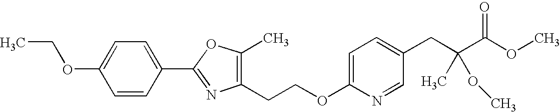 | (CDCl₃, 400 MHz): 7.91 (1H, d, J = 2.0 Hz), 7.89-7.86 (2H, m), 7.59 (1H, dd, J = 8.5, 2.2 Hz), 6.92-6.90 (2H, m), 6.62 (1H, d, J = 8.3 Hz), 4.51 (2H, t, J = 6.8 Hz), 4.06 (2H, q, J = 7.1 Hz), 3.70 (3H, s), 3.28 (3H, s), 2.95 (2H, t, J = 6.8 Hz), 2.96 (1H, d, J = 13.9 Hz), 2.87 (1H, d, J =13.9 Hz), 2.30 (3H, s), 1.42 (3H, t, J = 7.1 Hz), 1.33 (3H, s) | LR 455 (M+H)⁺ |
| d-33 | 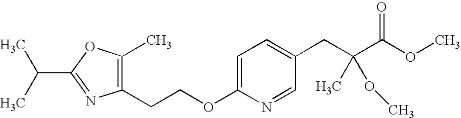 | (CDCl₃, 400 MHz): 7.90 (1H, d, J = 2.3 Hz), 7.41 (1H, dd, J =8.5, 2.4 Hz), 6.61 (1H, d, J =8.3 Hz), 4.42 (2H, t, J = 6.8 Hz), 3.70 (3H, s), 3.28 (3H, s), 3.00-2.93 (1H, m), 2.95 (1H, d, J = 14.2 Hz), 2.86 (2H, t, J =6.8 Hz), 2.86 (1H, d, J = 14.2 Hz), 2.20 (3H, s), 1.33 (3H, s), 1.30 (3H, s), 1.28 (3H, s) | LR 377 (M+H)⁺ |
| d-34 | 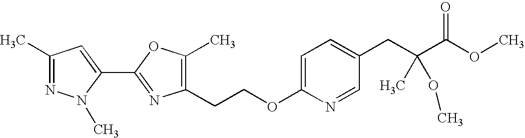 | (CDCl₃, 400 MHz): 7.91 (1H, d, J = 2.3 Hz), 7.42 (1H, dd, J =8.5, 2.4 Hz), 6.61 (1H, d, J =8.3 Hz), 6.48 (1H, s), 4.51 (2H, t, J = 6.7 Hz), 4.15 (3H, s), 3.71 (3H, s), 3.28 (3H, s), 2.96 (1H, d, J = 13.9 Hz), 2.94 (2H, t, J = 6.6 Hz), 2.87 (1H, d, J =13.9 Hz), 2.30 (3H, s), 2.27 (3H, s), 1.33 (3H, s) | LR 429 (M+H)⁺ |
| d-35 | 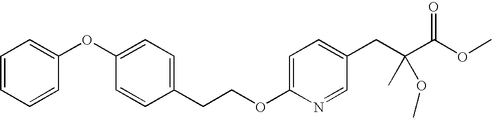 | (CDCl₃, 400 MHz): 7.91 (1H, d, J = 2.3 Hz), 7.42 (1H, dd, J =8.0, 1.9 Hz), 7.33-7.28 (2H, m), 7.23 (2H, d, J = 8.6 Hz), 7.08-7.04 (1H, m), 6.99-6.92 (4H, m), 6.64 (1H, d, J = 8.6 Hz), 4.45 (2H, t, J = 7.1 Hz), 3.70 (3H, s), 3.20 (3H, s), 3.04 (2H, t, J = 7.0 Hz), 2.92 (1H, d, J =14.2 Hz), 2.87 (1H, d, J = 14.2 Hz), 1.33 (3H, s) | LR 422 (M+H)⁺ |
| d-36 | 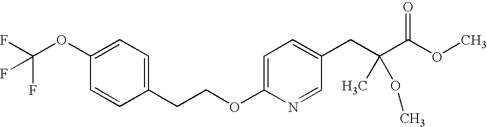 | (CDCl₃, 400 MHz): 7.90 (1H, d, J = 2.3 Hz), 7.42 (1H, dd, J =8.5, 2.4 Hz), 7.28 (2H, d, J =8.6 Hz), 7.13 (2H, d, J = 8.1 Hz), 6.63 (1H, d, J = 8.3 Hz), 4.46 (2H, t, J = 6.8 Hz), 3.71 (3H, s), 3.28 (3H, s), 3.06 (2H, t, J = 6.8 Hz), 2.95 (1H, d, J =14.2 Hz), 2.86 (1H, d, J = 13.9 Hz), 1.33 (3H, s) | LR 414 (M+H)⁺ |
| d-37 | 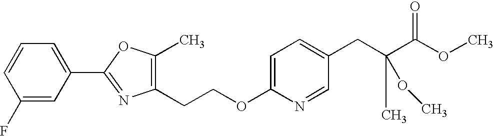 | (CDCl₃, 400 MHz): 7.91 (1H, d, J = 2.0 Hz), 7.74 (1H, d, J =7.8 Hz), 7.41 (1H, dd, J = 8.2, 2.5 Hz), 7.34 (1H, t, J = 7.7 Hz), 7.38 (1H, s), 7.18 (1H, d, J = 8.1 Hz), 6.62 (1H, d, J = 8.3 Hz), 4.51 (2H, t, J = 6.7 Hz), 3.70 (3H, s), 3.28 (3H, s), 2.96 (2H, t, J = 6.8 Hz), 2.95 (1H, d, J = 13.9 Hz), 2.86 (1H, d, J =13.9 Hz), 2.32 (3H, s), 1.32 (3H, s) | LR 429 (M+H)⁺ |
| d-38 | 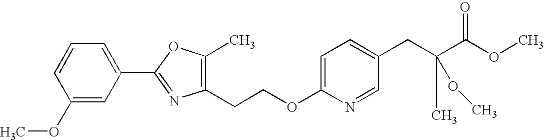 | (CDCl₃, 400 MHz): 7.91 (1H, d, J = 2.0 Hz), 7.41 (1H, dd, J =8.5, 2.4 Hz), 7.34-7.29 (2H, m), 6.96-6.93 (2H, m), 6.62 (1H, d, J = 8.5 Hz), 4.52 (2H, t, J = 6.8 Hz), 3.70 (3H, s), 3.28 (3H, s), 2.96 (2H, t, J = 6.8 Hz), 2.95 (1H, d, J = 13.9 Hz), 2.86 (1H, d, J = 13.9 Hz), 2.32 (3H, s), 2.16 (3H, s), 1.32 (3H, s) | LR 441 (M+H)⁺ |

The compounds of the invention have been tested for activities against PPAR-gamma and PPAR-alpha. The activities are tabulated below in Ki (μm).

| | PPAR- | PPAR- |
|---|---|---|
| | gamma | alpha |
| Example # | Ki (μm) | Ki (μm) |
| A-02 | | 0.55 |
| A-03 | 10 | 19 |
| A-05 E/M | 0.33 | 2.6 |
| A-05 S/E | 3.4 | 7.5 |
| A-05 S/E | 0.19 | 1.1 |
| A-07 | | 0.12 |
| A-10 | 1.5 | 4.4 |
| A-11 | 0.58 | 0.12 |
| A-12 | 0.051 | 0.35 |
| A-13 | 3.5 | 10 |
| A-14 | 1.3 | 0.15 |
| A-15 | 1.2 | 0.16 |
| A-16 | 2.8 | 1.5 |
| A-17 | 1.9 | 1.6 |
| A-18 | 1.7 | 14 |
| A-19 | 0.059 | 0.7 |
| A-20 | 0.18 | 0.14 |
| A-21 | 0.088 | 0.31 |
| A-22 | 0.17 | 0.85 |
| A-23 | 0.39 | 0.18 |
| A-24 | 0.78 | 0.018 |
| A-25 | 3.2 | 4.2 |
| A-26 | 0.15 | 0.33 |
| A-27 | 2.4 | 1.3 |
| A-28 E/M | 0.044 | 0.93 |
| A-28 S/E | 0.081 | 1.1 |
| A-28 S/E | 0.94 | 2.9 |
| B-02 | | 2.2 |
| B-03 | 31 | 9 |
| B-04 | 1.5 | 48 |
| B-05 | 1.33 | 2.04 |
| B-05 | 2.9 | 3.2 |
| B-05 | 2 | 3.6 |
| B-06 | 0.37 | 0.13 |
| B-07 | 0.65 | 12 |
| B-08 | 0.48 | 3.1 |
| B-09 | 1.9 | 2.2 |
| B-10 | 0.17 | 0.19 |
| B-11 | 7.9 | 0.96 |
| B-12 | 33 | 0.3 |
| B-13 | | 9 |
| B-14 | 2.3 | 0.021 |
| B-16 | 1.8 | 0.084 |
| B-17 | 1.2 | 0.047 |
| B-18 | | 0.082 |
| B-19 | 0.74 | 0.34 |
| B-20 | | 1.6 |
| B-21 | 0.99 | 4 |
| B-22 | 0.15 | 0.46 |
| B-23 | 3.4 | 2.7 |
| B-24 | 1.6 | 1 |
| B-27 | | 0.22 |
| B-28 | | 7.8 |
| C-02 | | 0.39 |
| C-03 | | 2.9 |
| C-05 | | 15 |
| C-06 | 9.5 | 41 |
| C-07 | 11 | 22 |
| C-09 | 1.4 | 4.6 |
| C-10 | 2.1 | 19 |
| C-11 | 2 | 25 |
| C-12 | 1.6 | 9.4 |
| C-14 | 8 | 3.2 |
| C-15 | 14 | 3.2 |
| C-17 | 26 | 3.2 |
| C-18 | 3.5 | 0.065 |
| C-20 | 1.5 | 3.1 |
| C-21 | 0.053 | 0.066 |
| C-22 | 0.8 | 2.1 |
| C-23 | 0.19 | 2.5 |
| C-24 | 0.083 | 0.022 |
| C-25 | 0.066 | 0.018 |
| C-26 | 0.068 | 0.016 |
| C-27 | 0.026 | 0.015 |
| C-28 | 0.03 | 0.088 |
| C-29 | 0.006 | 0.12 |
| C-30 | 0.033 | 0.11 |
| C-31 | 0.026 | 0.093 |
| C-32 | 0.035 | 0.15 |
| C-33 | 0.05 | 0.01 |
| C-36 | 1.7 | 21 |
| C-38 | 7.2 | |
| C-40 | 3.7 | 0.7 |
| C-42 | 33 | 13 |
| C-44 | | 36 |
| C-45 | 0.22 | 7.7 |
| C-46 | 2 | 3.5 |
| C-47 | 3.5 | 8.3 |
| C-48 | 0.018 | 0.24 |
| C-48 E/M | 0.15 | 0.31 |
| C-48 S/E | 0.014 | 0.047 |
| C-48 S/E | | 2.4 |
| C-49 | 0.21 | 1.6 |
| C-49 | 21 | 41 |
| C-49 | 0.043 | 0.34 |
| C-50 | 0.043 | 1.1 |
| C-51 | 0.18 | 2.9 |
| C-52 | 0.3 | 0.15 |
| C-53 | 0.093 | 0.64 |
| C-53 S/E | 3.6 | |
| C-53 S/E | 0.027 | 0.4 |
| C-54 | 0.02 | 1.2 |
| C-55 | 6 | |
| C-56 | 0.081 | 0.078 |
| C-57 | 1.1 | 1.8 |
| C-59 | 0.009 | 0.054 |
| C-60 | 0.015 | 0.065 |
| C-61 | 0.31 | 0.24 |
| C-63 | 0.094 | 0.021 |
| C-64 | 0.052 | 0.014 |
| C-66 | 0.023 | 0.031 |
| C-67 | 0.044 | 0.25 |
| C-68 | 4.2 | |
| C-69 | 1.1 | 2.5 |
| C-70 | | 6.4 |
| C-71 | | 9.7 |
| C-72 | | 7.1 |
| C-73 | 0.042 | |
| C-74 | 0.64 | 0.9 |
| C-74 S/E | | |
| C-74 S/E | 0.47 | 4.2 |
| C-75 | 0.82 | |
| C-76 | 2.3 | 7.3 |
| C-77 | 0.15 | 2.9 |
| C-78 | 0.006 | 0.015 |
| C-78 S/E | 3.9 | |
| C-78 S/E | 0.003 | 0.014 |
| C-79 | 0.007 | 0.015 |
| C-80 | 0.062 | 0.053 |
| C-81 | 0.015 | 0.75 |
| C-82 | 0.016 | |
| C-83 | 0.008 | 0.004 |
| C-84 | 0.021 | 0.064 |
| C-85 | 0.004 | 0.013 |
| C-86 | 0.005 | 0.013 |
| C-87 | 0.012 | 0.025 |
| C-89 | 0.04 | 0.03 |
| C-90 | 0.017 | 0.11 |
| C-91 | 0.39 | 1.8 |
| C-92 | 1.1 | 2.8 |
| C-93 | 0.011 | 0.3 |
| C-94 | 7 | |
| C-95 | 0.97 | 8.6 |
| D-02 | 0.46 | 0.29 |
| D-03 | 0.011 | 0.3 |
| D-04 | 0.44 | 0.29 |
| D-05 | 2.8 | |
| D-06 | 0.027 | 0.13 |
| D-07 | 0.017 | 0.32 |
| D-08 | 0.002 | 0.015 |
| D-09 | 0.061 | 0.05 |
| D-10 | 0.019 | 0.033 |
| D-11 | 6.2 | 5.1 |
| D-12 | 8.1 | |
| D-13 | 0.36 | 0.19 |
| D-14 | 1.5 | 2.6 |
| D-15 | 0.35 | 0.22 |
| D-16 | 6.4 | |
| D-17 | 0.031 | 0.91 |
| D-18 | 0.45 | 0.62 |
| D-19 | 1.7 | 6.4 |
| D-20 | 0.48 | 0.84 |
| D-21 | 2.1 | 10 |
| D-23 E/M | 0.073 | 6.5 |
| D-23 S/E | 5 | |
| D-23 S/E | 0.039 | 1.1 |
| D-24 | no SPA | no SPA |
| D-24 | 0.58 | 0.93 |
| D-25 | 2.9 | |
| D-26 | no SPA | no SPA |
| D-26 S/E | 1.9 | |
| D-26 S/E | 0.042 | 0.67 |
| D-27 | 7.8 | |
| D-28 | 1.6 | 4.8 |
| D-29 | 0.4 | 0.8 |
| D-29 S/E | 1.2 | |
| D-29 S/E | 0.69 | 3 |
| D-31 | 1 | 0.088 |
| D-32 | 1.5 | 0.059 |
| D-33 | 0.69 | 0.55 |
| D-34 | 9.1 | 3.2 |
| D-35 | 0.56 | 0.33 |
| D-35 S/E | 0.27 | 0.57 |
| D-35 S/E | 4.8 | 9 |
| D-36 | 0.23 | 0.73 |
| D-37 | 0.53 | 4.7 |
| D-38 | 9.6 | |
| D-39 | 5 | |
| D-40 | 9.6 | 1.8 |
| D-41 | | 5.9 |
| D-42 | 0.7 | 1 |
| D-44 | 0.15 | 2.6 |
| D-45 | 0.058 | 0.09 |

E/M is defined as enantiomeric mixture, including racemic mixture.
S/E is defined as single enantiomer.

While the invention has been illustrated by reference to specific and preferred embodiments, those skilled in the art will recognize that variations and modifications may be made through routine experimentation and practice of the invention. Thus, the invention is intended not to be limited by the foregoing description, but to be defined by the appended claims and their equivalents.

1-24. (canceled)

25. A compound, which is:
2-Methyl-2-{3-[({[2-(5-methyl-2-phenyl-1,3-oxazol-4-yl)ethoxy]carbonyl}amino)methyl]phenoxy}propanoic acid (Example B-5);
and the pharmaceutically acceptable salts thereof.

26-35. (canceled)

36. A method of treating non-insulin dependent diabetes mellitus in a mammal comprising administering to the mammal in need thereof a therapeutically effective amount of an alpha substituted carboxylic acid compound according to claim 25.

37. The method according to claim 36 wherein said mammal has an impaired glucose tolerance.

38. A method of treating polycystic ovarian syndrome in a mammal comprising administering to the mammal in need thereof a therapeutically effective amount of an alpha substituted carboxylic acid compound according to claim 25.

39. A method of treating polycystic obesity in a mammal comprising administering to the mammal in need thereof a therapeutically effective amount of an alpha substituted carboxylic acid compound according to claim 25.

40. A method of reducing body weight in an obese mammal comprising administering to the mammal in need thereof a therapeutically effective amount of an alpha substituted carboxylic acid compound according to claim 25.

41. A method of treating hyperglycemia in a mammal comprising administering to the mammal in need thereof a therapeutically effective amount of an alpha substituted carboxylic acid compound according to claim 25.

42. A method of treating hyperlipidemia in a mammal comprising administering to the mammal in need thereof a therapeutically effective amount of an alpha substituted carboxylic acid compound according to claim 25.

43. A method of treating hypercholesteremia in a mammal comprising administering to the mammal in need thereof a therapeutically effective amount of an alpha substituted carboxylic acid compound according to claim 25.

44. A method of treating atherosclerosis in a mammal comprising administering to the mammal in need thereof a therapeutically effective amount of an alpha substituted carboxylic acid compound according to claim 25.

45. A method of treating hypertriglyceridemia in a mammal comprising administering to the mammal in need thereof a therapeutically effective amount of an alpha substituted carboxylic acid compound according to claim 25.

46. A method of treating hyperinsulinemia in a mammal comprising administering to the mammal in need thereof a therapeutically effective amount of an alpha substituted carboxylic acid compound according to claim 25.

47. A method of treating a patient suffering from abnormal insulin and/or evidence of glucose disorders associated with circulating glucocorticoids, growth hormone, catecholamines, glucagon, or parathyroid hormone, comprising administering to said patient a therapeutically effective amount of an alpha substituted carboxylic acid compound according to claim 25.

48. A method of treating insulin resistance syndrome in humans comprising administering to a patient in need of treatment a therapeutically effective amount of an alpha substituted carboxylic acid compound according to claim 25.

49. A method of treating PPAR-related disorders in humans comprising administering to a patient in need of treatment a therapeutically effective amount of an alpha substituted carboxylic acid compound according to claim 25.

50. A method of modulating PPAR activity in a mammal, comprising administering to a mammal a therapeutically effective amount of an alpha substituted carboxylic acid compound according to claim 25.

51. A method of lowering blood glucose in a mammal, comprising administering to a mammal an amount of an alpha substituted carboxylic acid compound according to claim 25 therapeutically effective to lower blood glucose levels.

52. A method of modulating fat cell differentiation in a mammal, comprising administering to a mammal a therapeutically effective amount of an alpha substituted carboxylic acid compound according to claim 25.

53. A method of modulating processes mediated by PPAR in a mammal, comprising administering to a mammal a therapeutically effective amount of an alpha substituted carboxylic acid compound according to claim 25.

54. A method of increasing insulin sensitivity in mammals, comprising administering to a mammal a therapeutically effective amount of an alpha substituted carboxylic acid compound according to claim 25.

55. A method of treating metabolic syndromes selected from the group consisting of galactosemia, maple syrup urine disease, phenylketonuria, hypersarcosinemia, thymine uraciluria, sulfinuria, isovaleric acidemia, saccharopinuria, 4-hydroxybutyric aciduria, glucose-6-phosphate dehydrogenase deficiency, and pyruvate dehydrogenase deficiency comprising administering to a mammal a therapeutically effective amount of an alpha substituted carboxylic acid compound according to claim 25.

56. A composition comprising the compound according to claim 25 and a pharmaceutically acceptable carrier thereof.
